(19) United States
(12) Patent Application Publication
(10) Pub. No.: US 2023/0002332 A1
(43) Pub. Date: Jan. 5, 2023

(54) ORGANIC ELECTROLUMINESCENT COMPOUND, A PLURALITY OF HOST MATERIALS, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: So-Young JUNG, Gyeonggi-do (KR); Su-Hyun Lee, Gyeonggi-do (KR); Jin-Ri Hong, Gyeonggi-do (KR); Jin-Man Kim, Gyeonggi-do (KR); Hyo-Nim Shin, Gyeonggi-do (KR); Sang-Hee Cho, Gyeonggi-do (KR)

(21) Appl. No.: 17/824,449

(22) Filed: May 25, 2022

(30) Foreign Application Priority Data
May 28, 2021 (KR) 10-2021-0068934

(51) Int. Cl. C07D 263/57; C07D 277/64; C07D 413/12; C07D 413/14; C07F 7/08; C07D 405/04; C07D 409/04; H01L 51/00

(52) U.S. Cl. CPC C07D 263/57; C07D 277/64; C07D 413/12; C07D 413/14; C07F 7/0812; C07D 405/04; C07D 409/04; H01L 51/0094; H01L 51/0085; H01L 51/008; H01L 51/0077; H01L 51/0074; H01L 51/0073; H01L 51/0071; H01L 51/0069; H01L 51/007; H01L 51/0059; H01L 51/005; H01L 51/0052; C07B 2200/05; H01L 51/5012

(57) ABSTRACT

The present disclosure relates to a plurality of host materials comprising a first host material including a compound represented by formula 1 and a second host material including a compound represented by formula 2, and an organic electroluminescent device comprising the same. In addition, the present disclosure relates to an organic electroluminescent compound, and an organic electroluminescent device comprising the same. By comprising the organic electroluminescent compound and/or a specific combination of compounds according to the present disclosure as host materials, an organic electroluminescent device having high luminous efficiency and long lifespan property can be provided.

### TECHNICAL FIELD

The present disclosure relates to an organic electroluminescent compound, a plurality of host materials, and an organic electroluminescent device comprising the same.

### BACKGROUND ART

The TPD/Alq₃ bilayer small molecule organic electroluminescent device (OLED) with green-emission, which is constituted with a light-emitting layer and a charge transport layer, was first developed by Tang, et al., of Eastman Kodak in 1987. Thereafter, the studies on an organic electroluminescent device have been rapidly effected, and OLEDs have been commercialized. At present, OLEDs primarily use phosphorescent materials having excellent luminous efficiency in panel implementation. In many applications such as TVs and lightings, OLED lifetime is insufficient, and high efficiency of OLEDs is still required. Typically, the higher the luminance of an OLED corresponds to a shorter lifetime of the OLED. Therefore, an OLED having high luminous efficiency and/or long lifespan property is required for long time use and high resolution of a display.

Various materials or concepts have been proposed for the organic layer of an organic electroluminescent device in order to improve luminous efficiency, driving voltage and/or lifespan, but they have not been satisfactory for practical use.

Korean Patent Application Laid-Open No. 10-2017-0022865 discloses an organic electroluminescent device using phenanthrooxazole and phenanthrothiazole compounds as hosts. However, said reference does not specifically disclose an organic electroluminescent device using the specific combination of a plurality of host materials as described in the present disclosure. In addition, there is still a need to develop a host material for improving OLED performance.

Korean Patent Application Laid-Open No. 10-2020-0026079 discloses a compound comprising a nitrogen-containing heteroaryl as one of a plurality of host materials, but a compound in which a silyl group is substituted is not disclosed in said reference.

### DISCLOSURE OF THE INVENTION

### Technical Problem

The object of the present disclosure is firstly, to provide a plurality of host materials which is able to produce an organic electroluminescent device having high luminous efficiency and long lifespan property, and secondly, to provide an organic electroluminescent compound having a novel structure suitable for use as an organic electroluminescent material. In addition, the other object of the present disclosure is to provide an organic electroluminescent device with high luminous efficiency and/or improved lifespan characteristics by comprising a compound according to the present disclosure as a single host material or a specific combination of compounds according to the present disclosure as a plurality of host materials.

### Solution to Problems

As a result of intensive studies to solve the technical problem above, the present inventors found that the aforementioned objective can be achieved by a plurality of host materials comprising a first host material comprising a compound represented by the following formula 1 and a second host material comprising a compound represented by the following formula 2, or an organic electroluminescent compound represented by the following formula 3, so that the present invention was completed.

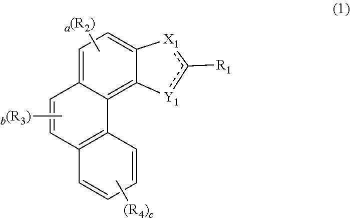

in formula 1,

X₁ and Y₁ each independently represent —N═, —NR₅—, —O— or —S—; provided that any one of X₁ and Y₁ is —N═, and other of X₁ and Y₁ is —NR₅—, —O— or —S—;

R₁ represents a substituted or unsubstituted (C6-C30)aryl or a substituted or unsubstituted (3- to 30-membered)heteroaryl;

R₂ to R₅ each independently represent hydrogen, deuterium, cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted fused ring of (C3-C30) aliphatic ring and a (C6-C30) aromatic ring, —NR₁₁R₁₂, —SiR₁₃R₁₄R₁₅ or a combination thereof; or may be linked to the adjacent substituents to form a ring(s);

R₁₁ to R₁₅ each independently represent a substituted or unsubstituted (C1-C30)alkyl, a substituted or unsubstituted (C6-C30)aryl, or a substituted or unsubstituted (3- to 30-membered)heteroaryl;

a and b each independently represent an integer of 1 or 2, and c represents an integer of 1 to 4; and

when a to c are an integer of 2 or more, each of R₂, each of R₃, and each of R₄ may be the same or different;

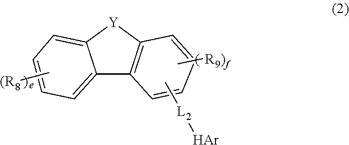

in formula 2,

Y represents —O— or —S—;

L₂ represents a single bond, a substituted or unsubstituted (C6-C30)arylene, or a substituted or unsubstituted (3- to 30-membered)heteroarylene;

HAr represents a substituted or unsubstituted (3- to 30-membered)heteroaryl comprising at least one nitrogen atom;

R₈ and R₉ each independently represent hydrogen, deuterium, cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₁₆R₁₇, —SiR₁₈R₁₉R₂₀, or a combination thereof; or may be linked to the adjacent substituents to form a ring(s);

R₁₆ to R₂₀ each independently represent a substituted or unsubstituted (C1-C30)alkyl, a substituted or unsubstituted (C6-C30)aryl, or a substituted or unsubstituted (3- to 30-membered)heteroaryl;

e represents an integer of 1 to 4, and f represents an integer of 1 to 3; and

when e and f are an integer of 2 or more, each of R₈ and each of R₉ may be the same or different;

provided that at least one of HAr, R₈, and R₉ includes -L₃-SiR′R″R′″ or -L₃-CR′R″R′″;

L₃ represents a single bond, a substituted or unsubstituted (C1-C30)alkylene, a substituted or unsubstituted (C3-C30)cycloalkylene, a substituted or unsubstituted (C6-C30)arylene, or a substituted or unsubstituted (3- to 30-membered)heteroarylene; and

R′, R″, and R′″ each independently represent a substituted or unsubstituted (C6-C30)aryl or a substituted or unsubstituted (3- to 30-membered)heteroaryl.

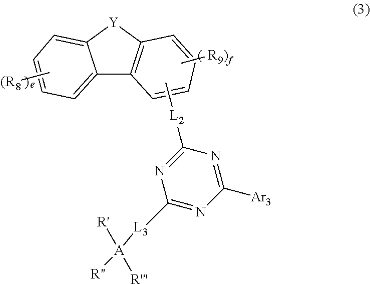

in formula 3,

Y represents O or S;

A represents Si or C;

L₂ represents a single bond, or phenylene, biphenylene, or naphthylene unsubstituted or substituted by deuterium;

R₈ and R₉ each independently represent hydrogen or deuterium;

Ar₃ represents phenyl unsubstituted or substituted by deuterium, biphenyl unsubstituted or substituted by deuterium, terphenyl unsubstituted or substituted by deuterium, naphthyl unsubstituted or substituted by deuterium, triphenylenyl unsubstituted or substituted by deuterium, phenanthrenyl unsubstituted or substituted by deuterium, or a combination thereof;

L₃ represents phenylene unsubstituted or substituted by deuterium, biphenylene unsubstituted or substituted by deuterium, or naphthylene unsubstituted or substituted by deuterium;

R′, R″, and R′″ each independently represent a substituted or unsubstituted (C6-C30)aryl or a substituted or unsubstituted (3- to 30-membered)heteroaryl;

e represents an integer of 1 to 4, and f represents an integer of 1 to 3; and

when e and f are an integer of 2 or more, each of R₈ and each of R₉ may be the same or different.

### Advantageous Effects of Invention

By comprising an organic electroluminescent compound or the specific combination of the compound according to the present disclosure as host materials, an organic electroluminescent device having a high luminous efficiency and long lifespan property can be provided.

### EMBODIMENTS OF THE INVENTION

Hereinafter, the present disclosure will be described in detail. However, the following description is intended to explain the invention, and is not meant in any way to restrict the scope of the invention.

The present disclosure relates to a plurality of host materials comprising a first host material including at least one compound represented by formula 1 and a second host material including at least one compound represented by formula 2, and an organic electroluminescent device comprising the host materials.

The present disclosure relates to an organic electroluminescent compound represented by formula 3, an organic electroluminescent material comprising the same, and an organic electroluminescent device comprising the organic electroluminescent material.

The term “organic electroluminescent compound” in the present disclosure means a compound that may be used in an organic electroluminescent device, and may be comprised in any material layer constituting an organic electroluminescent device, as necessary.

Herein, the term “organic electroluminescent material” means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, or an electron injection material, etc.

The term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. Such at least two compounds may be comprised in the same layer or in different layers, and may be mixture-evaporated or co-evaporated, or may be individually evaporated.

Herein, the term “a plurality of host materials” means an organic electroluminescent material comprising a combination of at least two host materials. It may mean both a material before being comprised in an organic electroluminescent device (e.g., before vapor deposition) and a material after being comprised in an organic electroluminescent device (e.g., after vapor deposition). A plurality of host materials of the present disclosure may be comprised in any light-emitting layer constituting an organic electroluminescent device. The at least two compounds comprised in a plurality of host materials may be comprised together in one light-emitting layer, or may each be comprised in separate light-emitting layers. When at least two compounds are comprised in one light-emitting layer, the at least two compounds may be mixture-evaporated to form a layer or may be individually and simultaneously co-evaporated to form a layer.

Herein, “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. Herein, the term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. Herein, “(C6-C30)aryl(ene)” is a monocyclic or fused ring radical derived from an aromatic hydrocarbon having 6 to 30 ring backbone carbon atoms, in which the number of the ring backbone carbon atoms is preferably 6 to 20, more preferably 6 to 15, may be partially saturated, and may include a spiro structure. Examples of the aryl specifically may be phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluoren-fluoren]yl, spiro[fluoren-benzofluoren]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. Herein, “(3- to 30-membered)heteroaryl(ene)” is an aryl having 3 to 30 ring backbone atoms including at least one, preferably 1 to 4 heteroatoms selected from the group consisting of B, N, O, S, Si, P, Se, and Ge, in which the number of the ring backbone carbon atoms is preferably 3 to 30, and more preferably 5 to 20. The above heteroaryl(ene) may be a monocyclic ring, or a fused ring condensed with at least one benzene ring; and may be partially saturated. Also, the above heteroaryl or heteroarylene herein may be one formed by linking at least one heteroaryl or aryl group to a heteroaryl group via a single bond(s), and may comprise a spiro structure. Examples of the heteroaryl specifically may be a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthiridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzopyrimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Herein, the term “a fused ring of (C3-C30) aliphatic ring and (C6-C30) aromatic ring” means a ring formed by fusing at least one aliphatic ring having 3 to 30 ring backbone carbon atoms in which the carbon atoms number is preferably 3 to 25, more preferably 3 to 18, and at least one aromatic ring having 6 to 30 ring backbone carbon atoms in which the carbon atoms number is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of (C3-C30) aliphatic ring and (C6-C30) aromatic ring may be replaced with at least one heteroatom selected from B, N, O, S, Si and P, preferably at least one heteroatom selected from N, O and S. The term “Halogen” in the present disclosure includes F, Cl, Br, and I.

In addition, “ortho (o),” “meta (m),” and “para (p)” are meant to signify the substitution position of all substituents. Ortho position is a compound with substituents, which are adjacent to each other, e.g., at the 1 and 2 positions on benzene. Meta position is the next substitution position of the immediately adjacent substitution position, e.g., a compound with substituents at the 1 and 3 positions on benzene. Para position is the next substitution position of the meta position, e.g., a compound with substituents at the 1 and 4 positions on benzene.

Herein, the term “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, preferably may be a substituted or unsubstituted (5- to 25-membered) mono- or polycyclic, alicyclic, aromatic ring, or a combination thereof. Further, the formed ring may include at least one heteroatom selected from the group consisting of B, N, O, S, Si and P, preferably, N, O and S. According to one embodiment of the present disclosure, the number of atoms in the ring skeleton is 5 to 20; according to another embodiment of the present disclosure, the number of atoms in the ring skeleton is 5 to 15. In one embodiment, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzofluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, “substituted” in the expression “substituted or unsubstituted” means that a hydrogen atom in a certain functional group is replaced with another atom or functional group, i.e., a substituent, and substituted with a group to which two or more substituents are connected among the substituents. For example, “a substituent to which two or more substituents are connected” may be pyridine-triazine. That is, pyridine-triazine may be heteroaryl or may be interpreted as one substituent in which two heteroaryls are connected. The substituents of the substituted alkyl, the substituted cycloalkyl, the substituted cycloalkenyl, the substituted heterocycloalkyl, the substituted aryl(ene), the substituted heteroaryl(ene), and the substituted nitrogen-containing heteroaryl in the formulas of the present disclosure, each independently represent at least one selected from the group consisting of deuterium; halogen; cyano; carboxyl; nitro; hydroxyl; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3- to 7-membered)heterocycloalkyl; (C6-C30)aryloxy; (C6-C30)arylthio; (3- to 30-membered)heteroaryl unsubstituted or substituted by (C6-C30)aryl; (C6-C30)aryl unsubstituted or substituted by at least one of (C1-C30)alkyl and (3- to 30-membered)heteroaryl; tri(C1-C30)alkylsilyl; tri(C6-C30)arylsilyl; di(C1-C30)alkyl(C6-C30)arylsilyl; (C1-C30)alkyldi(C6-C30)arylsilyl; amino; mono- or di-(C1-C30)alkylamino; mono- or di-(C6-C30)arylamino; (C1-C30)alkyl(C6-C30)arylamino; (C1-C30)alkylcarbonyl; (C1-C30)alkoxycarbonyl; (C6-C30)arylcarbonyl; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl. For example, the substituents may be deuterium, cyano, methyl, phenyl, biphenyl, naphthyl, carbazolyl, dibenzofuranyl, etc.

Hereinafter, the plurality of host materials according to one embodiment will be described.

The plurality of host materials according to one embodiment comprise a first host compound comprising a compound represented by formula 1 and a second host compound comprising a compound represented by formula 2; and the plurality of host materials may be comprised in the light-emitting layer of an organic electroluminescent device according to one embodiment.

The first host material as the host materials according to one embodiment comprises a compound represented by the following formula 1.

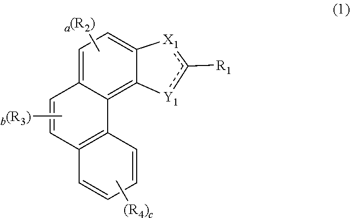

in formula 1,

X₁ and Y₁ each independently represent —N═, —NR₅—, —O— or —S—; provided that any one of X₁ and Y₁ is —N═, and other of X₁ and Y₁ is —NR₅—, —O— or —S—;

R₁ represents a substituted or unsubstituted (C6-C30)aryl or a substituted or unsubstituted (3- to 30-membered)heteroaryl;

R₂ to R₅ each independently represent hydrogen, deuterium, cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted fused ring of (C3-C30) aliphatic ring and a (C6-C30) aromatic ring, —NR₁₁R₁₂, —SiR₁₃R₁₄R₁₅ or a combination thereof; or may be linked to the adjacent substituents to form a ring(s);

R₁₁ to R₁₅ each independently represent a substituted or unsubstituted (C1-C30)alkyl, a substituted or unsubstituted (C6-C30)aryl, or a substituted or unsubstituted (3- to 30-membered)heteroaryl;

a and b each independently represent an integer of 1 or 2, and c represents an integer of 1 to 4; and

when a to c are an integer of 2 or more, each of R₂, each of R₃, and each of R₄ may be the same or different.

In one embodiment, X₁ and Y₁ each independently may be —N═, —NR₅—, —O— or —S—, provided that any one of X₁ and Y₁ is —N═, and other of X₁ and Y₁ is —NR₅—, —O— or —S—. For example, when X₁ is —N═, Y₁ may be —O—, or when Y₁ is —N═, X₁ may be —O— or —S—.

In one embodiment, R₁ may be a substituted or unsubstituted (C6-C30)aryl, preferably a substituted or unsubstituted (C6-C25)aryl, more preferably a substituted or unsubstituted (C6-C18)aryl. For example, R₁ may be a substituted or unsubstituted phenyl or a substituted or unsubstituted biphenyl.

In one embodiment, at least one of R₂ to R₄ may be a substituted or unsubstituted (C6-C30)aryl, a substituted or unsubstituted (5- to 30-membered)heteroaryl, a substituted or unsubstituted fused ring of (C3-C30) aliphatic ring and a (C6-C30) aromatic ring, or —NR₁₁R₁₂, preferably at least one of R₂ to R₄ may be a substituted or unsubstituted (C6-C25)aryl, a substituted or unsubstituted (5- to 25-membered)heteroaryl, a substituted or unsubstituted fused ring of (C5-C25) aliphatic ring and a (C6-C25) aromatic ring, or —NR₁₁R₁₂, more preferably at least one of R₂ to R₄ may be a substituted or unsubstituted (C6-C18)aryl, a substituted or unsubstituted (5- to 18-membered)heteroaryl, a substituted or unsubstituted fused ring of (C5-C10) aliphatic ring and a (C6-C18) aromatic ring, or —NR₁₁R₁₂.

In one embodiment, all of R₂, R₃, and R₅ may be hydrogen.

In one embodiment, R₄ may be hydrogen, a substituted or unsubstituted (C6-C30)aryl, a substituted or unsubstituted (5- to 30-membered)heteroaryl, a substituted or unsubstituted fused ring of (C3-C30) aliphatic ring and a (C6-C30) aromatic ring, or —NR₁₁R₁₂, preferably hydrogen, a substituted or unsubstituted (C6-C25)aryl, a substituted or unsubstituted (5- to 25-membered)heteroaryl, a substituted or unsubstituted fused ring of (C5-C25) aliphatic ring and a (C6-C25) aromatic ring, or —NR₁₁R₁₂, more preferably hydrogen, a substituted or unsubstituted (C6-C18)aryl, a substituted or unsubstituted (5- to 18-membered)heteroaryl, a substituted or unsubstituted fused ring of (C5-C10) aliphatic ring and a (C6-C18) aromatic ring, or —NR₁₁R₁₂.

In one embodiment, R₁₁ to R₁₅ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, R₁₁ to R₁₅ each independently may be, a substituted or unsubstituted phenyl, a substituted or unsubstituted naphthyl, a substituted or unsubstituted p-biphenyl, a substituted or unsubstituted m-biphenyl, a substituted or unsubstituted p-terphenyl, a substituted or unsubstituted o-terphenyl, a substituted or unsubstituted phenanthrenyl, a substituted or unsubstituted fluorenyl, a substituted or unsubstituted carbazolyl, a substituted or unsubstituted dibenzofuranyl, a substituted or unsubstituted dibenzothiophenyl, or a substituted or unsubstituted benzonaphthofuranyl. For example, the substituents of the substituted groups may be methyl or phenyl.

The compound represented by formula 1 according to one embodiment may be represented by the following formula 1-1 or 1-2.

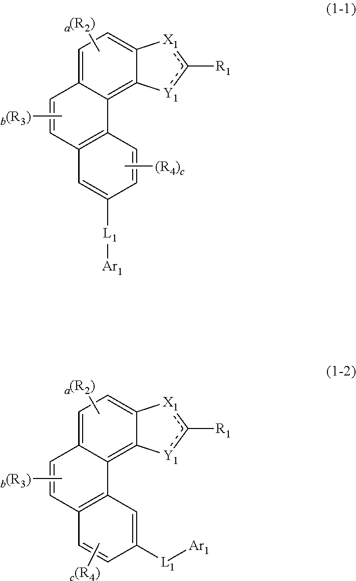

in formulas 1-1 and 1-2,

L₁ represents a single bond or a substituted or unsubstituted (C6-C30)arylene;

Ar₁ represents deuterium, halogen, cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted fused ring of (C3-C30) aliphatic ring and a (C6-C30) aromatic ring, —NR₁₁R₁₂, —SiR₁₃R₁₄R₁₅, or a combination thereof;

c represents an integer of 1 to 3; and

X₁, Y₁, R₁ to R₄, R₁₁ to R₁₅, a, and b are as defined in formula 1.

In one embodiment, L₁ may be a single bond or a substituted or unsubstituted (C6-C30)arylene, preferably a single bond or a substituted or unsubstituted (C6-C25)arylene, more preferably a single bond or a substituted or unsubstituted (C6-C18)arylene. For example, L₁ may be a single bond, a substituted or unsubstituted phenylene, a substituted or unsubstituted p-phenylene, or a substituted or unsubstituted m-biphenylene.

In one embodiment, Ar₁ may be a substituted or unsubstituted (C6-C30)aryl, a substituted or unsubstituted fused ring of (C3-C30) aliphatic ring and a (C6-C30) aromatic ring, or —NR₁₁R₁₂, preferably a substituted or unsubstituted (C6-C25)aryl, a substituted or unsubstituted fused ring of (C5-C30) aliphatic ring and a (C6-C25) aromatic ring, or —NR₁₁R₁₂, more preferably a substituted or unsubstituted (C6-C25)aryl, a substituted or unsubstituted fused ring of (C5-C10) aliphatic ring and a (C6-C18) aromatic ring, a substituted or unsubstituted di(C6-C30)arylamino, a substituted or unsubstituted di(5- to 30-membered)heteroarylamino, or a substituted or unsubstituted (C6-C30)aryl(5- to 30-membered)heteroarylamino. For example, Ar₁ may be a substituted or unsubstituted phenyl, a substituted or unsubstituted naphthyl, a substituted or unsubstituted biphenyl, a substituted or unsubstituted naphthylphenyl, a substituted or unsubstituted phenylnaphthyl, a substituted or unsubstituted terphenyl, a substituted or unsubstituted phenanthrenyl, a substituted or unsubstituted benzophenanthrenyl, a substituted or unsubstituted chrysenyl, a substituted or unsubstituted triphenylenyl, a substituted or unsubstituted fluorenyl, a substituted or unsubstituted benzofluorenyl, a substituted or unsubstituted fluoranthenyl, a substituted or unsubstituted spirobifluorenyl, a substituted or unsubstituted spiro[cyclopentan-fluoren]yl, a substituted or unsubstituted spiro[fluoren-benzofluoren]yl, or a substituted or unsubstituted spiro[indan-fluoren]yl; or an amino group substituted by at least one of phenyl; naphthyl; biphenyl; terphenyl; a substituted or unsubstituted fluorenyl; phenanthrenyl; dibenzofuranyl unsubstituted or substituted by phenyl; dibenzothiophenyl; carbazolyl unsubstituted or substituted by phenyl; and benzonaphthofuranyl. Specifically, Ar₁ may be for example a substituted or unsubstituted phenyl, a substituted or unsubstituted naphthyl, a substituted or unsubstituted p-biphenyl, a substituted or unsubstituted m-biphenyl, a substituted or unsubstituted o-biphenyl, a substituted or unsubstituted phenanthrenyl, a substituted or unsubstituted benzophenanthrenyl, a substituted or unsubstituted chrysenyl, a substituted or unsubstituted triphenylenyl, a substituted or unsubstituted fluorenyl, a substituted or unsubstituted benzofluorenyl, a substituted or unsubstituted fluoranthenyl, a substituted or unsubstituted sprirobifluorenyl, a substituted or unsubstituted spiro[cyclopentan-fluoren]yl, a substituted or unsubstituted spiro[indan-fluoren]yl; or a substituted or unsubstituted diphenylamino, a substituted or unsubstituted phenylbiphenylamino, a substituted or unsubstituted phenylnaphthylamino, a substituted or unsubstituted phenylterphenylamino, a substituted or unsubstituted phenylfluorenylamino, a substituted or unsubstituted phenylphenanthrenylamino, a substituted or unsubstituted naphthylbiphenylamino, a substituted or unsubstituted naphthylterphenylamino, a substituted or unsubstituted naphthylphenanthrenylamino, a substituted or unsubstituted dinaphthylamino, a substituted or unsubstituted dibiphenylamino, a substituted or unsubstituted biphenylfluorenylamino, a substituted or unsubstituted difluorenylamino, a substituted or unsubstituted phenylcarbazolylamino, a substituted or unsubstituted biphenyldibenzofuranylamino, a substituted or unsubstituted biphenyldibenzothiophenylamino, a substituted or unsubstituted dibenzofuranyldibenzothiophenylamino, a substituted or unsubstituted dibenzofuranylterphenylamino, a substituted or unsubstituted dibenzofuranyinaphthylamino, a substituted or unsubstituted dibenzofuranyl-dibenzofuranylamino, a substituted or unsubstituted dibenzothiophenyl-dibenzothiophenylamino, a substituted or unsubstituted dibenzofuranylphenanthrenylamino, a substituted or unsubstituted dibenzofuranylbenzonaphthofuranylamino. For example, the substituents of the substituted groups may be methyl, phenyl, or naphthyl.

According to one embodiment, the first host material comprising a compound represented by formula 1 may be more specifically illustrated by the following compounds, but is not limited thereto.

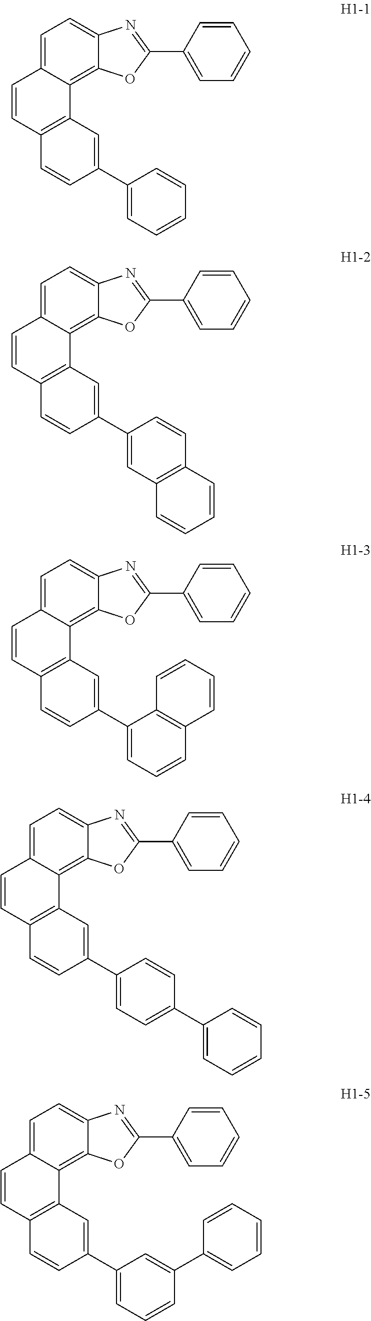

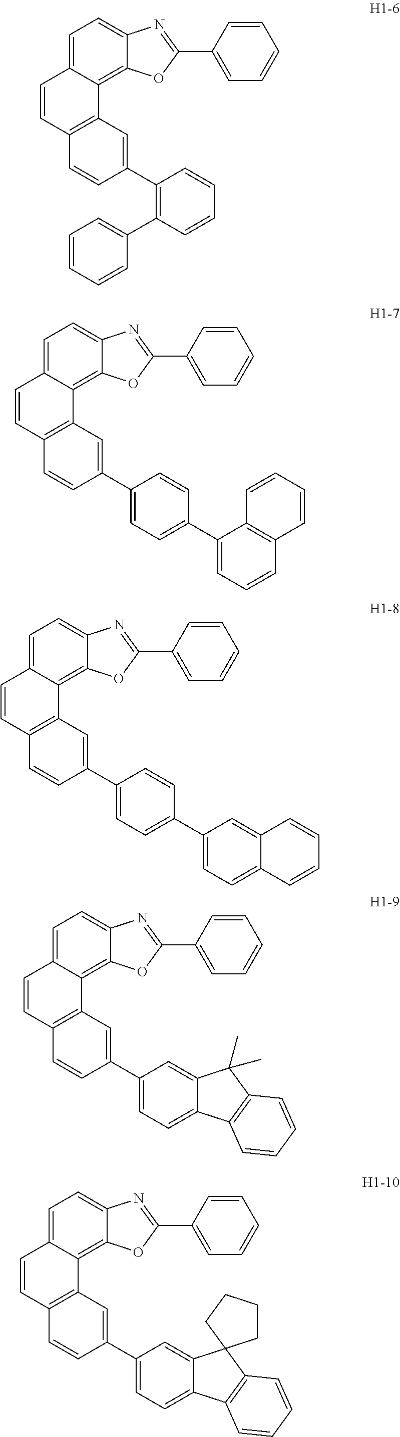

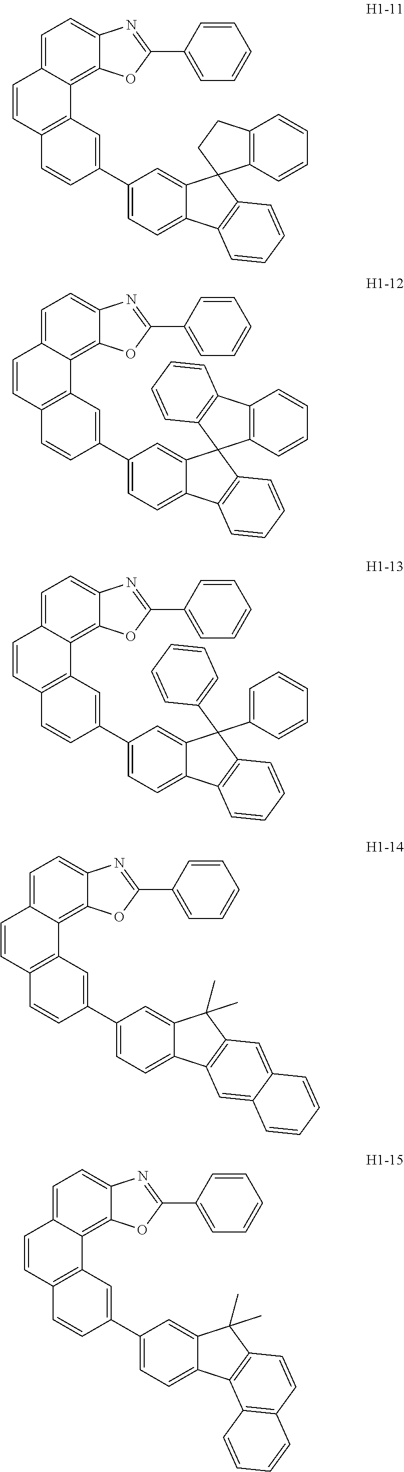

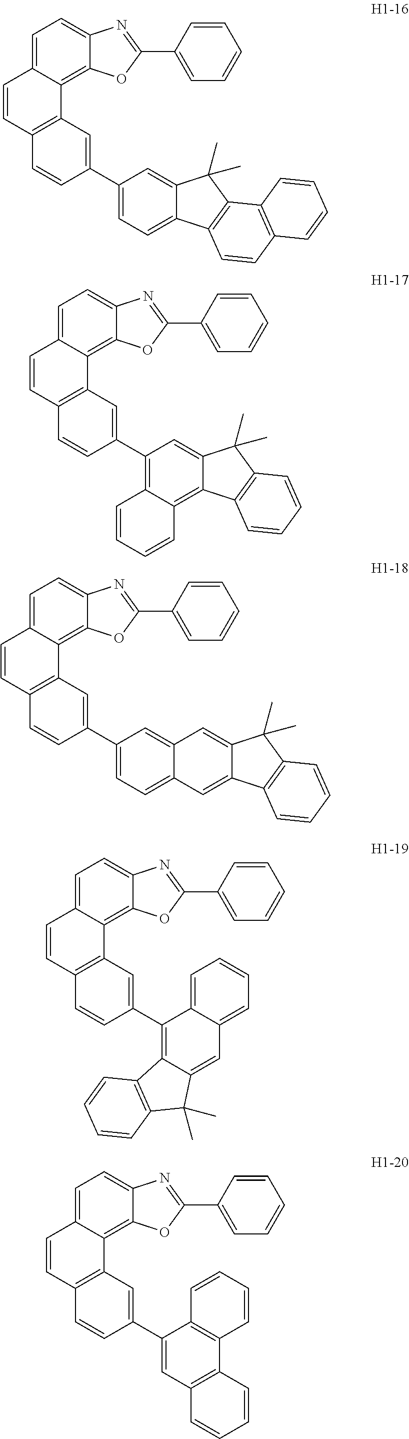

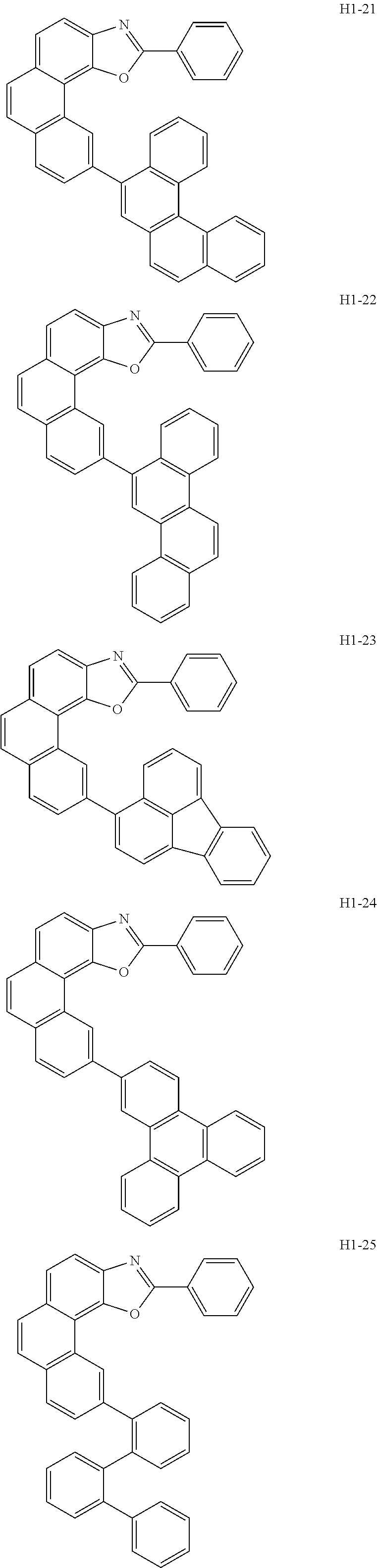

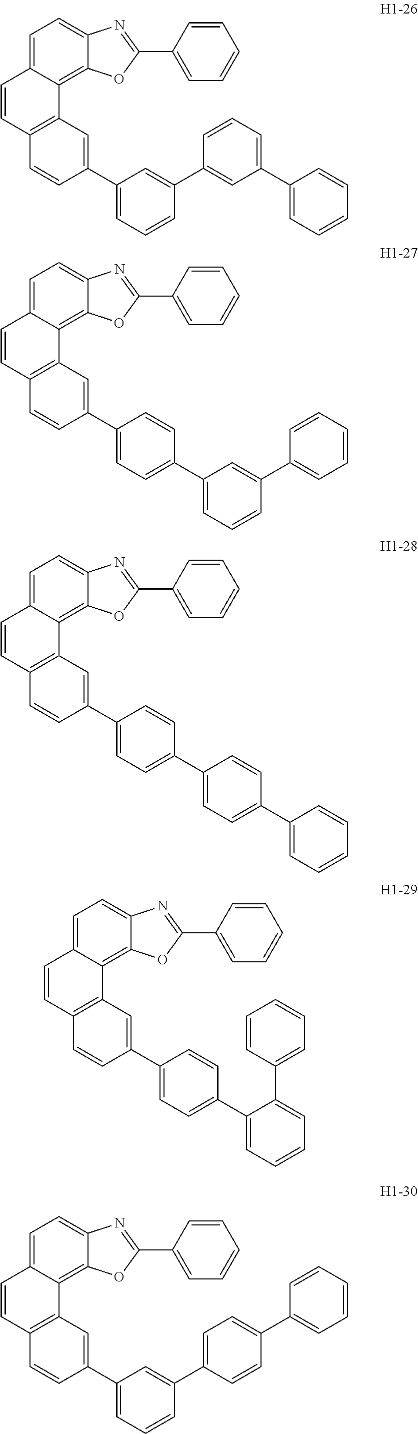

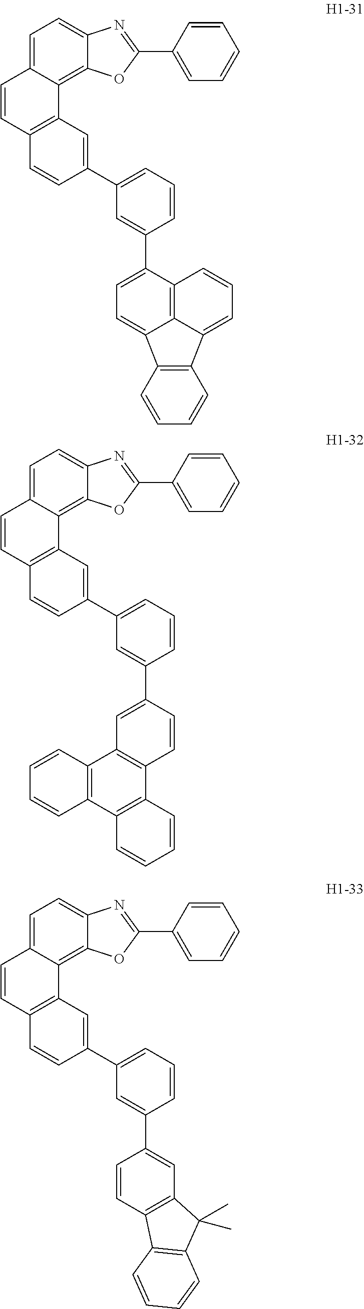

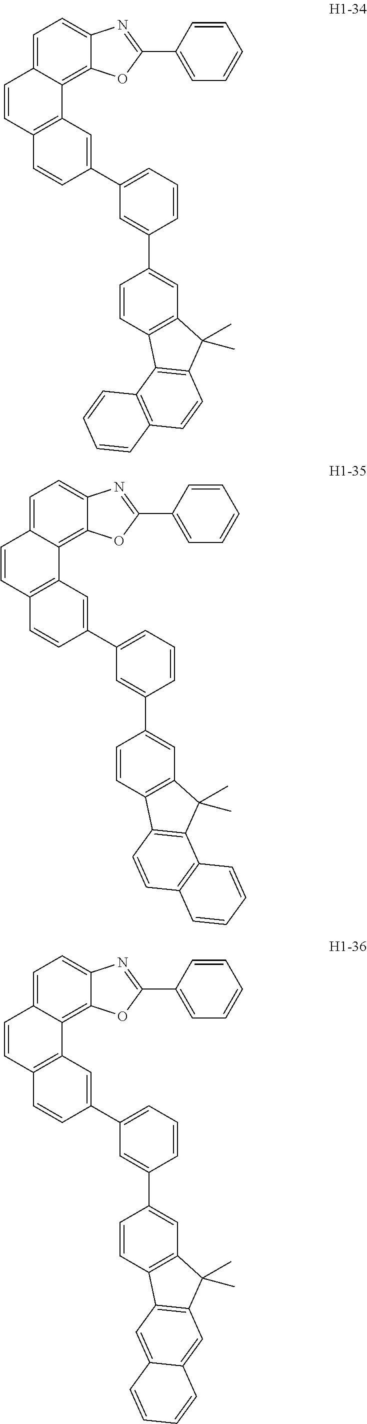

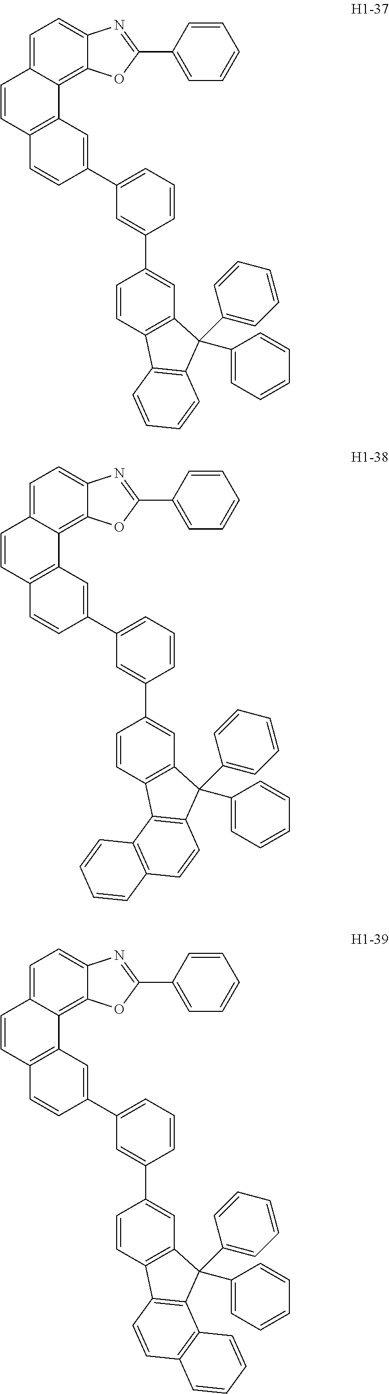

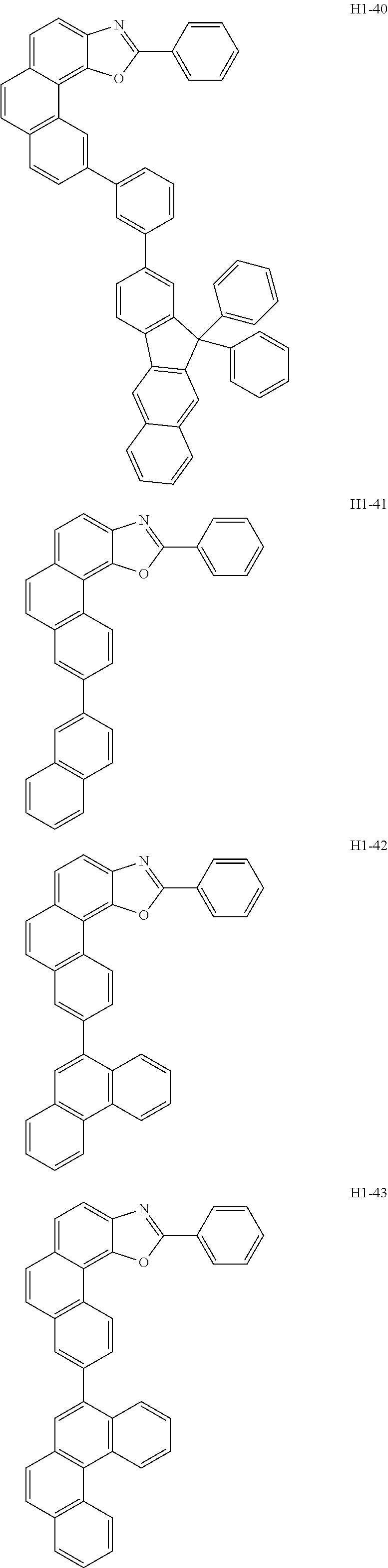

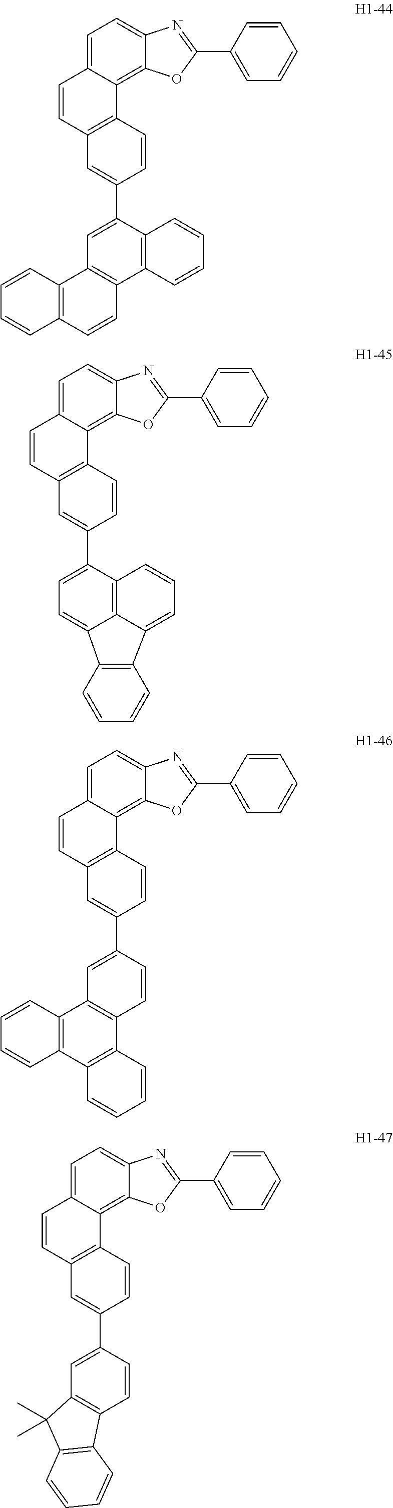

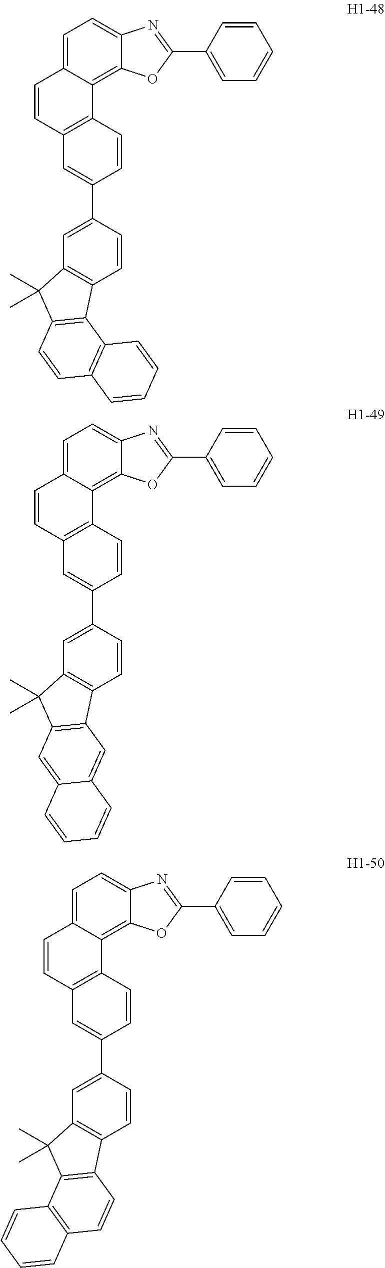

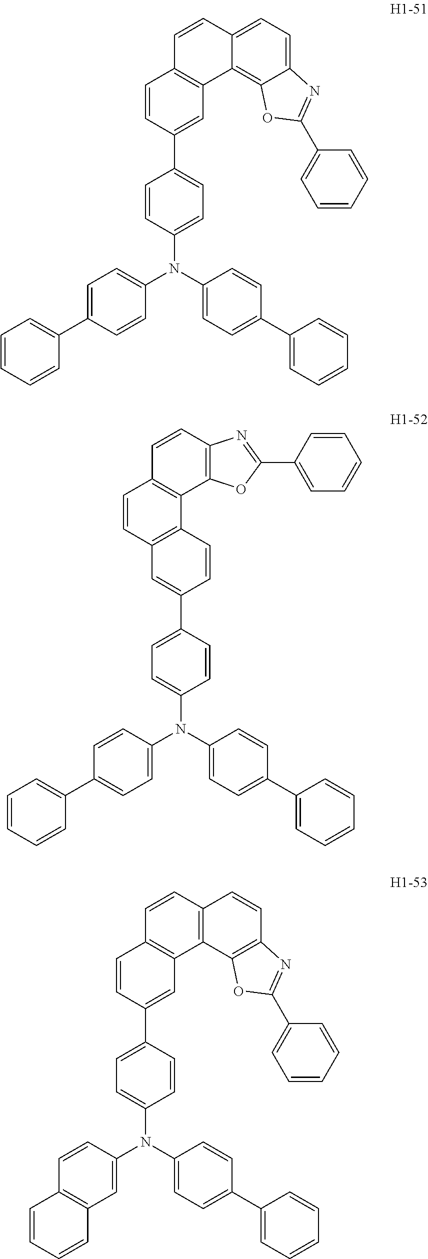 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7nc(-c8ccccc8)oc7c65)c4)cc3)cc2)cc1 ; c1ccc(-c2ccc(N(c3ccc(-c4ccc5ccc6ccc7nc(-c8ccccc8)oc7c6c5c4)cc3)c3ccc4ccccc4c3)cc2)cc1]

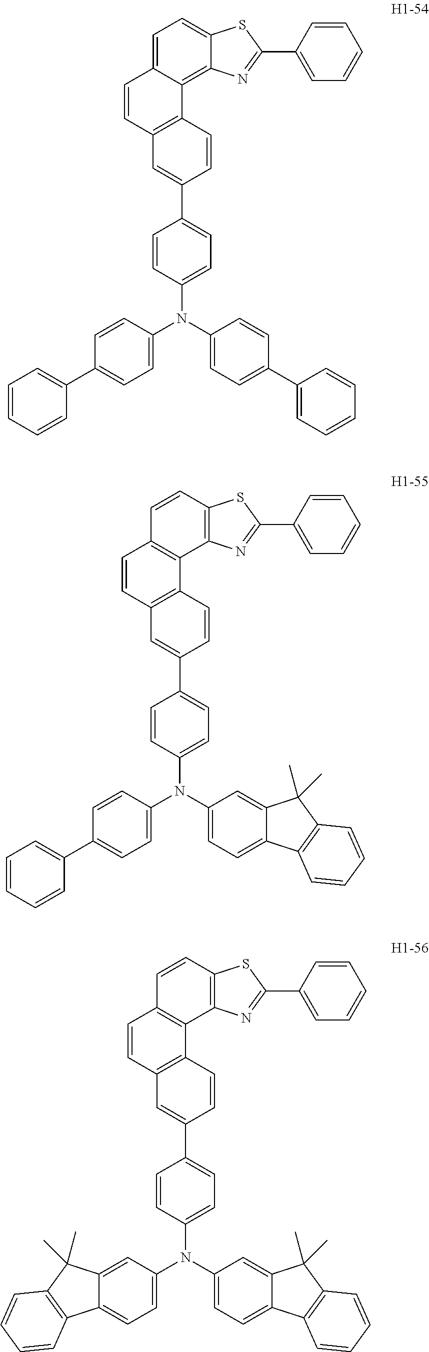 [SMILES: c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21]

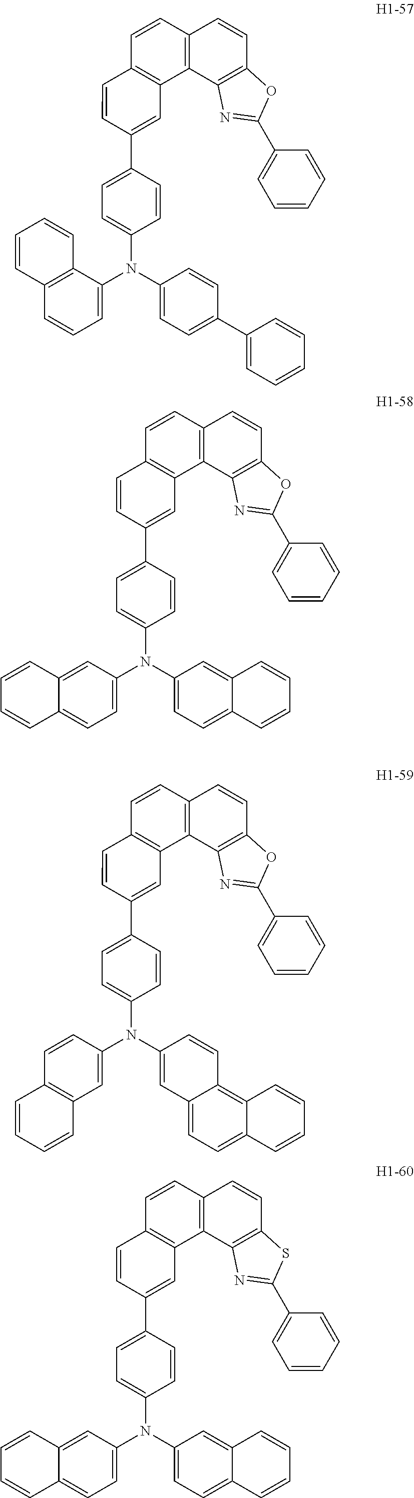

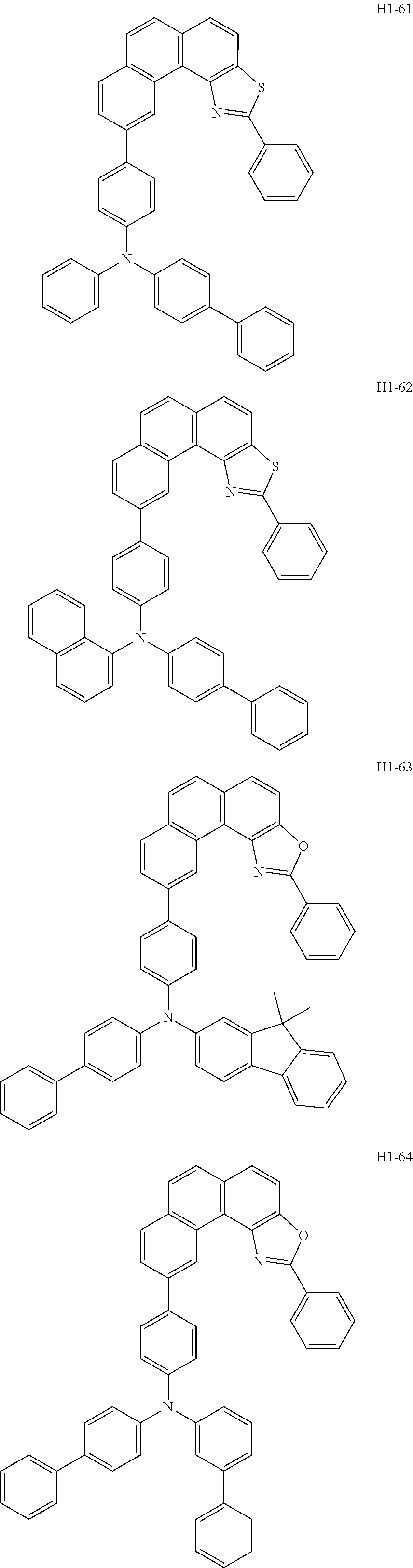

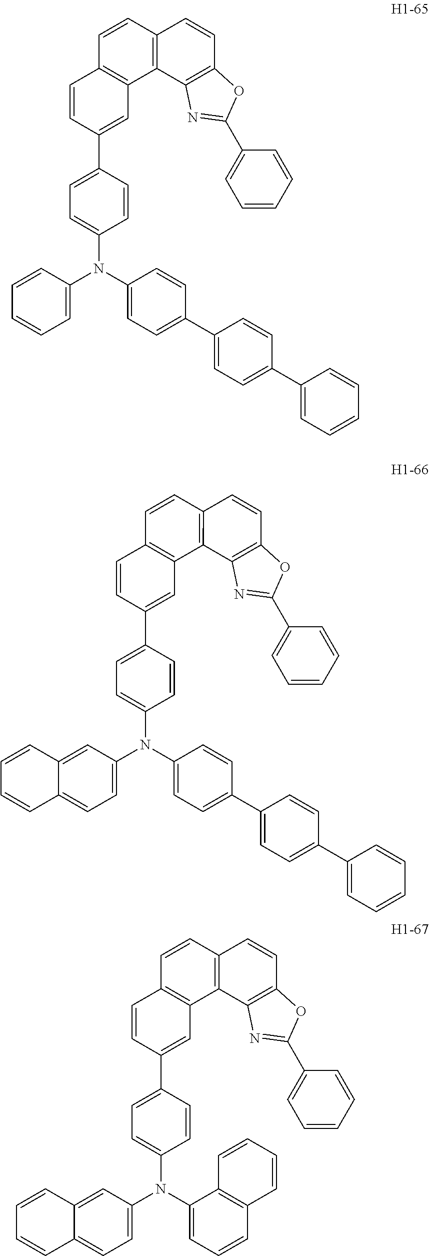 [SMILES: c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1]

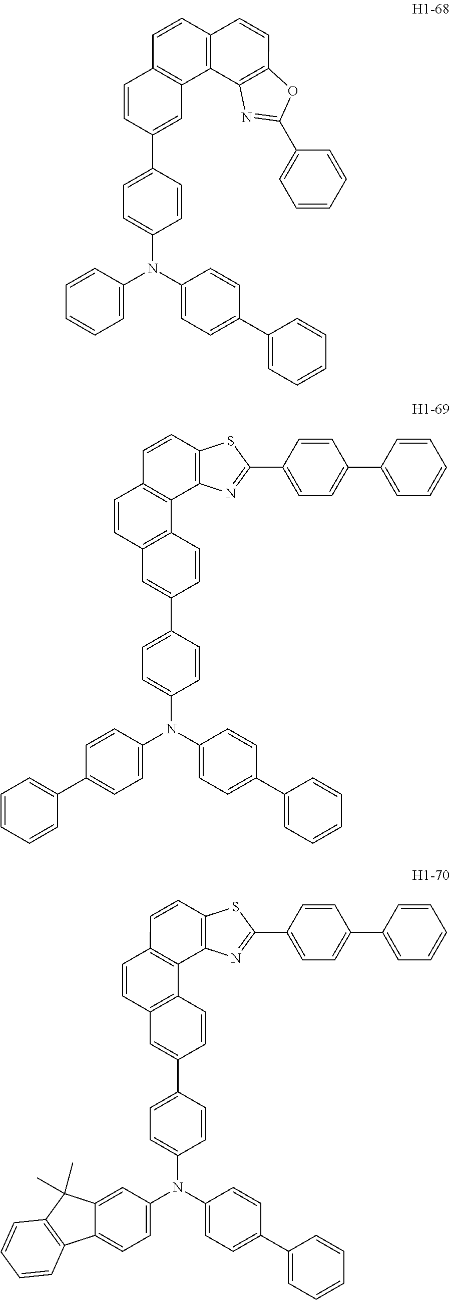 [SMILES: c1ccc(-c2ccc(N(c3ccccc3)c3ccc(-c4ccc5ccc6ccc7oc(-c8ccccc8)nc7c6c5c4)cc3)cc2)cc1 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21]

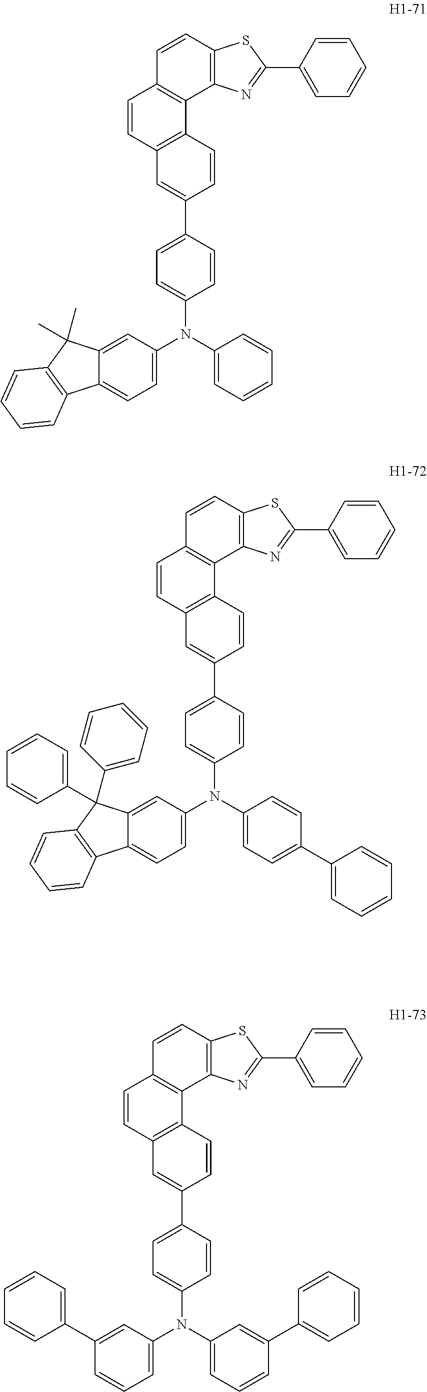 [SMILES: CC1(C)c2ccccc2-c2ccc(N(c3ccccc3)c3ccc(-c4ccc5c(ccc6ccc7sc(-c8ccccc8)nc7c65)c4)cc3)cc21 ; c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1]

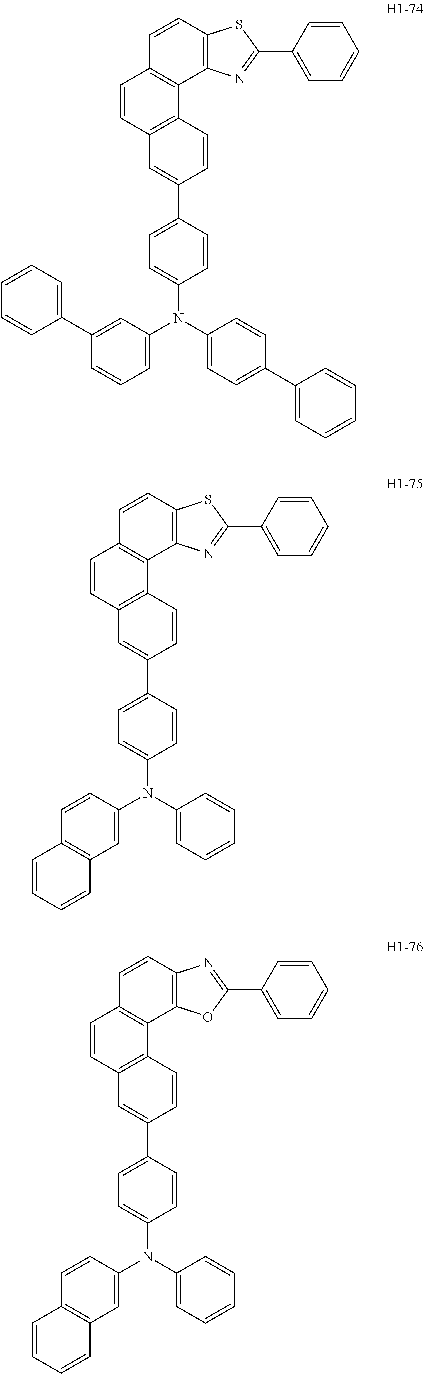 [SMILES: c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2nc3c(ccc4ccc5cc(-c6ccc(N(c7ccccc7)c7ccc8ccccc8c7)cc6)ccc5c43)s2)cc1 ; c1ccc(-c2nc3ccc4ccc5cc(-c6ccc(N(c7ccccc7)c7ccc8ccccc8c7)cc6)ccc5c4c3o2)cc1]

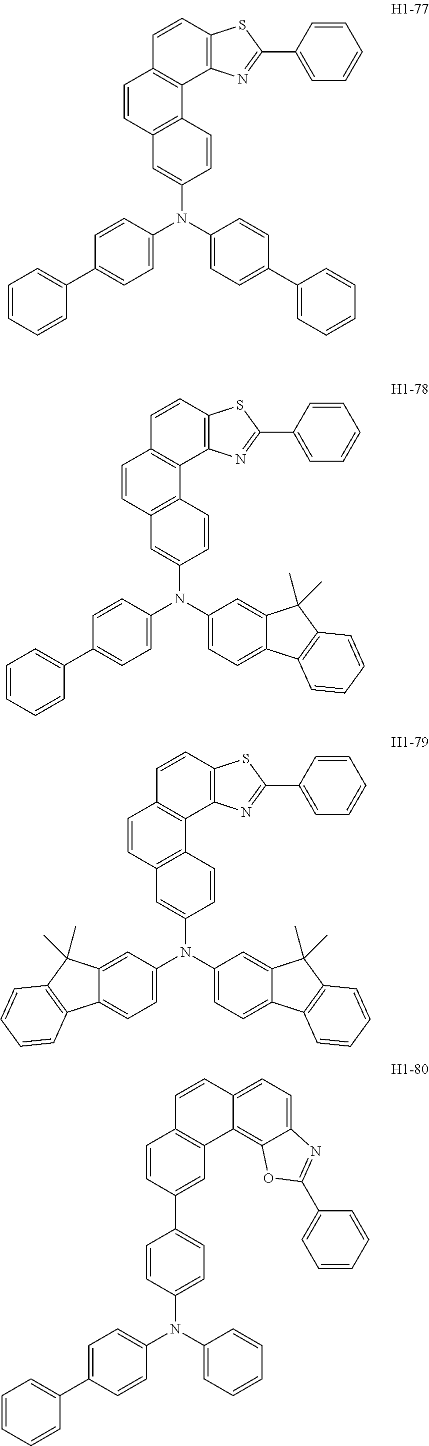 [SMILES: c1ccc(-c2ccc(N(c3ccc(-c4ccccc4)cc3)c3ccc4c(ccc5ccc6sc(-c7ccccc7)nc6c54)c3)cc2)cc1 ; CC1(C)c2ccccc2-c2ccc(N(c3ccc(-c4ccccc4)cc3)c3ccc4c(ccc5ccc6sc(-c7ccccc7)nc6c54)c3)cc21 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1]

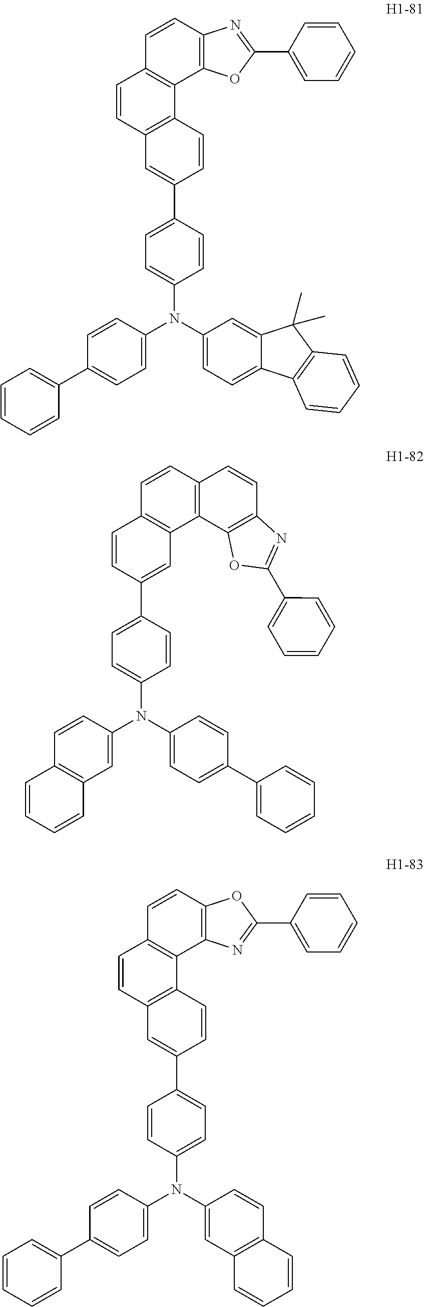 [SMILES: CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1]

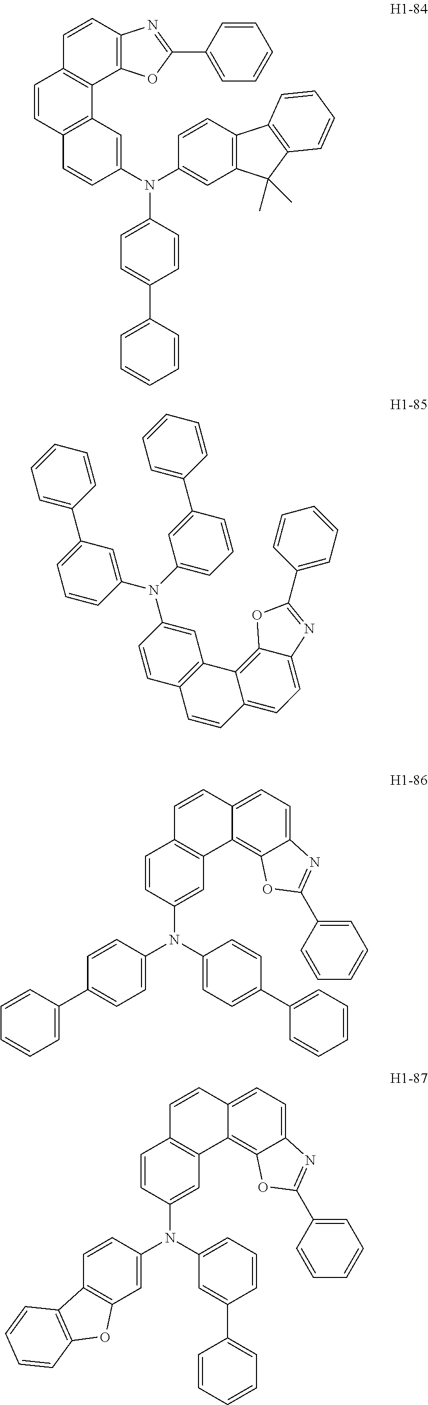

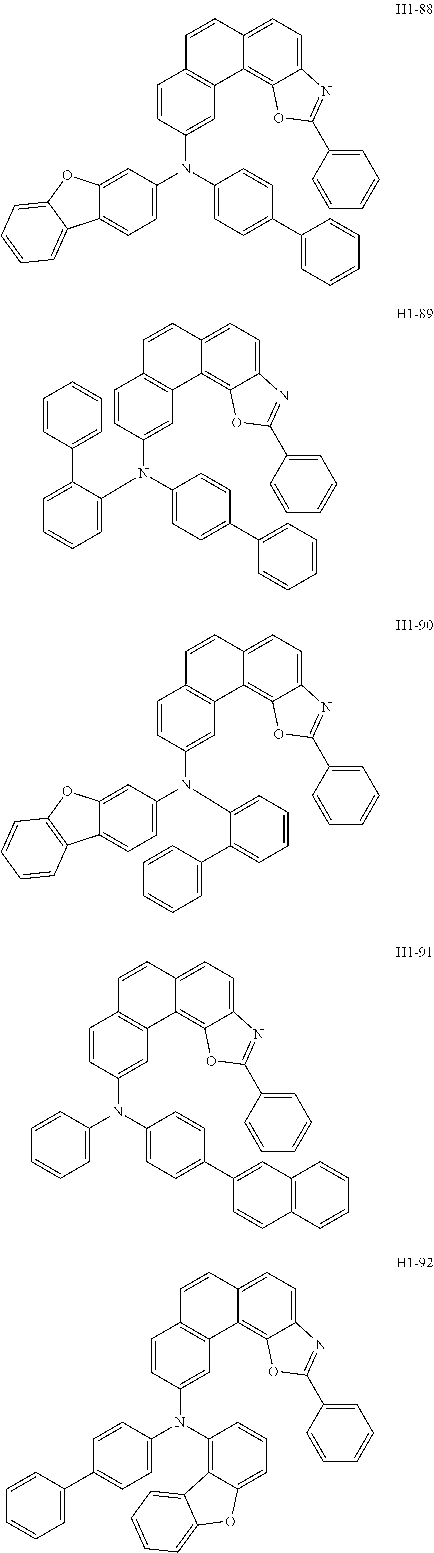

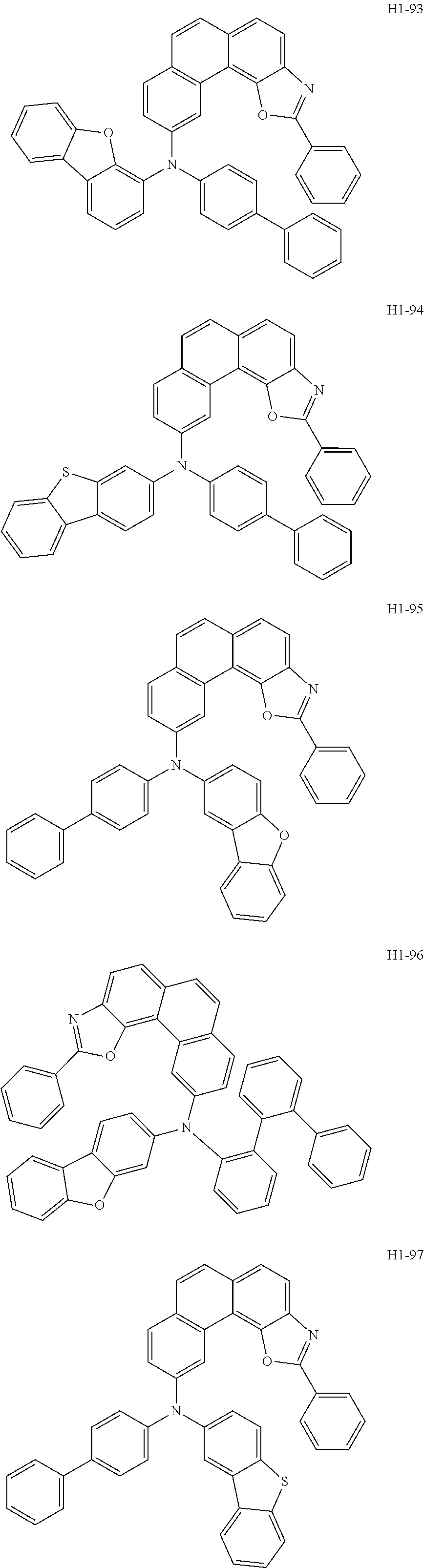 [SMILES: c1ccc(-c2ccc(N(c3ccc4ccc5ccc6nc(-c7ccccc7)oc6c5c4c3)c3cccc4c3oc3ccccc34)cc2)cc1 ; c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2ccc(N(c3ccc4oc5ccccc5c4c3)c3ccc4ccc5ccc6nc(-c7ccccc7)oc6c5c4c3)cc2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2ccc(N(c3ccc4sc5ccccc5c4c3)c3ccc4ccc5ccc6nc(-c7ccccc7)oc6c5c4c3)cc2)cc1]

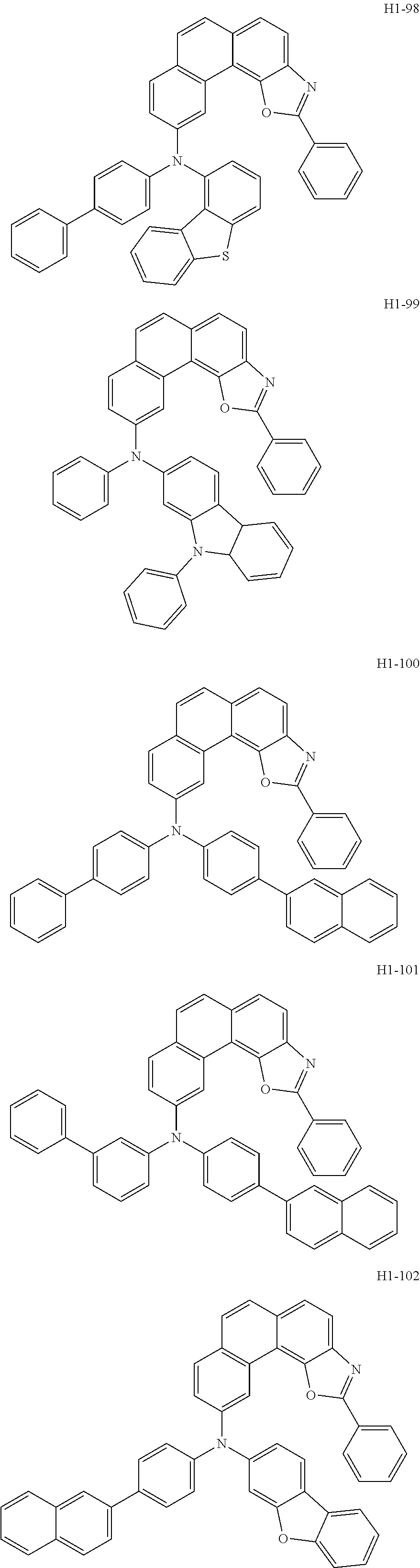

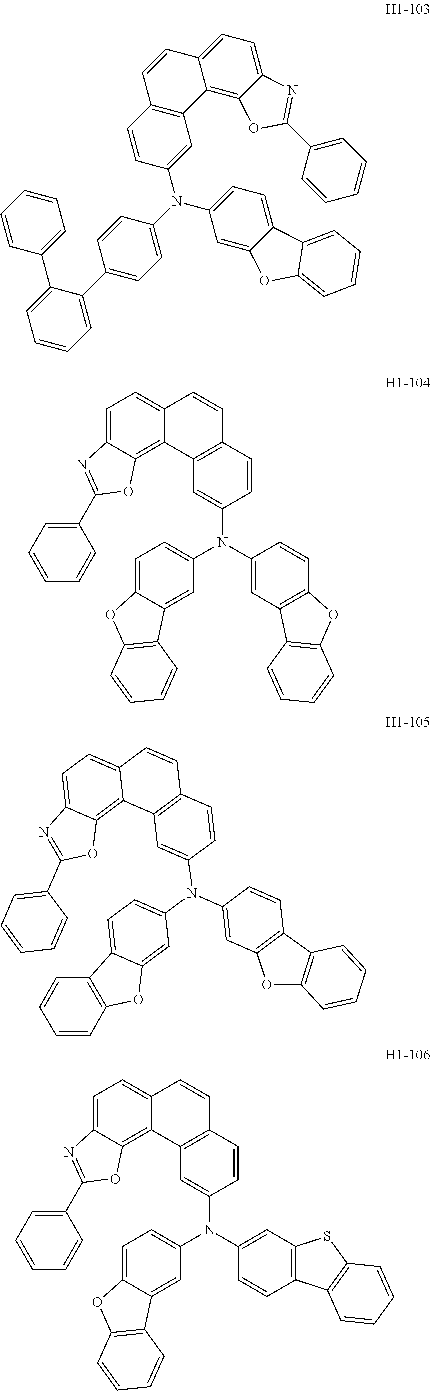 [SMILES: c1ccc(-c2nc3ccc4ccc5ccc(N(c6ccc(-c7ccccc7-c7ccccc7)cc6)c6ccc7c(c6)oc6ccccc67)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7c(c6)oc6ccccc67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1]

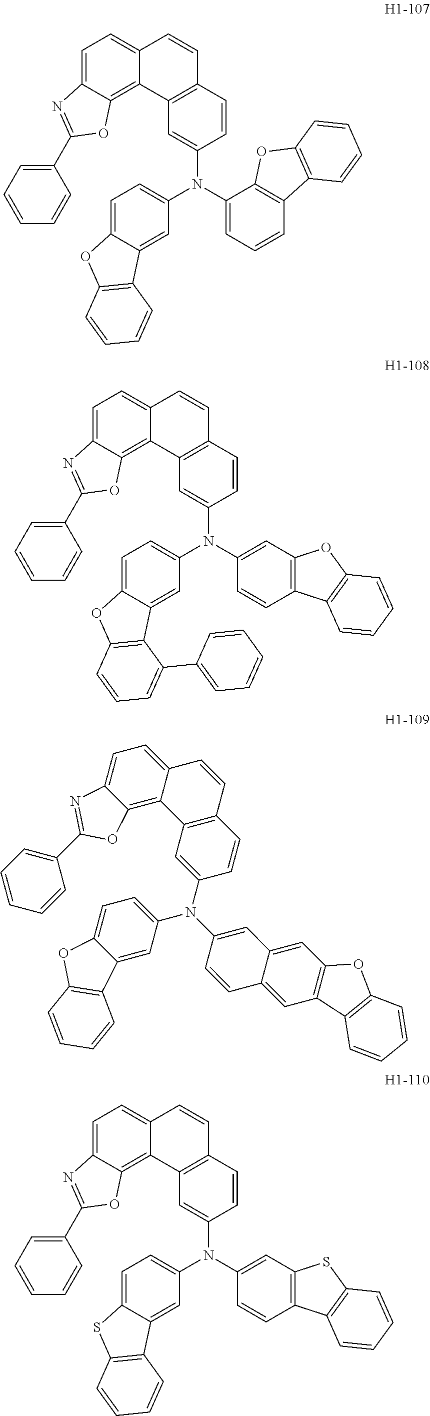 [SMILES: c1ccc(-c2nc3ccc4ccc5ccc(N(c6ccc7oc8ccccc8c7c6)c6cccc7c6oc6ccccc67)cc5c4c3o2)cc1 ; c1ccc(-c2nc3ccc4ccc5ccc(N(c6ccc7c(c6)oc6ccccc67)c6ccc7oc8cccc(-c9ccccc9)c8c7c6)cc5c4c3o2)cc1 ; c1ccc(-c2nc3ccc4ccc5ccc(N(c6ccc7cc8c(cc7c6)oc6ccccc68)c6ccc7oc8ccccc8c7c6)cc5c4c3o2)cc1 ; c1ccc(-c2nc3ccc4ccc5ccc(N(c6ccc7c(c6)sc6ccccc67)c6ccc7sc8ccccc8c7c6)cc5c4c3o2)cc1]

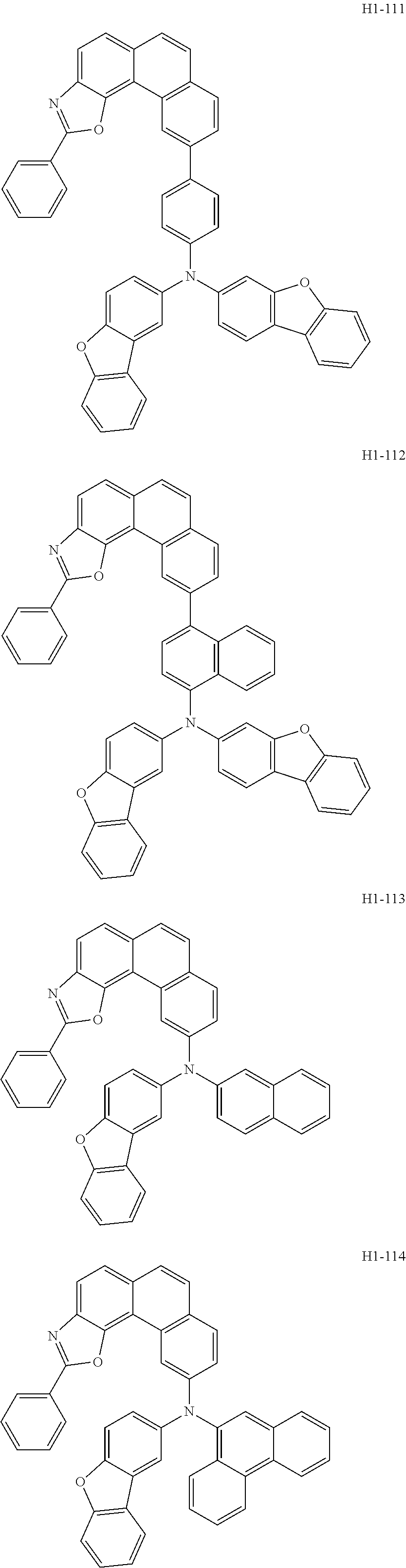

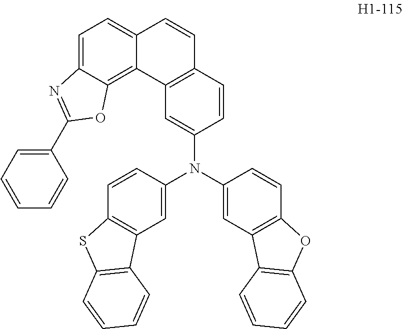 [SMILES: c1ccc(-c2nc3ccc4ccc5ccc(N(c6ccc7oc8ccccc8c7c6)c6ccc7sc8ccccc8c7c6)cc5c4c3o2)cc1]

The compound represented by formula 1 according to the present disclosure may be produced by a synthetic method known to a person skilled in the art, for example, the compound represented by formula 1 may be prepared by referring to Korean Patent Application Laid-Open No. 2017-0022865 (Mar. 2, 2017), but is not limited thereto:

The second host material as another host material according to one embodiment comprises a compound represented by the following formula 2.

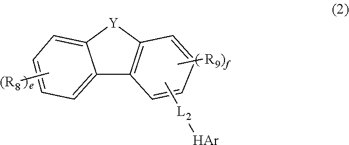

in formula 2,

Y represents —O— or —S—;

L₂ represents a single bond, a substituted or unsubstituted (C6-C30)arylene, or a substituted or unsubstituted (3- to 30-membered)heteroarylene;

HAr represents a substituted or unsubstituted (3- to 30-membered)heteroaryl containing at least one nitrogen atom;

R₈ and R₉ each independently represent hydrogen, deuterium, cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₁₆R₁₇, —SiR₁₈R₁₉R₂₀, or a combination thereof; or may be linked to the adjacent substituents to form a ring(s);

R₁₆ to R₂₀ each independently represent a substituted or unsubstituted (C1-C30)alkyl, a substituted or unsubstituted (C6-C30)aryl, or a substituted or unsubstituted (3- to 30-membered)heteroaryl;

e represents an integer of 1 to 4, and f represents an integer of 1 to 3; and

when e and f are an integer of 2 or more, each of R₈ and each of R₉ may be the same or different;

provided that at least one of HAr, R₈, and R₉ includes -L₃-SiR′R″R′″ or -L₃-CR′R″R′″;

L₃ represents a single bond, a substituted or unsubstituted (C1-C30)alkylene, a substituted or unsubstituted (C3-C30)cycloalkylene, a substituted or unsubstituted (C6-C30)arylene, or a substituted or unsubstituted (3- to 30-membered)heteroarylene; and

R′, R″, and R′″ each independently represent a substituted or unsubstituted (C6-C30)aryl or a substituted or unsubstituted (3- to 30-membered)heteroaryl.

In one embodiment, L₂ may be a single bond or a substituted or unsubstituted (C6-C30)arylene, preferably a single bond or a substituted or unsubstituted (C6-C25)arylene, more preferably a single bond or a substituted or unsubstituted (C6-C18)arylene. For example, L₂ may be a single bond, a substituted or unsubstituted phenylene, a substituted or unsubstituted p-biphenylene, a substituted or unsubstituted m-biphenylene, a substituted or unsubstituted o-biphenylene, or a substituted or unsubstituted naphthylene.

In one embodiment, at least one of HAr, R₈, and R₉ includes -L₃-SiR′R″R′″ or -L₃-CR′R″R′″. For example, R₈ and R₉ are each independently hydrogen or deuterium, and HAr may includes -L₃-SiR′R″R′″ or -L₃-CR′R″R′″.

In one embodiment, HAr may be a substituted or unsubstituted (5- to 30-membered)heteroaryl comprising at least one nitrogen atom, preferably a substituted or unsubstituted (5- to 30-membered)heteroaryl comprising at least two nitrogen atoms, more preferably (5- to 30-membered)heteroaryl comprising at least three nitrogen atoms and substituted with -L₃-SiR′R″R′″. For example, HAr may be a substituted or unsubstituted pyridyl, a substituted or unsubstituted pyrimidinyl, a substituted or unsubstituted triazinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthyridinyl, a substituted or unsubstituted benzofuropyrimidinyl, a substituted or unsubstituted benzothienopyrimidinyl, a substituted or unsubstituted carbazolyl, or a substituted or unsubstituted pyridopyrazinyl. For example, the substituents of the substituted groups may be -L₃-SiR′R″R′″ or -L₃-CR′R″R′″.

In one embodiment, L₃ may be a substituted or unsubstituted (C6-C30)arylene, preferably a substituted or unsubstituted (C6-C25)arylene, more preferably a substituted or unsubstituted (C6-C18)arylene. For example, L₃ may be a substituted or unsubstituted phenylene, a substituted or unsubstituted naphthalenylene, a substituted or unsubstituted p-biphenylene, or a substituted or unsubstituted m-biphenylene.

In one embodiment, R′, R″, and R′″ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, R′, R″, and R′″ each independently may be, a substituted or unsubstituted phenyl, a substituted or unsubstituted p-biphenyl, a substituted or unsubstituted fluorenyl, a substituted or unsubstituted pyridyl, a substituted or unsubstituted carbazolyl, a substituted or unsubstituted isoquinolinyl, a substituted or unsubstituted dibenzofuranyl, or a substituted or unsubstituted dibenzothiophenyl. For example, the substituents of the substituted groups may be cyano, methyl, or phenyl.

The compound represented by the formula 2 according to one embodiment may be represented by the following formula 2-1.

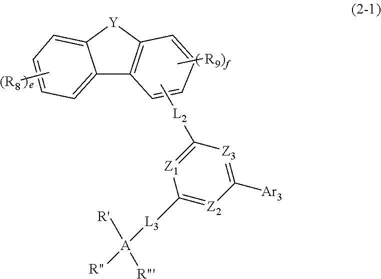

in formula 2-1,

Z₁ to Z₃ each independently represent N or CH; provided that at least one of Z₁ to Z₃ is N;

A represents Si or C;

Ar₃ represents a substituted or unsubstituted (C6-C30)aryl or a substituted or unsubstituted (3- to 30-membered)heteroaryl; and

Y, R₈, R₉, L₂, L₃, R′, R″, R′″, e, and f are as defined in formula 2.

In one embodiment, at least two of Z₁ to Z₃ may be N, preferably all of Z₁ to Z₃ may be N.

In one embodiment, Ar₃ may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, Ar₃ may be a substituted or unsubstituted phenyl, a substituted or unsubstituted naphthyl, a substituted or unsubstituted p-biphenyl, a substituted or unsubstituted m-biphenyl, a substituted or unsubstituted o-biphenyl, a substituted or unsubstituted m-terphenyl, a substituted or unsubstituted o-terphenyl, a substituted or unsubstituted p-terphenyl, a substituted or unsubstituted phenanthrenyl, a substituted or unsubstituted chrysenyl, a substituted or unsubstituted triphenylenyl, a substituted or unsubstituted fluorenyl, a substituted or unsubstituted benzofluorenyl, a substituted or unsubstituted carbazolyl, a substituted or unsubstituted dibenzofuranyl, a substituted or unsubstituted benzonaphthofuranyl, a substituted or unsubstituted dibenzothiophenyl, a substituted or unsubstituted phenanthrooxazolyl, or a substituted or unsubstituted naphthoisoxazolyl. For example, the substituents of the substituted groups may be deuterium, cyano, methyl, phenyl, naphthyl, or dibenzofuranyl.

In one embodiment, the second host material comprising a compound represented by formula 2 may be more specifically illustrated by the following compounds, but is not limited thereto.

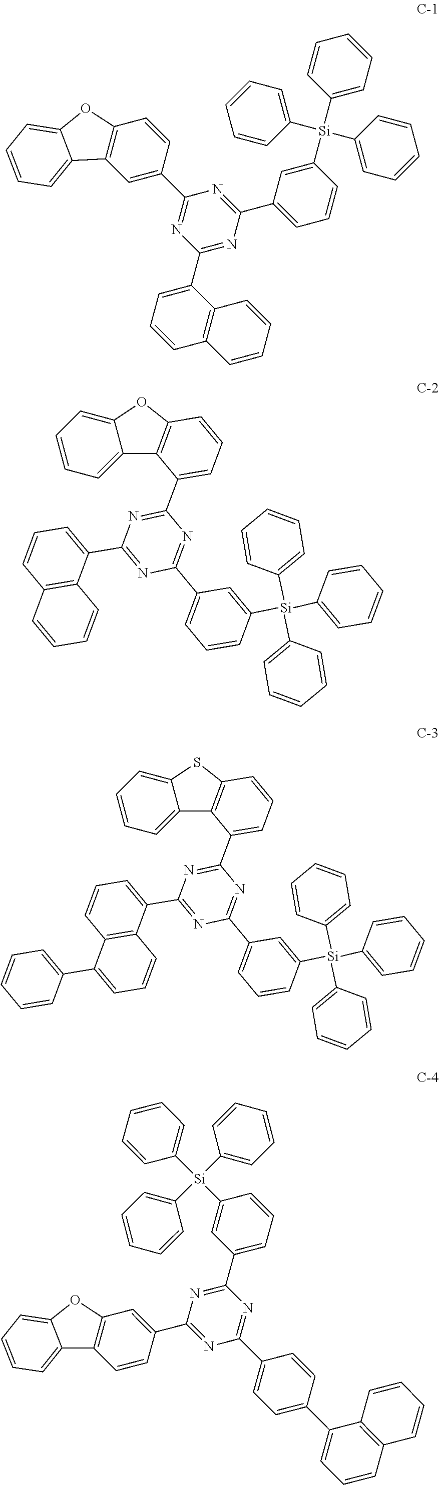 [SMILES: c1ccc([Si](c2ccccc2)(c2ccccc2)c2cccc(-c3nc(-c4ccc5oc6ccccc6c5c4)nc(-c4cccc5ccccc45)n3)c2)cc1 ; c1ccc([Si](c2ccccc2)(c2ccccc2)c2cccc(-c3nc(-c4cccc5ccccc45)nc(-c4cccc5oc6ccccc6c45)n3)c2)cc1 ; c1ccc(-c2cccc3c(-c4nc(-c5cccc([Si](c6ccccc6)(c6ccccc6)c6ccccc6)c5)nc(-c5cccc6sc7ccccc7c56)n4)cccc23)cc1 ; c1ccc([Si](c2ccccc2)(c2ccccc2)c2cccc(-c3nc(-c4ccc(-c5cccc6ccccc56)cc4)nc(-c4ccc5c(c4)oc4ccccc45)n3)c2)cc1]

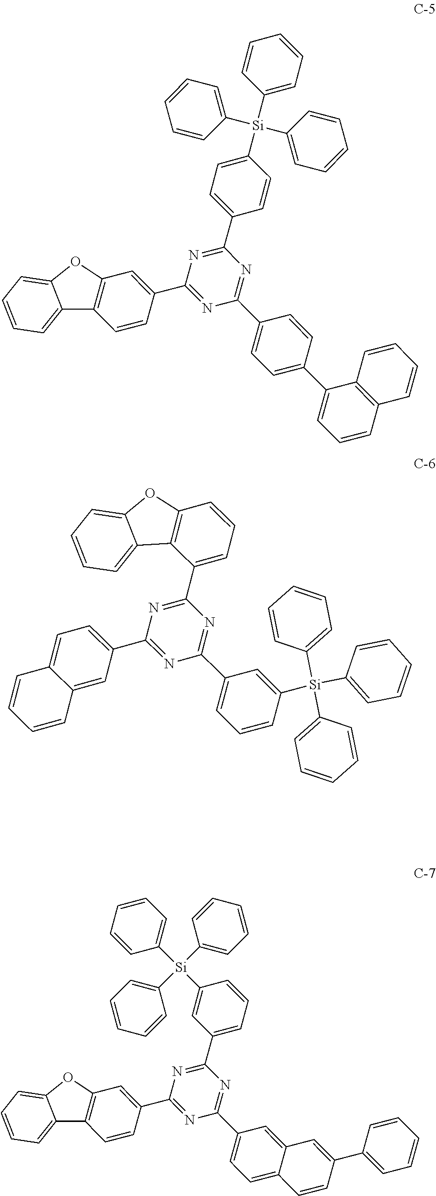 [SMILES: c1ccc([Si](c2ccccc2)(c2ccccc2)c2ccc(-c3nc(-c4ccc(-c5cccc6ccccc56)cc4)nc(-c4ccc5c(c4)oc4ccccc45)n3)cc2)cc1 ; c1ccc([Si](c2ccccc2)(c2ccccc2)c2cccc(-c3nc(-c4ccc5ccccc5c4)nc(-c4cccc5oc6ccccc6c45)n3)c2)cc1 ; c1ccc(-c2ccc3ccc(-c4nc(-c5cccc([Si](c6ccccc6)(c6ccccc6)c6ccccc6)c5)nc(-c5ccc6c(c5)oc5ccccc56)n4)cc3c2)cc1]

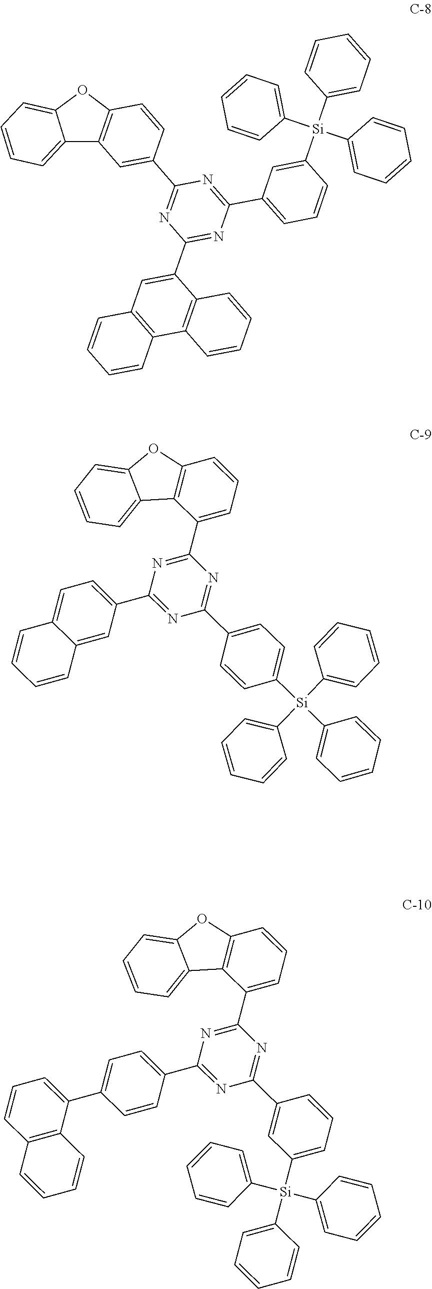 [SMILES: c1ccc([Si](c2ccccc2)(c2ccccc2)c2cccc(-c3nc(-c4ccc5oc6ccccc6c5c4)nc(-c4cc5ccccc5c5ccccc45)n3)c2)cc1 ; c1ccc([Si](c2ccccc2)(c2ccccc2)c2ccc(-c3nc(-c4ccc5ccccc5c4)nc(-c4cccc5oc6ccccc6c45)n3)cc2)cc1 ; c1ccc([Si](c2ccccc2)(c2ccccc2)c2cccc(-c3nc(-c4ccc(-c5cccc6ccccc56)cc4)nc(-c4cccc5oc6ccccc6c45)n3)c2)cc1]

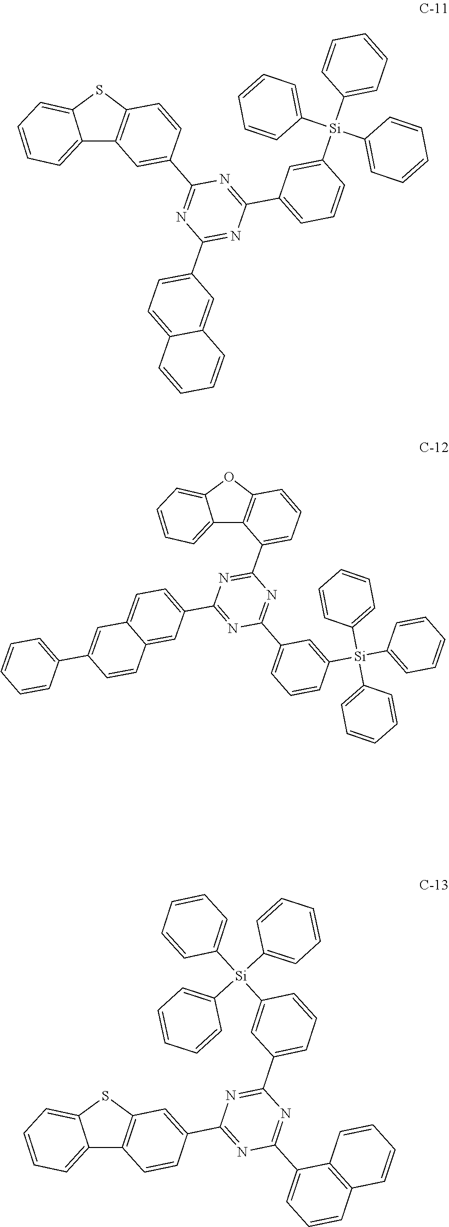 [SMILES: c1ccc([Si](c2ccccc2)(c2ccccc2)c2cccc(-c3nc(-c4ccc5ccccc5c4)nc(-c4ccc5sc6ccccc6c5c4)n3)c2)cc1 ; c1ccc(-c2ccc3cc(-c4nc(-c5cccc([Si](c6ccccc6)(c6ccccc6)c6ccccc6)c5)nc(-c5cccc6oc7ccccc7c56)n4)ccc3c2)cc1 ; c1ccc([Si](c2ccccc2)(c2ccccc2)c2cccc(-c3nc(-c4ccc5c(c4)sc4ccccc45)nc(-c4cccc5ccccc45)n3)c2)cc1]

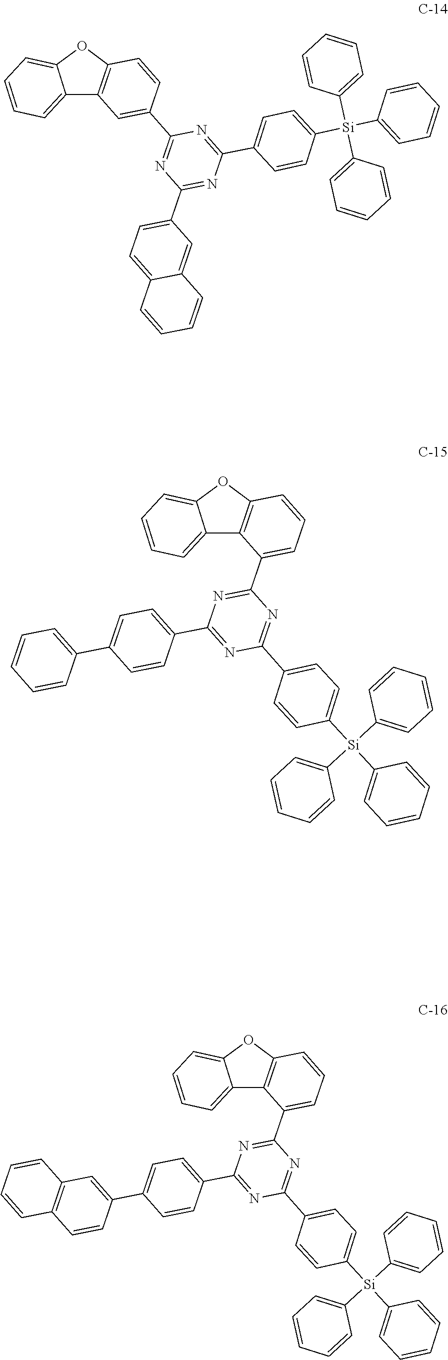 [SMILES: c1ccc([Si](c2ccccc2)(c2ccccc2)c2ccc(-c3nc(-c4ccc5ccccc5c4)nc(-c4ccc5oc6ccccc6c5c4)n3)cc2)cc1 ; c1ccc(-c2ccc(-c3nc(-c4ccc([Si](c5ccccc5)(c5ccccc5)c5ccccc5)cc4)nc(-c4cccc5oc6ccccc6c45)n3)cc2)cc1 ; c1ccc([Si](c2ccccc2)(c2ccccc2)c2ccc(-c3nc(-c4ccc(-c5ccc6ccccc6c5)cc4)nc(-c4cccc5oc6ccccc6c45)n3)cc2)cc1]

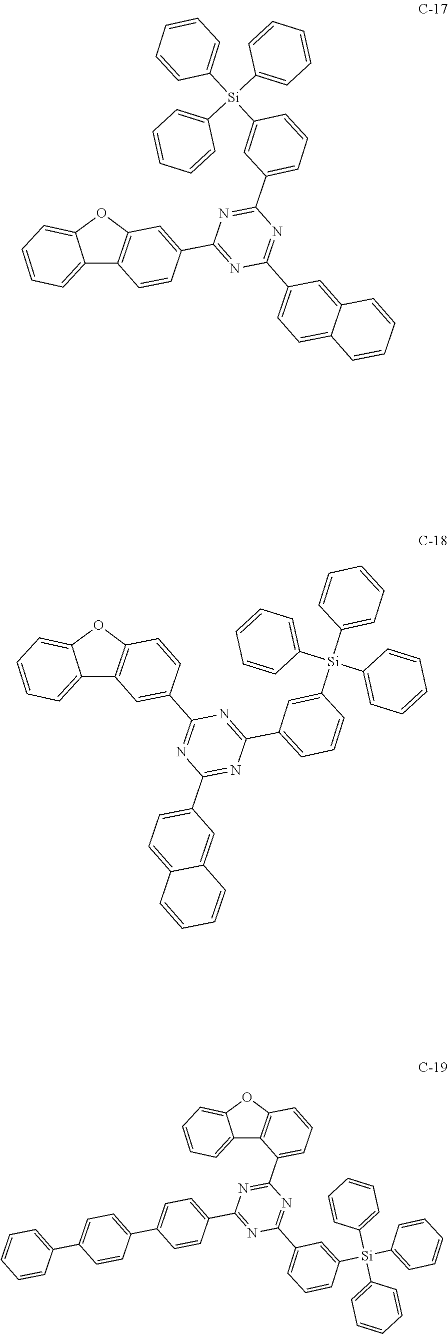 [SMILES: c1ccc([Si](c2ccccc2)(c2ccccc2)c2cccc(-c3nc(-c4ccc5ccccc5c4)nc(-c4ccc5c(c4)oc4ccccc45)n3)c2)cc1 ; c1ccc([Si](c2ccccc2)(c2ccccc2)c2cccc(-c3nc(-c4ccc5ccccc5c4)nc(-c4ccc5oc6ccccc6c5c4)n3)c2)cc1 ; c1ccc(-c2ccc(-c3ccc(-c4nc(-c5cccc([Si](c6ccccc6)(c6ccccc6)c6ccccc6)c5)nc(-c5cccc6oc7ccccc7c56)n4)cc3)cc2)cc1]

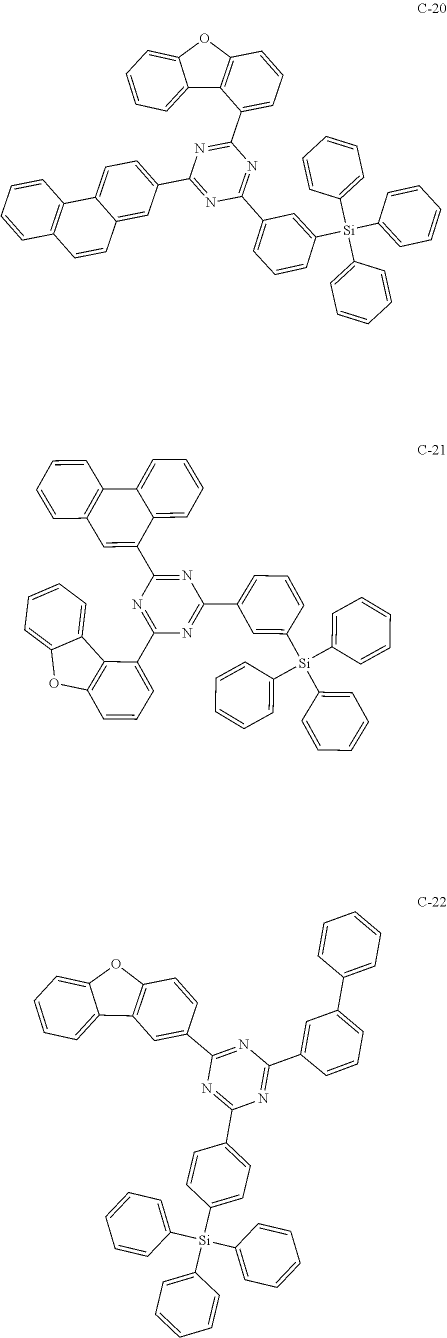 [SMILES: c1ccc([Si](c2ccccc2)(c2ccccc2)c2cccc(-c3nc(-c4ccc5c(ccc6ccccc65)c4)nc(-c4cccc5oc6ccccc6c45)n3)c2)cc1 ; c1ccc([Si](c2ccccc2)(c2ccccc2)c2cccc(-c3nc(-c4cc5ccccc5c5ccccc45)nc(-c4cccc5oc6ccccc6c45)n3)c2)cc1 ; c1ccc(-c2cccc(-c3nc(-c4ccc([Si](c5ccccc5)(c5ccccc5)c5ccccc5)cc4)nc(-c4ccc5oc6ccccc6c5c4)n3)c2)cc1]

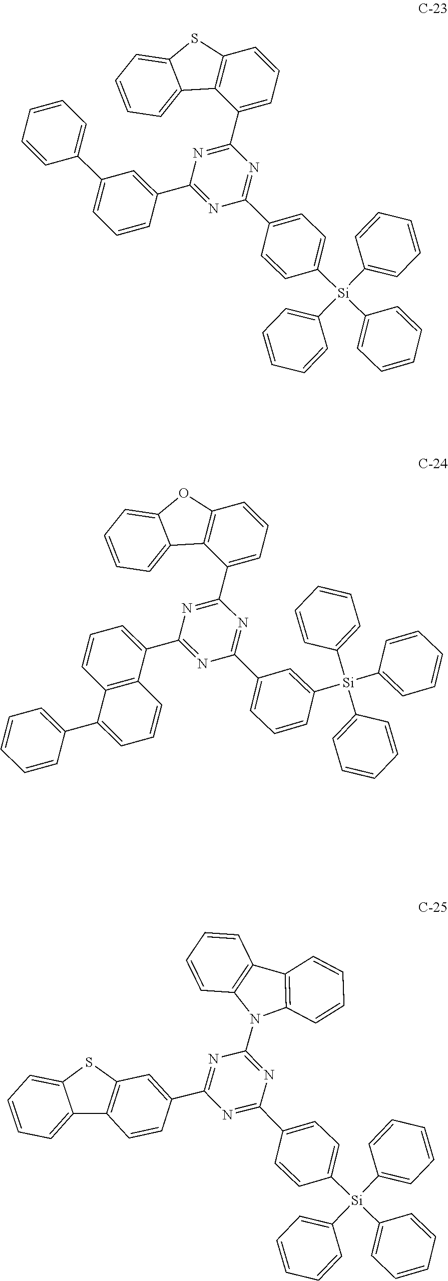 [SMILES: c1ccc(-c2cccc(-c3nc(-c4ccc([Si](c5ccccc5)(c5ccccc5)c5ccccc5)cc4)nc(-c4cccc5sc6ccccc6c45)n3)c2)cc1 ; c1ccc(-c2cccc3c(-c4nc(-c5cccc([Si](c6ccccc6)(c6ccccc6)c6ccccc6)c5)nc(-c5cccc6oc7ccccc7c56)n4)cccc23)cc1 ; c1ccc([Si](c2ccccc2)(c2ccccc2)c2ccc(-c3nc(-c4ccc5c(c4)sc4ccccc45)nc(-n4c5ccccc5c5ccccc54)n3)cc2)cc1]

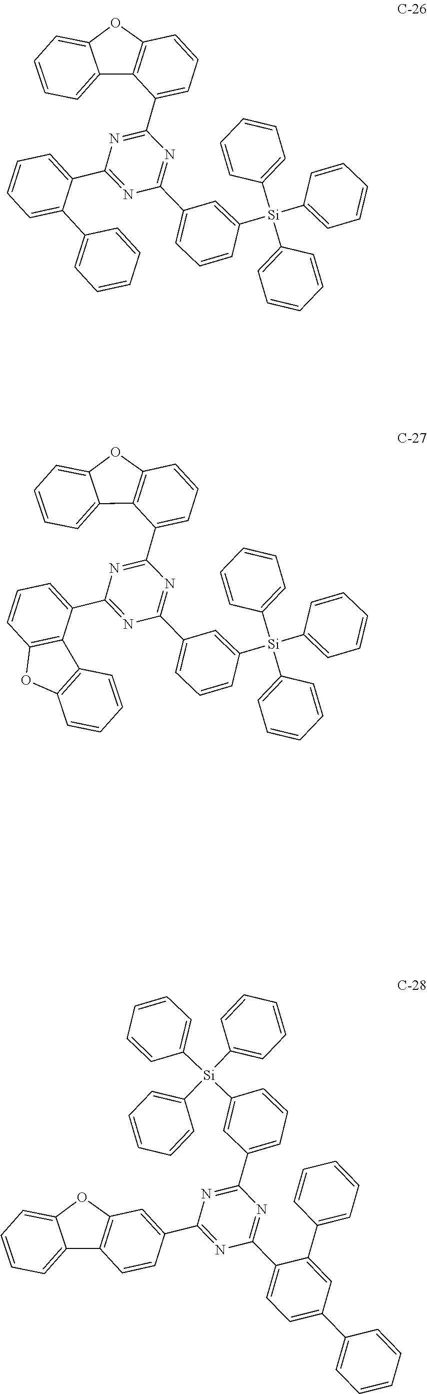 [SMILES: c1ccc(-c2ccccc2-c2nc(-c3cccc([Si](c4ccccc4)(c4ccccc4)c4ccccc4)c3)nc(-c3cccc4oc5ccccc5c34)n2)cc1 ; c1ccc([Si](c2ccccc2)(c2ccccc2)c2cccc(-c3nc(-c4cccc5oc6ccccc6c45)nc(-c4cccc5oc6ccccc6c45)n3)c2)cc1 ; c1ccc(-c2ccc(-c3nc(-c4cccc([Si](c5ccccc5)(c5ccccc5)c5ccccc5)c4)nc(-c4ccc5c(c4)oc4ccccc45)n3)c(-c3ccccc3)c2)cc1]

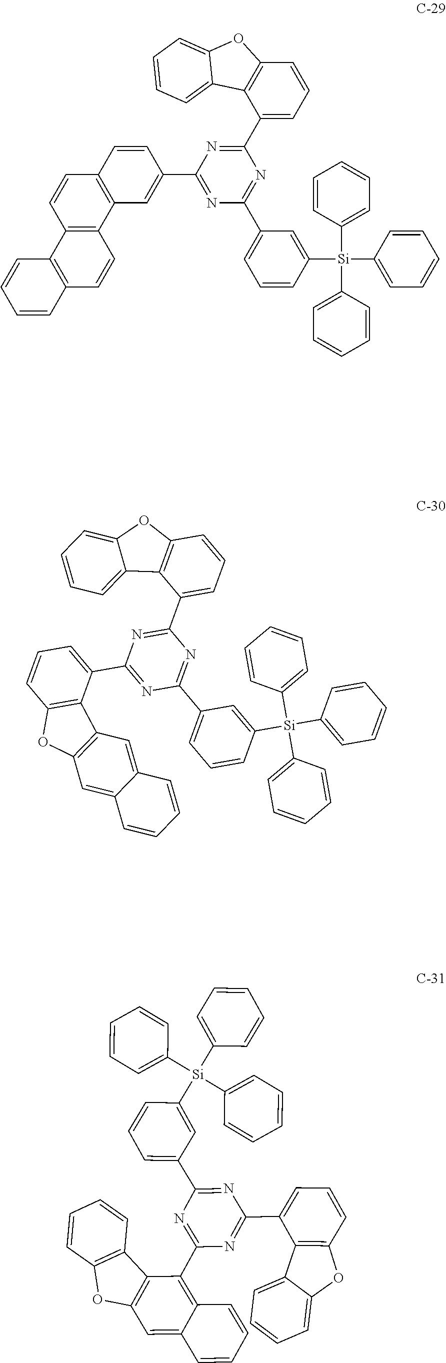 [SMILES: c1ccc([Si](c2ccccc2)(c2ccccc2)c2cccc(-c3nc(-c4ccc5ccc6c7ccccc7ccc6c5c4)nc(-c4cccc5oc6ccccc6c45)n3)c2)cc1 ; c1ccc([Si](c2ccccc2)(c2ccccc2)c2cccc(-c3nc(-c4cccc5oc6ccccc6c45)nc(-c4cccc5oc6cc7ccccc7cc6c45)n3)c2)cc1 ; c1ccc([Si](c2ccccc2)(c2ccccc2)c2cccc(-c3nc(-c4cccc5oc6ccccc6c45)nc(-c4c5ccccc5cc5oc6ccccc6c45)n3)c2)cc1]

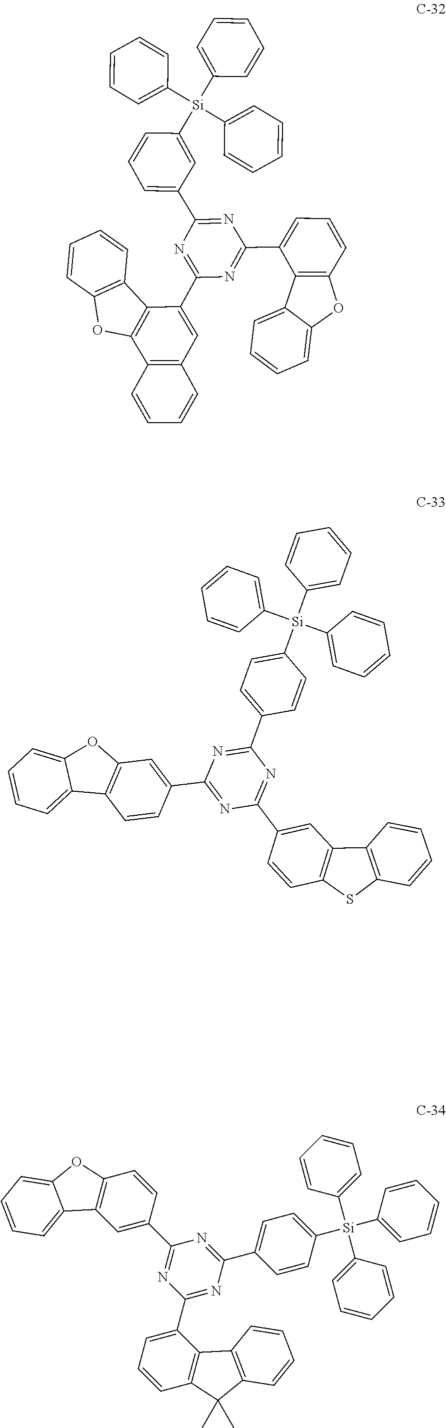 [SMILES: c1ccc([Si](c2ccccc2)(c2ccccc2)c2cccc(-c3nc(-c4cccc5oc6ccccc6c45)nc(-c4cc5ccccc5c5oc6ccccc6c45)n3)c2)cc1 ; c1ccc([Si](c2ccccc2)(c2ccccc2)c2ccc(-c3nc(-c4ccc5c(c4)oc4ccccc45)nc(-c4ccc5sc6ccccc6c5c4)n3)cc2)cc1 ; CC1(C)c2ccccc2-c2c(-c3nc(-c4ccc([Si](c5ccccc5)(c5ccccc5)c5ccccc5)cc4)nc(-c4ccc5oc6ccccc6c5c4)n3)cccc21]

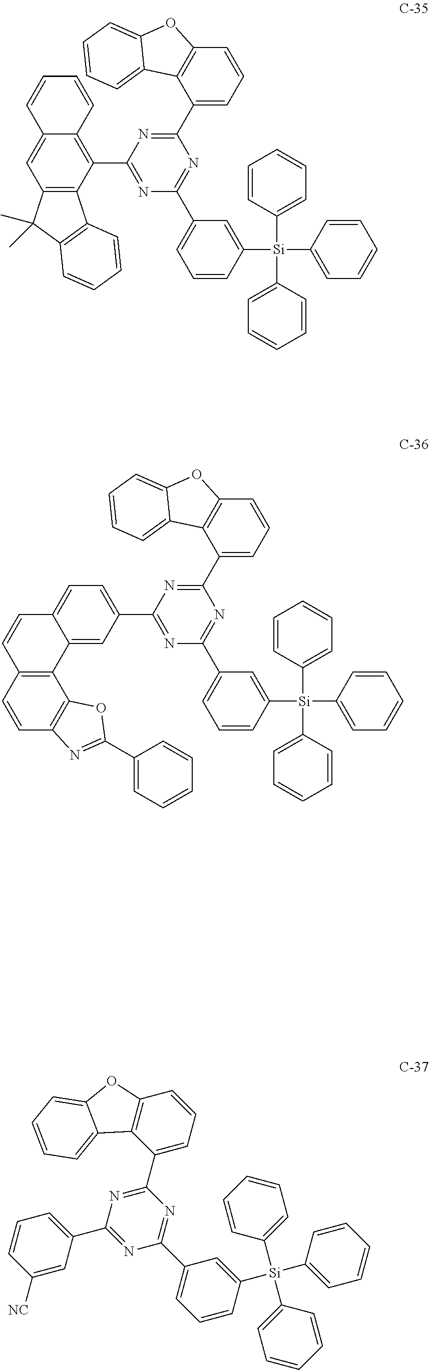 [SMILES: CC1(C)c2ccccc2-c2c1cc1ccccc1c2-c1nc(-c2cccc([Si](c3ccccc3)(c3ccccc3)c3ccccc3)c2)nc(-c2cccc3oc4ccccc4c23)n1 ; c1ccc(-c2nc3ccc4ccc5ccc(-c6nc(-c7cccc([Si](c8ccccc8)(c8ccccc8)c8ccccc8)c7)nc(-c7cccc8oc9ccccc9c78)n6)cc5c4c3o2)cc1 ; N#Cc1cccc(-c2nc(-c3cccc([Si](c4ccccc4)(c4ccccc4)c4ccccc4)c3)nc(-c3cccc4oc5ccccc5c34)n2)c1]

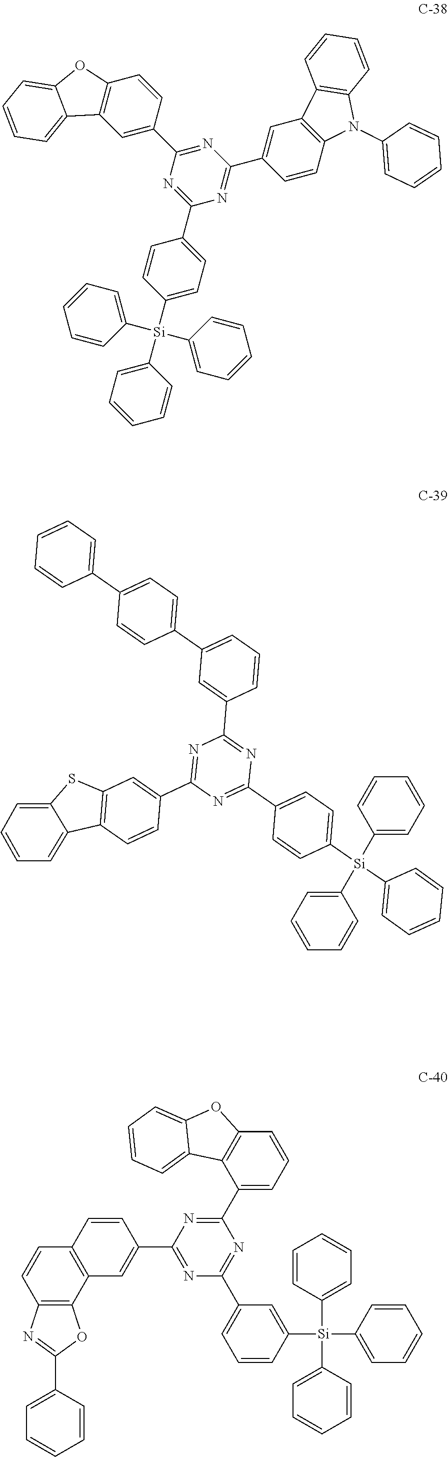 [SMILES: c1ccc(-n2c3ccccc3c3cc(-c4nc(-c5ccc([Si](c6ccccc6)(c6ccccc6)c6ccccc6)cc5)nc(-c5ccc6oc7ccccc7c6c5)n4)ccc32)cc1 ; c1ccc(-c2ccc(-c3cccc(-c4nc(-c5ccc([Si](c6ccccc6)(c6ccccc6)c6ccccc6)cc5)nc(-c5ccc6c(c5)sc5ccccc56)n4)c3)cc2)cc1 ; c1ccc(-c2nc3ccc4ccc(-c5nc(-c6cccc([Si](c7ccccc7)(c7ccccc7)c7ccccc7)c6)nc(-c6cccc7oc8ccccc8c67)n5)cc4c3o2)cc1]

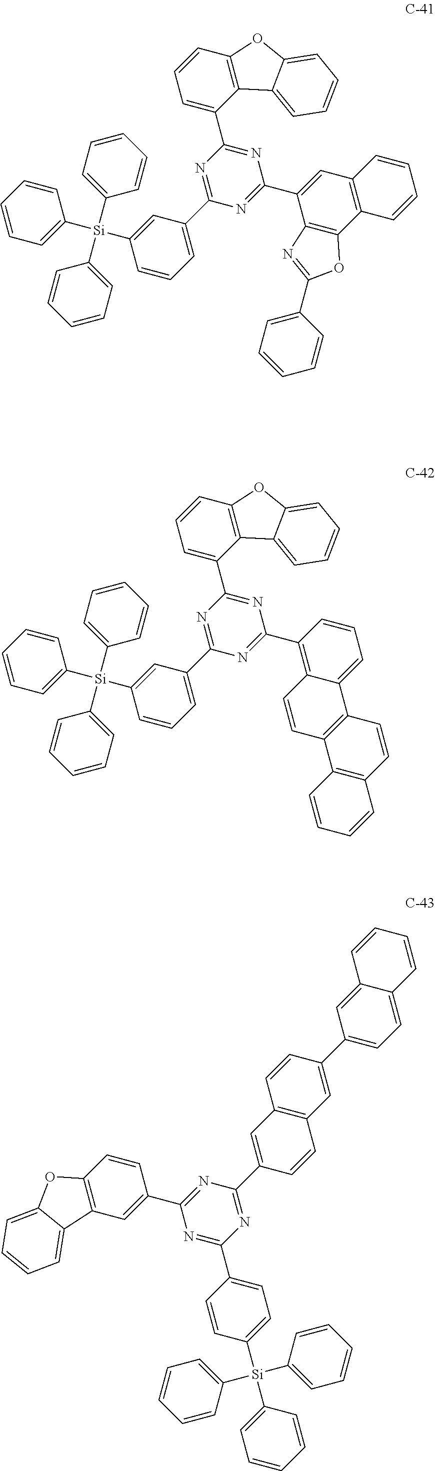 [SMILES: c1ccc(-c2nc3c(-c4nc(-c5cccc([Si](c6ccccc6)(c6ccccc6)c6ccccc6)c5)nc(-c5cccc6oc7ccccc7c56)n4)cc4ccccc4c3o2)cc1 ; c1ccc([Si](c2ccccc2)(c2ccccc2)c2cccc(-c3nc(-c4cccc5c4ccc4c6ccccc6ccc54)nc(-c4cccc5oc6ccccc6c45)n3)c2)cc1 ; c1ccc([Si](c2ccccc2)(c2ccccc2)c2ccc(-c3nc(-c4ccc5cc(-c6ccc7ccccc7c6)ccc5c4)nc(-c4ccc5oc6ccccc6c5c4)n3)cc2)cc1]

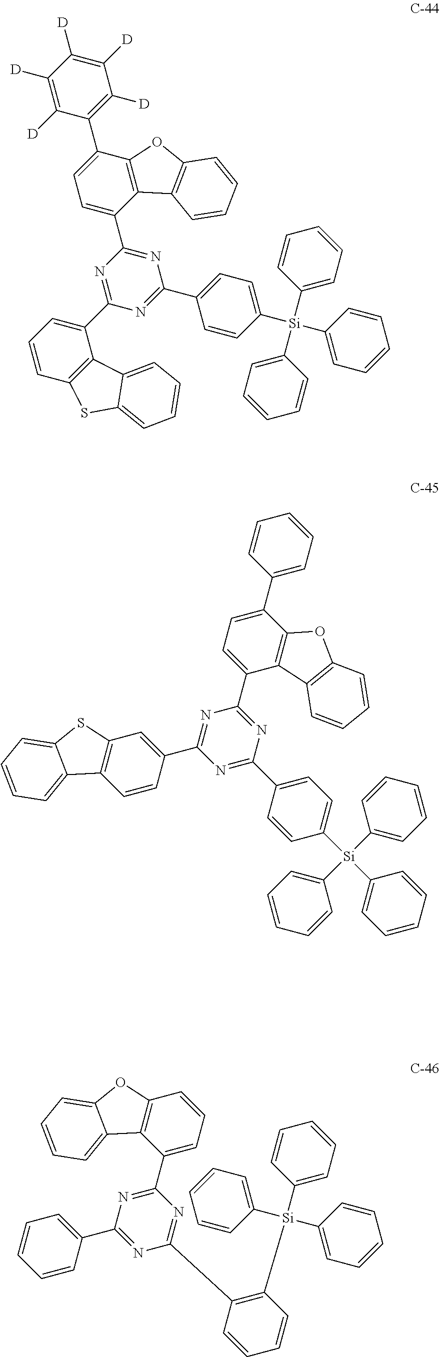 [SMILES: [2H]c1c([2H])c([2H])c(-c2ccc(-c3nc(-c4ccc([Si](c5ccccc5)(c5ccccc5)c5ccccc5)cc4)nc(-c4cccc5sc6ccccc6c45)n3)c3c2oc2ccccc23)c([2H])c1[2H] ; c1ccc(-c2ccc(-c3nc(-c4ccc([Si](c5ccccc5)(c5ccccc5)c5ccccc5)cc4)nc(-c4ccc5c(c4)sc4ccccc45)n3)c3c2oc2ccccc23)cc1 ; c1ccc(-c2nc(-c3ccccc3[Si](c3ccccc3)(c3ccccc3)c3ccccc3)nc(-c3cccc4oc5ccccc5c34)n2)cc1]

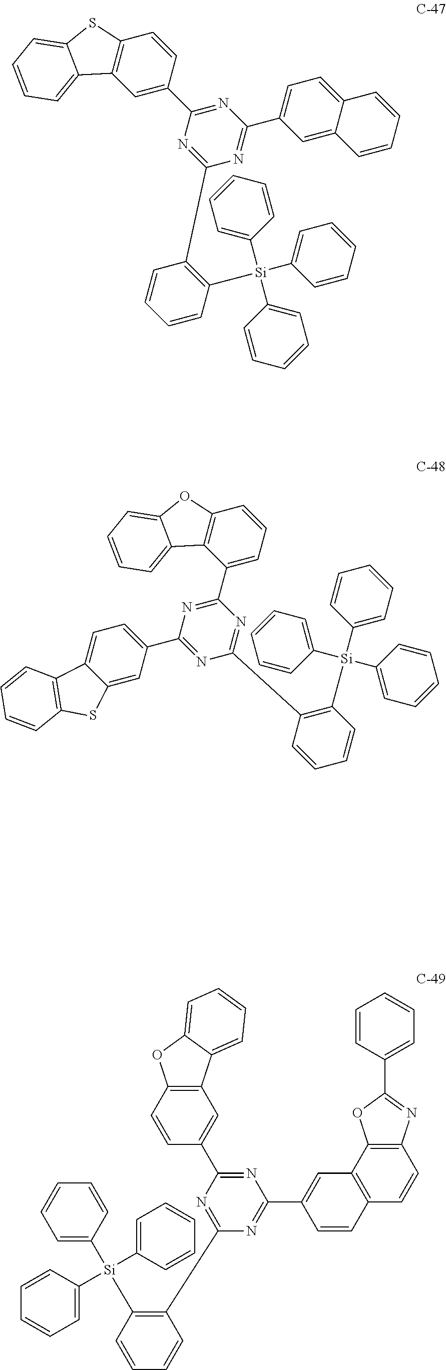 [SMILES: c1ccc([Si](c2ccccc2)(c2ccccc2)c2ccccc2-c2nc(-c3ccc4ccccc4c3)nc(-c3ccc4sc5ccccc5c4c3)n2)cc1 ; c1ccc([Si](c2ccccc2)(c2ccccc2)c2ccccc2-c2nc(-c3ccc4c(c3)sc3ccccc34)nc(-c3cccc4oc5ccccc5c34)n2)cc1 ; c1ccc(-c2nc3ccc4ccc(-c5nc(-c6ccc7oc8ccccc8c7c6)nc(-c6ccccc6[Si](c6ccccc6)(c6ccccc6)c6ccccc6)n5)cc4c3o2)cc1]

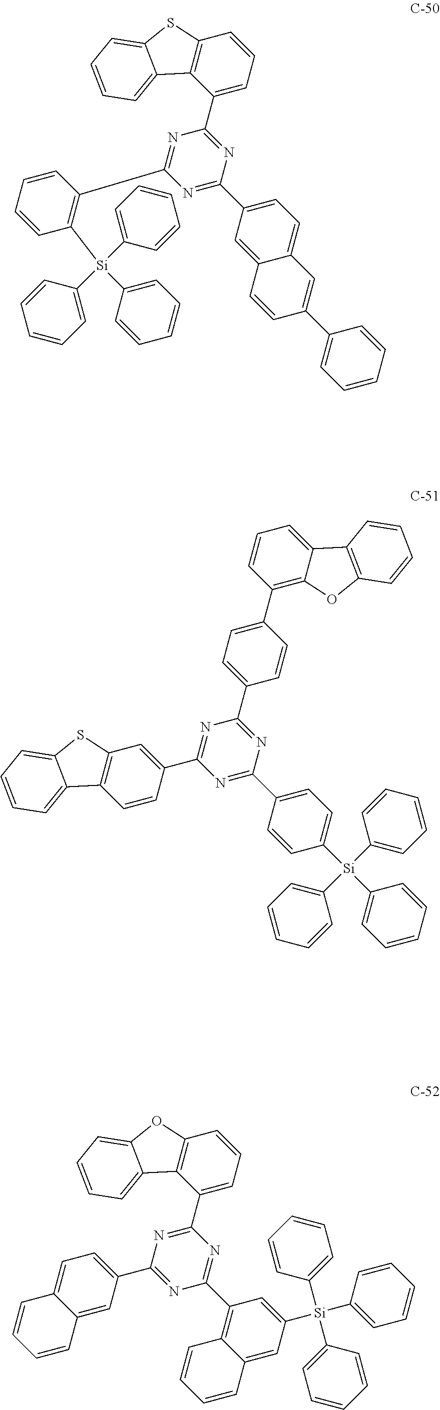 [SMILES: c1ccc(-c2ccc3cc(-c4nc(-c5ccccc5[Si](c5ccccc5)(c5ccccc5)c5ccccc5)nc(-c5cccc6sc7ccccc7c56)n4)ccc3c2)cc1 ; c1ccc([Si](c2ccccc2)(c2ccccc2)c2ccc(-c3nc(-c4ccc(-c5cccc6c5oc5ccccc56)cc4)nc(-c4ccc5c(c4)sc4ccccc45)n3)cc2)cc1 ; c1ccc([Si](c2ccccc2)(c2ccccc2)c2cc(-c3nc(-c4ccc5ccccc5c4)nc(-c4cccc5oc6ccccc6c45)n3)c3ccccc3c2)cc1]

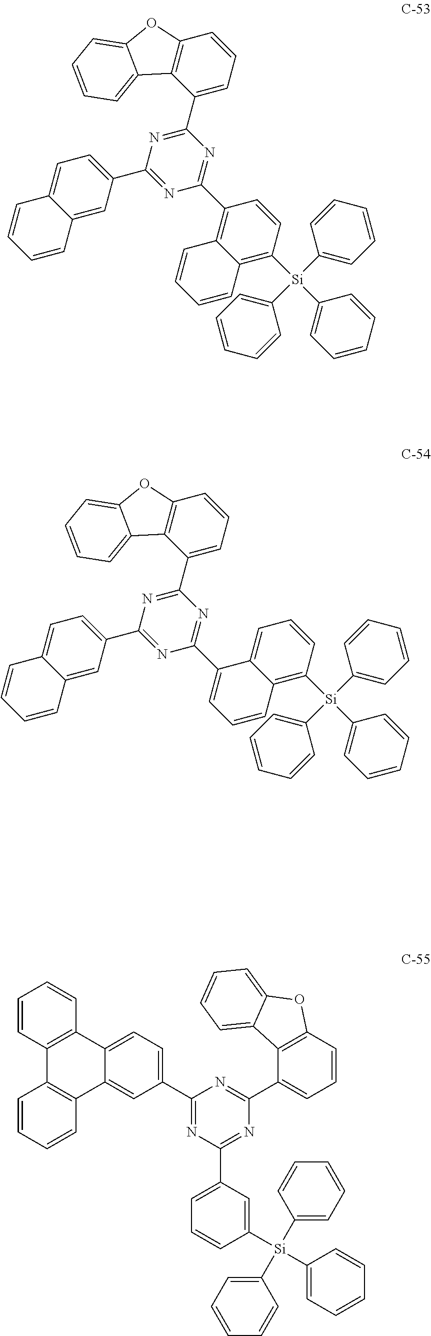 [SMILES: c1ccc([Si](c2ccccc2)(c2ccccc2)c2ccc(-c3nc(-c4ccc5ccccc5c4)nc(-c4cccc5oc6ccccc6c45)n3)c3ccccc23)cc1 ; c1ccc([Si](c2ccccc2)(c2ccccc2)c2cccc3c(-c4nc(-c5ccc6ccccc6c5)nc(-c5cccc6oc7ccccc7c56)n4)cccc23)cc1 ; c1ccc([Si](c2ccccc2)(c2ccccc2)c2cccc(-c3nc(-c4ccc5c6ccccc6c6ccccc6c5c4)nc(-c4cccc5oc6ccccc6c45)n3)c2)cc1]

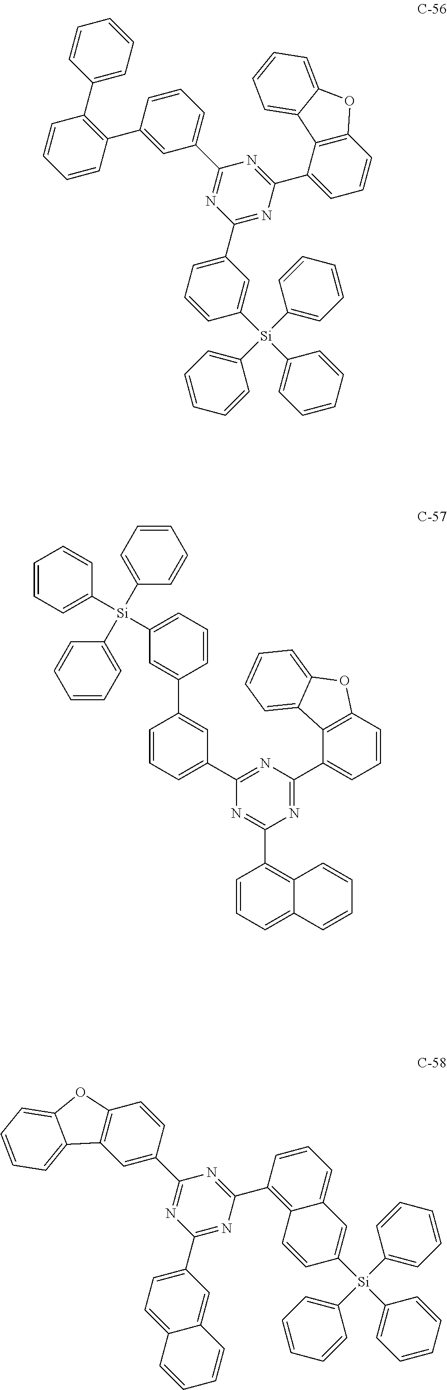 [SMILES: c1ccc(-c2ccccc2-c2cccc(-c3nc(-c4cccc([Si](c5ccccc5)(c5ccccc5)c5ccccc5)c4)nc(-c4cccc5oc6ccccc6c45)n3)c2)cc1 ; c1ccc([Si](c2ccccc2)(c2ccccc2)c2cccc(-c3cccc(-c4nc(-c5cccc6ccccc56)nc(-c5cccc6oc7ccccc7c56)n4)c3)c2)cc1 ; c1ccc([Si](c2ccccc2)(c2ccccc2)c2ccc3c(-c4nc(-c5ccc6ccccc6c5)nc(-c5ccc6oc7ccccc7c6c5)n4)cccc3c2)cc1]

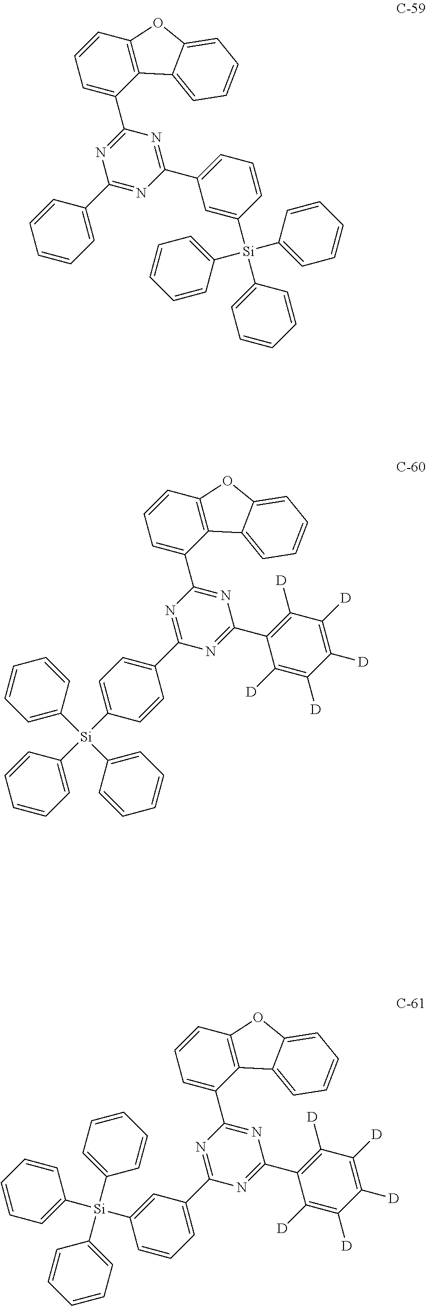

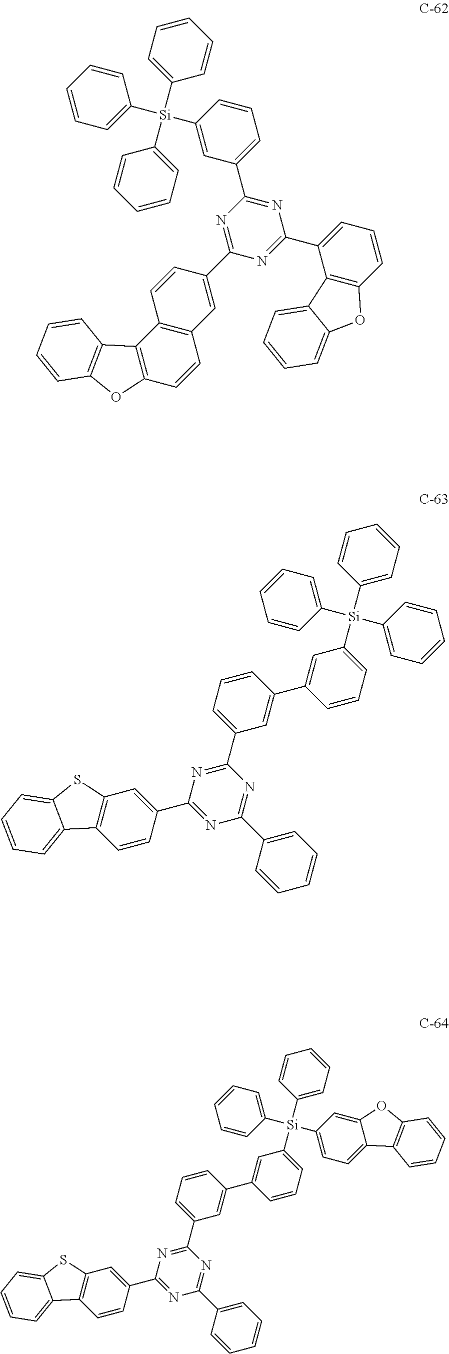 [SMILES: c1ccc([Si](c2ccccc2)(c2ccccc2)c2cccc(-c3nc(-c4ccc5c(ccc6oc7ccccc7c65)c4)nc(-c4cccc5oc6ccccc6c45)n3)c2)cc1 ; c1ccc(-c2nc(-c3cccc(-c4cccc([Si](c5ccccc5)(c5ccccc5)c5ccccc5)c4)c3)nc(-c3ccc4c(c3)sc3ccccc34)n2)cc1 ; c1ccc(-c2nc(-c3cccc(-c4cccc([Si](c5ccccc5)(c5ccccc5)c5ccc6c(c5)oc5ccccc56)c4)c3)nc(-c3ccc4c(c3)sc3ccccc34)n2)cc1]

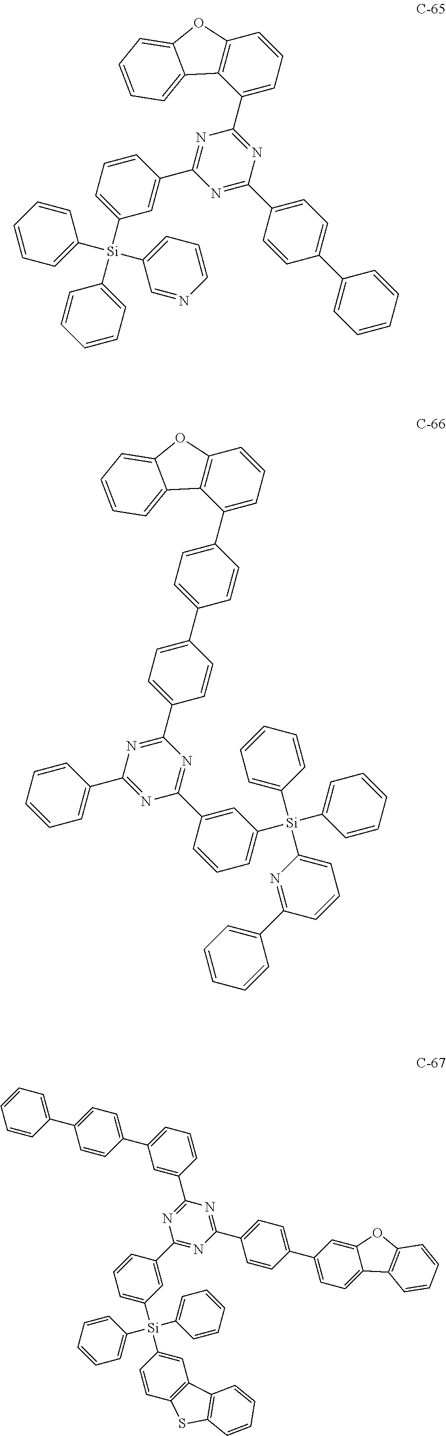 [SMILES: c1ccc(-c2ccc(-c3nc(-c4cccc([Si](c5ccccc5)(c5ccccc5)c5cccnc5)c4)nc(-c4cccc5oc6ccccc6c45)n3)cc2)cc1 ; c1ccc(-c2cccc([Si](c3ccccc3)(c3ccccc3)c3cccc(-c4nc(-c5ccccc5)nc(-c5ccc(-c6ccc(-c7cccc8oc9ccccc9c78)cc6)cc5)n4)c3)n2)cc1 ; c1ccc(-c2ccc(-c3cccc(-c4nc(-c5ccc(-c6ccc7c(c6)oc6ccccc67)cc5)nc(-c5cccc([Si](c6ccccc6)(c6ccccc6)c6ccc7sc8ccccc8c7c6)c5)n4)c3)cc2)cc1]

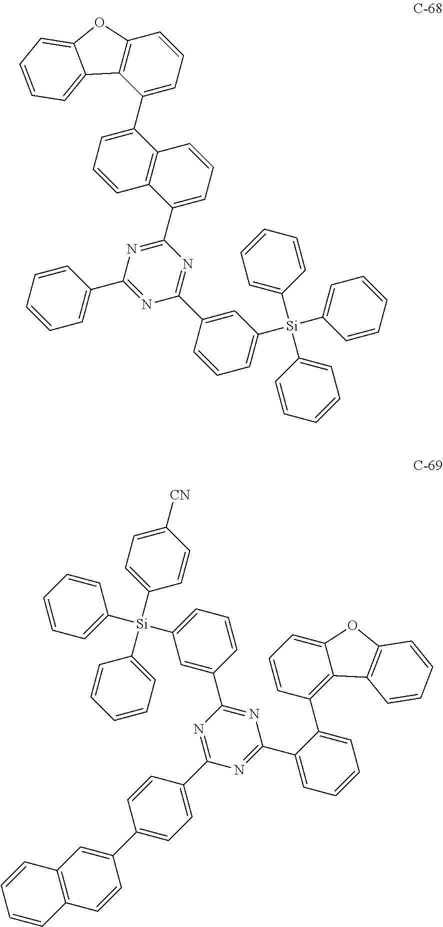 [SMILES: c1ccc(-c2nc(-c3cccc([Si](c4ccccc4)(c4ccccc4)c4ccccc4)c3)nc(-c3cccc4c(-c5cccc6oc7ccccc7c56)cccc34)n2)cc1 ; N#Cc1ccc([Si](c2ccccc2)(c2ccccc2)c2cccc(-c3nc(-c4ccc(-c5ccc6ccccc6c5)cc4)nc(-c4ccccc4-c4cccc5oc6ccccc6c45)n3)c2)cc1]

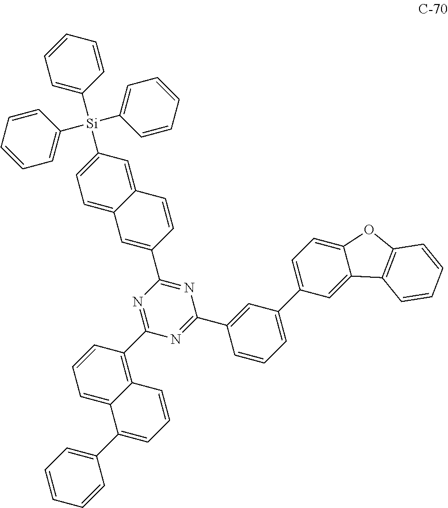 [SMILES: c1ccc(-c2cccc3c(-c4nc(-c5cccc(-c6ccc7oc8ccccc8c7c6)c5)nc(-c5ccc6cc([Si](c7ccccc7)(c7ccccc7)c7ccccc7)ccc6c5)n4)cccc23)cc1]

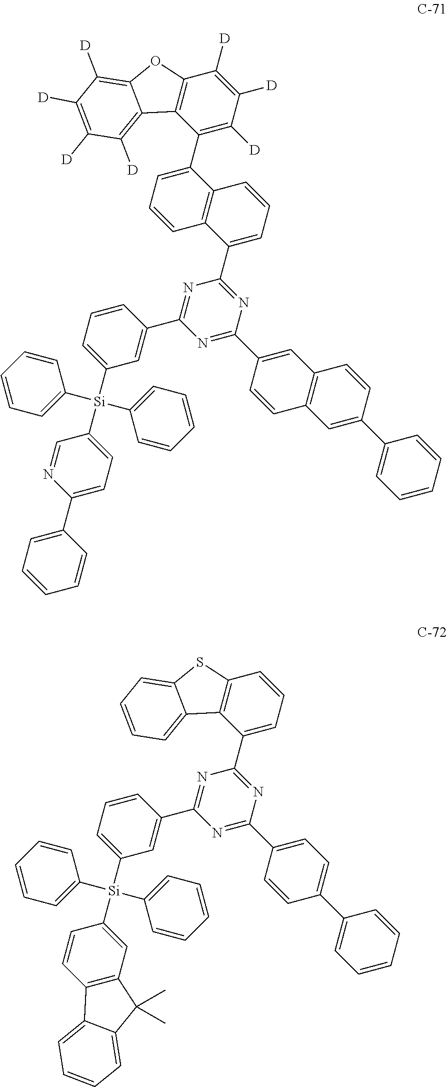 [SMILES: [2H]c1c([2H])c([2H])c2c(oc3c([2H])c([2H])c([2H])c(-c4cccc5c(-c6nc(-c7cccc([Si](c8ccccc8)(c8ccccc8)c8ccc(-c9ccccc9)nc8)c7)nc(-c7ccc8cc(-c9ccccc9)ccc8c7)n6)cccc45)c32)c1[2H] ; CC1(C)c2ccccc2-c2ccc([Si](c3ccccc3)(c3ccccc3)c3cccc(-c4nc(-c5ccc(-c6ccccc6)cc5)nc(-c5cccc6sc7ccccc7c56)n4)c3)cc21]

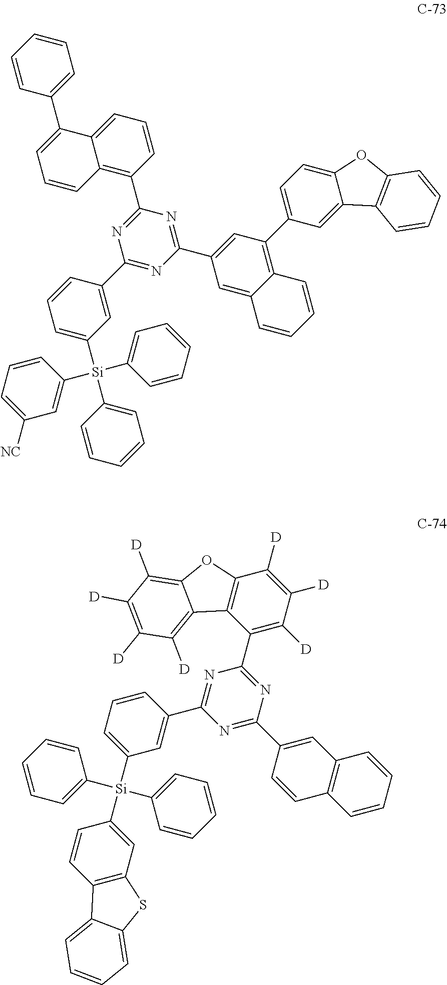 [SMILES: N#Cc1cccc([Si](c2ccccc2)(c2ccccc2)c2cccc(-c3nc(-c4cc(-c5ccc6oc7ccccc7c6c5)c5ccccc5c4)nc(-c4cccc5c(-c6ccccc6)cccc45)n3)c2)c1 ; [2H]c1c([2H])c([2H])c2c(oc3c([2H])c([2H])c([2H])c(-c4nc(-c5cccc([Si](c6ccccc6)(c6ccccc6)c6ccc7c(c6)sc6ccccc67)c5)nc(-c5ccc6ccccc6c5)n4)c32)c1[2H]]

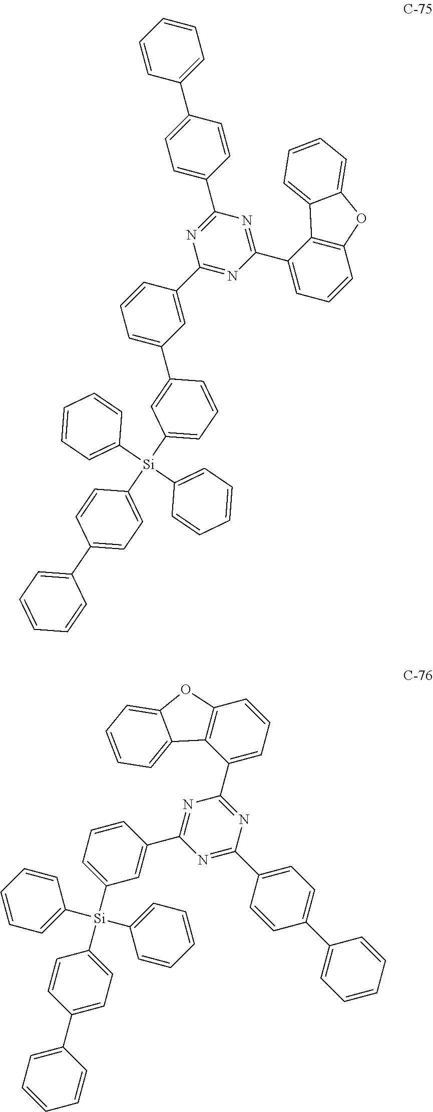 [SMILES: c1ccc(-c2ccc(-c3nc(-c4cccc(-c5cccc([Si](c6ccccc6)(c6ccccc6)c6ccc(-c7ccccc7)cc6)c5)c4)nc(-c4cccc5oc6ccccc6c45)n3)cc2)cc1 ; c1ccc(-c2ccc(-c3nc(-c4cccc([Si](c5ccccc5)(c5ccccc5)c5ccc(-c6ccccc6)cc5)c4)nc(-c4cccc5oc6ccccc6c45)n3)cc2)cc1]

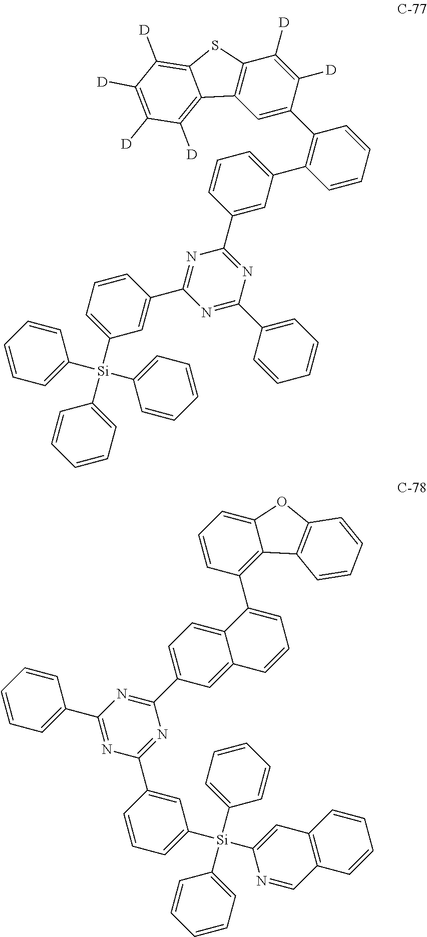 [SMILES: [2H]c1c(-c2ccccc2-c2cccc(-c3nc(-c4ccccc4)nc(-c4cccc([Si](c5ccccc5)(c5ccccc5)c5ccccc5)c4)n3)c2)cc2c(sc3c([2H])c([2H])c([2H])c([2H])c32)c1[2H] ; c1ccc(-c2nc(-c3cccc([Si](c4ccccc4)(c4ccccc4)c4cc5ccccc5cn4)c3)nc(-c3ccc4c(-c5cccc6oc7ccccc7c56)cccc4c3)n2)cc1]

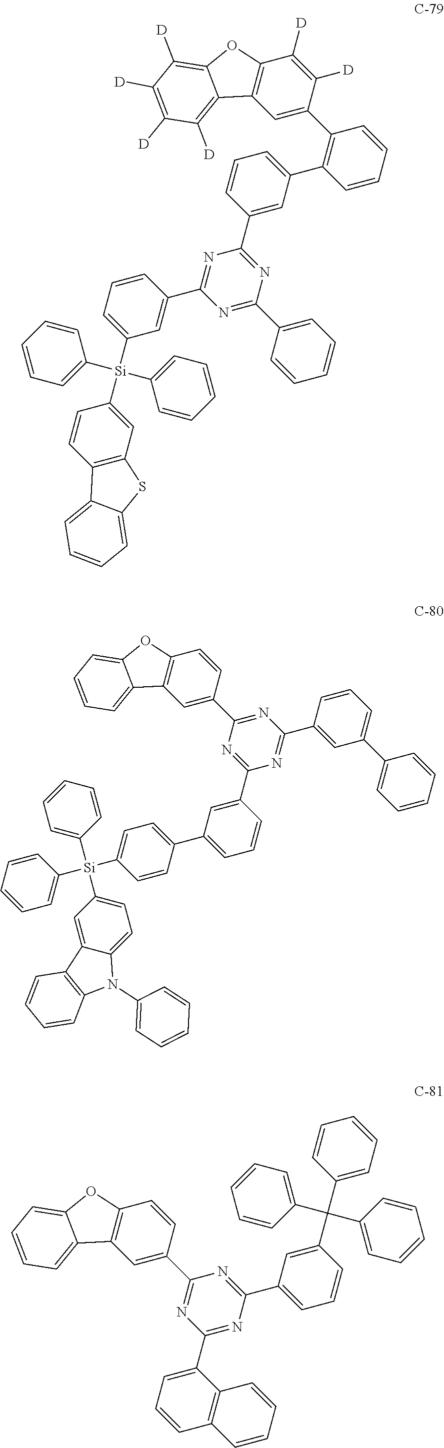 [SMILES: [2H]c1c(-c2ccccc2-c2cccc(-c3nc(-c4ccccc4)nc(-c4cccc([Si](c5ccccc5)(c5ccccc5)c5ccc6c(c5)sc5ccccc56)c4)n3)c2)cc2c(oc3c([2H])c([2H])c([2H])c([2H])c32)c1[2H] ; c1ccc(-c2cccc(-c3nc(-c4cccc(-c5ccc([Si](c6ccccc6)(c6ccccc6)c6ccc7c(c6)c6ccccc6n7-c6ccccc6)cc5)c4)nc(-c4ccc5oc6ccccc6c5c4)n3)c2)cc1 ; c1ccc(C(c2ccccc2)(c2ccccc2)c2cccc(-c3nc(-c4ccc5oc6ccccc6c5c4)nc(-c4cccc5ccccc45)n3)c2)cc1]

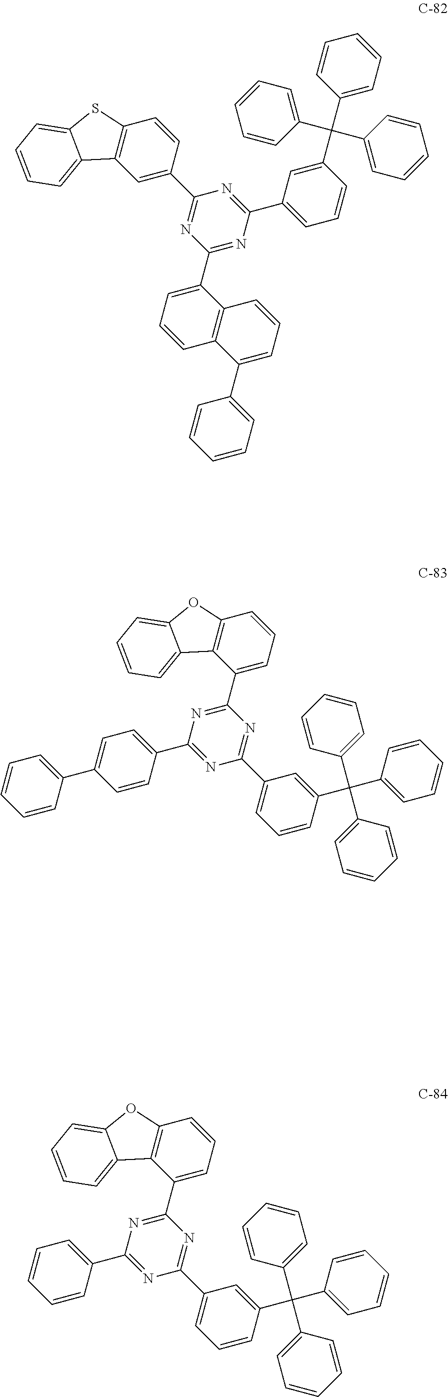 [SMILES: c1ccc(-c2cccc3c(-c4nc(-c5cccc(C(c6ccccc6)(c6ccccc6)c6ccccc6)c5)nc(-c5ccc6sc7ccccc7c6c5)n4)cccc23)cc1 ; c1ccc(-c2ccc(-c3nc(-c4cccc(C(c5ccccc5)(c5ccccc5)c5ccccc5)c4)nc(-c4cccc5oc6ccccc6c45)n3)cc2)cc1 ; c1ccc(-c2nc(-c3cccc(C(c4ccccc4)(c4ccccc4)c4ccccc4)c3)nc(-c3cccc4oc5ccccc5c34)n2)cc1]

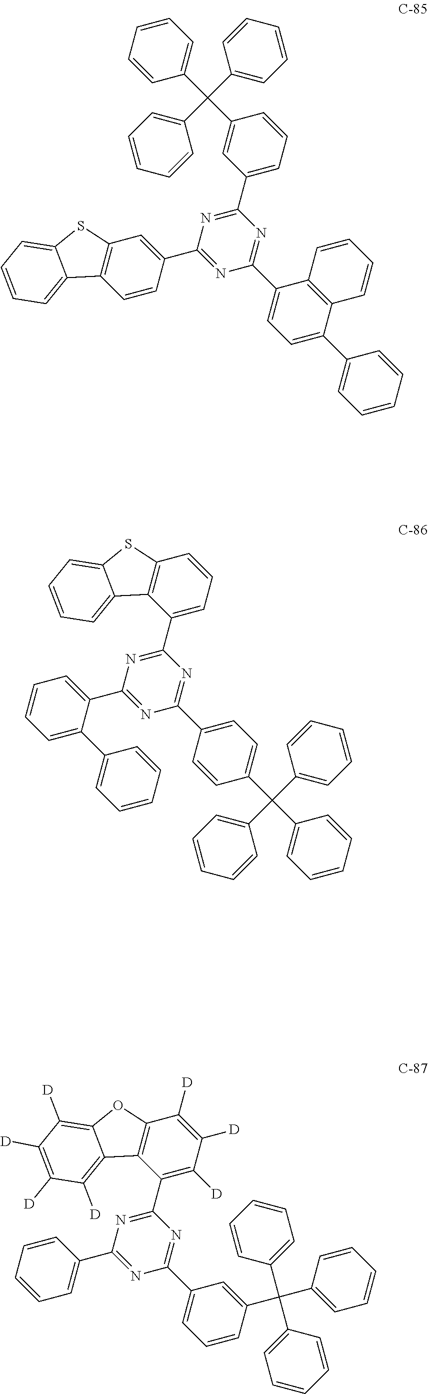 [SMILES: c1ccc(-c2ccc(-c3nc(-c4cccc(C(c5ccccc5)(c5ccccc5)c5ccccc5)c4)nc(-c4ccc5c(c4)sc4ccccc45)n3)c3ccccc23)cc1 ; c1ccc(-c2ccccc2-c2nc(-c3ccc(C(c4ccccc4)(c4ccccc4)c4ccccc4)cc3)nc(-c3cccc4sc5ccccc5c34)n2)cc1 ; [2H]c1c([2H])c([2H])c2c(oc3c([2H])c([2H])c([2H])c(-c4nc(-c5ccccc5)nc(-c5cccc(C(c6ccccc6)(c6ccccc6)c6ccccc6)c5)n4)c32)c1[2H]]

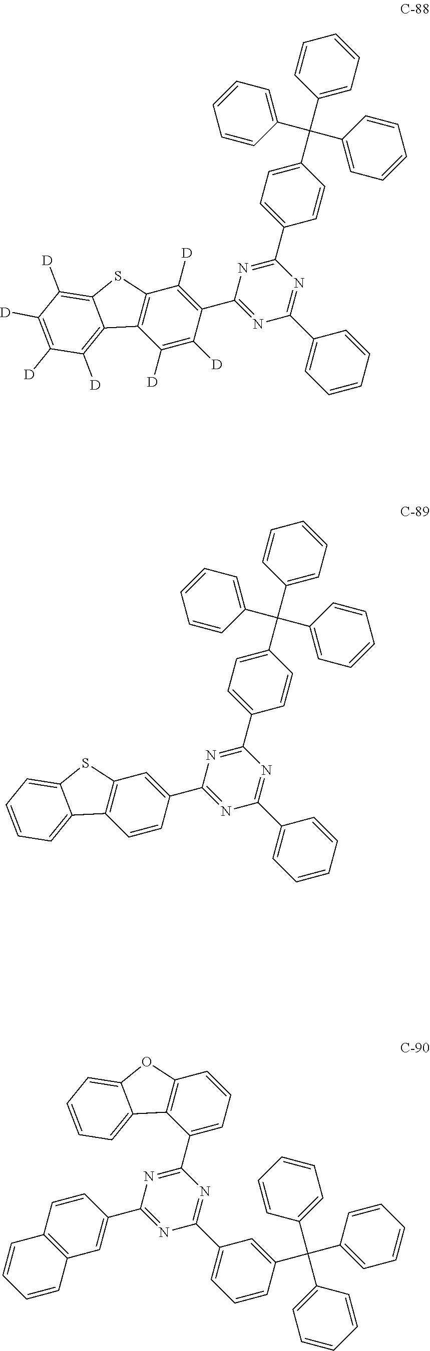 [SMILES: [2H]c1c([2H])c([2H])c2c(sc3c([2H])c(-c4nc(-c5ccccc5)nc(-c5ccc(C(c6ccccc6)(c6ccccc6)c6ccccc6)cc5)n4)c([2H])c([2H])c32)c1[2H] ; c1ccc(-c2nc(-c3ccc(C(c4ccccc4)(c4ccccc4)c4ccccc4)cc3)nc(-c3ccc4c(c3)sc3ccccc34)n2)cc1 ; c1ccc(C(c2ccccc2)(c2ccccc2)c2cccc(-c3nc(-c4ccc5ccccc5c4)nc(-c4cccc5oc6ccccc6c45)n3)c2)cc1]

The compound represented by formula 2 according to one embodiment may be produced by referring to a synthetic method known to a person skilled in the art.

According to another embodiment of the present disclosure, the present disclosure provides an organic electroluminescent compound represented by the following formula 3.

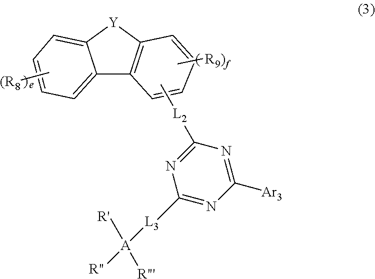

in formula 3,

Y represents O or S;

A represents Si or C;

L₂ represents a single bond, phenylene unsubstituted or substituted by deuterium, biphenylene unsubstituted or substituted by deuterium, or naphthylene unsubstituted or substituted by deuterium;

R₈ and R₉ each independently represent hydrogen or deuterium;

Ar₃ represents phenyl unsubstituted or substituted by deuterium, biphenyl unsubstituted or substituted by deuterium, terphenyl unsubstituted or substituted by deuterium, naphthyl unsubstituted or substituted by deuterium, triphenylenyl unsubstituted or substituted by deuterium, phenanthrenyl unsubstituted or substituted by deuterium, or a combination thereof;

L₃ represents phenylene unsubstituted or substituted by deuterium, biphenylene unsubstituted or substituted by deuterium, or naphthylene unsubstituted or substituted by deuterium;

R′, R″, and R′″ each independently represent a substituted or unsubstituted (C6-C30)aryl or a substituted or unsubstituted (3- to 30-membered)heteroaryl;

e represents an integer of 1 to 4, and f represents an integer of 1 to 3; and

when e and f are an integer of 2 or more, each of R₈ and each of R₉ may be the same or different.

According to one embodiment, the organic electroluminescent compound represented by formula 3 may be more specifically illustrated by the following compounds, but is not limited thereto.

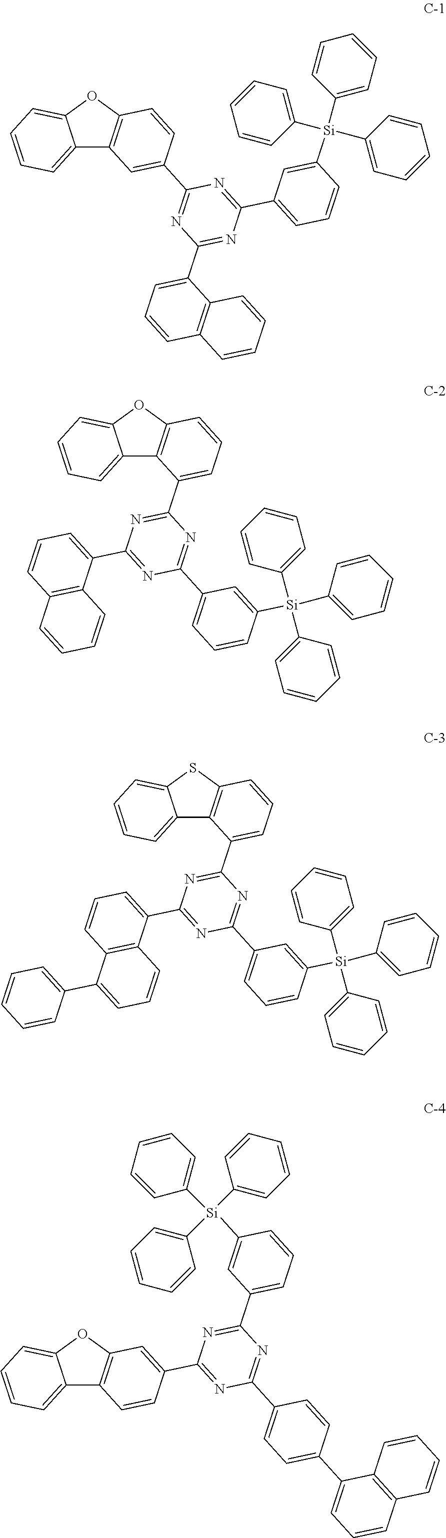 [SMILES: c1ccc([Si](c2ccccc2)(c2ccccc2)c2cccc(-c3nc(-c4ccc5oc6ccccc6c5c4)nc(-c4cccc5ccccc45)n3)c2)cc1 ; c1ccc([Si](c2ccccc2)(c2ccccc2)c2cccc(-c3nc(-c4cccc5ccccc45)nc(-c4cccc5oc6ccccc6c45)n3)c2)cc1 ; c1ccc(-c2cccc3c(-c4nc(-c5cccc([Si](c6ccccc6)(c6ccccc6)c6ccccc6)c5)nc(-c5cccc6sc7ccccc7c56)n4)cccc23)cc1 ; c1ccc([Si](c2ccccc2)(c2ccccc2)c2cccc(-c3nc(-c4ccc(-c5cccc6ccccc56)cc4)nc(-c4ccc5c(c4)oc4ccccc45)n3)c2)cc1]

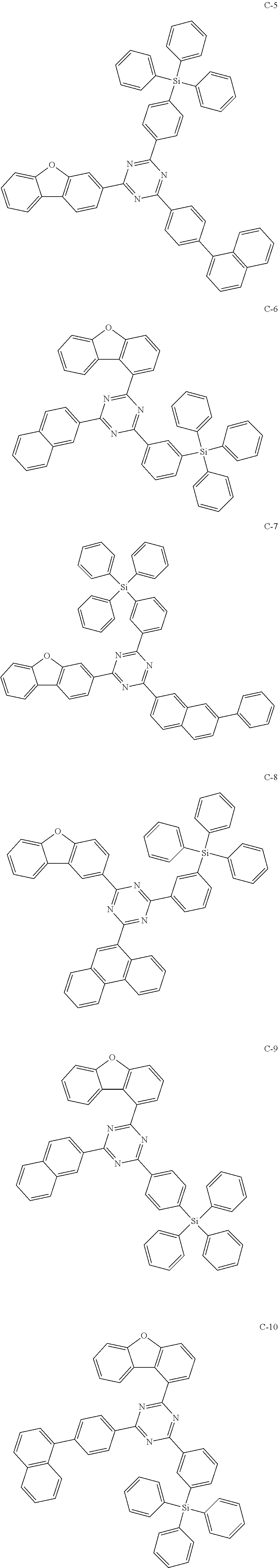

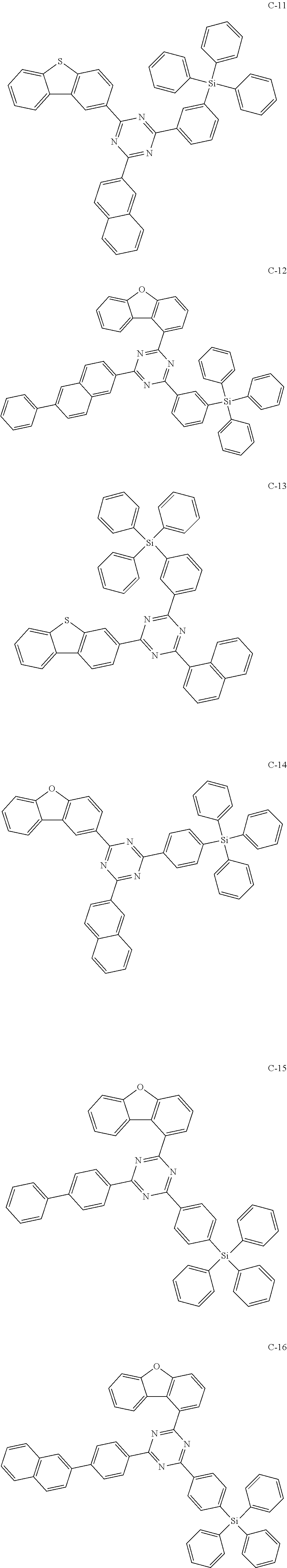

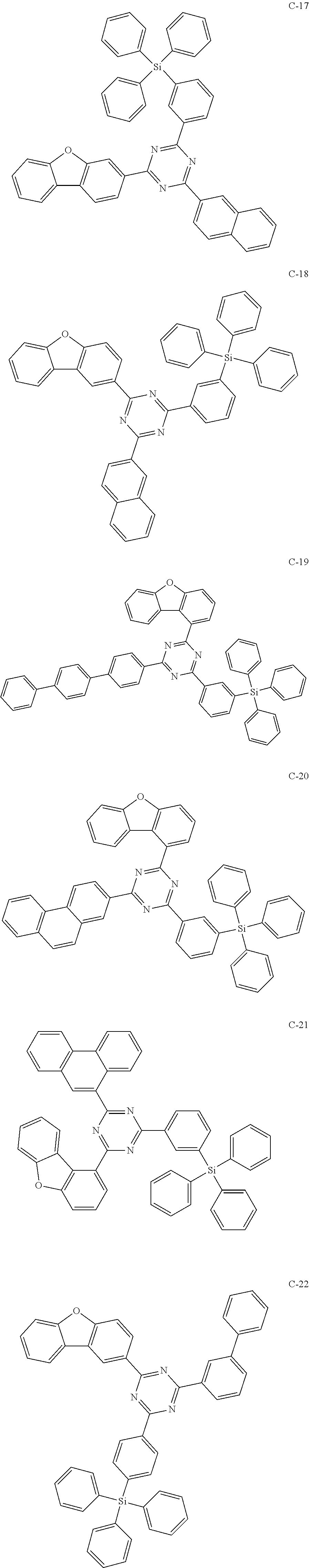

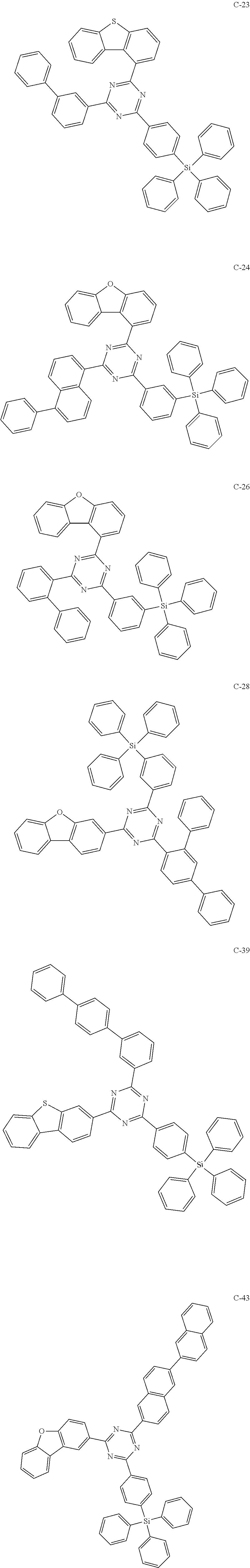

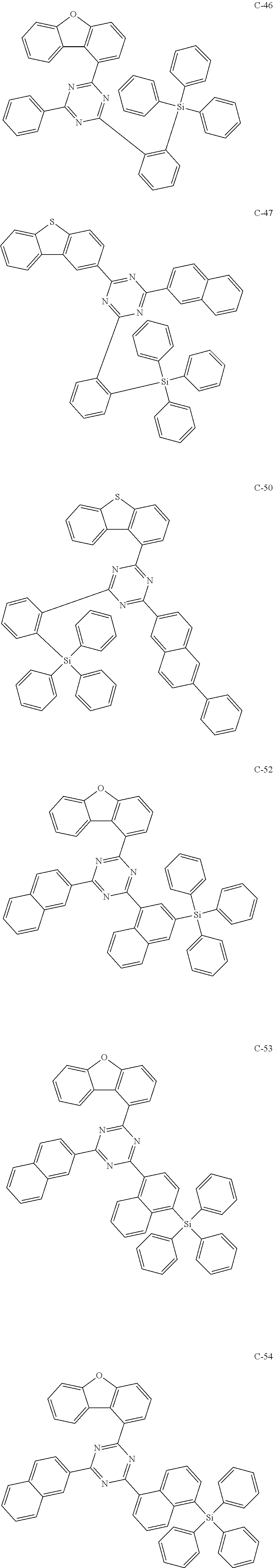

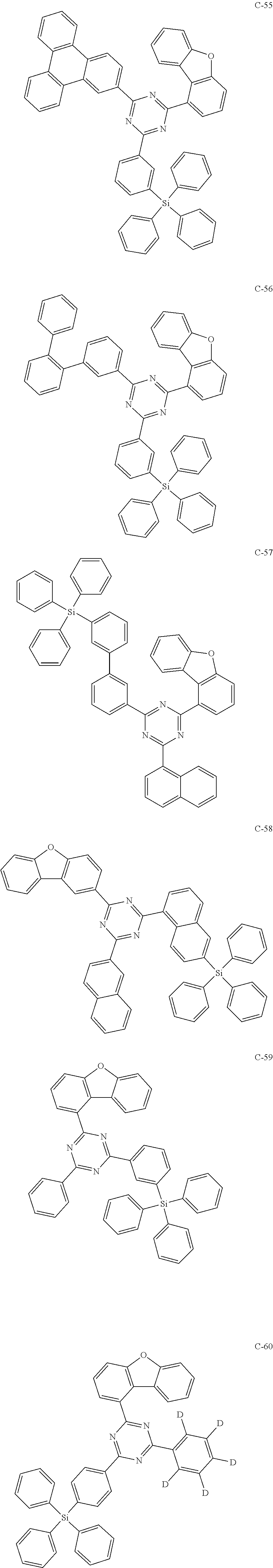

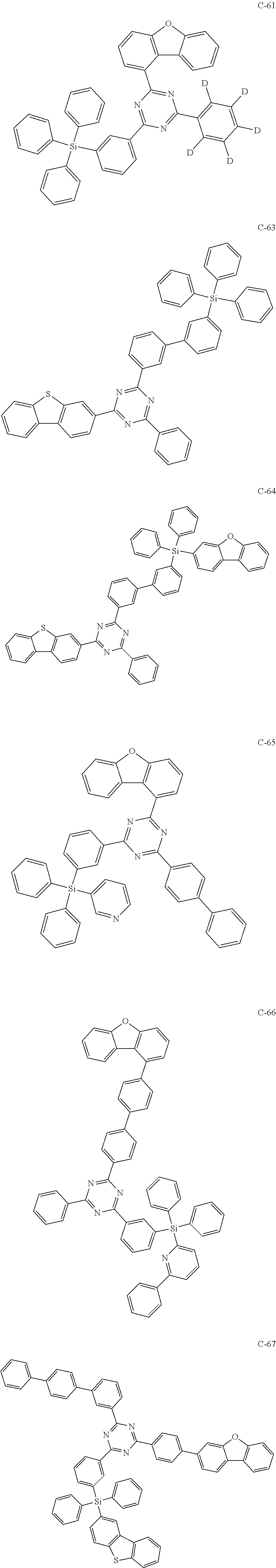

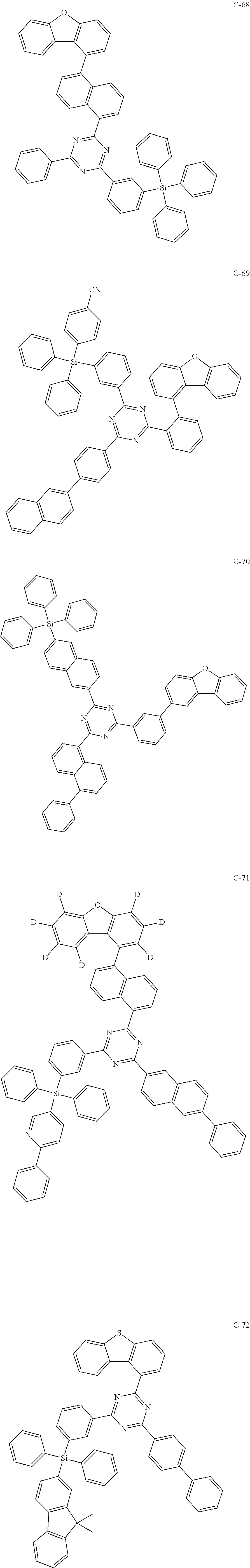

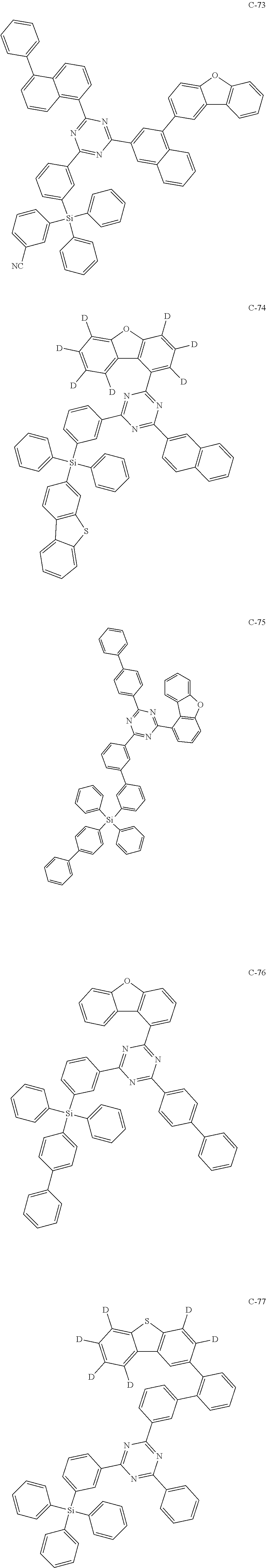

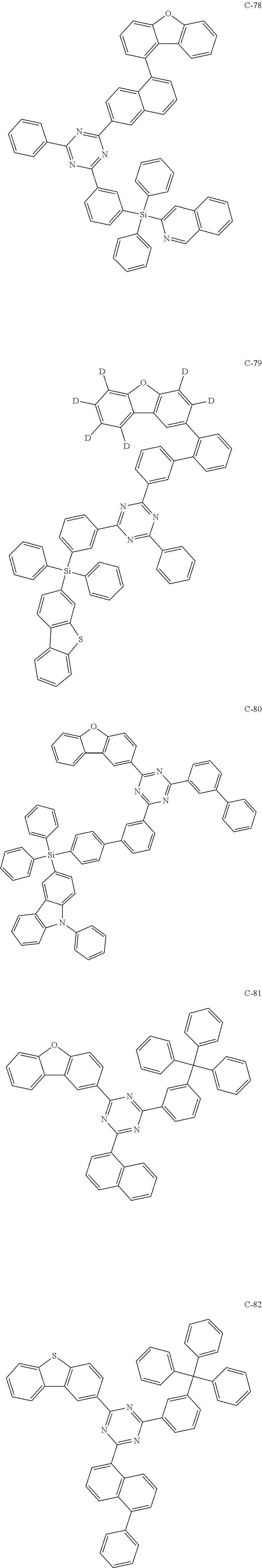

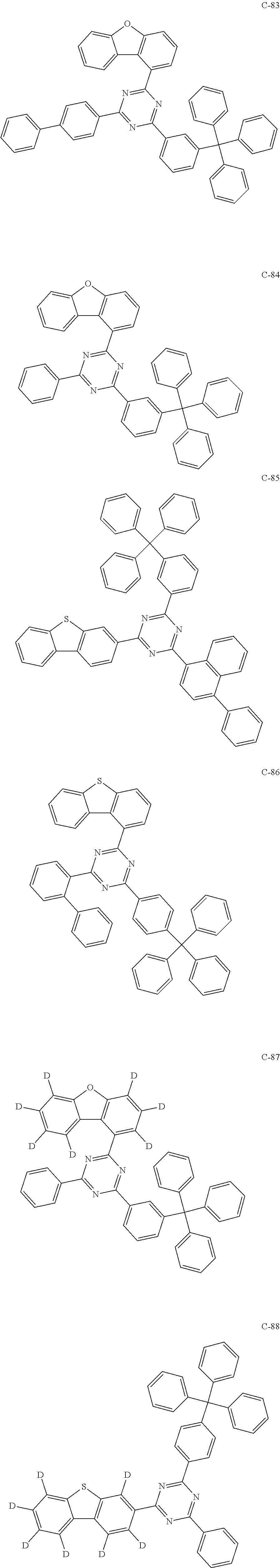

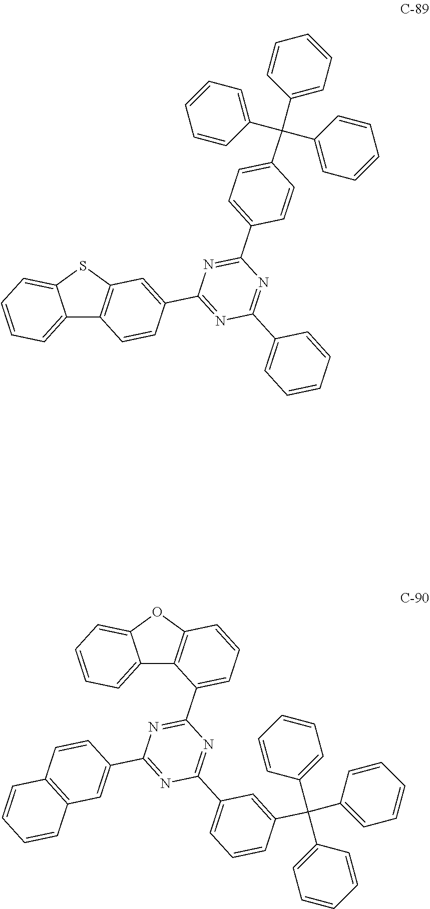

Hereinafter, the aforementioned plurality of host materials, and/or an organic electroluminescent device to which the organic electroluminescent compound is applied, will be described.

The organic electroluminescent device according to one embodiment includes a first electrode; a second electrode; and at least one organic layer interposed between the first electrode and the second electrode. The organic layer may include a light-emitting layer, and the light-emitting layer may comprise a plurality of host materials comprising at least one first host material represented by formula 1 and at least one second host material represented by formula 2. According to another embodiment, the light-emitting layer may comprise an organic electroluminescent compound represented by formula 3 alone.

According to one embodiment, the organic electroluminescent material of the present disclosure comprises at least one compound(s) of compounds H1-1 to H1-115 as the first host material and at least one compound(s) of compounds C-1 to C-90 as the second host material, and the plurality of host materials may be included in the same organic layer, for example a light-emitting layer, or may be included in different light-emitting layers, respectively.

According to another embodiment, the organic electroluminescent material of the present disclosure comprises an organic electroluminescent compound represented by formula 3 alone or in combination of two or more, and the organic electroluminescent material may be included in the organic layer of an organic electroluminescent device, e.g., a light-emitting layer or an electron buffer layer.

The organic layer may further comprise at least one layer selected from a hole injection layer, a hole transport layer, a hole auxiliary layer, a light-emitting auxiliary layer, an electron transport layer, an electron injection layer, an interlayer, a hole blocking layer, and an electron blocking layer, in addition to the light-emitting layer and the electron buffer layer. The organic layer may further comprise an amine-based compound and/or an azine-based compound other than the light-emitting material according to the present disclosure. Specifically, the hole injection layer, the hole transport layer, the hole auxiliary layer, the light-emitting layer, the light-emitting auxiliary layer, or the electron blocking layer may contain the amine-based compound, e.g., an arylamine-based compound and a styrylarylamine-based compound, etc., as a hole injection material, a hole transport material, a hole auxiliary material, a light-emitting material, a light-emitting auxiliary material, or an electron blocking material. Also, the electron transport layer, the electron injection layer, the electron buffer layer, or the hole blocking layer may contain the azine-based compound as an electron transport material, an electron injection material, an electron buffer material, or a hole blocking material. Also, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

The organic electroluminescent compound and the plurality of host materials according to one embodiment may be used as light-emitting materials for a white organic light-emitting device. The white organic light-emitting device has suggested various structures such as a parallel side-by-side arrangement method, a stacking arrangement method, or CCM (color conversion material) method, etc., according to the arrangement of R (Red), G (Green), YG (yellowish green), or B (blue) light-emitting units. In addition, the organic electroluminescent material according to one embodiment may also be applied to the organic electroluminescent device comprising a QD (quantum dot).

One of the first electrode and the second electrode may be an anode and the other may be a cathode. Wherein, the first electrode and the second electrode may each be formed as a transmissive conductive material, a transflective conductive material, or a reflective conductive material. The organic electroluminescent device may be a top emission type, a bottom emission type, or a both-sides emission type according to the kinds of the material forming the first electrode and the second electrode.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. Also, the hole injection layer may be doped as a p-dopant. Also, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may be multi-layers, and wherein each layer may use a plurality of compounds.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer may be placed between the electron transport layer (or electron injection layer) and the light-emitting layer, and blocks the arrival of holes to the cathode, thereby improving the probability of recombination of electrons and holes in the light-emitting layer. The hole blocking layer or the electron transport layer may also be multi-layers, wherein each layer may use a plurality of compounds. Also, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron blocking layer. The light-emitting auxiliary layer, the hole auxiliary layer, or the electron blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiO_{X} (1≤X≤2), AlO_{X} (1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. Also, a reductive dopant layer may be employed as a charge generating layer to prepare an organic electroluminescent device having two or more light-emitting layers and emitting white light.

An organic electroluminescent device according to one embodiment may further comprise at least one dopant in the light-emitting layer.

The dopant comprised in the organic electroluminescent device of the present disclosure may be at least one phosphorescent or fluorescent dopant, preferably a phosphorescent dopant. The phosphorescent dopant material applied to the organic electroluminescent device of the present disclosure is not particularly limited, but may be preferably a metallated complex compound(s) of a metal atom(s) selected from iridium (Ir), osmium (Os), copper (Cu), and platinum (Pt), more preferably an ortho-metallated complex compound(s) of a metal atom(s) selected from iridium (Ir), osmium (Os), copper (Cu), and platinum (Pt), and even more preferably ortho-metallated iridium complex compound(s).

The dopant comprised in the organic electroluminescent device of the present disclosure may use the compound represented by the following formula 101, but is not limited thereto.

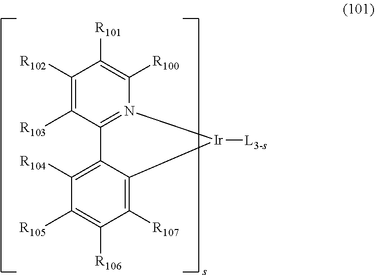

in formula 101,

L is selected from any one of the following structures 1 to 3;

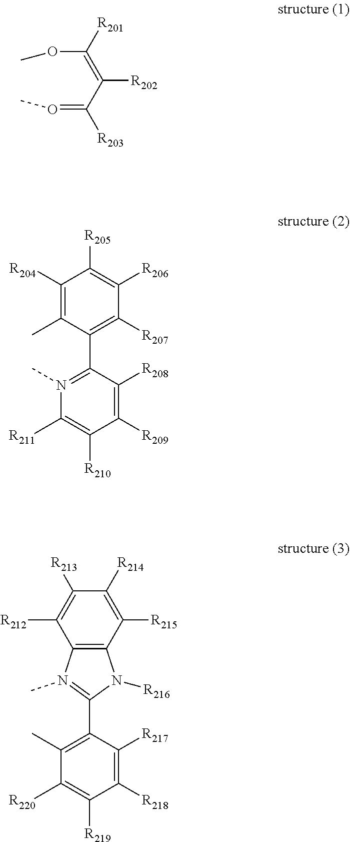

in structures 1 to 3,

R₁₀₀ to R₁₀₃ each independently represent hydrogen, deuterium, halogen, (C1-C30)alkyl unsubstituted or substituted by deuterium and/or halogen, a substituted or unsubstituted (C3-C30)cycloalkyl, a substituted or unsubstituted (C6-C30)aryl, cyano, a substituted or unsubstituted (3- to 30-membered)heteroaryl, or a substituted or unsubstituted (C1-C30)alkoxy; or may be linked to the adjacent substituents to form a ring(s), for example, to form a ring(s) with a pyridine, e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;

R₁₀₄ to R₁₀₇ each independently represent hydrogen, deuterium, halogen, (C1-C30)alkyl unsubstituted or substituted by deuterium and/or halogen, a substituted or unsubstituted (C3-C30)cycloalkyl, a substituted or unsubstituted (C6-C30)aryl, a substituted or unsubstituted (3- to 30-membered)heteroaryl, cyano, or a substituted or unsubstituted (C1-C30)alkoxy; or may be linked to an adjacent substituent(s) to form a substituted or unsubstituted ring(s), for example, to form a ring(s) with a benzene, e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;

R₂₀₁ to R₂₂₀ each independently represent hydrogen, deuterium, halogen, (C1-C30)alkyl unsubstituted or substituted by deuterium and/or halogen, a substituted or unsubstituted (C3-C30)cycloalkyl, or a substituted or unsubstituted (C6-C30)aryl; or may be linked to an adjacent substituent(s) to form a substituted or unsubstituted ring(s); and

s represents an integer of 1 to 3.

Specifically, the specific examples of the dopant compound include the following, but are not limited thereto.

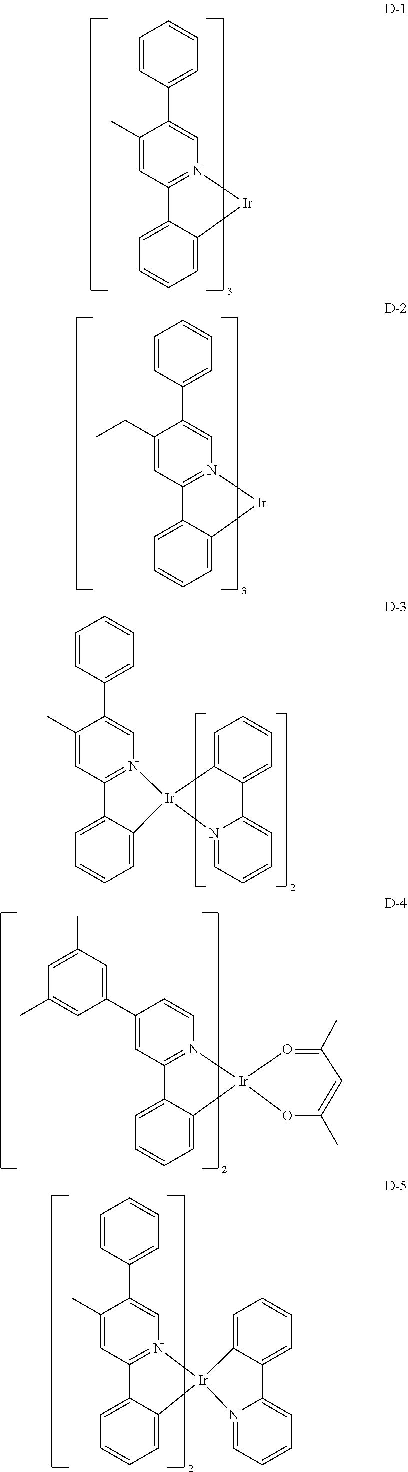

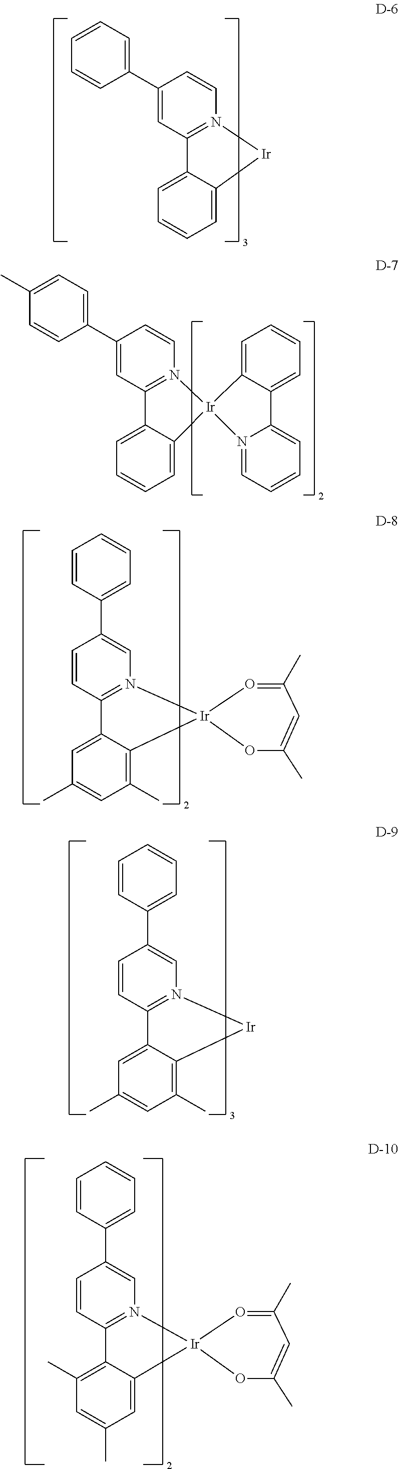

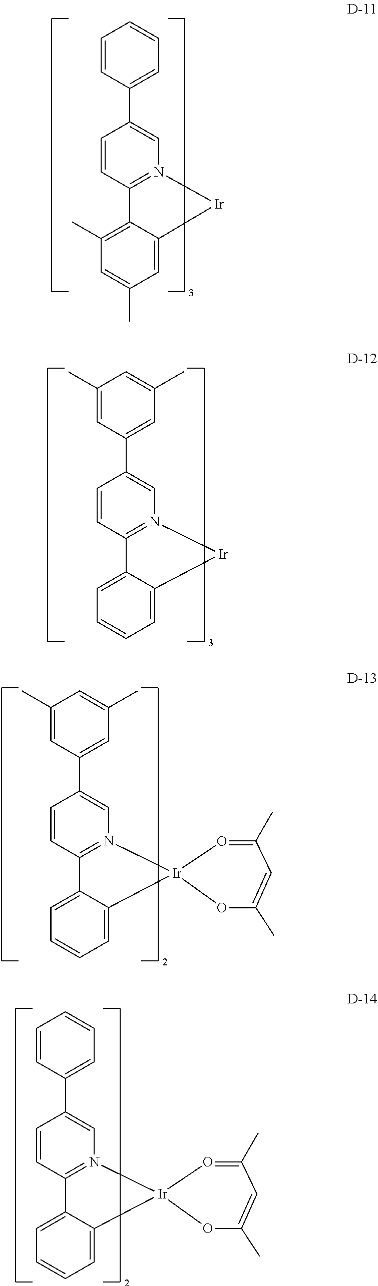

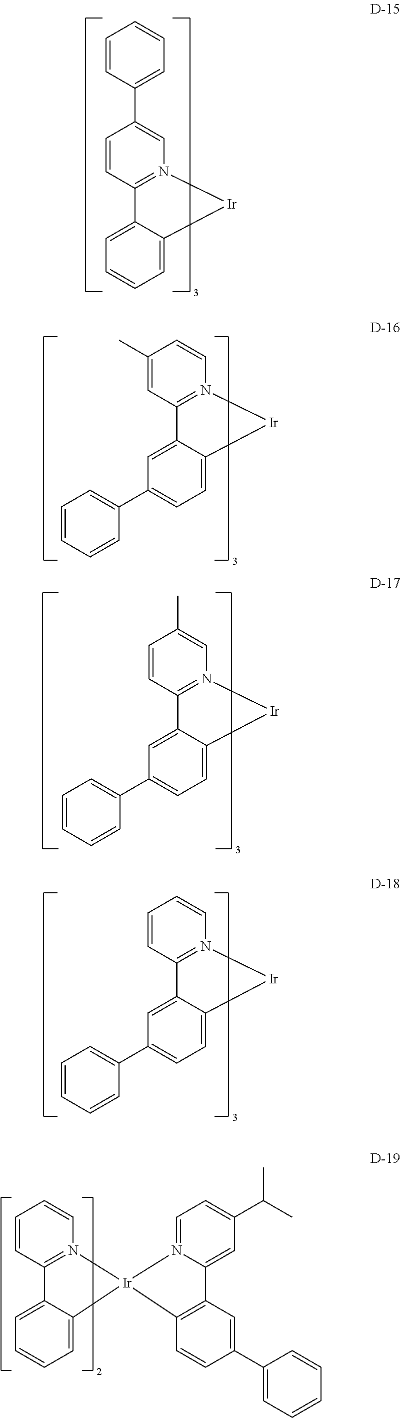

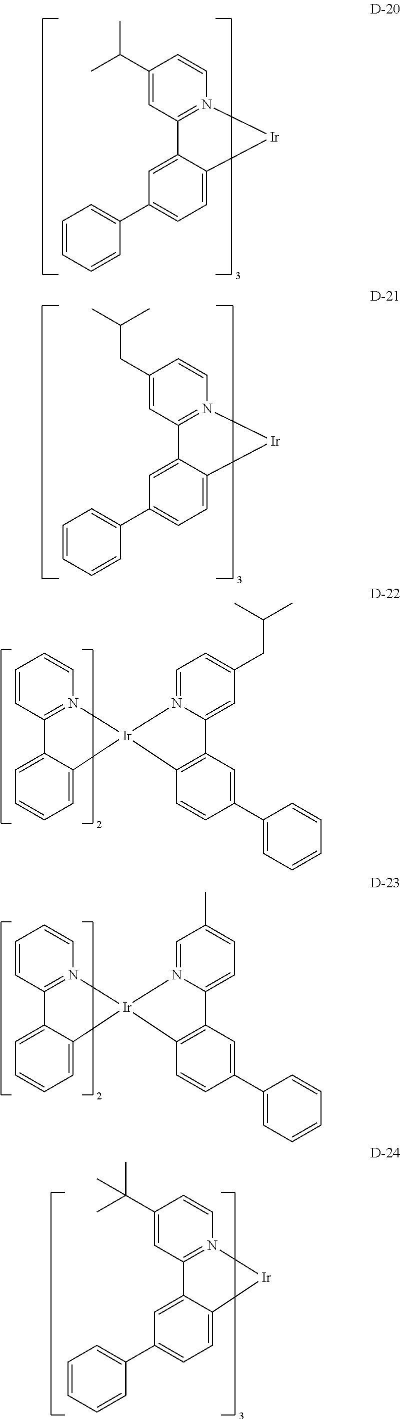

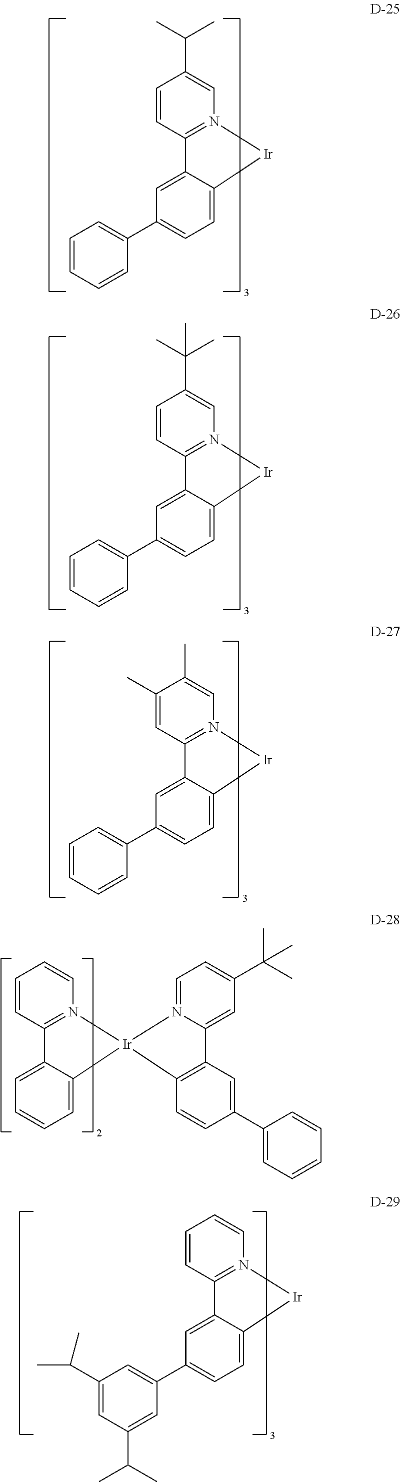

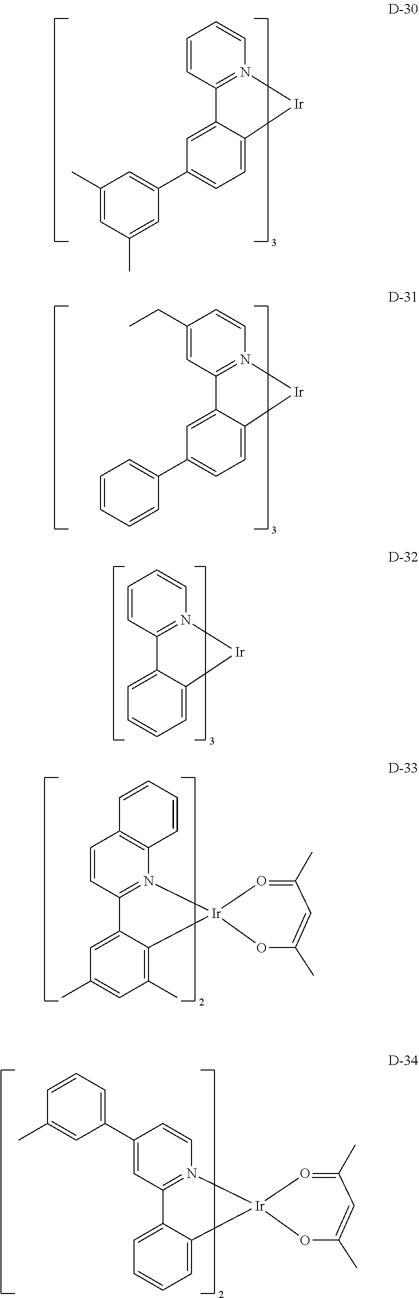

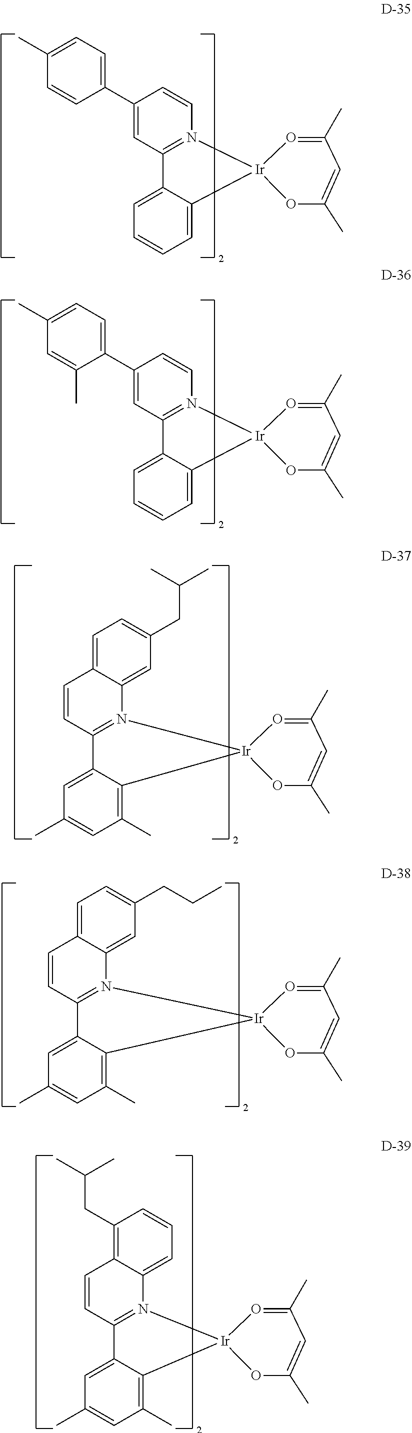

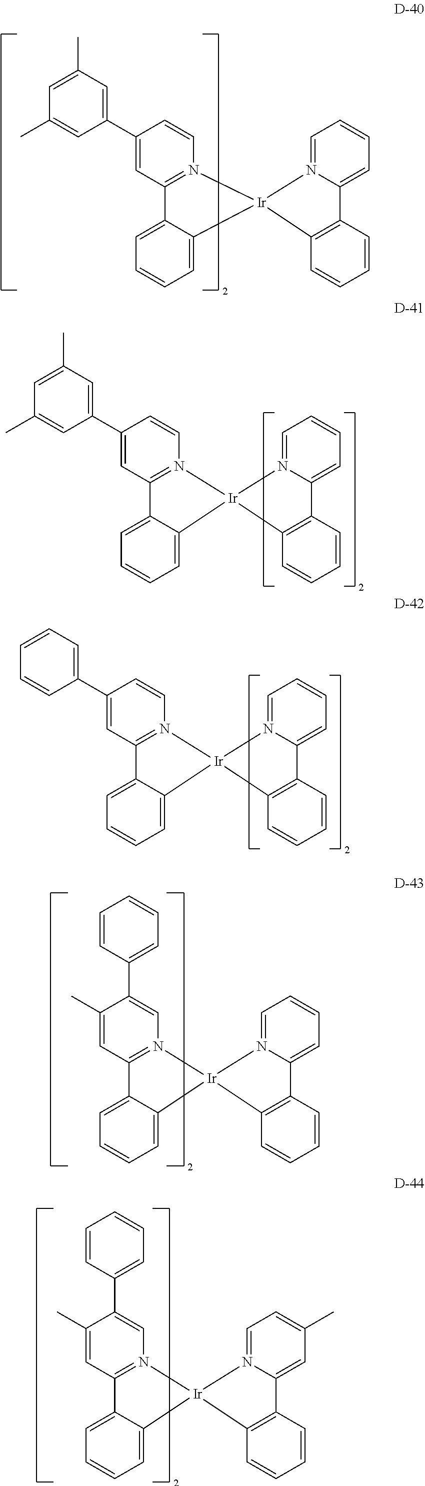

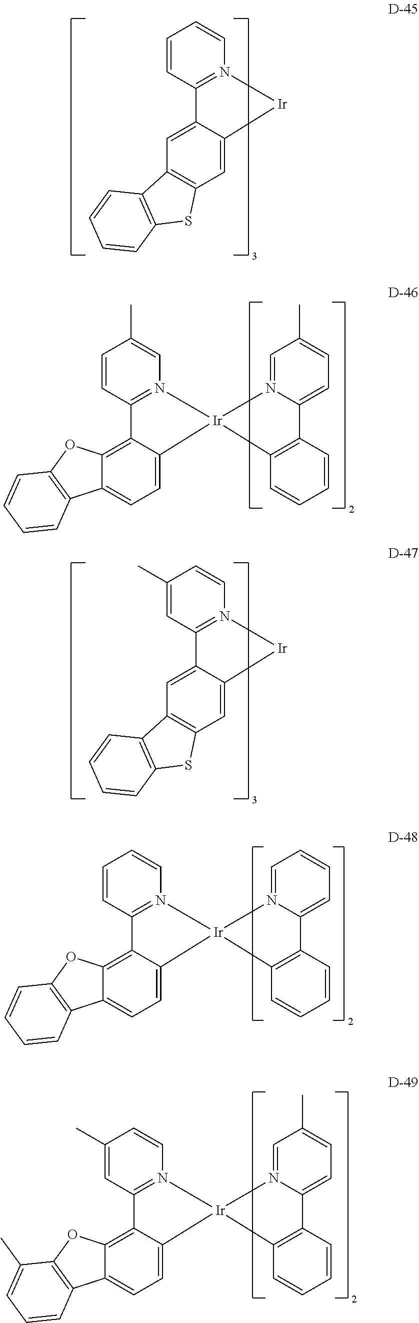

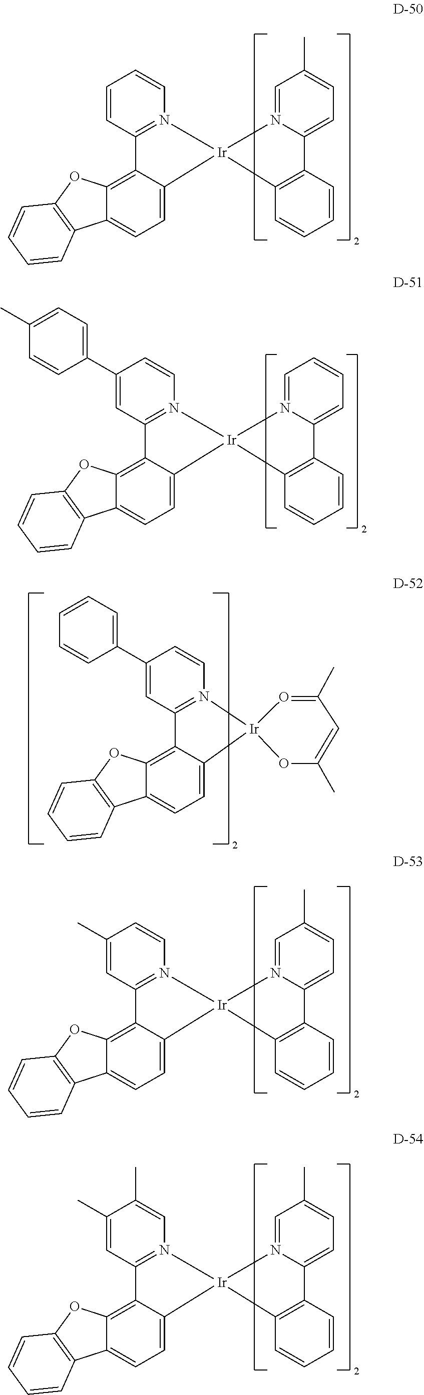

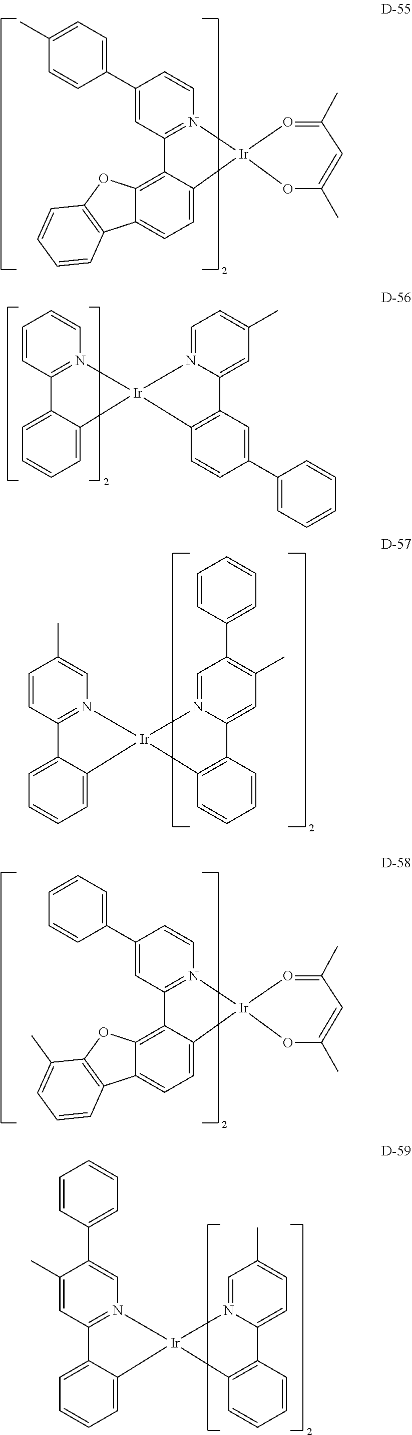

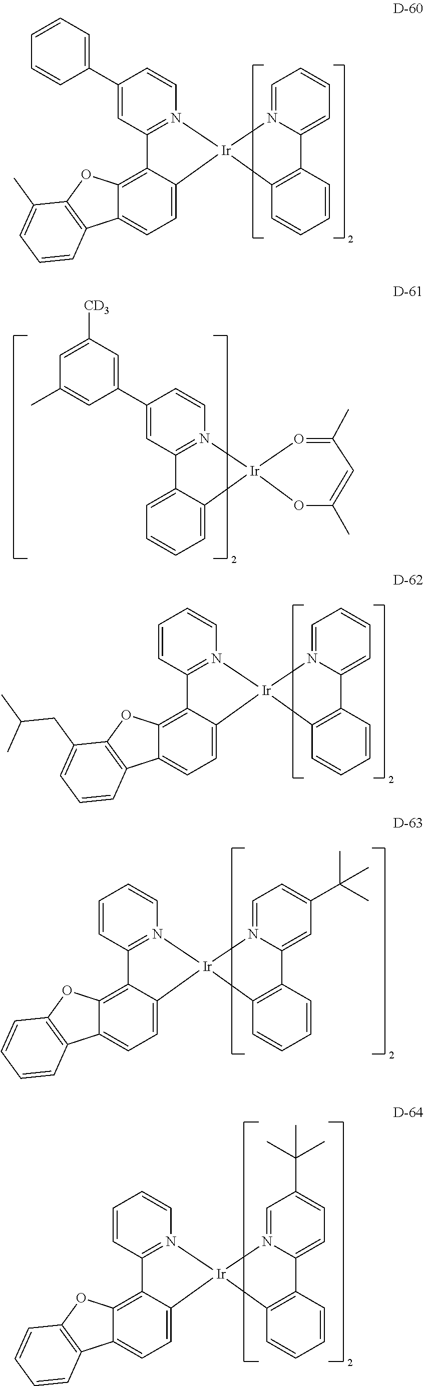

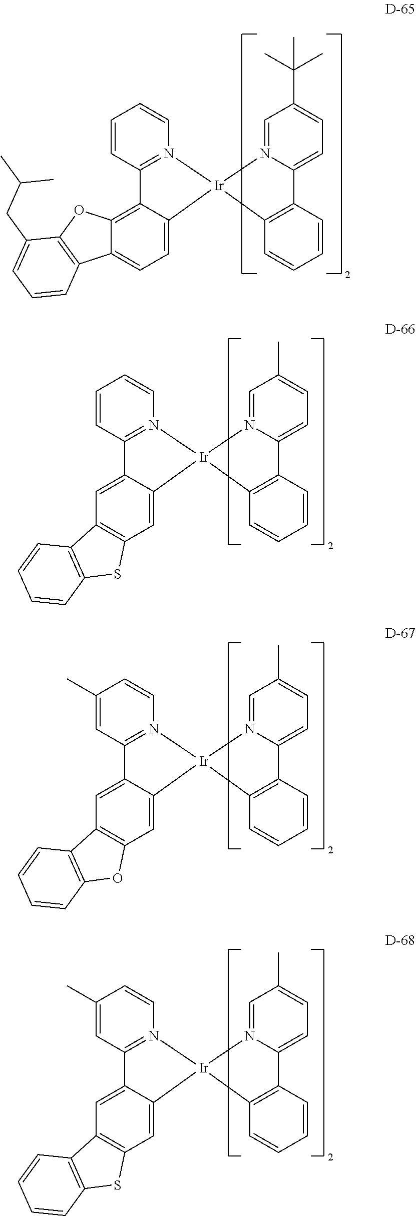

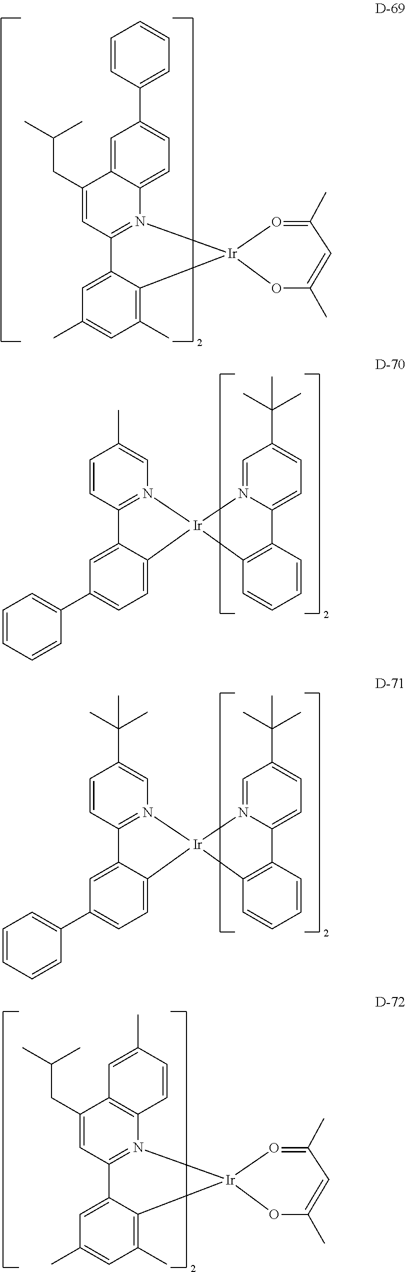

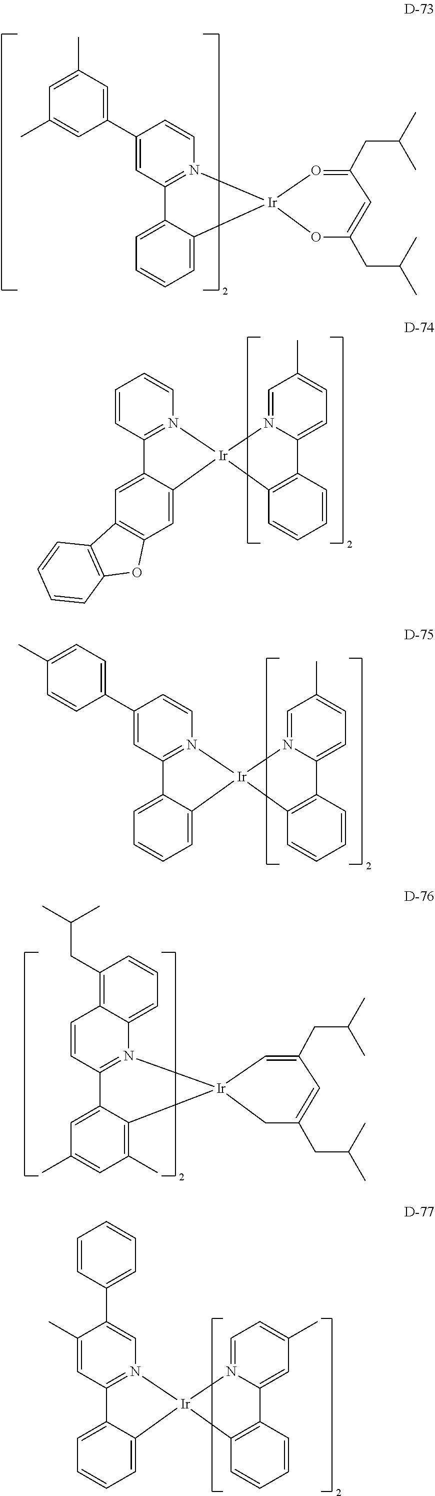

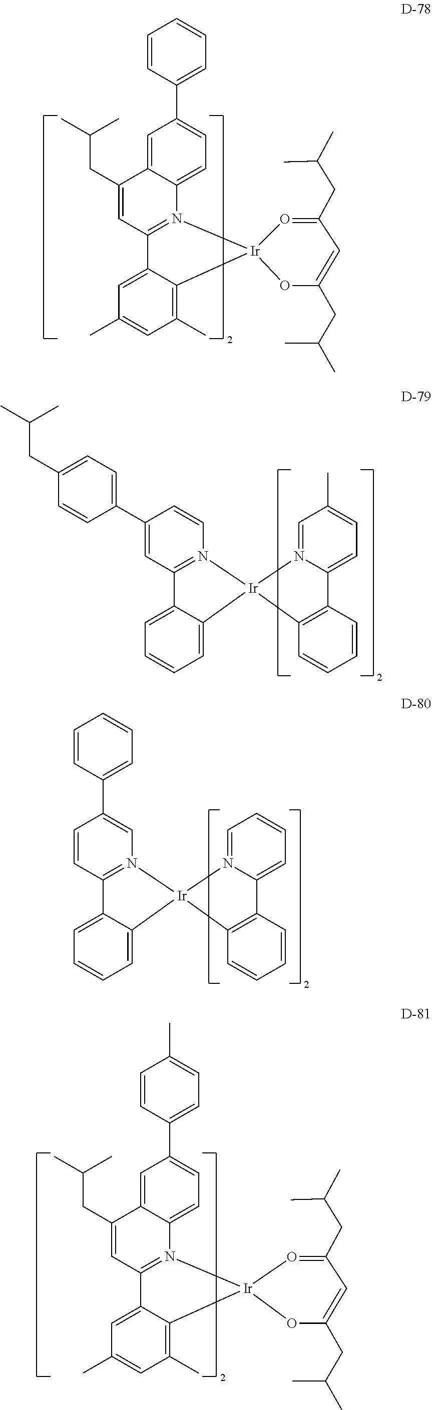

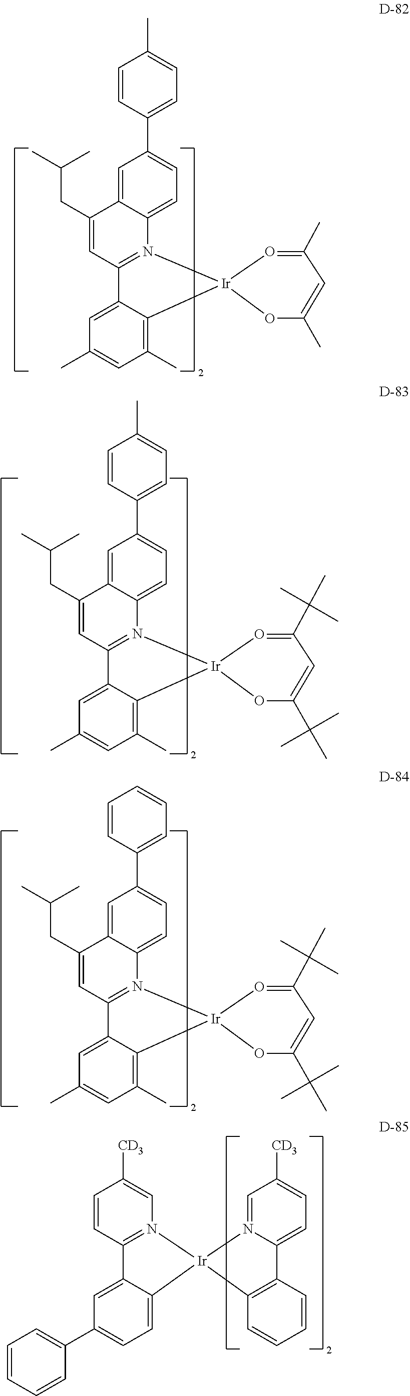

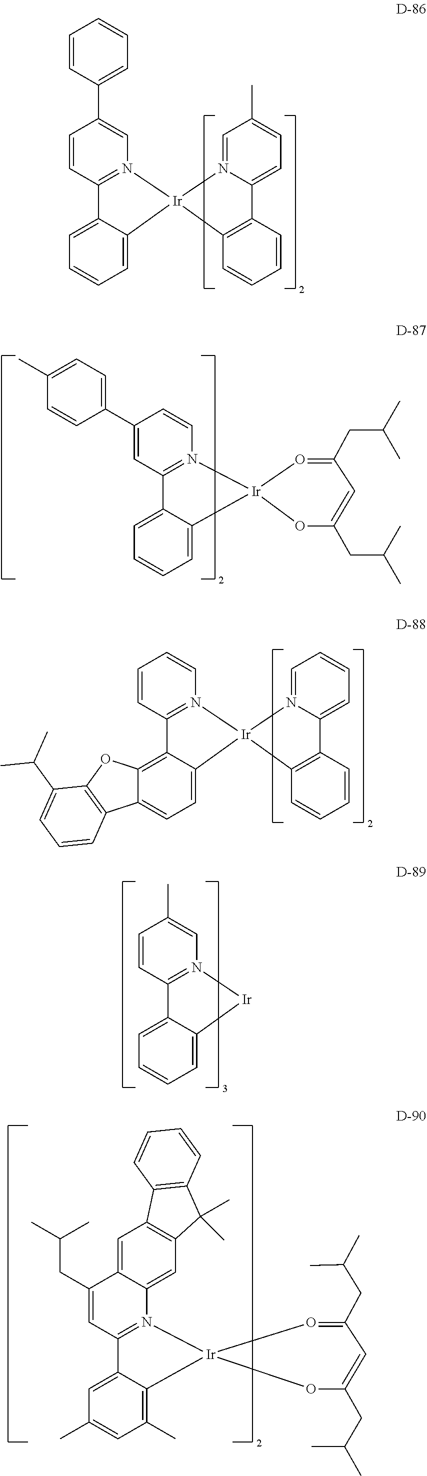

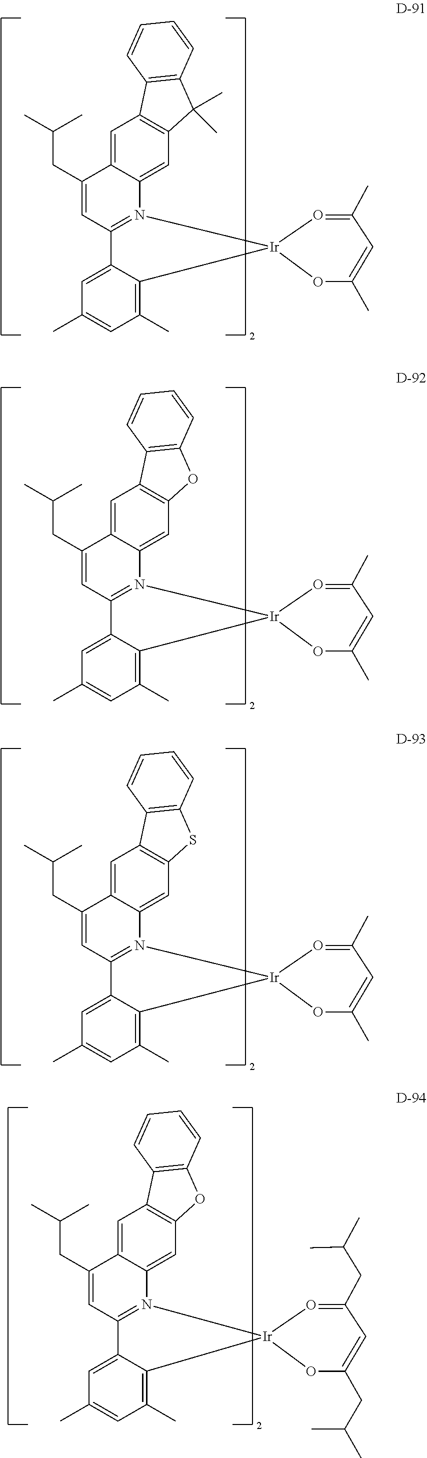

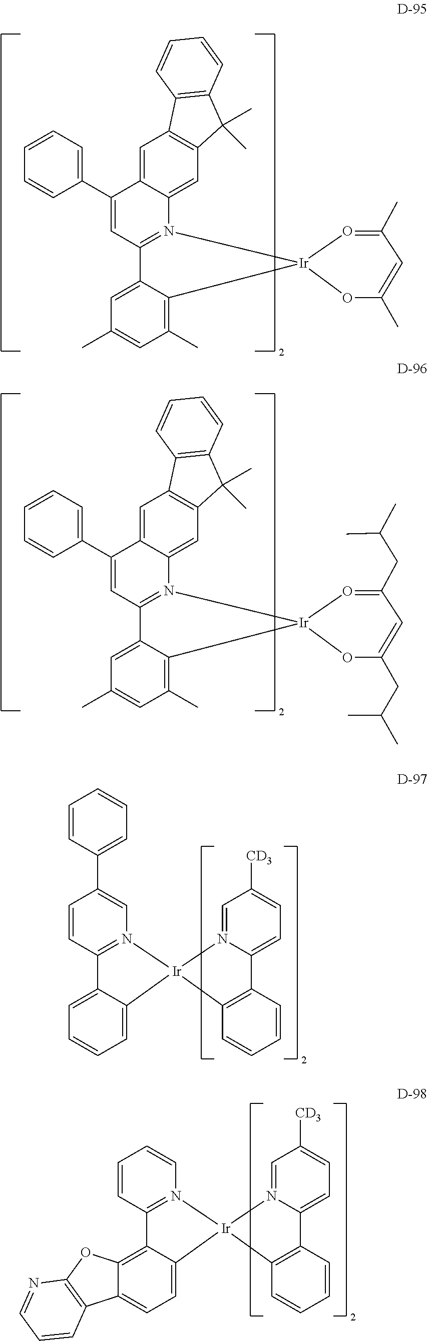

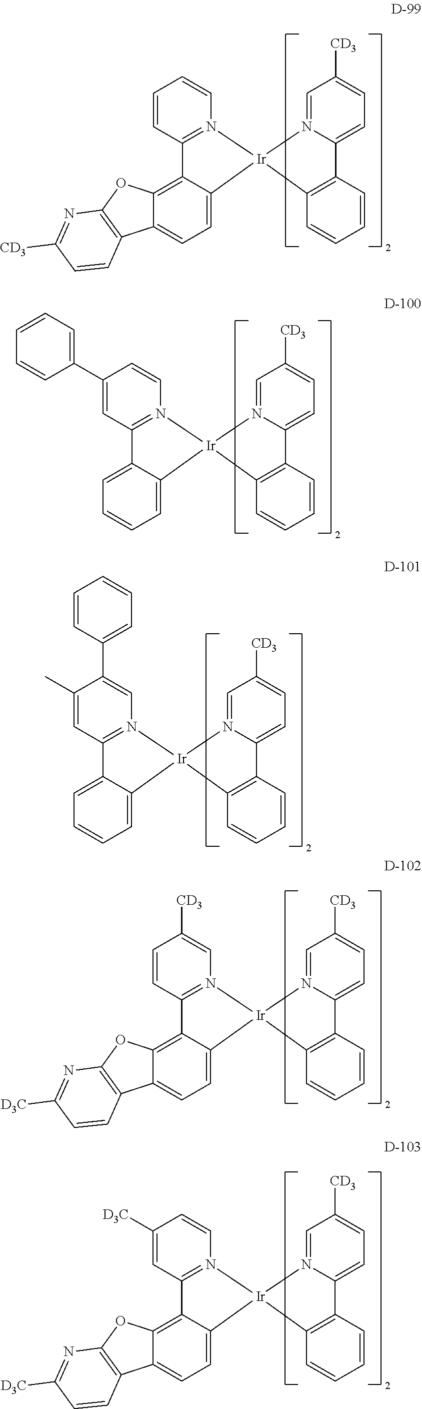

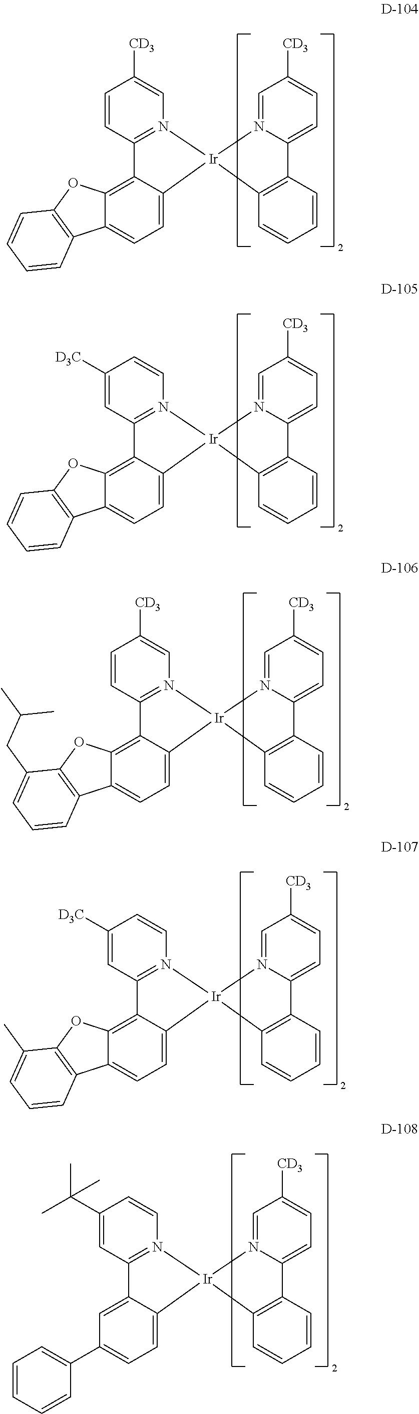

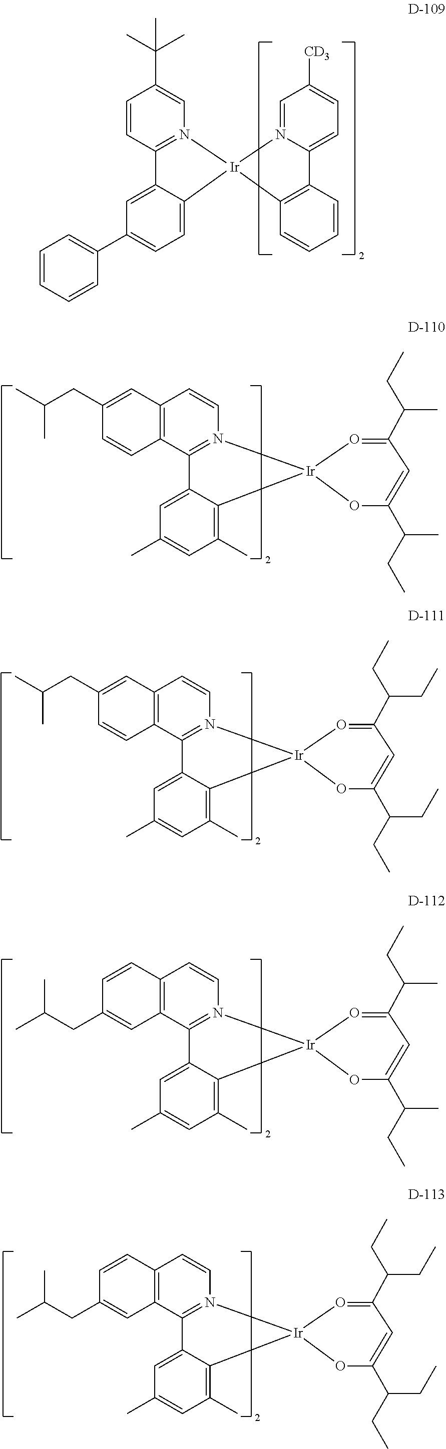

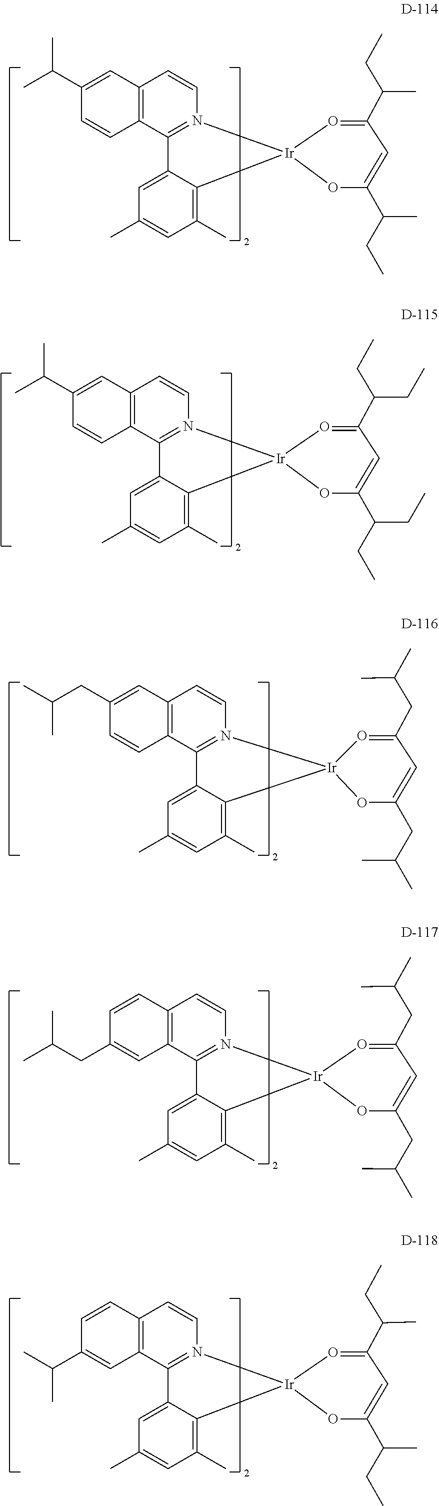

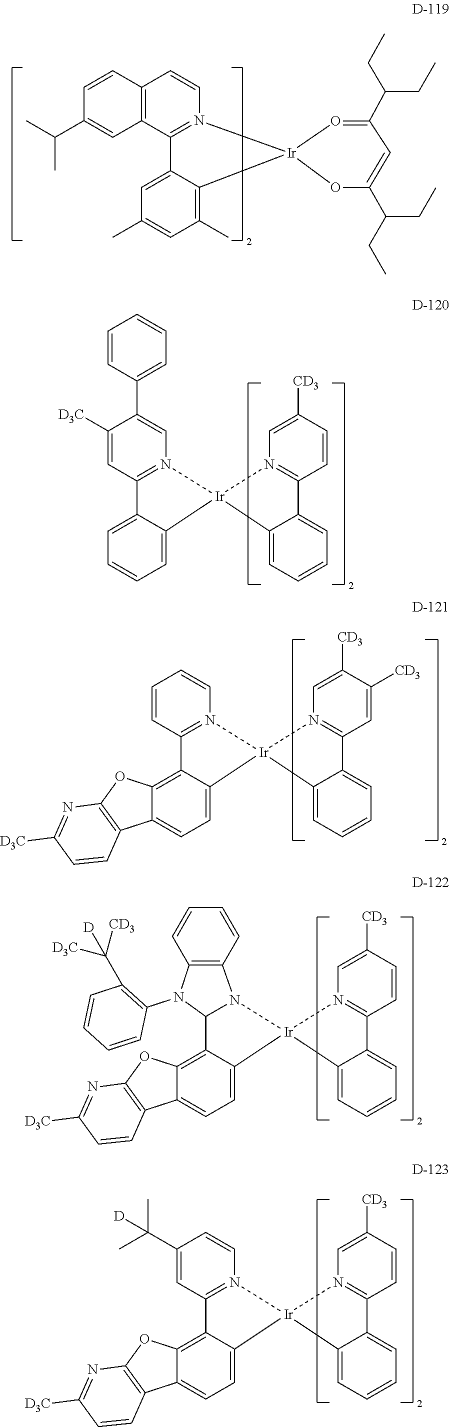

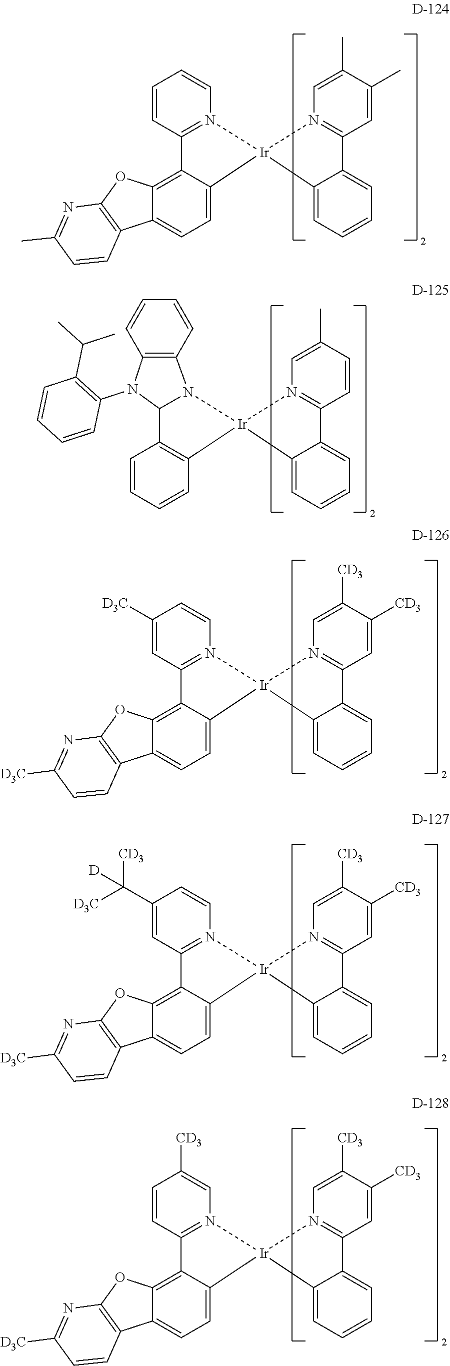

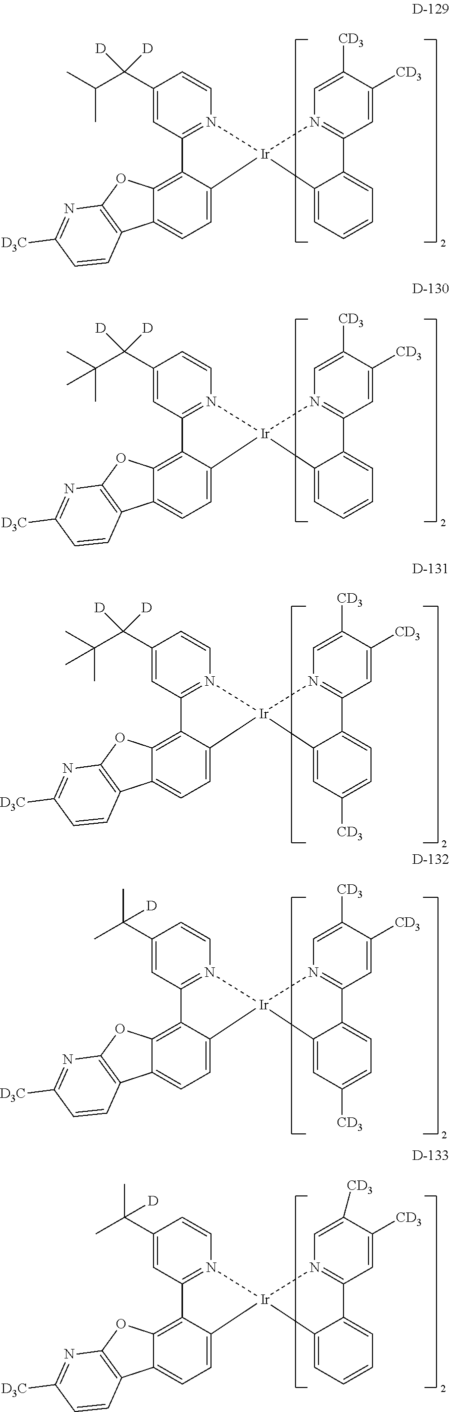

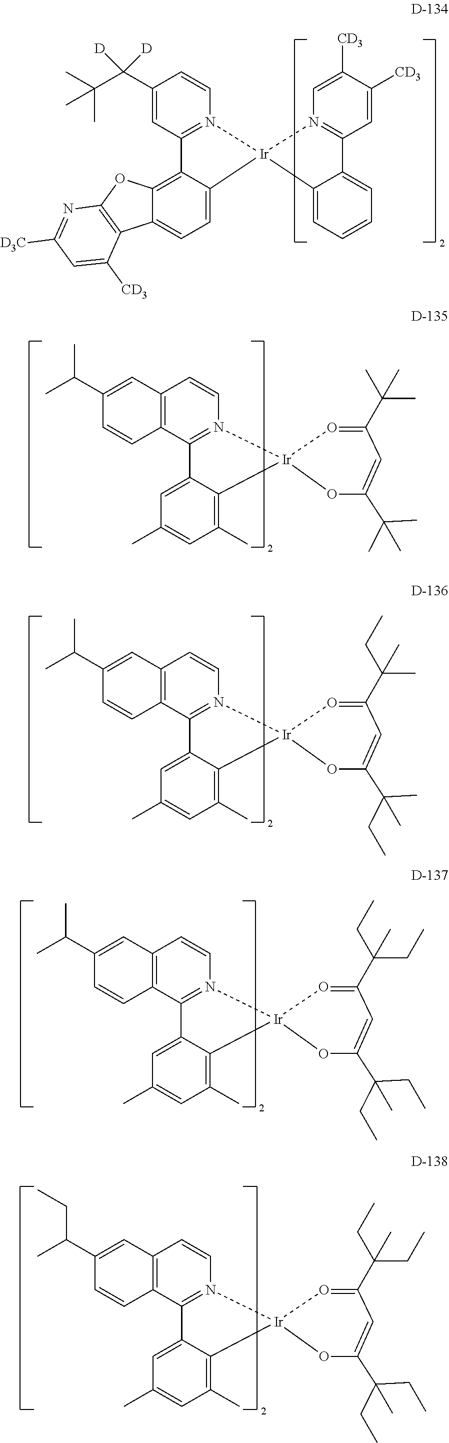

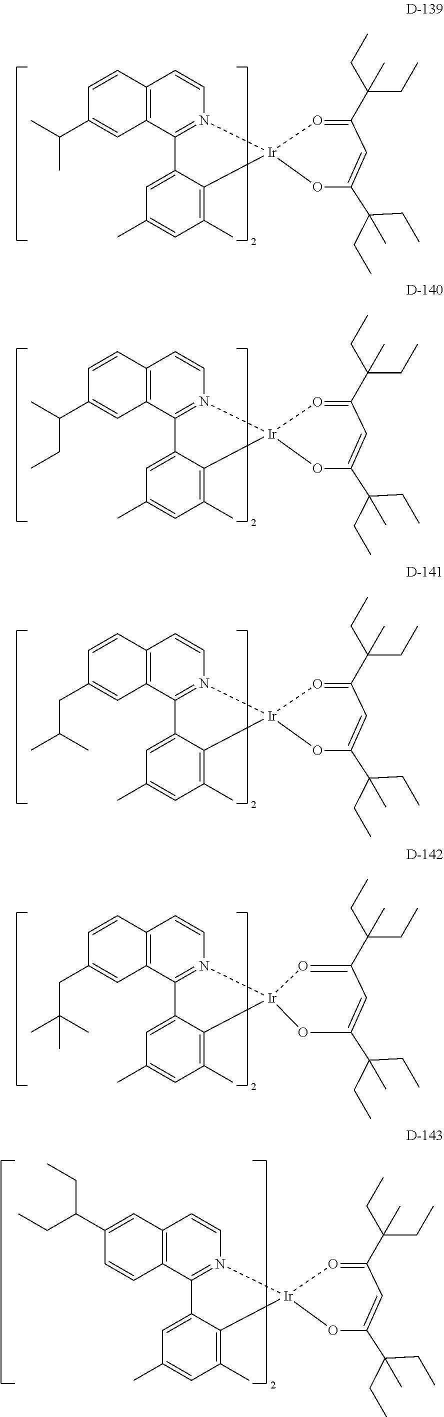

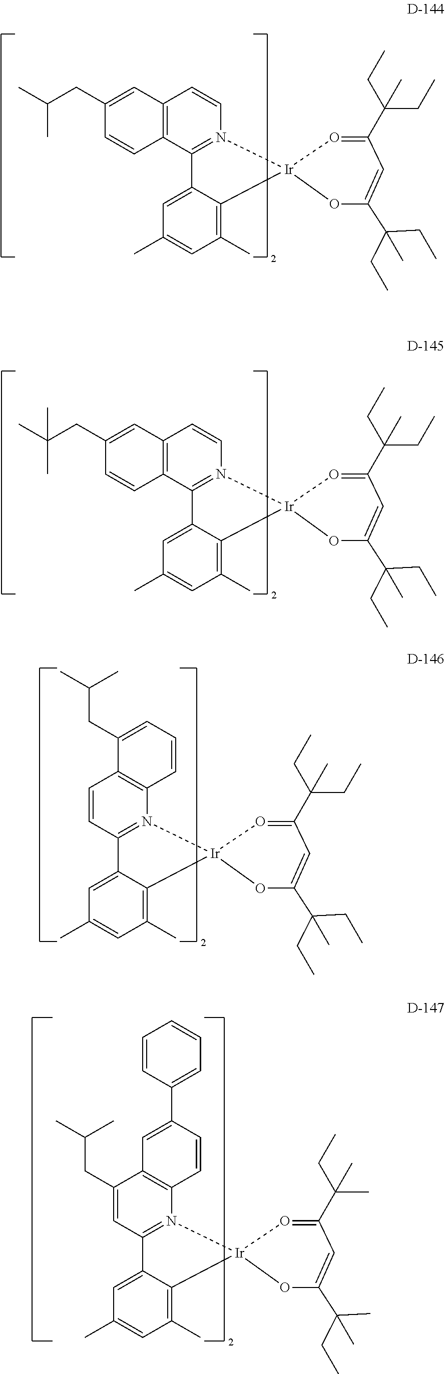

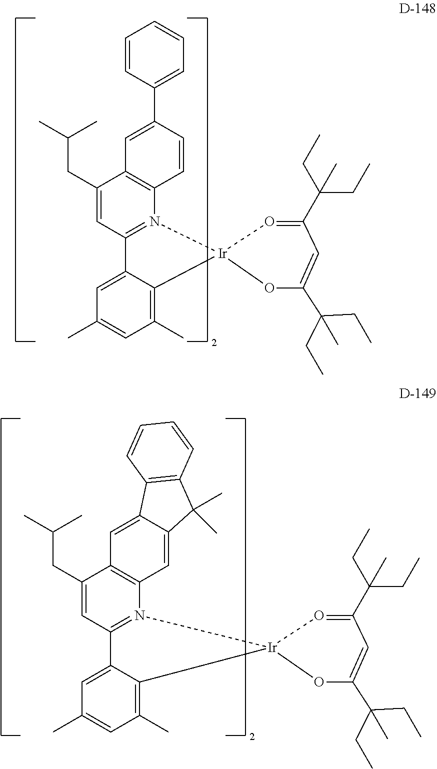

In order to form each layer of the organic electroluminescent device of the present disclosure, dry film-forming methods such as vacuum evaporation, sputtering, plasma, ion plating methods, etc., or wet film-forming methods such as spin coating, dip coating, flow coating methods, etc., can be used. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by the first host material and the second host material according to one embodiment, the layer can be formed by the above-listed methods, and can often be formed by co-deposition or mixture-deposition. The co-deposition is a mixed deposition method in which two or more materials are put into respective individual crucible sources and a current is applied to both cells simultaneously to evaporate the materials and to perform mixed deposition; and the mixed deposition is a mixed deposition method in which two or more materials are mixed in one crucible source before deposition, and then a current is applied to one cell to evaporate the materials.

According to one embodiment, when the first host material and the second host material exist in the same layer or different layers in the organic electroluminescent device, the layers by the two host compounds may be separately formed. For example, after depositing the first host material, a second host material may be deposited.

According to one embodiment, the present disclosure can provide display devices comprising a plurality of host materials comprising a first host material comprising a compound represented by formula 1 and a second host material comprising a compound represented by formula 2 or an organic electroluminescent compound represented by formula 3. In addition, the organic electroluminescent device of the present disclosure can be used for the manufacture of display devices such as smartphones, tablets, notebooks, PCs, TVs, or display devices for vehicles, or lighting devices such as outdoor or indoor lighting.

Hereinafter, the preparation method of compounds according to the present disclosure will be explained with reference to the synthesis method of a representative compound or intermediate compound in order to understand the present disclosure in detail.

### [Example 1] Synthesis of Compound H1-104

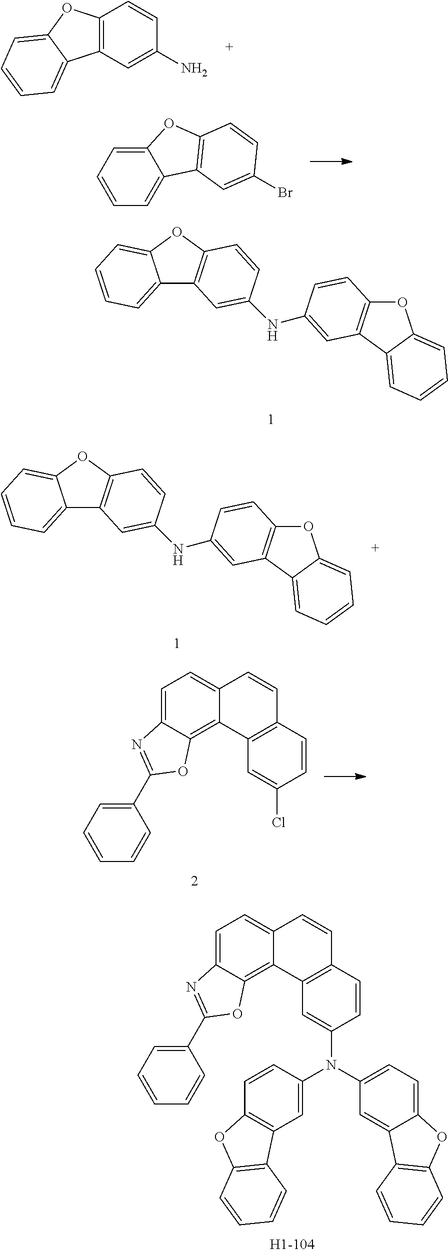

1) Synthesis of Compound 1

Dibenzofuran-2-amine (20 g, 144.7 mmol), 2-bromodibenzofuran (23.8 g, 96.47 mmol), palladium acetate(II)(Pd(OAc)₂) (1.1 g, 4.82 mmol), 2-dicyclohexylphosphino-2′,6′-dimethoxybiphenyl(S-Phos) (3.9 g, 9.65 mmol), sodium tert-butoxide (NaOt-Bu) (13.9 g, 144.7 mmol) and 485 mL of o-xylene were added to the flask, and stirred at 160° C. for 3 hours. After completion of the reaction, the mixture was cooled to room temperature, the organic layers were extracted with ethyl acetate, and the residual moisture was removed using magnesium sulfate, followed by drying. Next, it was separated by column chromatography to obtain Compound 1 (4.9 g, yield: 10%).

2) Synthesis of Compound H1-104

Compound 1 (4.9 g, 12.76 mmol), compound 2 (4.2 g, 14.0 mmol), tris(dibenzylideneacetone)dipalladium(0)(Pd₂(dba)₃) (0.584 g, 0.638 mmol), S-Phos (0.523 g, 1.276 mmol), NaOt-Bu (1.8 g, 19.14 mmol) and 65 mL of o-xylene were added to the flask and stirred at 160° C. for 2 hours. After completion of the reaction, the mixture was cooled to room temperature, the organic layers were extracted with ethyl acetate, and the residual moisture was removed using magnesium sulfate, followed by drying. Next, it was separated by column chromatography to obtain compound H1-104 (5.6 g, yield: 68.3%).

| Compound | MW | M.P. |
|---|---|---|
| H1-104 | 642.19 | 237° C. |

### [Example 2] Synthesis of Compound H1-88

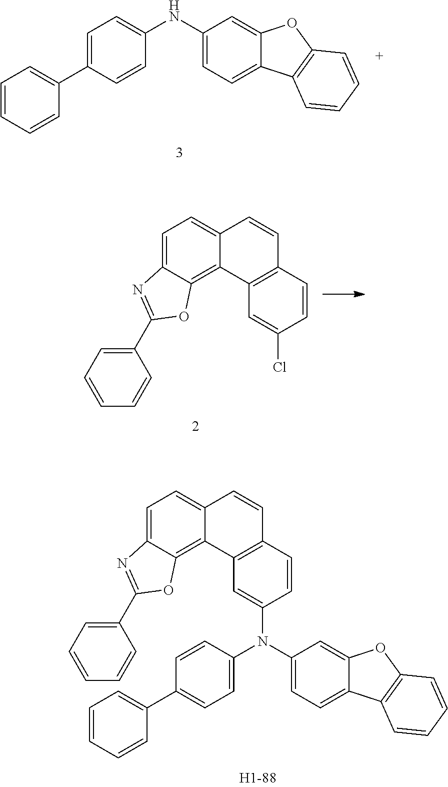 [SMILES: c1ccc(-c2ccc(Nc3ccc4c(c3)oc3ccccc34)cc2)cc1 ; Clc1ccc2ccc3ccc4nc(-c5ccccc5)oc4c3c2c1 ; c1ccc(-c2ccc(N(c3ccc4c(c3)oc3ccccc34)c3ccc4ccc5ccc6nc(-c7ccccc7)oc6c5c4c3)cc2)cc1]

Compound 3 (25 g, 74.48 mmol), compound 2 (42.58 g, 81.93 mmol), Pd(OAc)₂ (0.16 g, 7.5 mmol), tri-tert-butylphosphine (P(t-Bu)₃) (0.28 g, 7.5 mmol), NaOt-Bu (14.31 g, 150 mmol) and 284.09 mL of o-xylene were added to the flask and stirred at 160° C. for 2 hours. After completion of the reaction, the mixture was cooled to room temperature, the organic layers were extracted with ethyl acetate, and the residual moisture was removed using magnesium sulfate, followed by drying. Next, it was separated by column chromatography to obtain compound H1-88 (23.4 g, yield: 50%).

| Compound | MW | M.P. |
|---|---|---|
| H1-88 | 628.22 | 256.5° C. |

### [Example 3] Synthesis of Compound H1-94

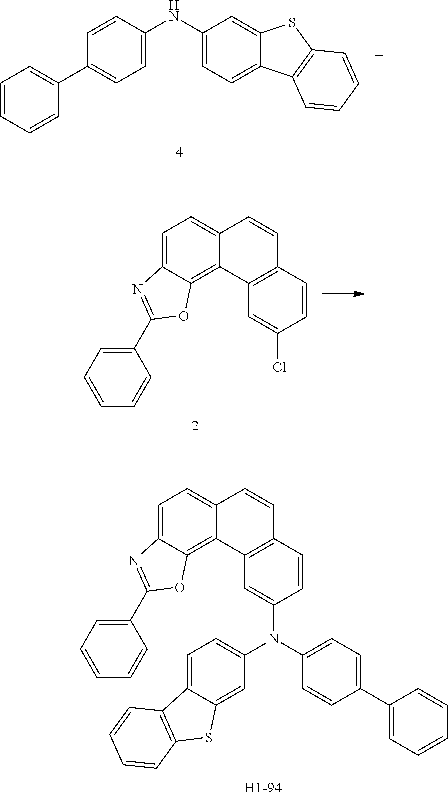 [SMILES: c1ccc(-c2ccc(Nc3ccc4c(c3)sc3ccccc34)cc2)cc1 ; Clc1ccc2ccc3ccc4nc(-c5ccccc5)oc4c3c2c1 ; c1ccc(-c2ccc(N(c3ccc4c(c3)sc3ccccc34)c3ccc4ccc5ccc6nc(-c7ccccc7)oc6c5c4c3)cc2)cc1]

Compound 4 (20 g, 56.96 mmol), compound 2 (18.8 g, 57.13 mmol), Pd(OAc)₂ (0.13 g, 5.7 mmol), P(t-Bu)₃ (0.22 g, 5.7 mmol), NaOt-Bu (11 g, 113.92 mmol) and 227.27 mL of o-xylene were added to the flask, and stirred at 160° C. for 2 hours. After completion of the reaction, the mixture was cooled to room temperature, the organic layers were extracted with ethyl acetate, and the residual moisture was removed using magnesium sulfate, followed by drying. Next, it was separated by column chromatography to obtain compound H1-94 (12.5 g, yield: 34%).

| Compound | MW | M.P. |
|---|---|---|
| H1-94 | 644.19 | 249° C. |

### [Example 4] Synthesis of Compound H1-85

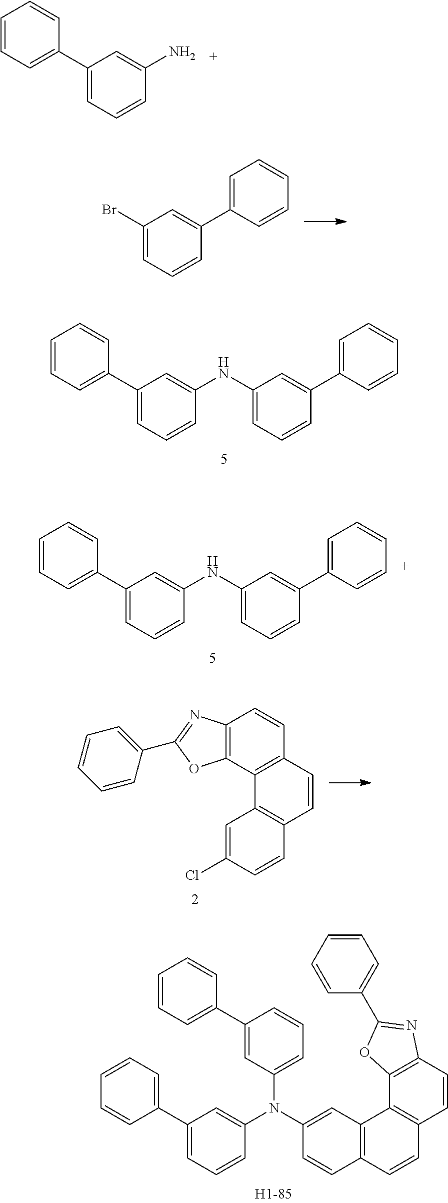 [SMILES: Nc1cccc(-c2ccccc2)c1 ; Brc1cccc(-c2ccccc2)c1 ; c1ccc(-c2cccc(Nc3cccc(-c4ccccc4)c3)c2)cc1 ; c1ccc(-c2cccc(Nc3cccc(-c4ccccc4)c3)c2)cc1 ; Clc1ccc2ccc3ccc4nc(-c5ccccc5)oc4c3c2c1 ; c1ccc(-c2cccc(N(c3cccc(-c4ccccc4)c3)c3ccc4ccc5ccc6nc(-c7ccccc7)oc6c5c4c3)c2)cc1]

1) Synthesis of Compound 5

3-Aminobiphenyl (54 g, 319 mmol), 3-bromobiphenyl (70 g, 301 mmol), Pd(OAc)₂ (0.33 g, 1.47 mmol), tricyclohexylphosphine (P(Cy)₃) (0.84 g, 2.8 mmol), NaOt-Bu (57 g, 593 mmol) and 280 mL of toluene were added to the flask, and stirred at 95° C. for 8 hours. After completion of the reaction, the mixture was cooled to room temperature, the organic layers were extracted with ethyl acetate, and the residual moisture was removed using magnesium sulfate, followed by drying. Next, it was separated by column chromatography to obtain compound 5 (60.23 g, yield: 85%).

2) Synthesis of Compound H1-85

Compound 5 (60.23 g, 187.5 mmol), compound 2 (60 g, 182.33 mmol), Pd(OAc)₂ (0.41 g, 1.83 mmol), S-phos (1.74 g, 4.23 mmol), NaOt-Bu (26.23 g, 272 mmol) and 300 mL of xylene were added to the flask and stirred at 110° C. for 10 hours. After completion of the reaction, the mixture was cooled to room temperature, the organic layers were extracted with ethyl acetate, and the residual moisture was removed using magnesium sulfate, followed by drying. Next, it was separated by column chromatography to obtain compound H1-85 (36.9 g, yield: 33%).

| Compound | MW | M.P. |
|---|---|---|
| H1-85 | 614.24 | 210° C. |

### [Example 5] Synthesis of Compound H1-115

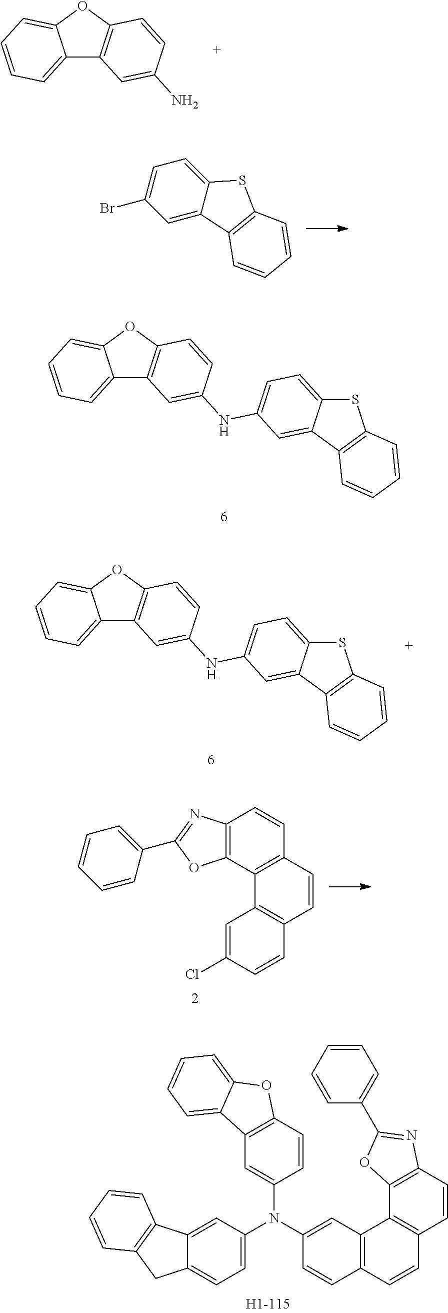 [SMILES: Nc1ccc2oc3ccccc3c2c1 ; Brc1ccc2sc3ccccc3c2c1 ; c1ccc2c(c1)oc1ccc(Nc3ccc4sc5ccccc5c4c3)cc12 ; c1ccc2c(c1)oc1ccc(Nc3ccc4sc5ccccc5c4c3)cc12 ; Clc1ccc2ccc3ccc4nc(-c5ccccc5)oc4c3c2c1 ; c1ccc(-c2nc3ccc4ccc5ccc(N(c6ccc7c(c6)-c6ccccc6C7)c6ccc7oc8ccccc8c7c6)cc5c4c3o2)cc1]

1) Synthesis of Compound 6

Dibenzofuran-2-amine (29.24 g, 159.7 mmol), 2-bromodibenzothiophene (40 g, 152.7 mmol), Pd(OAc)₂ (0.17 g, 0.75 mmol), P(Cy)₃ (0.43 g, 1.45 mmol), NaOt-Bu (29.22 g, 304 mmol) and 250 mL of toluene were added to the flask and stirred at 95° C. for 8 hours. After completion of the reaction, the mixture was cooled to room temperature, the organic layers were extracted with ethyl acetate and the residual moisture was removed using magnesium sulfate, followed by drying. Next, it was separated by column chromatography to obtain compound 6 (17.12 g, yield: 86%).

2) Synthesis of Compound H1-115

Compound 6 (17.12 g, 46.89 mmol), compound 2 (15 g, 45.58 mmol), Pd(OAc)₂ (0.05 g, 0.22 mmol), S-phos (0.22 g, 0.535 mmol), NaOt-Bu (6.56 g, 68.2 mmol) and 75 mL of xylene were added to the flask and stirred at 110° C. for 10 hours. After completion of the reaction, the mixture was cooled to room temperature, the organic layers were extracted with ethyl acetate, and the residual moisture was removed using magnesium sulfate, followed by drying. Next, it was separated by column chromatography to obtain compound H1-115 (10.2 g, yield: 34%).

| Compound | MW | M.P. |
|---|---|---|
| H1-115 | 658.17 | 254° C. |

### [Example 6] Synthesis of Compound H1-86

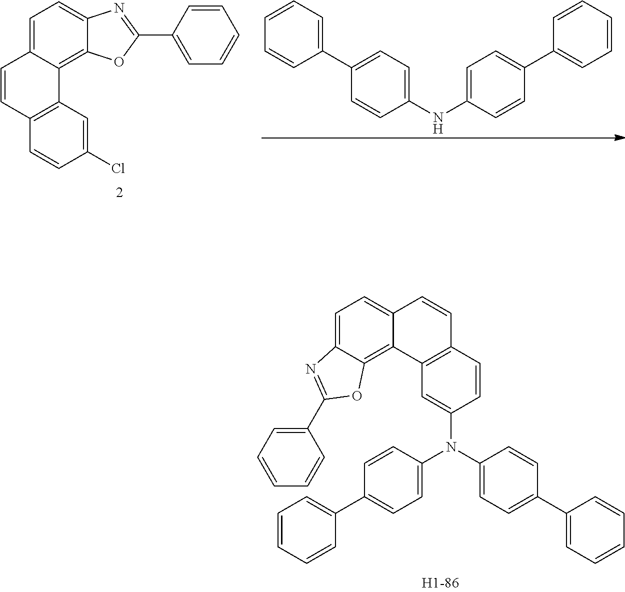

Compound 2 (5.0 g, 15.2 mmol), di([1,1′-biphenyl]-4-nyl)amine (4.9 g, 15.2 mmol), Pd(OAc)₂ (0.2 g, 0.8 mmol), P(t-Bu)₃ (0.8 mL, 1.5 mmol), NaOt-Bu (2.9 g, 30.4 mmol) and 76 mL of xylene were added to the flask and stirred at 160° C. for 5 hours. After completion of the reaction, the mixture was cooled to room temperature and the precipitated solid was washed with distilled water and methanol. Next, it was separated by column chromatography to obtain compound H1-86 (5.5 g, yield: 59%).

### [Example 7] Synthesis of Compound H1-51

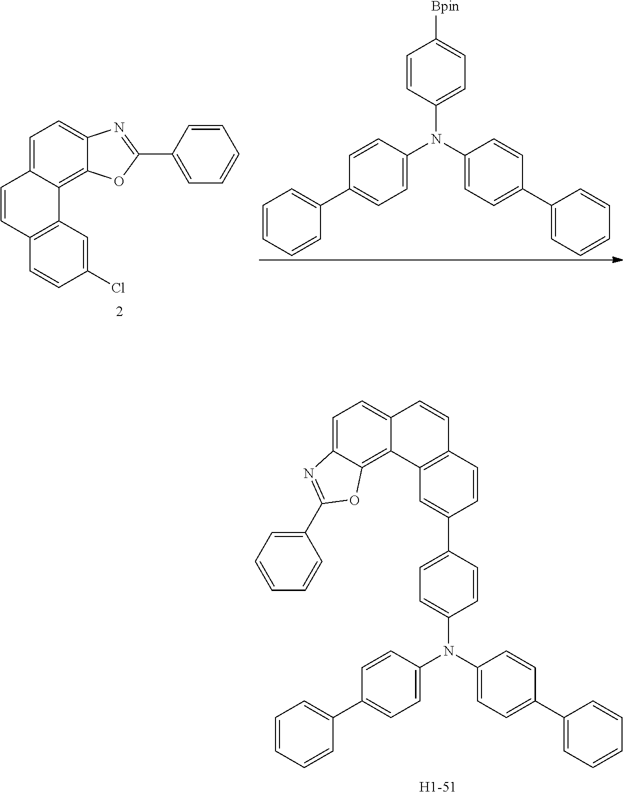

Compound 2 (4 g, 12 mmol), bis(biphenyl-4-yl)[4-(4,4,5,5-tetramethyl-[1,3,2]-dioxaborolan-2-yl)phenyl]amine (6.8 g, 13 mmol), Pd(OAc)₂ (0.3 g, 1 mmol), S-Phos (0.9 g, 2 mmol), cesium carbonate (Cs₂CO₃) (11.5 g, 35 mmol), 60 mL of o-xylene, 15 mL of EtOH, and 15 mL of distilled water were added to the flask and stirred under reflux at 150° C. for 3 hours. After completion of the reaction, the mixture was cooled to room temperature and washed with distilled water, the organic layer was extracted with ethyl acetate, and the residual moisture was removed using magnesium sulfate, followed by drying. Next, it was separated by column chromatography to obtain compound H1-51 (2.2 g, yield: 27%).

### [Example 8] Synthesis of Compound H1-68

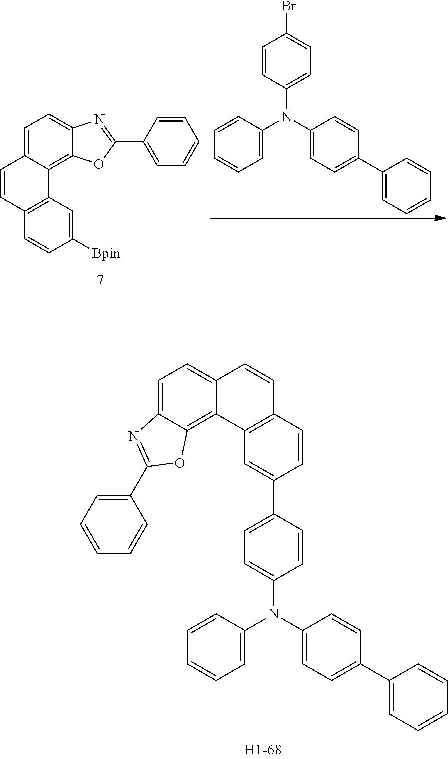

Compound 7 (4.8 g, 11.34 mmol), N-(4-bromophenyl)-N-phenyl-[1,1′-biphenyl]-4-amine (5 g, 12.47 mmol), tetrakis(triphenylphosphine)palladium(O)(Pd(PPh₃)₄) (0.4 g, 0.34 mmol), Na₂CO₃ (3.0 g, 28.35 mmol), 57 mL of toluene, 14 mL of ethanol, and 14 mL of distilled water were added to the flask and stirred at 120° C. for 4 hours. After completion of the reaction, the mixture was added dropwise to methanol and the resulting solid was filtered. The resulting solid was purified by column chromatography to obtain compound H1-68 (1.4 g, yield: 20.0%).

### [Example 9] Synthesis of Compound H1-87

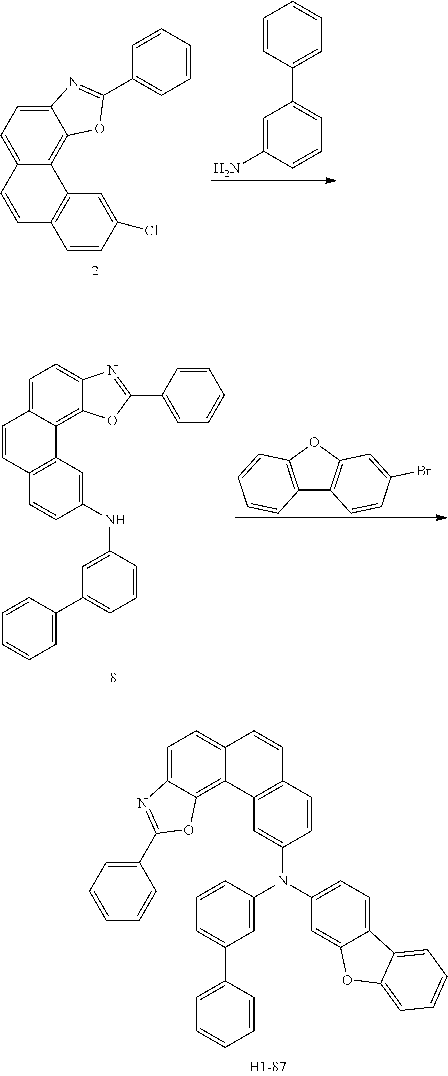 [SMILES: Clc1ccc2ccc3ccc4nc(-c5ccccc5)oc4c3c2c1 ; Nc1cccc(-c2ccccc2)c1 ; c1ccc(-c2cccc(Nc3ccc4ccc5ccc6nc(-c7ccccc7)oc6c5c4c3)c2)cc1 ; Brc1ccc2c(c1)oc1ccccc12 ; c1ccc(-c2cccc(N(c3ccc4c(c3)oc3ccccc34)c3ccc4ccc5ccc6nc(-c7ccccc7)oc6c5c4c3)c2)cc1]

1) Synthesis of Compound 8

Compound 2 (10.0 g, 30.3 mmol), [1,1′-biphenyl]-3-amine (6.7 g, 39.4 mmol), Pd(OAc)₂ (0.34 g, 1.5 mmol), P(t-Bu)₃ (1.5 mL, 3.03 mmol), NaOt-Bu (5.8 g, 60.6 mmol), and 150 mL of xylene were added to the flask and stirred at 160° C. for 6 hours. After completion of the reaction, the mixture was cooled to room temperature and washed with distilled water, the organic layer was extracted with ethyl acetate and dried over magnesium sulfate, and then the solvent was removed using a rotary evaporator. Next, it was separated by column chromatography to obtain compound 8 (10.8 g, yield: 36%).

2) Synthesis of Compound H1-87

Compound 8 (5.0 g, 10.8 mmol), 3-bromodibenzofuran (3.2 g, 12.9 mmol), Pd₂(dba)₃ (0.5 g, 0.54 mmol), S-Phos (0.45 g, 1.08 mmol), NaOt-Bu (2.0 g, 21.6 mmol), and 60 mL of o-xylene were added to the flask and stirred at 160° C. for 6 hours. After completion of the reaction, the mixture was cooled to room temperature, and then the organic layer was extracted with ethyl acetate and the residual moisture was removed using magnesium sulfate, followed by drying. Next, it was separated by column chromatography to obtain compound H1-87 (1.45 g, yield: 21%).

| Compound | MW | M.P. |
|---|---|---|
| H1-87 | 628.73 | 205° C. |

### [Example 10] Synthesis of Compound H1-96

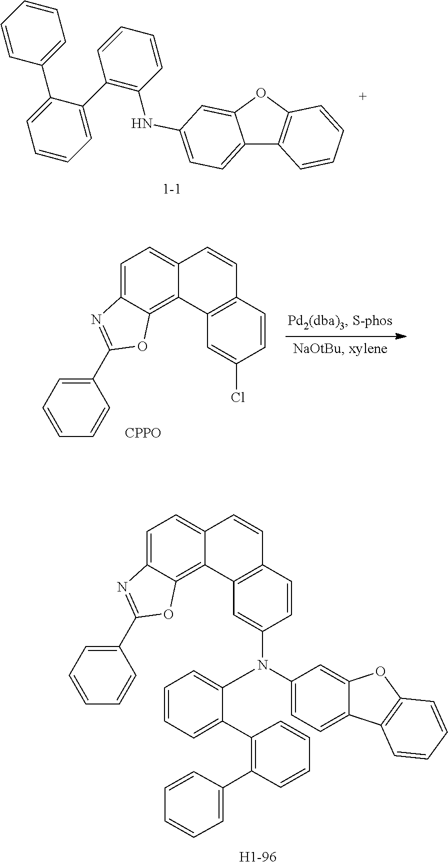

Compound 1-1 (33 g, 100 mmol), compound CPPO (45.3 g, 110 mmol), Pd₂(dba)₃ (4.5 g, 5 mmol), S-Phos (4.1 g, 10 mmol), NaOt-Bu (19.2 g, 200 mmol), and 500 mL of o-xylene were added to the flask and stirred at 160° C. for 2 hours. After completion of the reaction, the mixture was cooled to room temperature, and then the organic layers were extracted with ethyl acetate and the residual moisture was removed using magnesium sulfate, followed by drying. Next, it was separated by column chromatography to obtain compound H1-96 (52 g, yield: 73.7%).

| Compound | MW | M.P |
|---|---|---|
| H1-96 | 704.83 | 280° C. |

### [Example 11] Synthesis of Compound H1-103

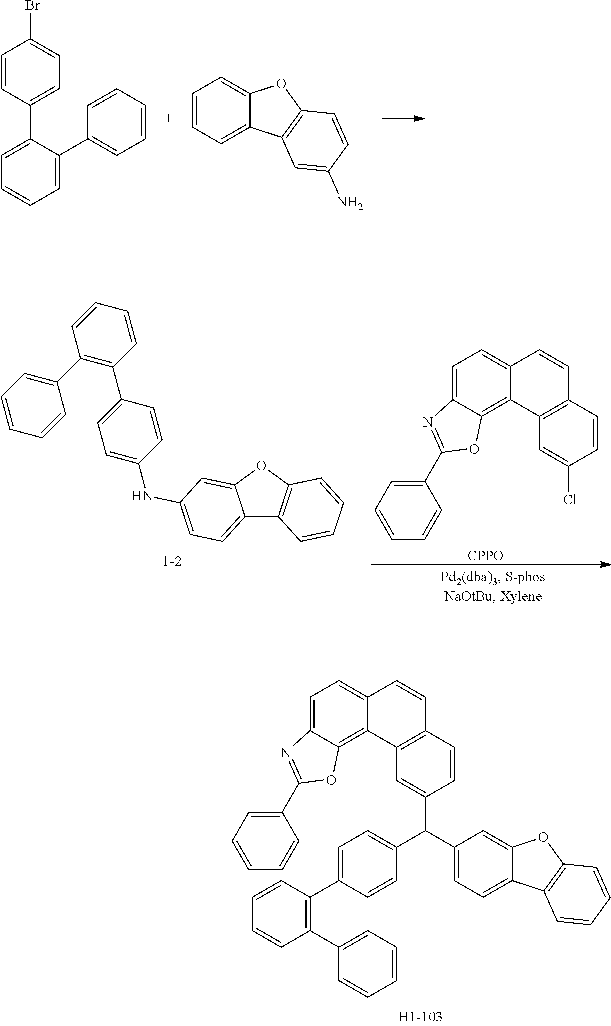

1) Synthesis of Compound 1-2

4-bromo-1,1′:2,1″-terphenyl (10.0 g, 32.34 mmol), 8-aminodibenzo[b,d]furan-2-yl (8.8 g, 48.51 mmol), bis(di-tert-butyl(4-dimethylaminophenyl)phosphine)dichloropalladium (II)(PdCl₂(Amphos)₂) (2.3 g, 3.23 mmol), and NaOt-Bu (4.6 g, 48.51 mmol) were dissolved in 161 mL of o-xylene and then stirred under reflux for 2 hours. After completion of the reaction, the mixture was cooled to room temperature and filtered with Celite-filter solid to obtain compound 1-2 (8.6 g, yield: 64.6%).

2) Synthesis of Compound H1-103

Compound 1-2 (8.6 g, 20.90 mmol), compound CPPO (8.3 g, 25.08 mmol), Pd₂(dba)₃ (1.0 g, 1.05 mmol), S-Phos (900 mg, 2.09 mmol), and NaOt-Bu (5.0 g, 52.25 mmol) were dissolved in 140 mL of o-xylene, and then stirred under reflux for 3 hours. After completion of the reaction, the mixture was cooled to room temperature and filtered with Celite filter to make a solid. Next, it was separated by column chromatography to obtain compound H1-103 (5.8 g, yield: 39.4%).

| Compound | MW | M.P |
|---|---|---|
| H1-103 | 704.83 | 168° C. |

### [Example 12] Synthesis of Compound H1-95

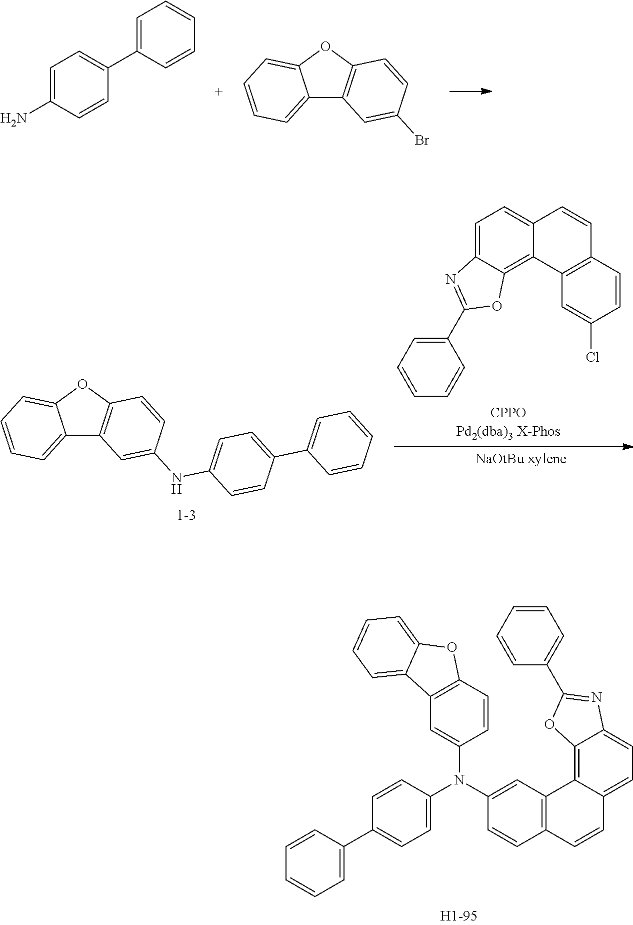

1) Synthesis of Compound 1-3

[1,1′-biphenyl]-4-amine (60 g, 354 mmol), 2-bromodibenzo[b,d]furan (58.5 g, 236 mmol), Pd(OAc)₂ (0.54 g, 3.23 mmol), P(Cy)₃ (1.35 g, 3 mmol), and NaOt-Bu (40.9 g, 425.5 mmol) were dissolved in 600 mL of toluene and then stirred under reflux for 2 hours. After completion of the reaction, the mixture was cooled to room temperature and filtered with Celite filter to make a solid. Next, it was separated by column chromatography to obtain compound 1-3 (65.7 g, yield: 83%).

2) Synthesis of Compound H1-95

Compound 1-3 (20 g g, 59.6 mmol), compound CPPO (19.7 g, 59.7 mmol), Pd₂(dba)₃ (0.55 g, 0.6 mmol), X-Phos (0.57 g, 1.2 mmol), NaOt-Bu (11.5 g, 119.6 mmol) were dissolved in 200 mL of o-xylene and then stirred under reflux for 3 hours. After completion of the reaction, the mixture was cooled to room temperature and filtered with Celite filter to make a solid. Next, it was separated by column chromatography to obtain compound H1-95 (12.7 g, yield: 34%).

| Compound | MW | M.P |
|---|---|---|
| H1-95 | 628.73 | 252° C. |

### [Example 13] Synthesis of Compound C-9

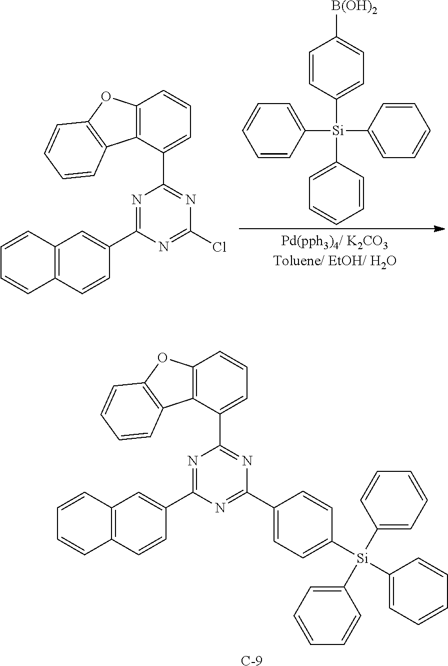

2-chloro-4-(dibenzo[b,d]furan-1-yl)-6-(naphthalene-2-yl)-1,3,5-triazine (10 g, 24.5 mmol), (4-(triphenylsilyl)phenyl)boronic acid (10.2 g, 26.9 mmol), Pd(pph₃)₄ (1.4 g, 1.23 mmol), potassium carbonate (K₂CO₃) (8.4 g, 61.2 mmol), 125 mL of toluene, 30 mL of EtOH, and 30 mL of distilled water (H₂O) were added to the flask, and then stirred at 160° C. After completion of the reaction, methanol (MeOH) and H₂O was added to the reaction mixture, followed by stirring. Next, the solvent was removed by filtration under reduced pressure, following by separating by column chromatography. Next, MeOH was added thereto, and the resulting solid was filtered under reduced pressure to obtain compound C-9 (16.9 g, yield: 97%).

| Compound | MW | color | M.P |
|---|---|---|---|
| C-9 | 707.91 | white | 223.4° C. |

### [Example 14] Synthesis of Compound C-15

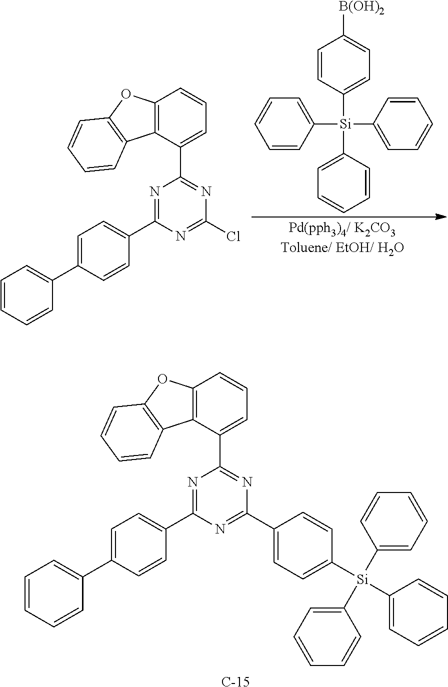

2-([1,1′-biphenyl]-4-yl)-4-chloro-6-(dibenzo[b,d]furan-1-yl)-1,3,5-triazine (10 g, 23.0 mmol), (4-(triphenylsilyl)phenyl)boronic acid (9.6 g, 25.3 mmol), Pd(pph₃)₄ (1.3 g, 1.15 mmol), K₂CO₃ (7.9 g, 57.5 mmol), 120 mL of toluene, 30 mL of EtOH, and 30 mL of H₂O were added to the flask, and then stirred at 160° C. After completion of the reaction, MeOH and H₂O were added to the reaction mixture, followed by stirring. Next, the solvent was removed by filtration under reduced pressure, followed by separating by column chromatography. Next, MeOH was added thereto, and the resulting solid was filtered under reduced pressure to obtain compound C-15 (17 g, yield: 97%).

| Compound | MW | color | M.P |
|---|---|---|---|
| C-15 | 733.95 | white | 220.8° C. |

### [Example 15] Synthesis of Compound C-59

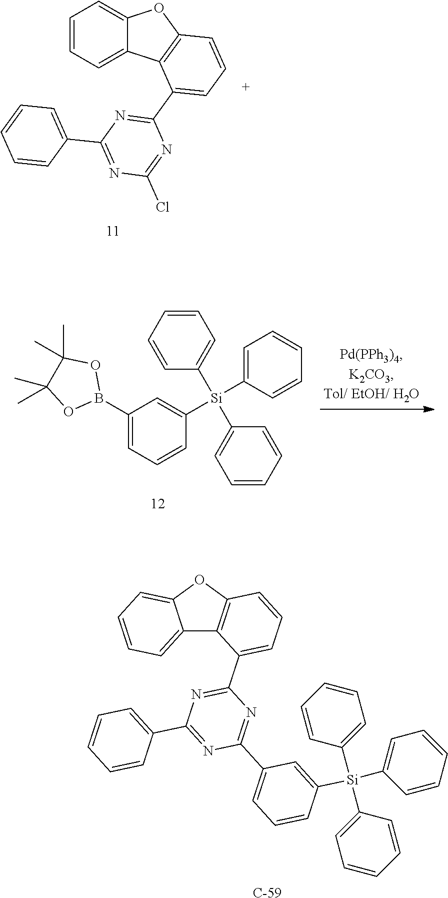

Compound 11 (8.0 g, 22.4 mmol), compound 12 (14.9 g, 32.1 mmol), Pd(PPh₃)₄ (1.3 g, 1.12 mmol), K₂CO₃ (6.2 g, 44.8 mmol), 114 mL of toluene (Tol), 30 mL of EtOH, and 31 mL of H₂O were added to the flask and dissolved, followed by stirring under reflux for 4 hours 20 minutes. After completion of the reaction, the mixture was cooled to room temperature and extracted with ethyl acetate (EA). Next, it was separated by column chromatography to obtain compound C-59 (9.5 g, yield: 65%).

| Compound | MW | M.P |
|---|---|---|
| C-59 | 657.85 | 193.5° C. |

### [Example 16] Synthesis of Compound C-6

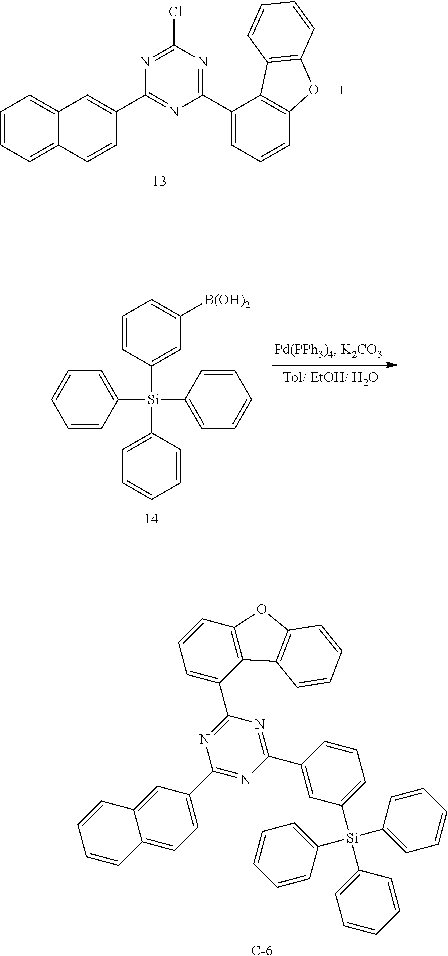

Compound 13 (9.5 g, 24.56 mmol), compound 14 (7 g, 292.56 mmol), Pd(PPh₃)₄ (1.4 g, 1.228 mmol), K₂CO₃ (8.48 g 61.4 mmol), 120 mL of Tol, 60 mL of EtOH, and 60 mL of H₂O were added to the flask and dissolved, followed by refluxing at 120° C. for 2 hours. After completion of the reaction, the organic layers were extracted with ethyl acetate, and the residual moisture was removed using magnesium sulfate, followed by drying. Next, it was separated by column chromatography to obtain compound C-6 (4.5 g, yield: 25.9%).

| Compound | MW | M.P |
|---|---|---|
| C-6 | 707.91 | 198.6° C. |

Hereinafter, the preparation method of an organic electroluminescent device comprising the plurality of host materials according to the present disclosure, and the device property thereof will be explained in order to understand the present disclosure in detail.

### [Device Examples 1 to 4] Preparation of OLEDs Co-Deposited with the First Host Material and the Second Host Material According to the Present Disclosure

OLEDs according to the present disclosure were prepared. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Then, compound HI-1 was introduced into a cell of the vacuum vapor deposition apparatus, and compound HT-1 was introduced into another cell. The two materials were evaporated at different rates and compound HI-1 was deposited in a doping amount of 3 wt % based on the total amount of compounds HI-1 and HT-1 to form a hole injection layer having a thickness of 10 nm. Next, compound HT-1 was deposited as a first hole transport layer having a thickness of 80 nm on the hole injection layer. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: each of the first host material and the second host material described in the following Table 1 were introduced into two cells of the vacuum vapor deposition apparatus as hosts, respectively, and compound D-39 was introduced into another cell as a dopant. The two host materials were evaporated at a rate of 1:1 and the dopant material was evaporated at a different rate, simultaneously, and was deposited in a doping amount of 3 wt % based on the total amount of the hosts and dopant to form a light-emitting layer having a thickness of 40 nm on the second hole transport layer. Next, compounds ET-1 and EI-1 as electron transport materials were deposited at a weight ratio of 50:50 to form an electron transport layer having a thickness of 35 nm on the light-emitting layer. After depositing compound EI-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. Each compound used for all the materials were purified by vacuum sublimation under 10⁻⁶ torr.

### [Device Comparative Example 1] Preparation of an OLED Comprising the Conventional Compound as a Host

An OLED was produced in the same manner as in Device Example 1, except that Compound A in the following Table 1 was used as one of the host materials of the light-emitting layer.

The driving voltage, the luminous efficiency, and the luminous color at a luminance of 1,000 nits, and the time taken for luminance to decrease from 100% to 95% at a luminance of 10,000 nits (lifespan; T95) of the OLEDs according to Device Examples 1 to 4 and Device Comparative Example 1 produced as described above, are measured, and the results thereof are shown in the following Table 1:

TABLE 1

| | | | Driving | Luminous | | |
|---|---|---|---|---|---|---|
| | First Host | Second Host | Voltage | Efficiency | Luminous | Lifespan |
| | Material | Material | (V) | (cd/A) | Color | T95(hr) |
| Device | H1-95 | A | 2.9 | 34.6 | Red | 246 |
| Comparative | | | | | | |
| Example 1 | | | | | | |
| Device | H1-95 | C-6 | 3.1 | 37.1 | Red | 348 |
| Example 1 | | | | | | |
| Device | H1-87 | C-6 | 3.2 | 38.0 | Red | 438 |
| Example 2 | | | | | | |
| Device | H1-96 | C-6 | 3.2 | 37.1 | Red | 394 |
| Example 3 | | | | | | |
| Device | H1-95 | C-59 | 3.0 | 37.1 | Red | 455 |
| Example 4 | | | | | | |

From Table 1 above, it can be confirmed that the organic electroluminescent device comprising a specific combination of compounds according to the present disclosure as a host material exhibits high luminous efficiency and, in particular, has significantly improved lifespan.

### [Device Examples 5 and 6] Preparation of OLEDs Comprising the Organic Electroluminescent Compound According to the Present Disclosure

OLEDs according to the present disclosure were prepared. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropanol and then used. Thereafter, the ITO substrate was mounted on a substrate holder of a vacuum vapor deposition apparatus. Then, compound HI-1 was introduced into a cell of the vacuum vapor deposition apparatus, and compound HT-1 was introduced into another cell. The two materials were evaporated at different rates and compound HI-1 was deposited in a doping amount of 3 wt % based on the total amount of compound HI-1 and compound HT-1 to form a hole injection layer having a thickness of 10 nm. Next, compound HT-1 was deposited as a first hole transport layer having a thickness of 80 nm on the hole injection layer. Compound HT-3 was then introduced into another cell of the vacuum vapor deposition apparatus and was evaporated by applying an electric current to the cell, thereby forming a second hole transport layer having a thickness of 5 nm on the first hole transport layer. After forming the hole injection layer and the hole transport layers, a light-emitting layer was formed thereon as follows: Compound BH was introduced into a cell of the vacuum vapor deposition apparatus as a host, and compound BD was introduced into another cell as a dopant. The two materials were evaporated at different rates, and the dopant was deposited in a doping amount of 8 wt % based on the total amount of the host and dopant to form a light-emitting layer having a thickness of 20 nm on the second hole transport layer. Next, the compound shown in the following Table 2 as an electron buffer layer was deposited to form an electron buffer layer having a thickness of 5 nm on the light-emitting layer. Next, compounds ET-1 and EI-1 as an electron transport layer were deposited at a weight ratio of 50:50 to form an electron transport layer having a thickness of 30 nm on the electron buffer layer. After depositing compound EI-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. Each compound used for all the materials were purified by vacuum sublimation under 10⁻⁶ torr.

### [Device Comparative Example 2] Preparation of an OLED Comprising the Conventional Compound as an Electron Buffer Layer Material

An OLED was produced in the same manner as in Device Example 5, except that the compound shown in the following Table 2 was used as the electron buffer layer material.

The driving voltage, the luminous efficiency, and the Luminous Color at a luminance of 1,000 nits of the OLEDs according to Device Examples 5 and 6 and Device Comparative Example 2 produced as described above, are measured, and the results thereof are shown in the following Table 2:

TABLE 2

| | | Driving | Luminous | |
|---|---|---|---|---|
| | Electron | Voltage | Efficiency | Luminous |
| | buffer layer | (V) | (cd/A) | Color |
| Device Comparative | A | 4.6 | 4.5 | Blue |
| Example 2 | | | | |
| Device Example 5 | C-6 | 4.2 | 5.2 | Blue |
| Device Example 6 | C-15 | 4.4 | 4.8 | Blue |

From Table 2 above, it can be confirmed that the organic electroluminescent device comprising the organic electroluminescent compound according to the present disclosure as an electron buffer layer material exhibits a lower driving voltage and higher luminous efficiency than the conventional organic electroluminescent device.

The compounds used in Device Examples 1 to 6 and Device Comparative Examples 1 and 2 are specifically shown in the following Table 3.

TABLE 3

| | |
|---|---|
| Hole Injection Layer/ Hole Transport Layer | 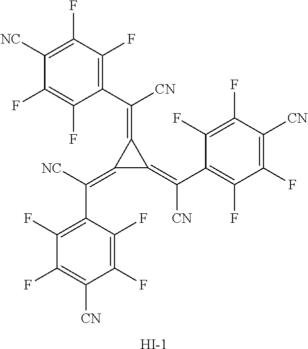 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 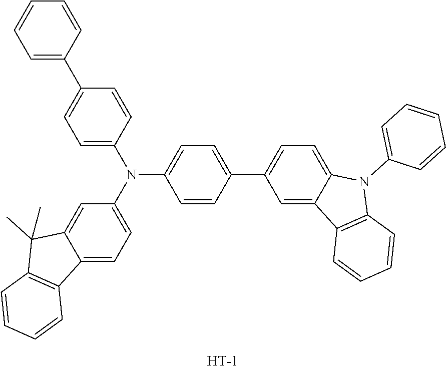 |
| | 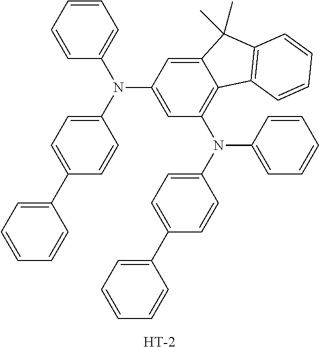 |
| | 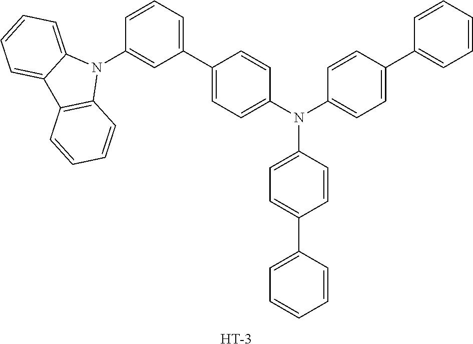 |
| Light-Emitting Layer/Electron Buffer Layer | 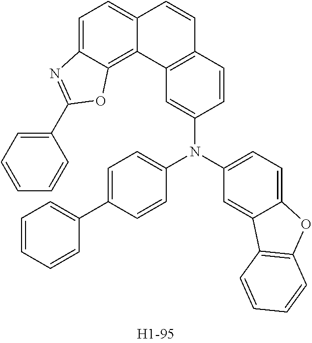 [SMILES: c1ccc(-c2ccc(N(c3ccc4oc5ccccc5c4c3)c3ccc4ccc5ccc6nc(-c7ccccc7)oc6c5c4c3)cc2)cc1] |
| | 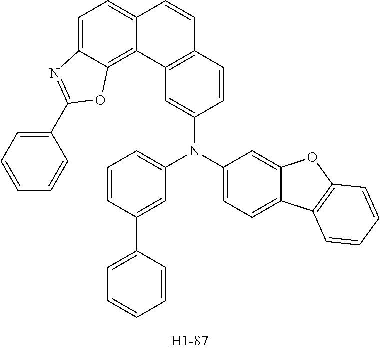 [SMILES: c1ccc(-c2cccc(N(c3ccc4c(c3)oc3ccccc34)c3ccc4ccc5ccc6nc(-c7ccccc7)oc6c5c4c3)c2)cc1] |
| | 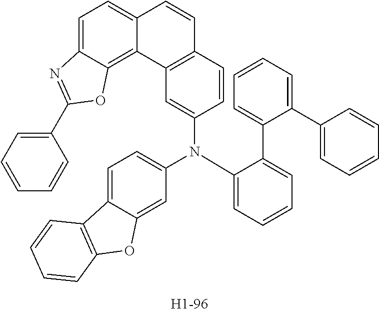 [SMILES: c1ccc(-c2nc3ccc4ccc5ccc(N(c6ccc7c(c6)oc6ccccc67)c6ccccc6-c6ccccc6-c6ccccc6)cc5c4c3o2)cc1] |
| | 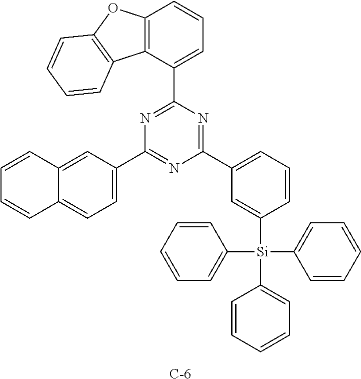 |
| | 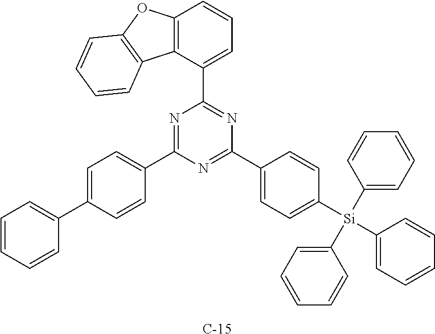 |
| | 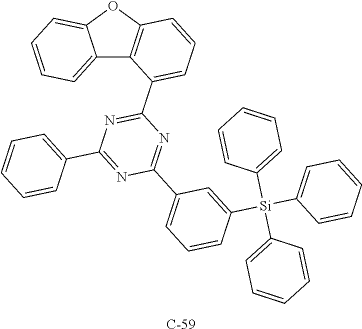 |
| | 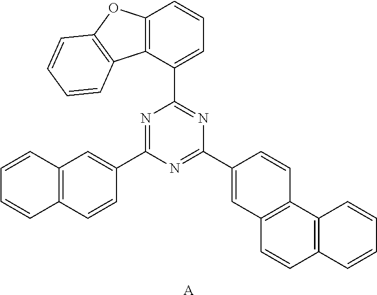 |
| | 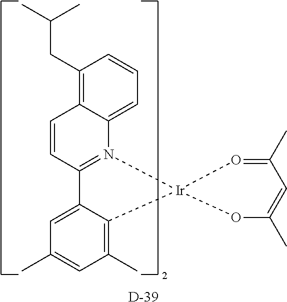 |
| | 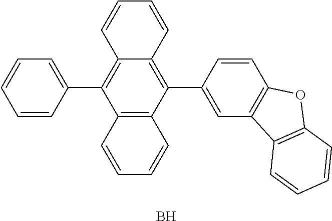 |
| | 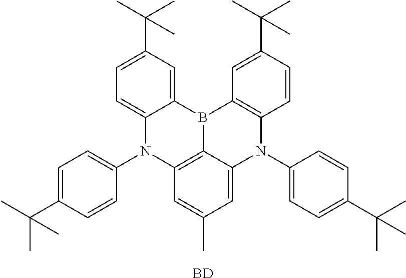 |
| Electron Transport Layer/ Electron Injection Layer | 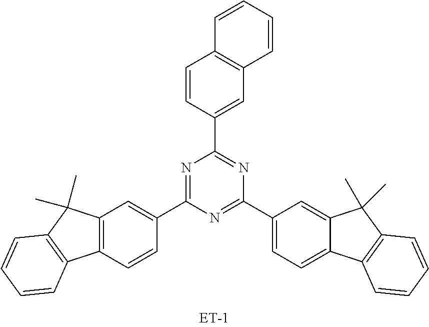 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 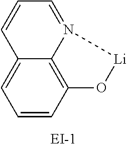 |

1. A plurality of host materials comprising a first host material and a second host material, wherein the first host material comprises a compound represented by the following formula 1 and the second host material comprises a compound represented by the following formula 2: 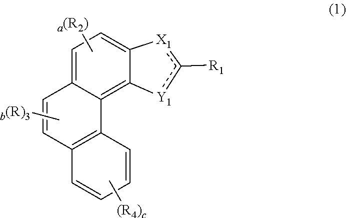
wherein,
X₁ and Y₁ each independently represent —N═, —NR₅—, —O— or —S—; provided that any one of X₁ and Y₁ is —N═, and other of X₁ and Y₁ is —NR₅—, —O— or —S—;
R₁ is a substituted or unsubstituted (C6-C30)aryl or a substituted or unsubstituted (3- to 30-membered)heteroaryl;
R₂ to R₅ each independently represent hydrogen, deuterium, cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted fused ring of (C3-C30) aliphatic ring and a (C6-C30) aromatic ring, —NR₁₁R_{12.} —SiR₁₃R₁₄R₁₅ or a combination thereof; or may be linked to the adjacent substituents to form a ring(s);
R₁₁ to R₁₅ each independently represent a substituted or unsubstituted (C1-C30)alkyl, a substituted or unsubstituted (C6-C30)aryl, or a substituted or unsubstituted (3- to 30-membered)heteroaryl;
a and b each independently represent an integer of 1 or 2, and c represents an integer of 1 to 4; and
when a to c are an integer of 2 or more, each R₂, each of R₃, and each of R₄ may be the same or different; 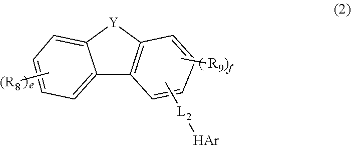
wherein,
Y represents —O— or —S—;
L₂ represents a single bond, a substituted or unsubstituted (C6-C30)arylene, or a substituted or unsubstituted (3- to 30-membered)heteroarylene;
HAr represents a substituted or unsubstituted (3- to 30-membered)heteroaryl comprising at least one nitrogen atom;
R₈ and R₉ each independently represent hydrogen, deuterium, cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₁₆R₁₇, —SiR₁₈R₁₉R₂₀, or a combination thereof; or may be linked to the adjacent substituents to form a ring(s);
R₁₆ to R₂₀ each independently represent a substituted or unsubstituted (C1-C30)alkyl, a substituted or unsubstituted (C6-C30)aryl, or a substituted or unsubstituted (3- to 30-membered)heteroaryl;
e represents an integer of 1 to 4, and f represents an integer of 1 to 3;
when e and f are an integer of 2 or more, each of R₈ and each of R₉ may be the same or different;
provided that at least one of HAr, R₈, and R₉ includes -L₃-SiR′R″R′″ or -L₃-CR′R″R′″;
L₃ represents a single bond, a substituted or unsubstituted (C1-C30)alkylene, a substituted or unsubstituted (C3-C30)cycloalkylene, a substituted or unsubstituted (C6-C30)arylene, or a substituted or unsubstituted (3- to 30-membered)heteroarylene; and
R′, R″, and R′″ each independently represent a substituted or unsubstituted (C6-C30)aryl or a substituted or unsubstituted (3- to 30-membered)heteroaryl.

2. The plurality of host materials according to claim 1, wherein substituents in the substituted alkyl, the substituted cycloalkyl, the substituted cycloalkenyl, the substituted heterocycloalkyl, the substituted aryl(ene), the substituted heteroaryl(ene), and the substituted nitrogen-comprising heteroaryl, each independently represent at least one selected from the group consisting of deuterium; halogen; cyano; carboxyl; nitro; hydroxyl; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3- to 7-membered)heterocycloalkyl; (C6-C30)aryloxy; (C6-C30)arylthio; (3- to 30-membered)heteroaryl unsubstituted or substituted by (C6-C30)aryl; (C6-C30)aryl unsubstituted or substituted by at least one of (C1-C30)alkyl and (3- to 30-membered)heteroaryl; tri(C1-C30)alkylsilyl; tri(C6-C30)arylsilyl; di(C1-C30)alkyl(C6-C30)arylsilyl; (C1-C30)alkyldi(C6-C30)arylsilyl; amino; mono- or di-(C1-C30)alkylamino; mono- or di-(C6-C30)arylamino; (C1-C30)alkyl(C6-C30)arylamino; (C1-C30)alkylcarbonyl; (C1-C30)alkoxycarbonyl; (C6-C30)arylcarbonyl; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl.

3. The plurality of host materials according to claim 1, wherein the compound represented by the formula 1 is represented by the following formula 1-1 or 1-2: 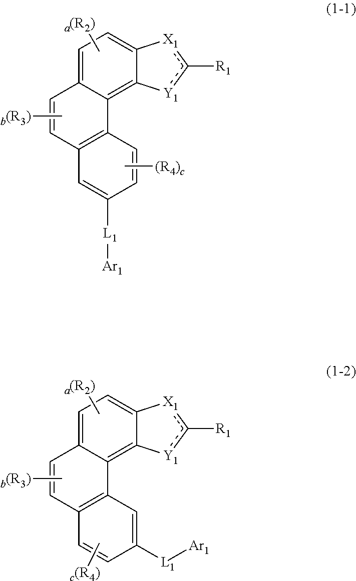
wherein,
L₁ represents a single bond or a substituted or unsubstituted (C6-C30)arylene;
Ar₁ represents deuterium, halogen, cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted fused ring of (C3-C30) aliphatic ring and a (C6-C30) aromatic ring, —NR₁₁R₁₂, —SiR₁₃R₁₄R₁₅, or a combination thereof;
c represents an integer of 1 to 3; and
X₁, Y₁, R₁ to R₄, R₁₁ to R₁₅, a, and b are as defined in claim 1.

4. The plurality of host materials according to claim 3, wherein Ar₁ represents a substituted or unsubstituted phenyl, a substituted or unsubstituted naphthyl, a substituted or unsubstituted biphenyl, a substituted or unsubstituted naphthylphenyl, a substituted or unsubstituted phenylnaphthyl, a substituted or unsubstituted terphenyl, a substituted or unsubstituted phenanthrenyl, a substituted or unsubstituted benzophenanthrenyl, a substituted or unsubstituted chrysenyl, a substituted or unsubstituted fluoranthenyl, a substituted or unsubstituted fluorenyl, a substituted or unsubstituted benzofluorenyl, a substituted or unsubstituted triphenylenyl, a substituted or unsubstituted spirobifluorenyl, a substituted or unsubstituted spiro[cyclopentan-fluoren]yl, a substituted or unsubstituted spiro[indan-fluoren]yl, a substituted or unsubstituted spiro[fluoren-benzofluoren]yl, or amino substituted by at least one of phenyl; naphthyl; biphenyl; terphenyl; a substituted or unsubstituted fluorenyl; phenanthrenyl; dibenzofuranyl unsubstituted or substituted by phenyl; dibenzothiophenyl; carbazolyl unsubstituted or substituted by phenyl; and benzonaphthofuranyl.

5. The plurality of host materials according to claim 1, wherein HAr represents a substituted or unsubstituted pyridyl, a substituted or unsubstituted pyrimidinyl, a substituted or unsubstituted triazinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthyridinyl, a substituted or unsubstituted benzofuropyrimidinyl, a substituted or unsubstituted benzothienopyrimidinyl, a substituted or unsubstituted carbazolyl, or a substituted or unsubstituted pyridopyrazinyl.

6. The plurality of host materials according to claim 1, wherein the compound represented by the formula 2 is represented by the following formula 2-1: 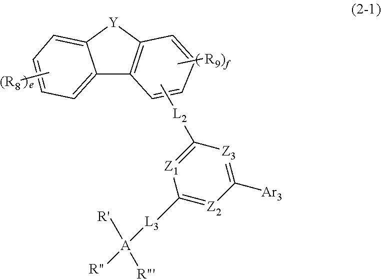
wherein,
Z₁ to Z₃ each independently represent N or CH; provided that at least one of Z₁ to Z₃ is N;
A represents Si or C;
Ar₃ represents a substituted or unsubstituted (C6-C30)aryl or a substituted or unsubstituted (3- to 30-membered)heteroaryl; and
Y, R₈, R₉, L₂, L₃, R′, R″, R′″, e, and f are as defined in claim 1.

7. The plurality of host materials according to claim 1, wherein the compound represented by the formula 1 is selected from the following compounds:

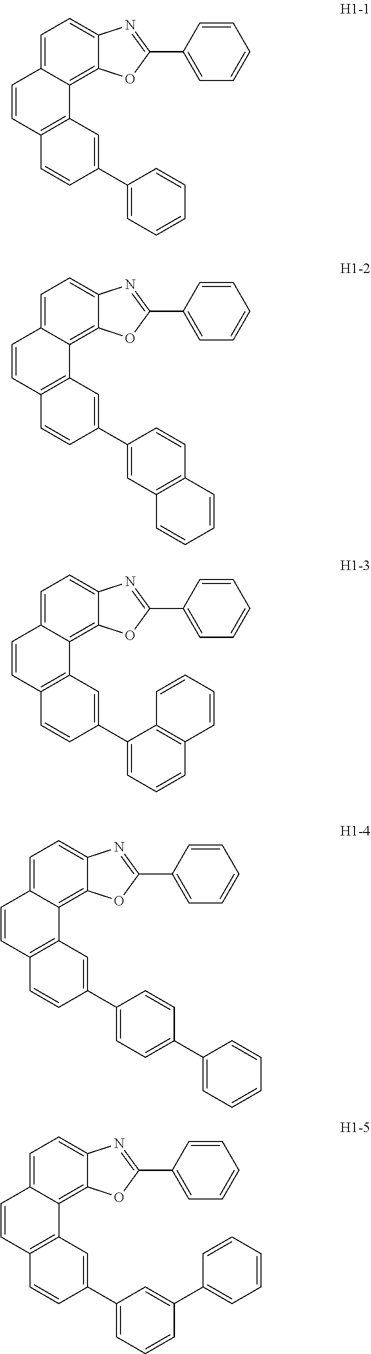

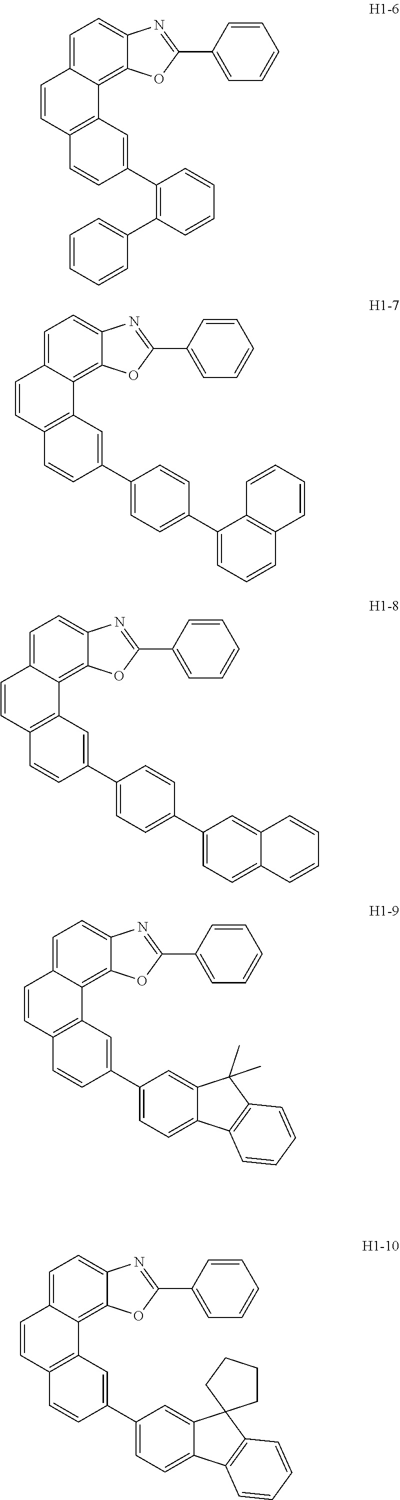

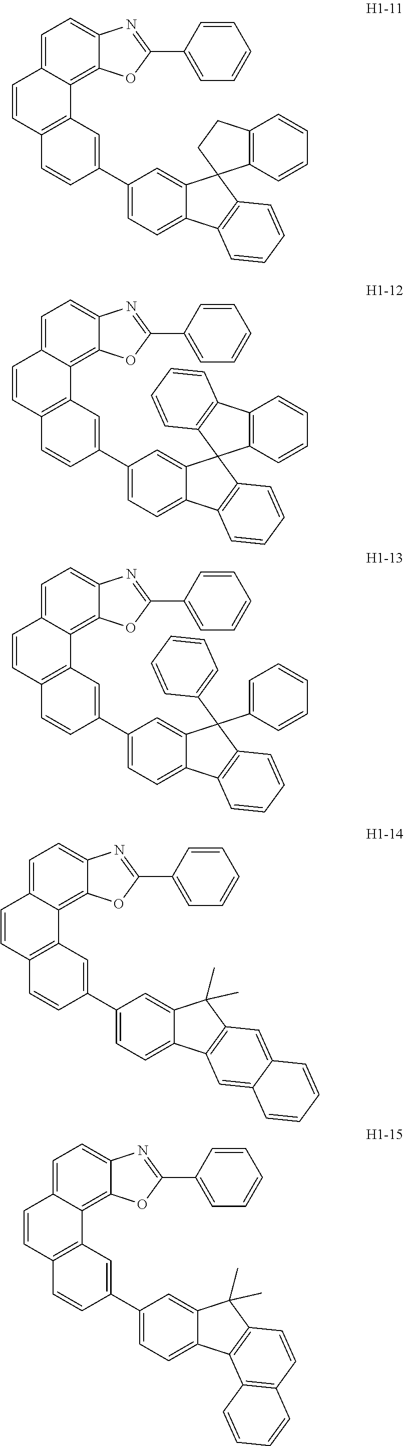

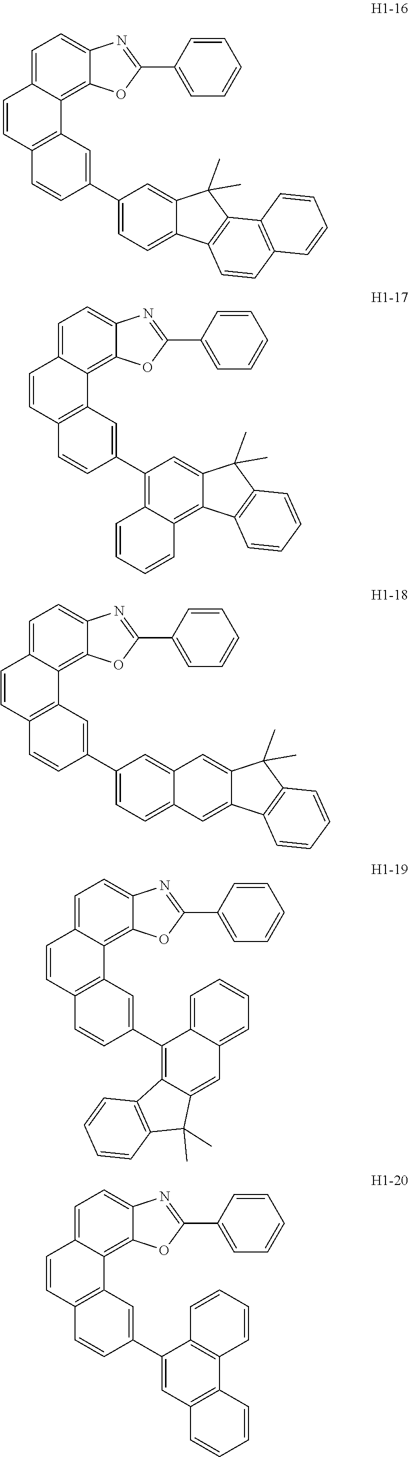

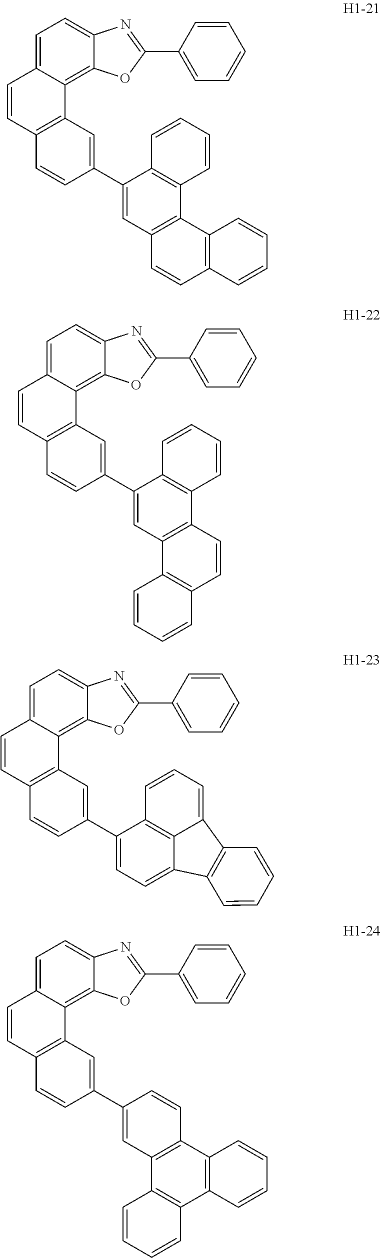

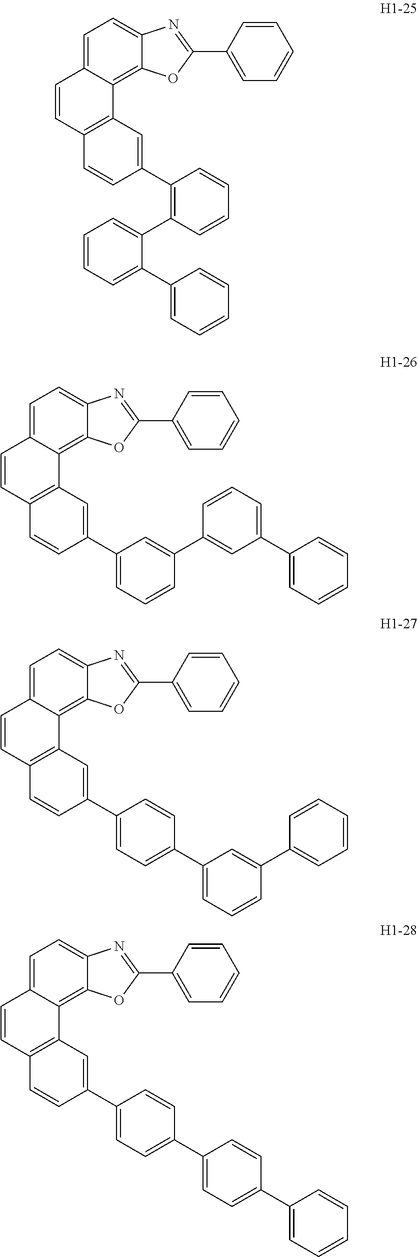

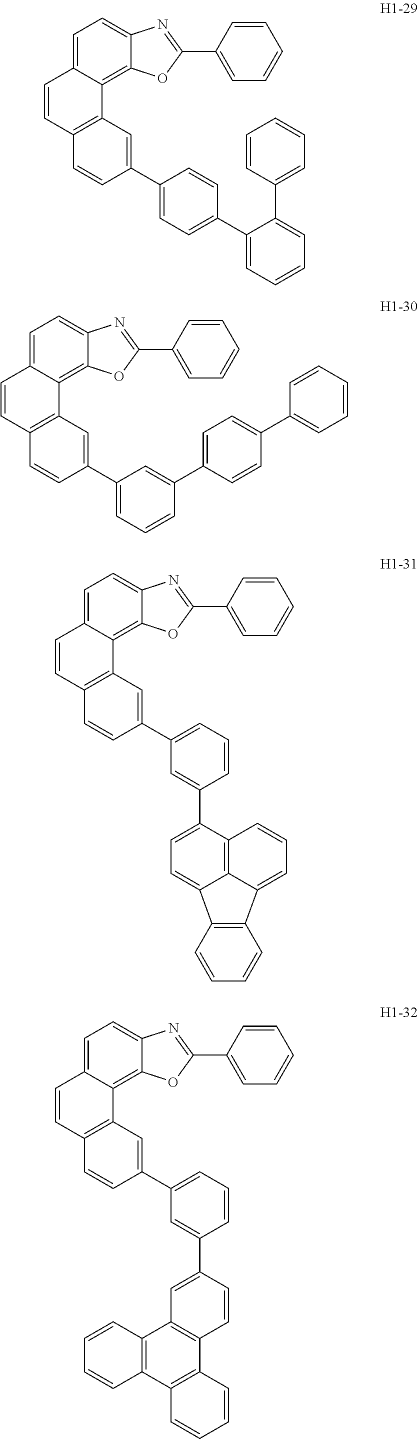

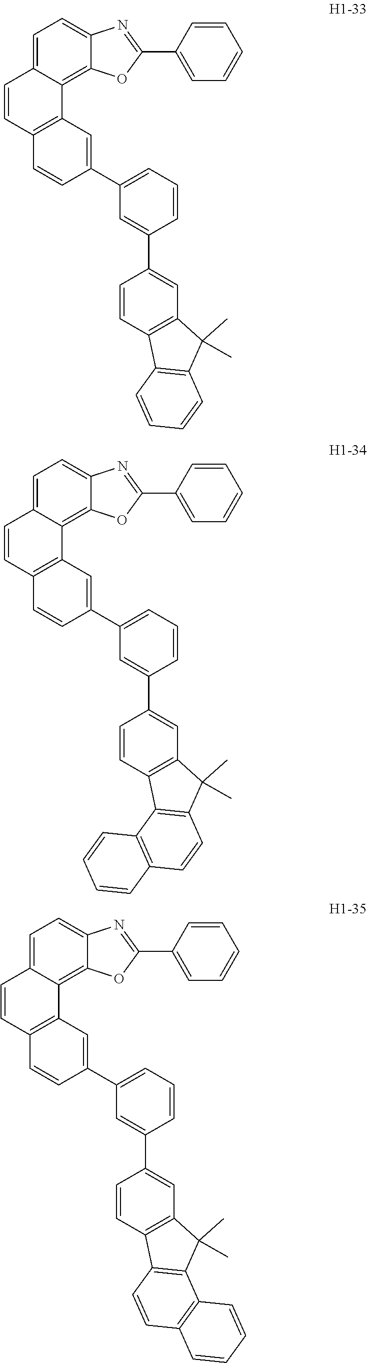

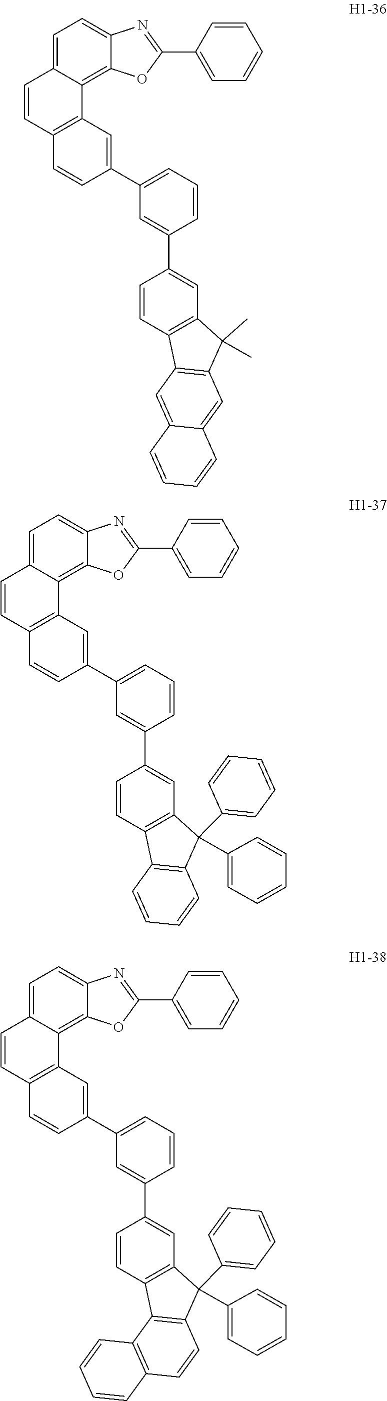

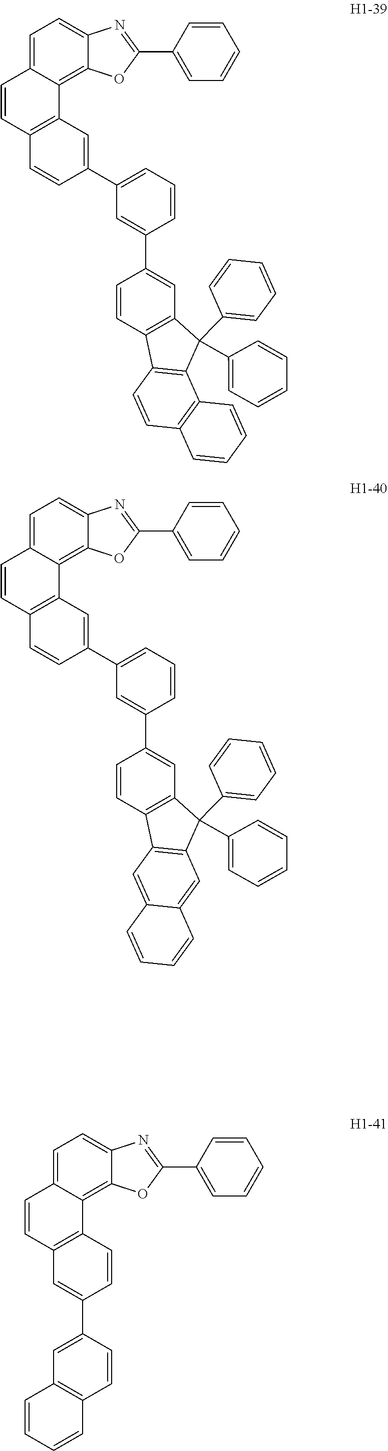

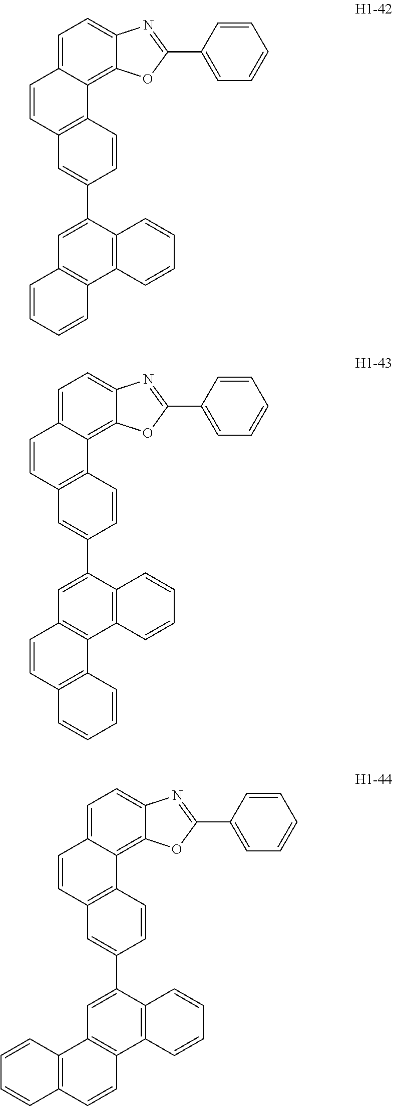 [SMILES: c1ccc(-c2nc3ccc4ccc5cc(-c6cc7ccccc7c7ccccc67)ccc5c4c3o2)cc1 ; c1ccc(-c2nc3ccc4ccc5cc(-c6cc7ccc8ccccc8c7c7ccccc67)ccc5c4c3o2)cc1 ; c1ccc(-c2nc3ccc4ccc5cc(-c6cc7c8ccccc8ccc7c7ccccc67)ccc5c4c3o2)cc1]

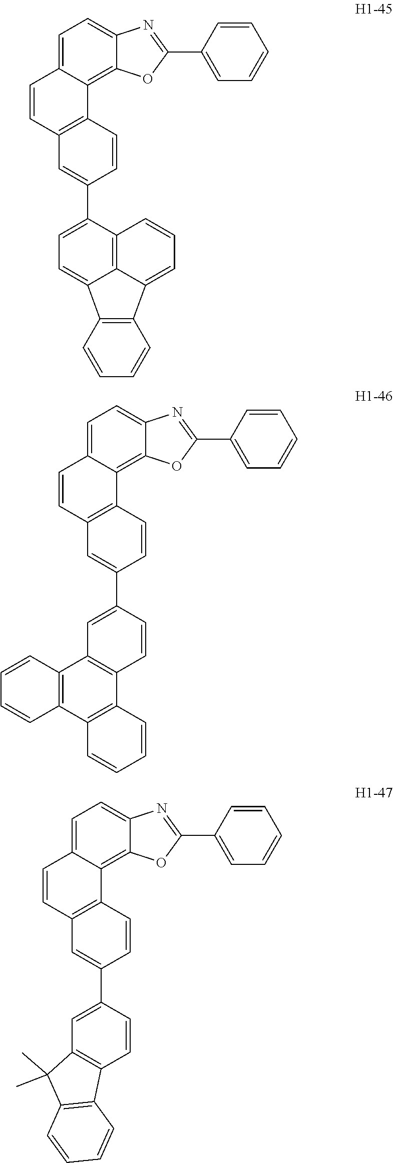

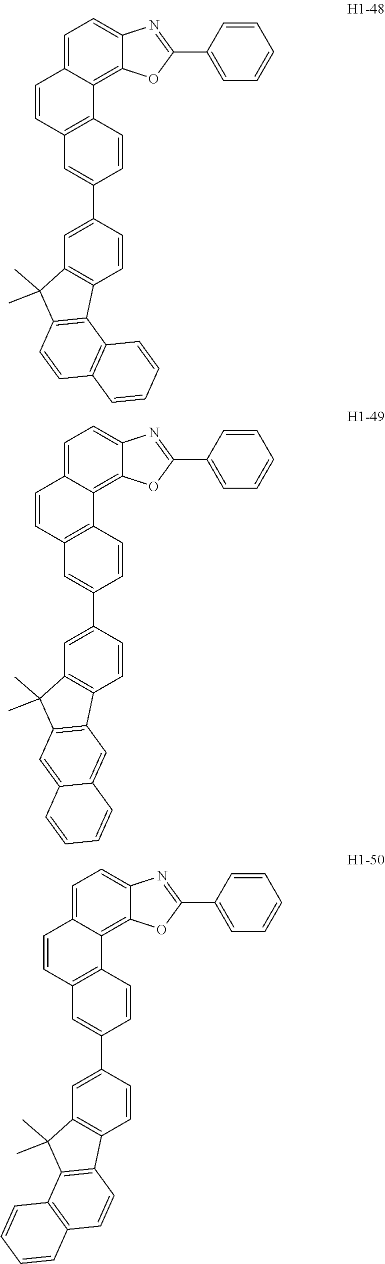

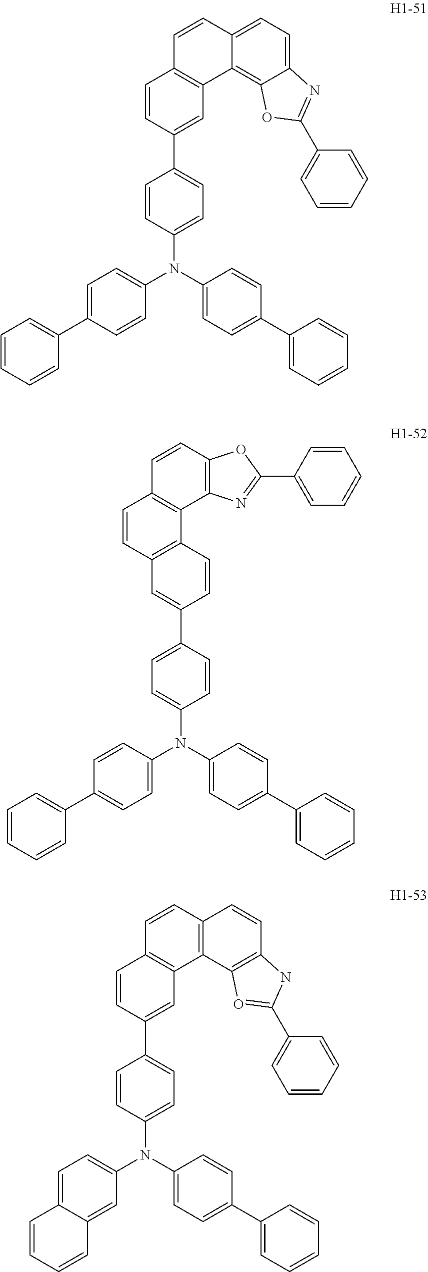

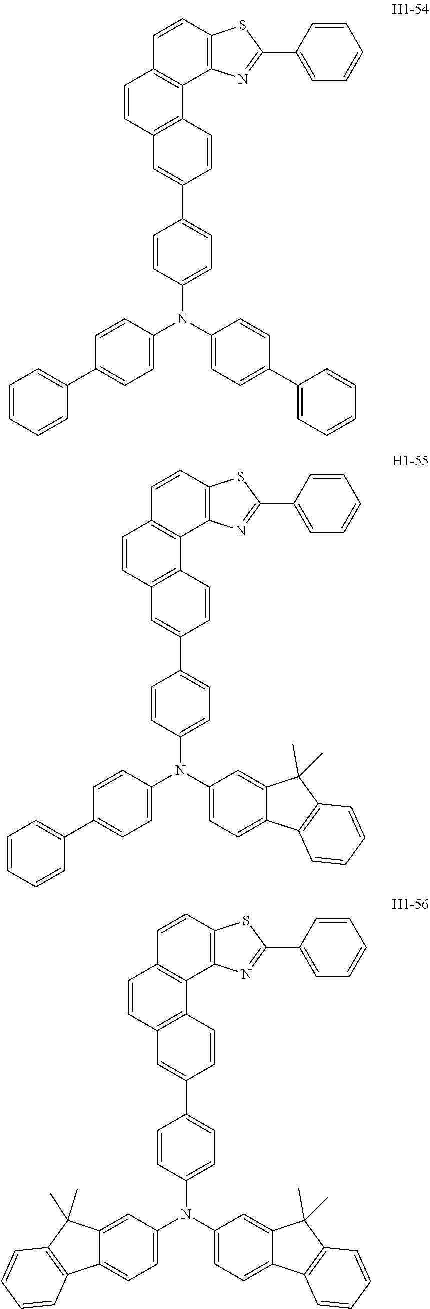

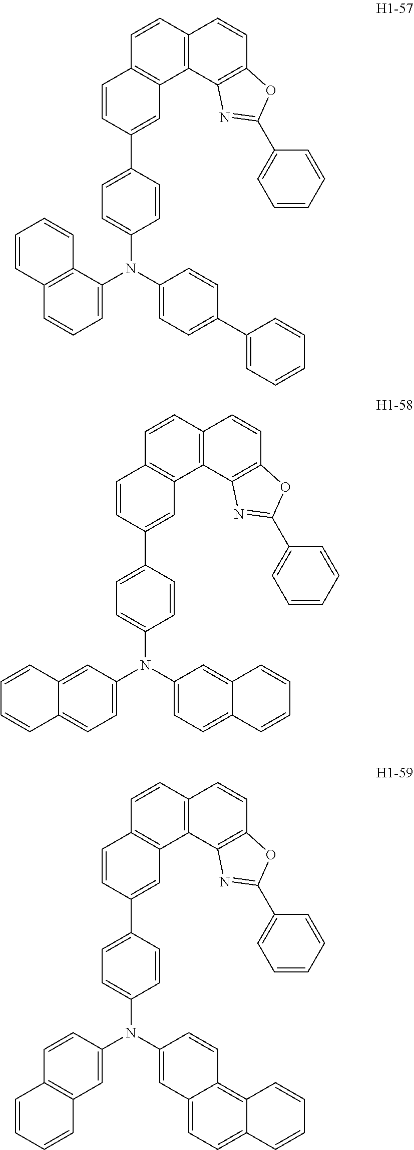 [SMILES: c1ccc(-c2ccc(N(c3ccc(-c4ccc5ccc6ccc7oc(-c8ccccc8)nc7c6c5c4)cc3)c3cccc4ccccc34)cc2)cc1 ; c1ccc(-c2nc3c(ccc4ccc5ccc(-c6ccc(N(c7ccc8ccccc8c7)c7ccc8ccccc8c7)cc6)cc5c43)o2)cc1 ; c1ccc(-c2nc3c(ccc4ccc5ccc(-c6ccc(N(c7ccc8ccccc8c7)c7ccc8c(ccc9ccccc98)c7)cc6)cc5c43)o2)cc1]

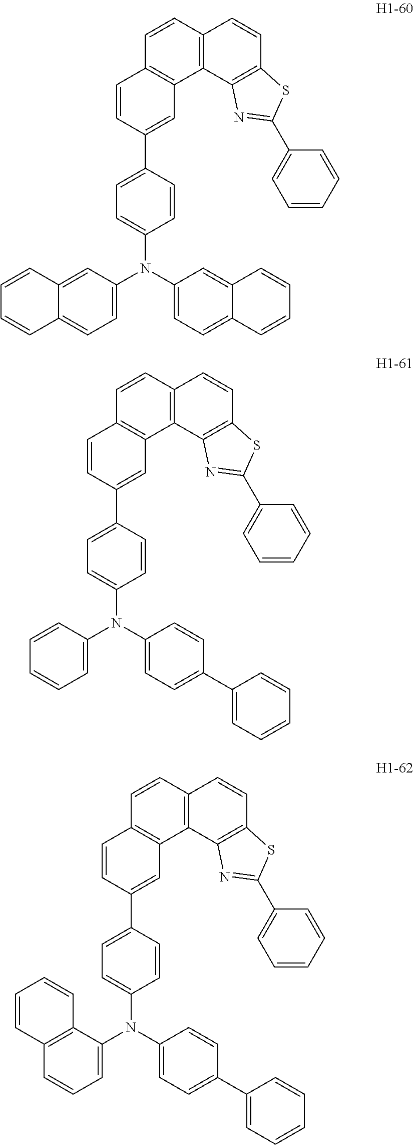

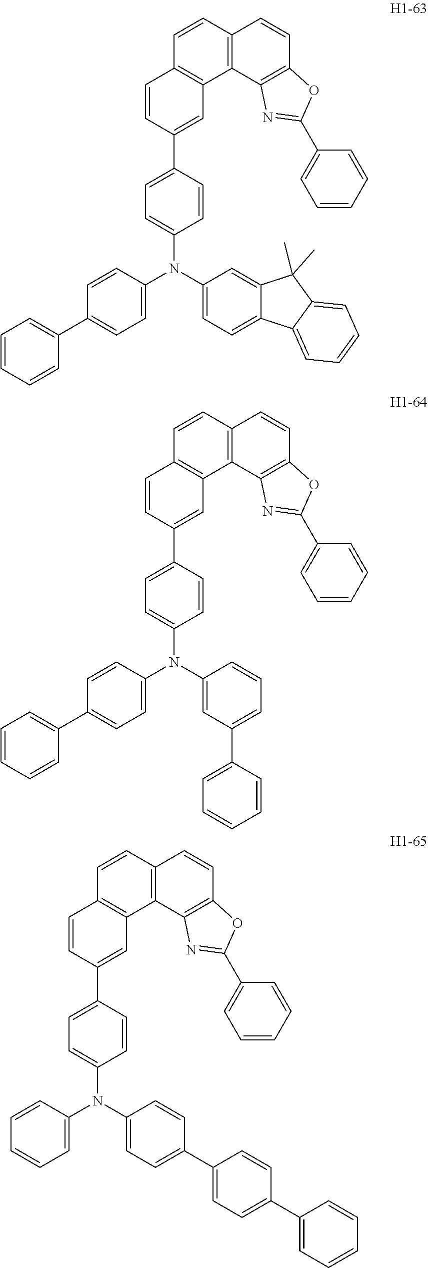

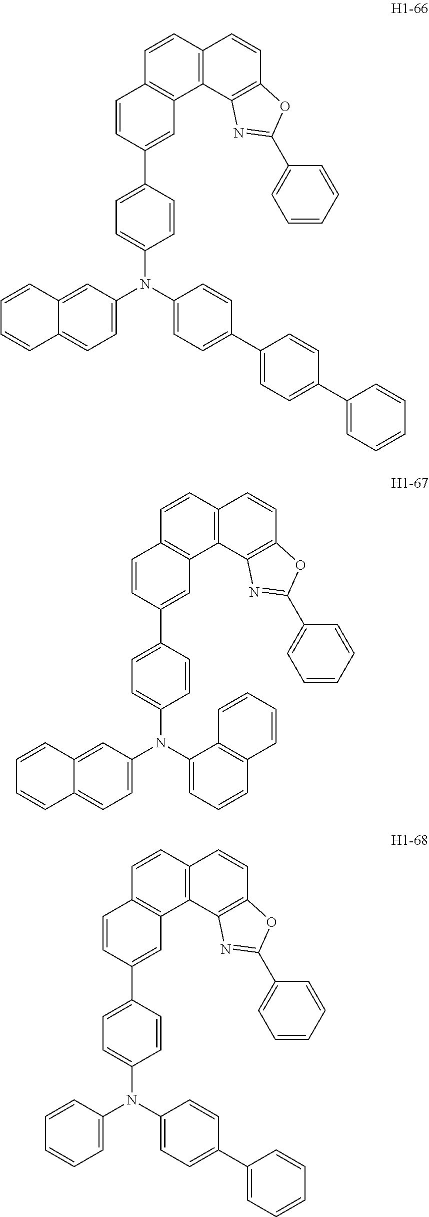

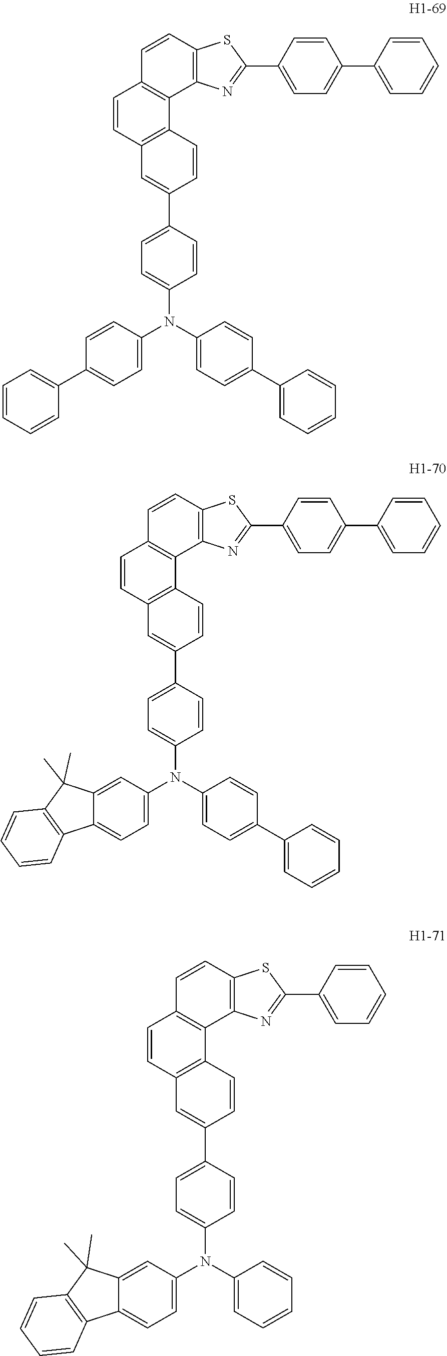 [SMILES: c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21]

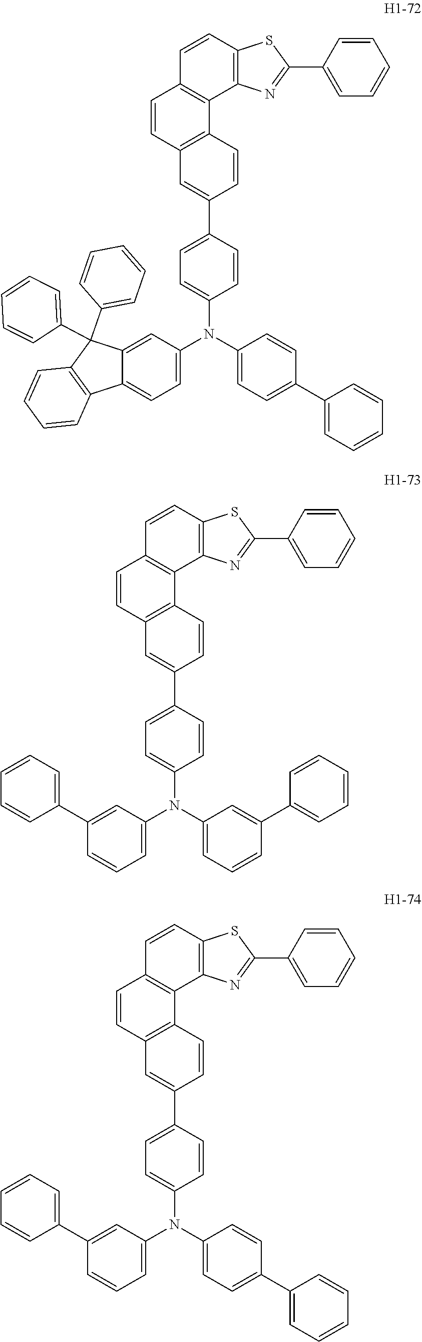 [SMILES: c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1]

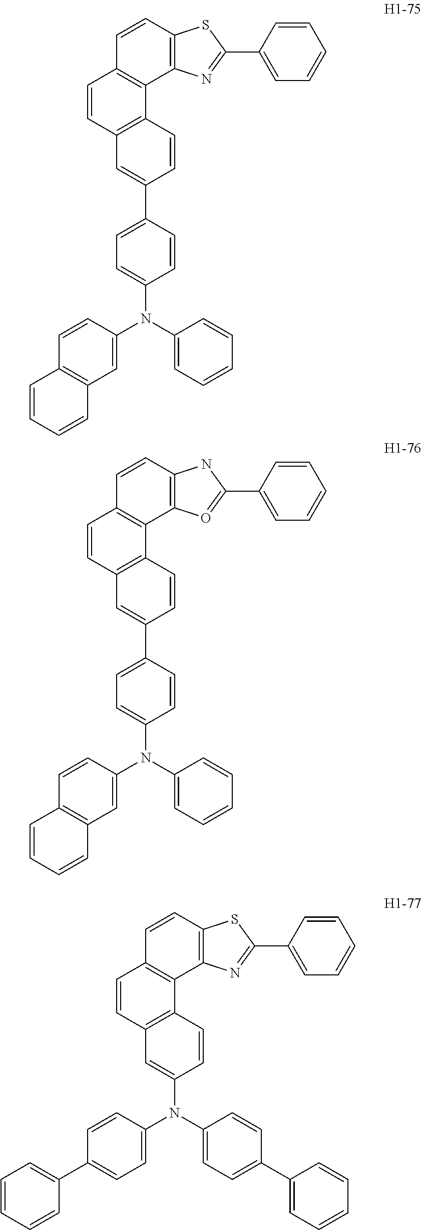

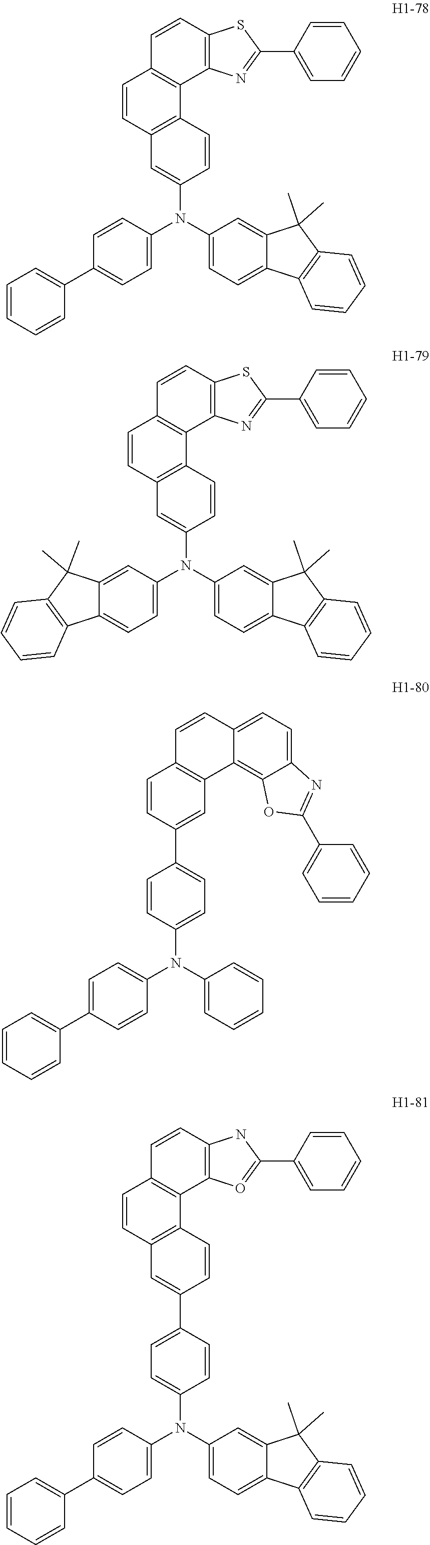

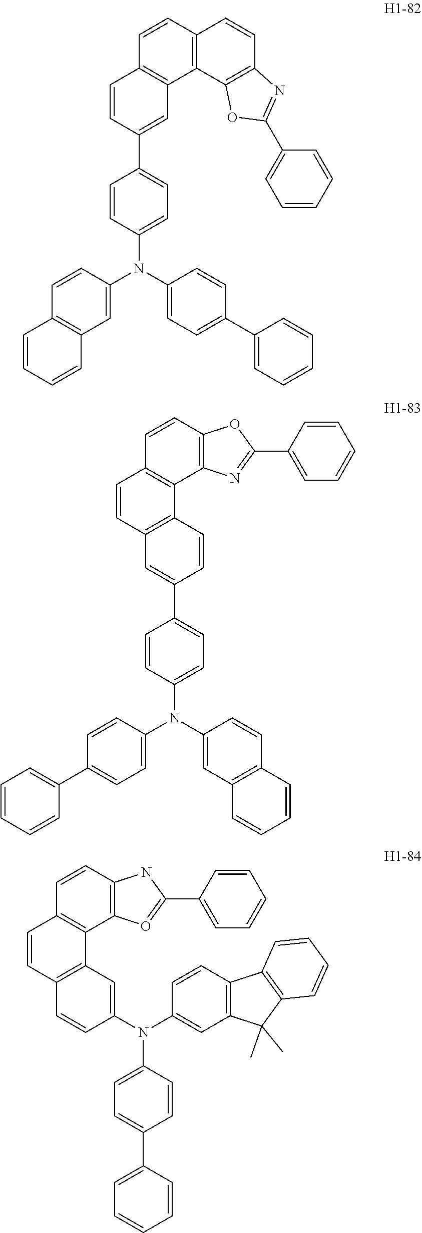

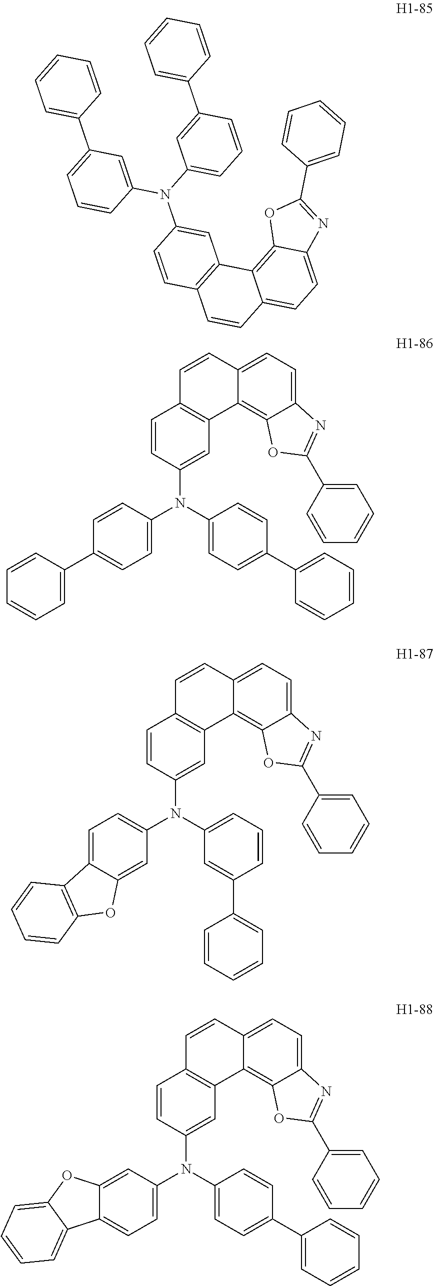

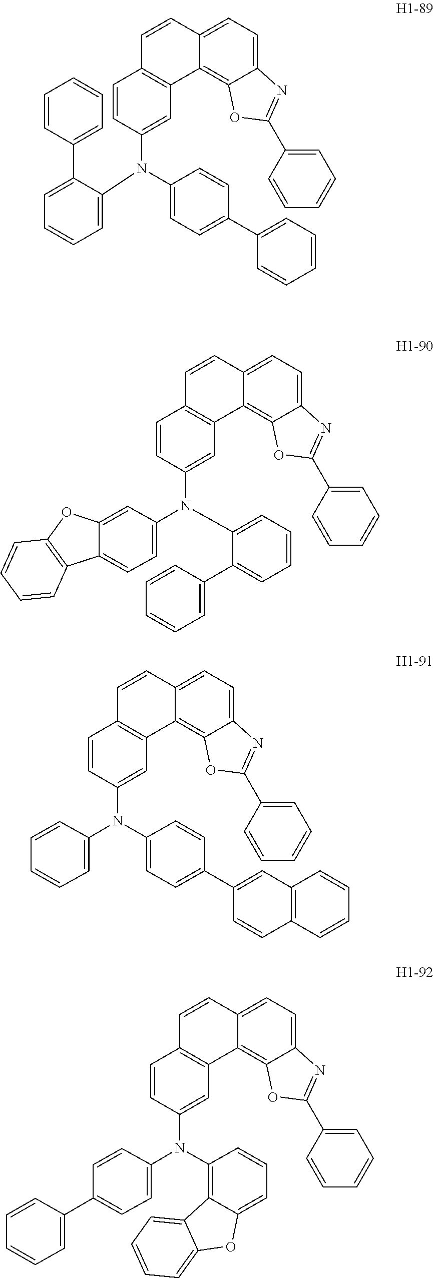 [SMILES: c1ccc(-c2ccc(N(c3ccc4ccc5ccc6nc(-c7ccccc7)oc6c5c4c3)c3ccccc3-c3ccccc3)cc2)cc1 ; c1ccc(-c2nc3ccc4ccc5ccc(N(c6ccc7c(c6)oc6ccccc67)c6ccccc6-c6ccccc6)cc5c4c3o2)cc1 ; c1ccc(-c2nc3ccc4ccc5ccc(N(c6ccccc6)c6ccc(-c7ccc8ccccc8c7)cc6)cc5c4c3o2)cc1 ; c1ccc(-c2ccc(N(c3ccc4ccc5ccc6nc(-c7ccccc7)oc6c5c4c3)c3cccc4oc5ccccc5c34)cc2)cc1]

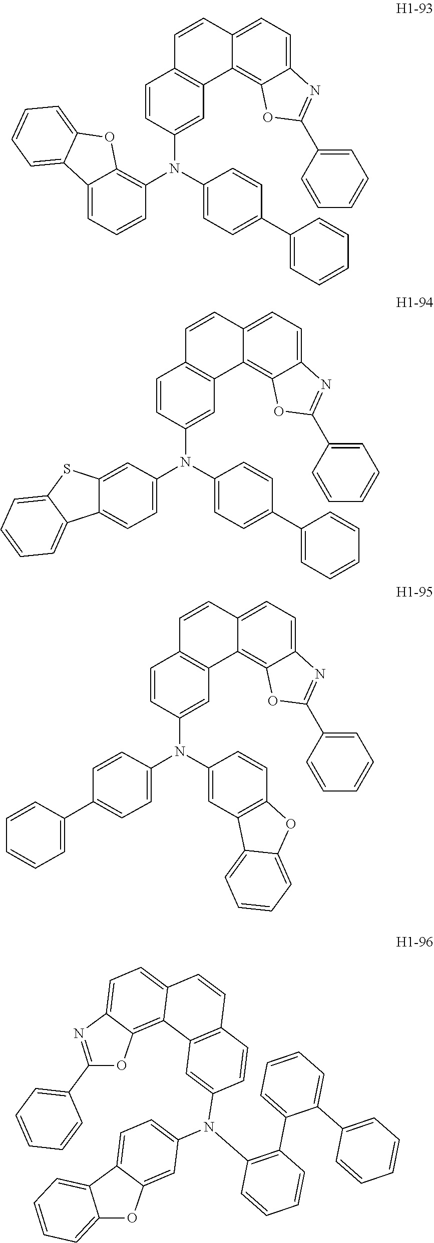 [SMILES: c1ccc(-c2ccc(N(c3ccc4ccc5ccc6nc(-c7ccccc7)oc6c5c4c3)c3cccc4c3oc3ccccc34)cc2)cc1 ; c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2ccc(N(c3ccc4oc5ccccc5c4c3)c3ccc4ccc5ccc6nc(-c7ccccc7)oc6c5c4c3)cc2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1]

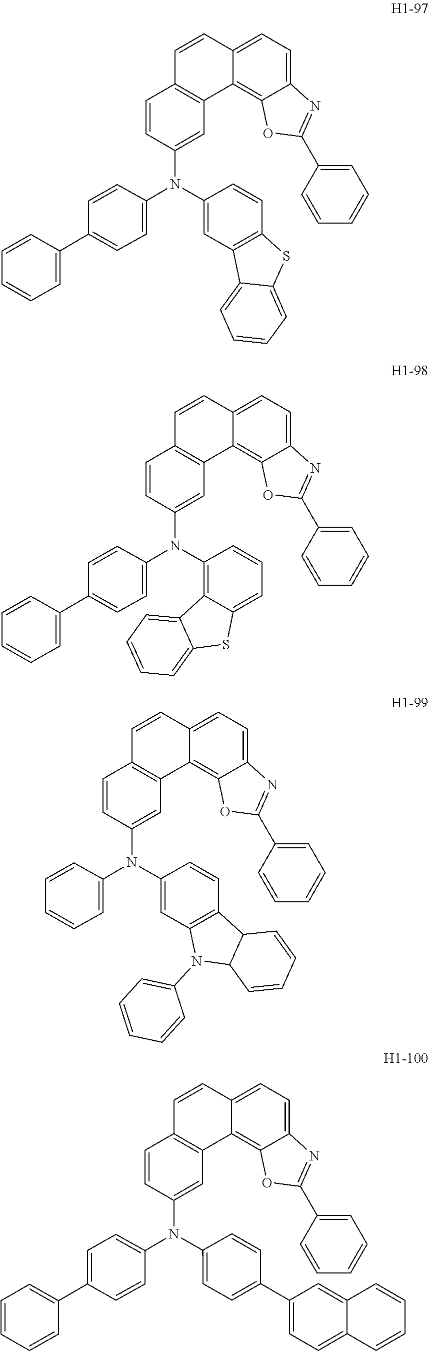

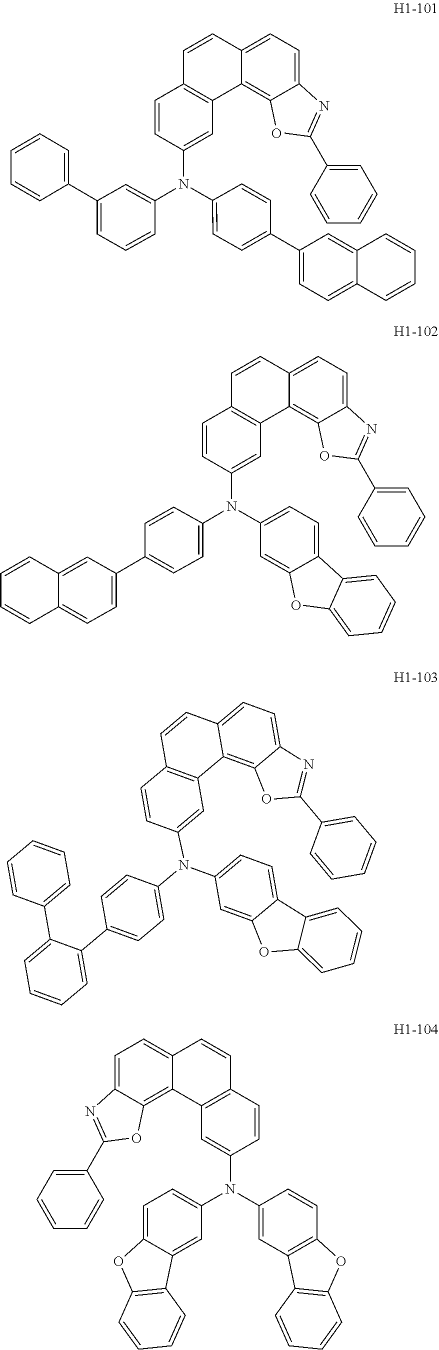 [SMILES: c1ccc(-c2cccc(N(c3ccc(-c4ccc5ccccc5c4)cc3)c3ccc4ccc5ccc6nc(-c7ccccc7)oc6c5c4c3)c2)cc1 ; c1ccc(-c2nc3ccc4ccc5ccc(N(c6ccc(-c7ccc8ccccc8c7)cc6)c6ccc7c(c6)oc6ccccc67)cc5c4c3o2)cc1 ; c1ccc(-c2nc3ccc4ccc5ccc(N(c6ccc(-c7ccccc7-c7ccccc7)cc6)c6ccc7c(c6)oc6ccccc67)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1]

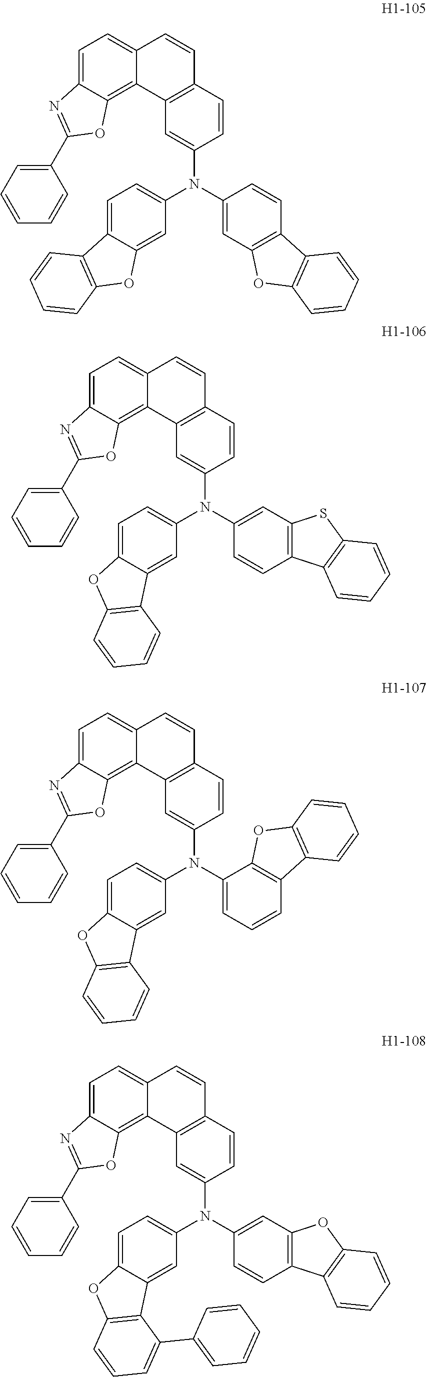

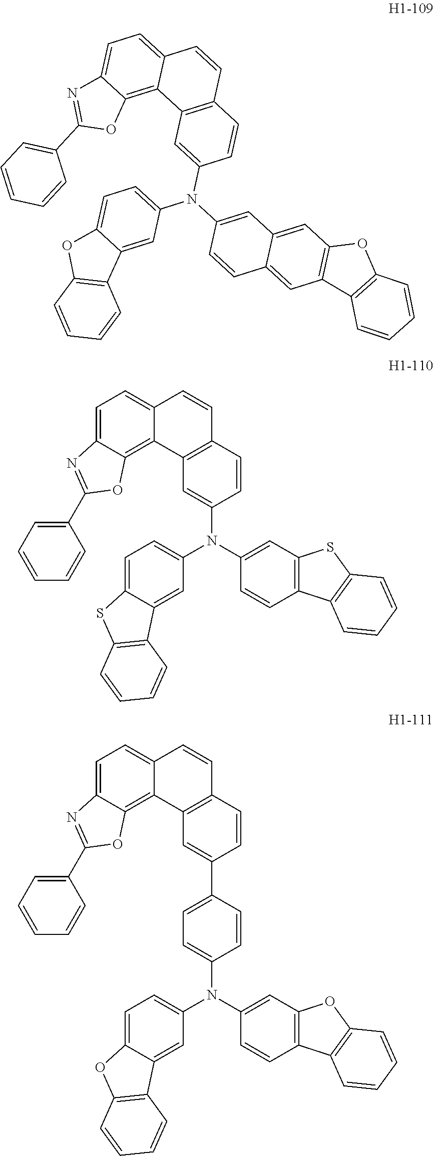

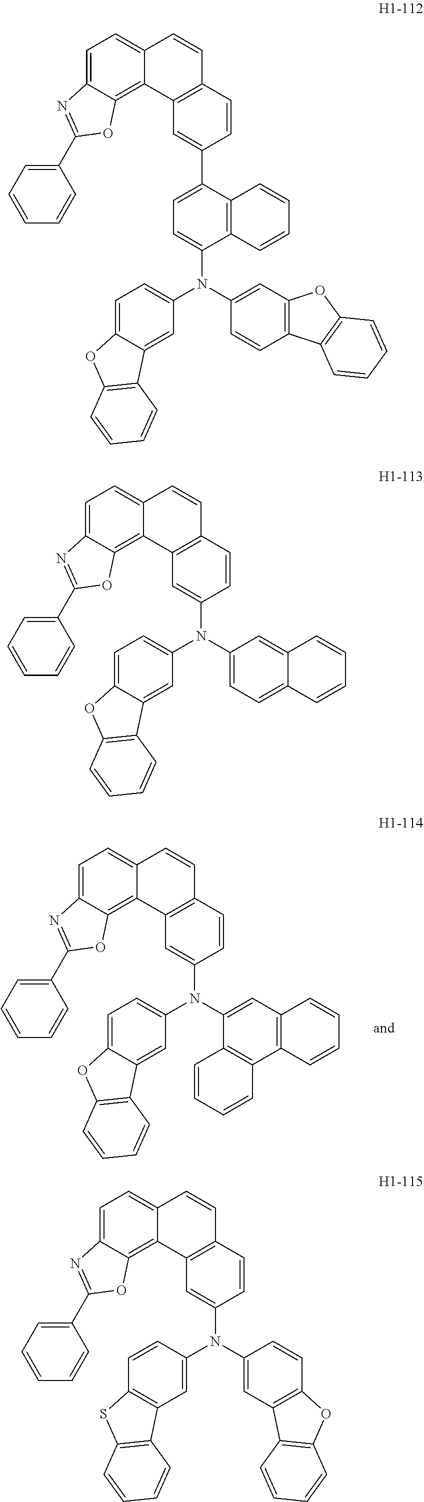

8. The plurality of host materials according to claim 1, wherein the compound represented by the formula 2 is selected from the following compounds:

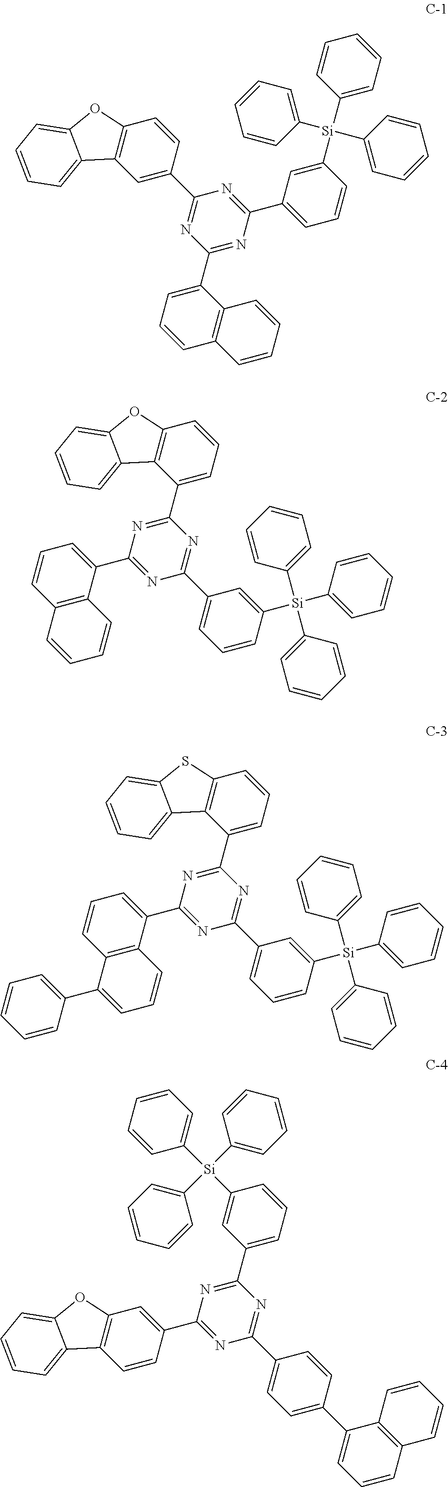 [SMILES: c1ccc([Si](c2ccccc2)(c2ccccc2)c2cccc(-c3nc(-c4ccc5oc6ccccc6c5c4)nc(-c4cccc5ccccc45)n3)c2)cc1 ; c1ccc([Si](c2ccccc2)(c2ccccc2)c2cccc(-c3nc(-c4cccc5ccccc45)nc(-c4cccc5oc6ccccc6c45)n3)c2)cc1 ; c1ccc(-c2cccc3c(-c4nc(-c5cccc([Si](c6ccccc6)(c6ccccc6)c6ccccc6)c5)nc(-c5cccc6sc7ccccc7c56)n4)cccc23)cc1 ; c1ccc([Si](c2ccccc2)(c2ccccc2)c2cccc(-c3nc(-c4ccc(-c5cccc6ccccc56)cc4)nc(-c4ccc5c(c4)oc4ccccc45)n3)c2)cc1]

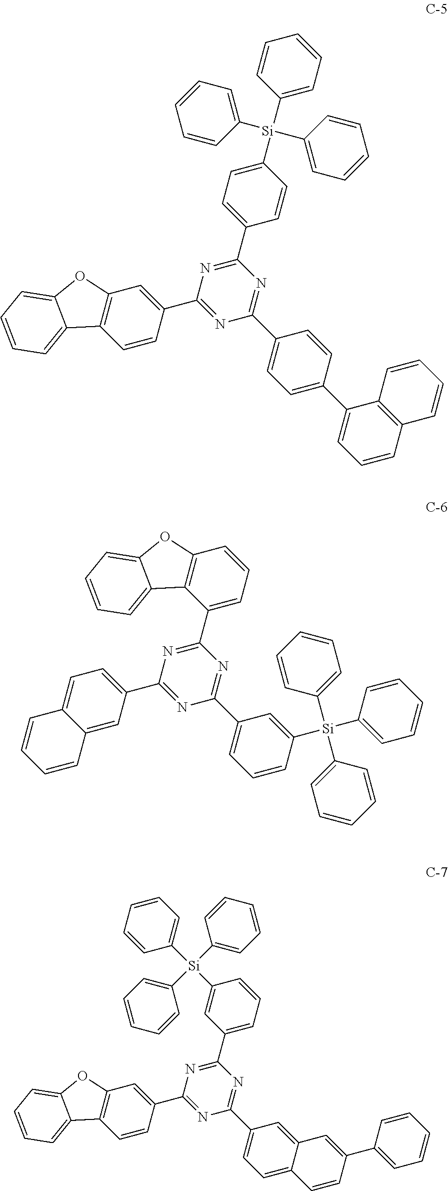 [SMILES: c1ccc([Si](c2ccccc2)(c2ccccc2)c2ccc(-c3nc(-c4ccc(-c5cccc6ccccc56)cc4)nc(-c4ccc5c(c4)oc4ccccc45)n3)cc2)cc1 ; c1ccc([Si](c2ccccc2)(c2ccccc2)c2cccc(-c3nc(-c4ccc5ccccc5c4)nc(-c4cccc5oc6ccccc6c45)n3)c2)cc1 ; c1ccc(-c2ccc3ccc(-c4nc(-c5cccc([Si](c6ccccc6)(c6ccccc6)c6ccccc6)c5)nc(-c5ccc6c(c5)oc5ccccc56)n4)cc3c2)cc1]

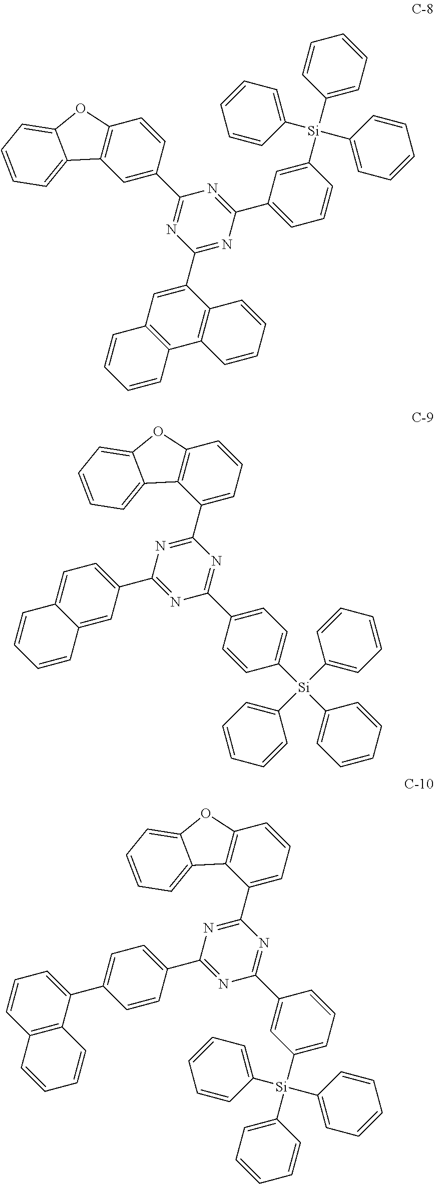 [SMILES: c1ccc([Si](c2ccccc2)(c2ccccc2)c2cccc(-c3nc(-c4ccc5oc6ccccc6c5c4)nc(-c4cc5ccccc5c5ccccc45)n3)c2)cc1 ; c1ccc([Si](c2ccccc2)(c2ccccc2)c2ccc(-c3nc(-c4ccc5ccccc5c4)nc(-c4cccc5oc6ccccc6c45)n3)cc2)cc1 ; c1ccc([Si](c2ccccc2)(c2ccccc2)c2cccc(-c3nc(-c4ccc(-c5cccc6ccccc56)cc4)nc(-c4cccc5oc6ccccc6c45)n3)c2)cc1]

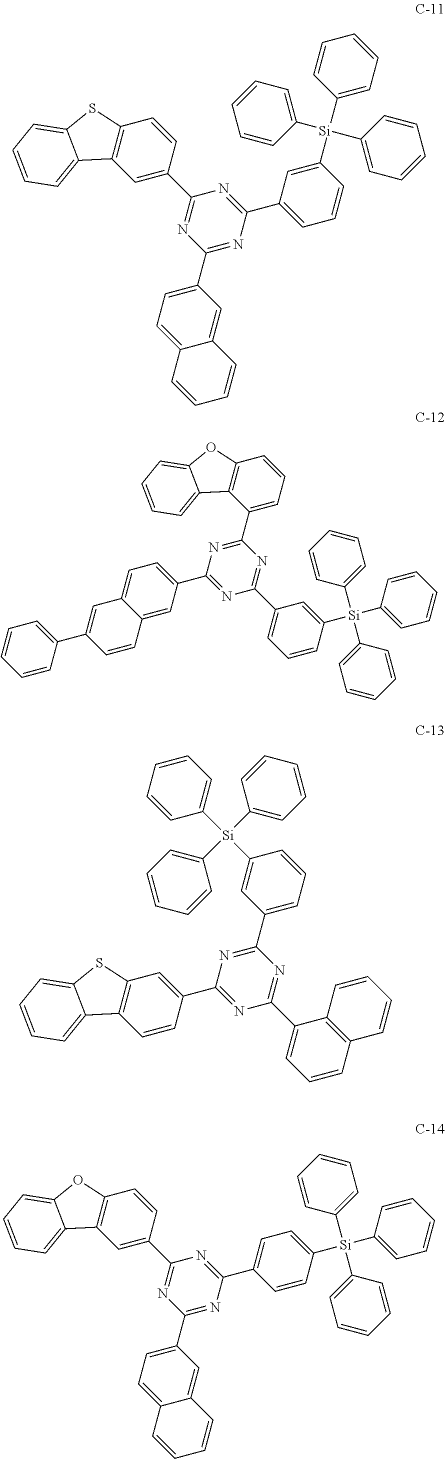 [SMILES: c1ccc([Si](c2ccccc2)(c2ccccc2)c2cccc(-c3nc(-c4ccc5ccccc5c4)nc(-c4ccc5sc6ccccc6c5c4)n3)c2)cc1 ; c1ccc(-c2ccc3cc(-c4nc(-c5cccc([Si](c6ccccc6)(c6ccccc6)c6ccccc6)c5)nc(-c5cccc6oc7ccccc7c56)n4)ccc3c2)cc1 ; c1ccc([Si](c2ccccc2)(c2ccccc2)c2cccc(-c3nc(-c4ccc5c(c4)sc4ccccc45)nc(-c4cccc5ccccc45)n3)c2)cc1 ; c1ccc([Si](c2ccccc2)(c2ccccc2)c2ccc(-c3nc(-c4ccc5ccccc5c4)nc(-c4ccc5oc6ccccc6c5c4)n3)cc2)cc1]

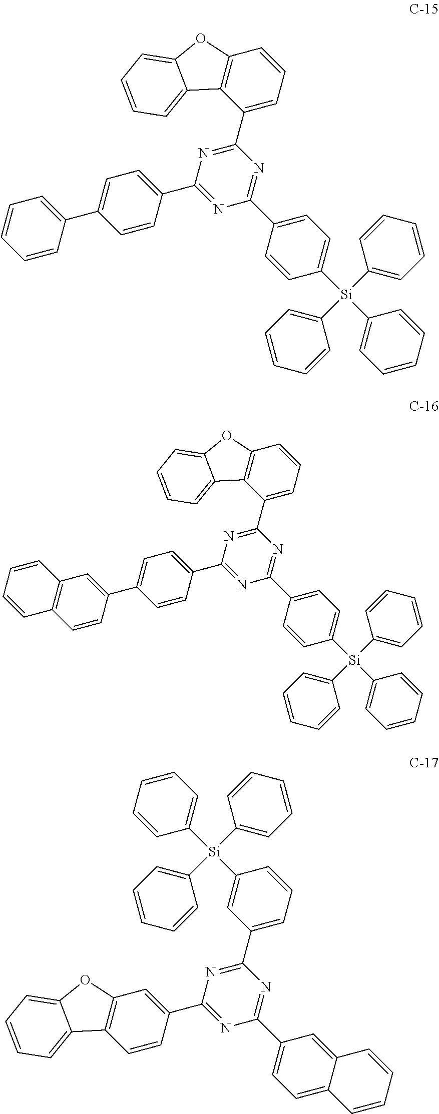 [SMILES: c1ccc(-c2ccc(-c3nc(-c4ccc([Si](c5ccccc5)(c5ccccc5)c5ccccc5)cc4)nc(-c4cccc5oc6ccccc6c45)n3)cc2)cc1 ; c1ccc([Si](c2ccccc2)(c2ccccc2)c2ccc(-c3nc(-c4ccc(-c5ccc6ccccc6c5)cc4)nc(-c4cccc5oc6ccccc6c45)n3)cc2)cc1 ; c1ccc([Si](c2ccccc2)(c2ccccc2)c2cccc(-c3nc(-c4ccc5ccccc5c4)nc(-c4ccc5c(c4)oc4ccccc45)n3)c2)cc1]

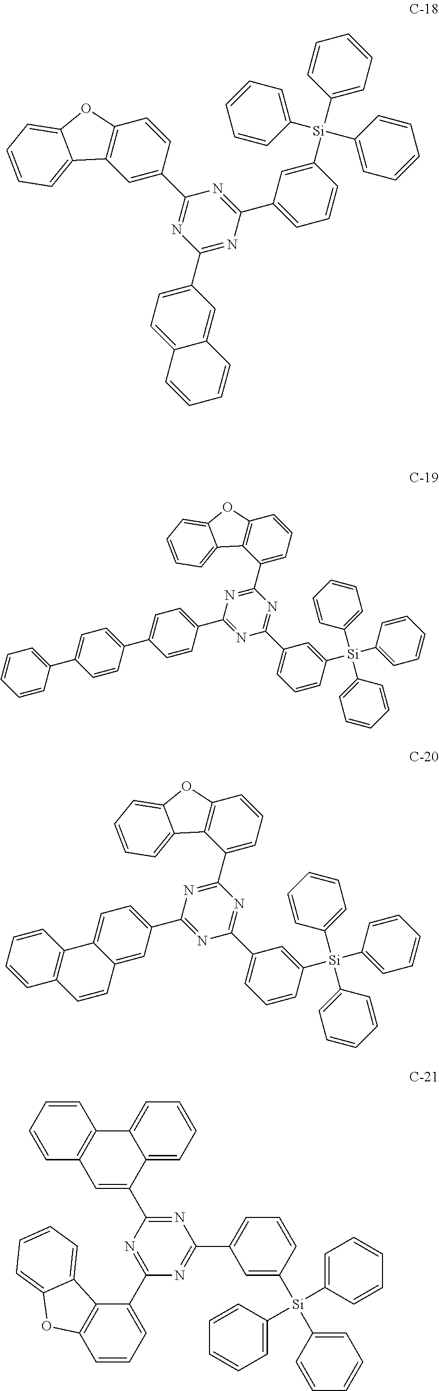 [SMILES: c1ccc([Si](c2ccccc2)(c2ccccc2)c2cccc(-c3nc(-c4ccc5ccccc5c4)nc(-c4ccc5oc6ccccc6c5c4)n3)c2)cc1 ; c1ccc(-c2ccc(-c3ccc(-c4nc(-c5cccc([Si](c6ccccc6)(c6ccccc6)c6ccccc6)c5)nc(-c5cccc6oc7ccccc7c56)n4)cc3)cc2)cc1 ; c1ccc([Si](c2ccccc2)(c2ccccc2)c2cccc(-c3nc(-c4ccc5c(ccc6ccccc65)c4)nc(-c4cccc5oc6ccccc6c45)n3)c2)cc1 ; c1ccc([Si](c2ccccc2)(c2ccccc2)c2cccc(-c3nc(-c4cc5ccccc5c5ccccc45)nc(-c4cccc5oc6ccccc6c45)n3)c2)cc1]

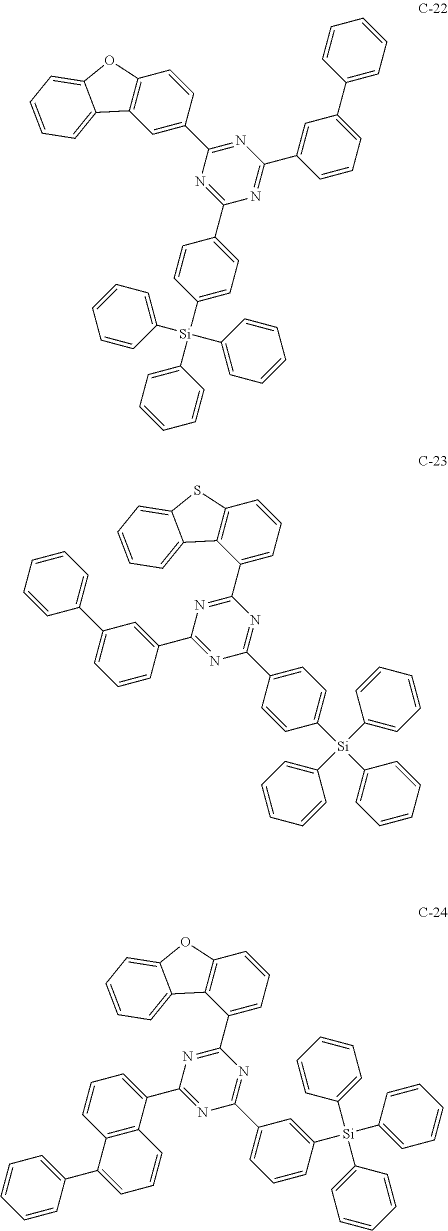 [SMILES: c1ccc(-c2cccc(-c3nc(-c4ccc([Si](c5ccccc5)(c5ccccc5)c5ccccc5)cc4)nc(-c4ccc5oc6ccccc6c5c4)n3)c2)cc1 ; c1ccc(-c2cccc(-c3nc(-c4ccc([Si](c5ccccc5)(c5ccccc5)c5ccccc5)cc4)nc(-c4cccc5sc6ccccc6c45)n3)c2)cc1 ; c1ccc(-c2cccc3c(-c4nc(-c5cccc([Si](c6ccccc6)(c6ccccc6)c6ccccc6)c5)nc(-c5cccc6oc7ccccc7c56)n4)cccc23)cc1]

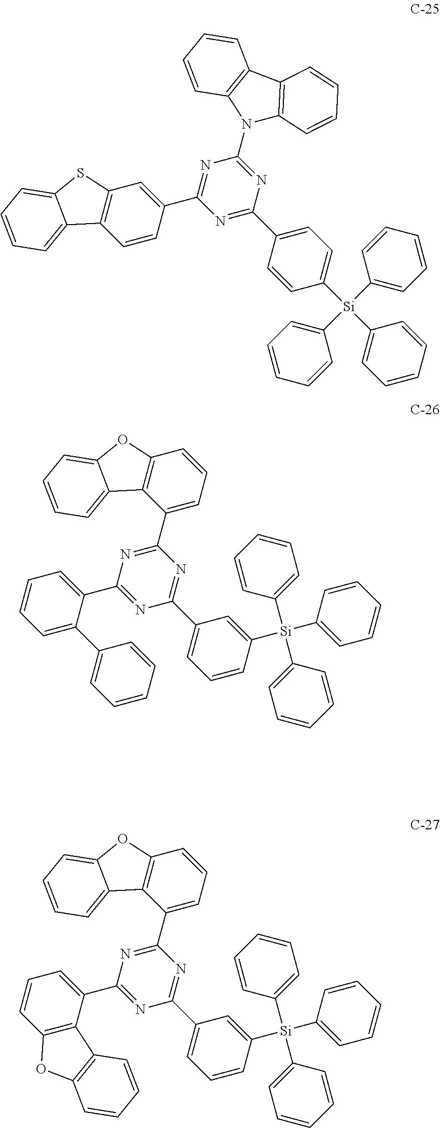 [SMILES: c1ccc([Si](c2ccccc2)(c2ccccc2)c2ccc(-c3nc(-c4ccc5c(c4)sc4ccccc45)nc(-n4c5ccccc5c5ccccc54)n3)cc2)cc1 ; c1ccc(-c2ccccc2-c2nc(-c3cccc([Si](c4ccccc4)(c4ccccc4)c4ccccc4)c3)nc(-c3cccc4oc5ccccc5c34)n2)cc1 ; c1ccc([Si](c2ccccc2)(c2ccccc2)c2cccc(-c3nc(-c4cccc5oc6ccccc6c45)nc(-c4cccc5oc6ccccc6c45)n3)c2)cc1]

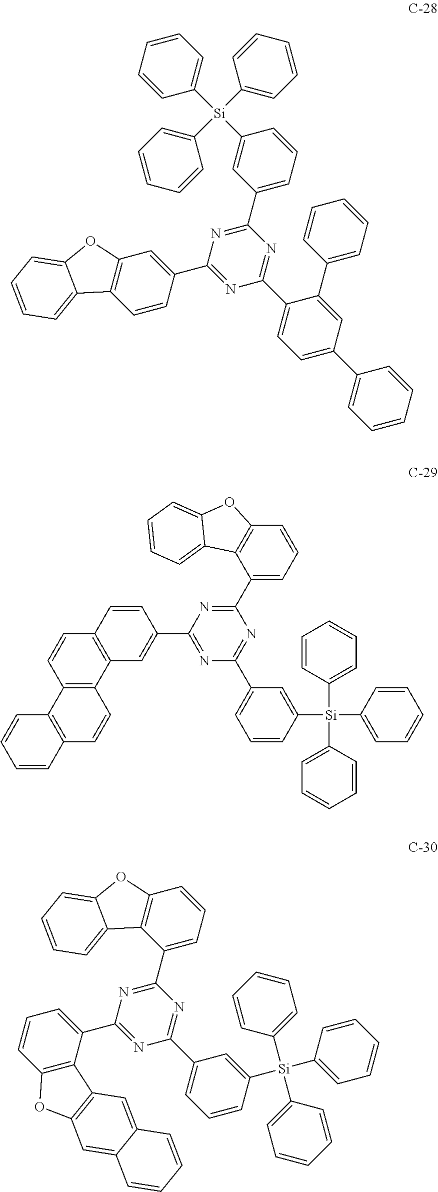 [SMILES: c1ccc(-c2ccc(-c3nc(-c4cccc([Si](c5ccccc5)(c5ccccc5)c5ccccc5)c4)nc(-c4ccc5c(c4)oc4ccccc45)n3)c(-c3ccccc3)c2)cc1 ; c1ccc([Si](c2ccccc2)(c2ccccc2)c2cccc(-c3nc(-c4ccc5ccc6c7ccccc7ccc6c5c4)nc(-c4cccc5oc6ccccc6c45)n3)c2)cc1 ; c1ccc([Si](c2ccccc2)(c2ccccc2)c2cccc(-c3nc(-c4cccc5oc6ccccc6c45)nc(-c4cccc5oc6cc7ccccc7cc6c45)n3)c2)cc1]

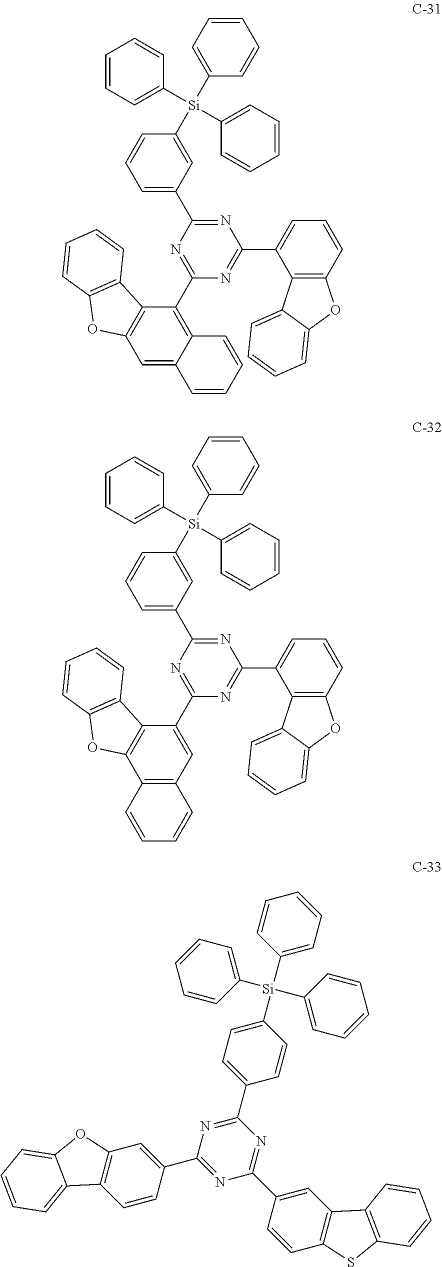

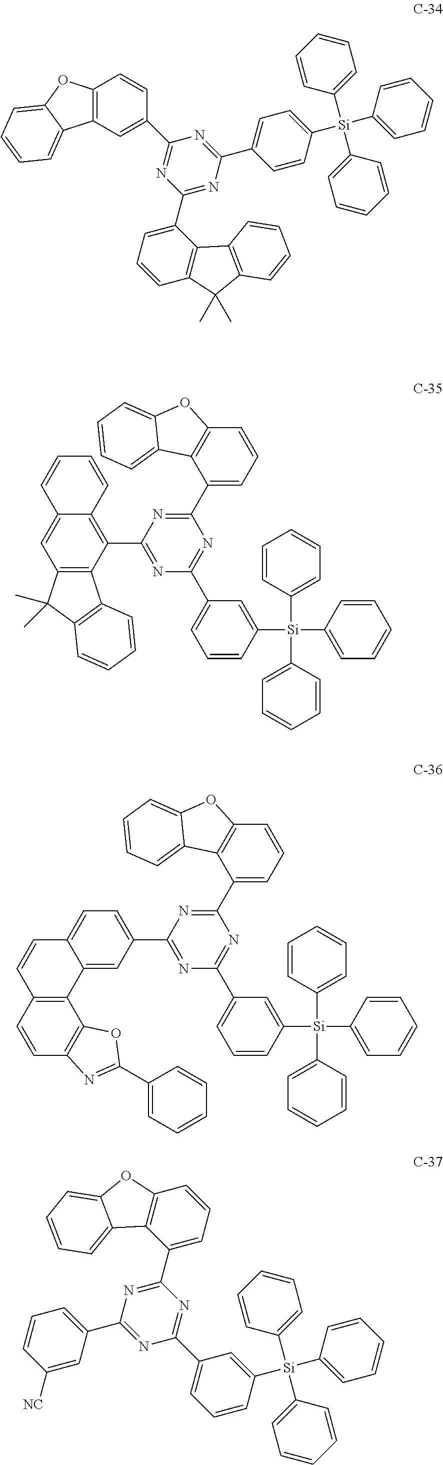 [SMILES: CC1(C)c2ccccc2-c2c(-c3nc(-c4ccc([Si](c5ccccc5)(c5ccccc5)c5ccccc5)cc4)nc(-c4ccc5oc6ccccc6c5c4)n3)cccc21 ; CC1(C)c2ccccc2-c2c1cc1ccccc1c2-c1nc(-c2cccc([Si](c3ccccc3)(c3ccccc3)c3ccccc3)c2)nc(-c2cccc3oc4ccccc4c23)n1 ; c1ccc(-c2nc3ccc4ccc5ccc(-c6nc(-c7cccc([Si](c8ccccc8)(c8ccccc8)c8ccccc8)c7)nc(-c7cccc8oc9ccccc9c78)n6)cc5c4c3o2)cc1 ; N#Cc1cccc(-c2nc(-c3cccc([Si](c4ccccc4)(c4ccccc4)c4ccccc4)c3)nc(-c3cccc4oc5ccccc5c34)n2)c1]

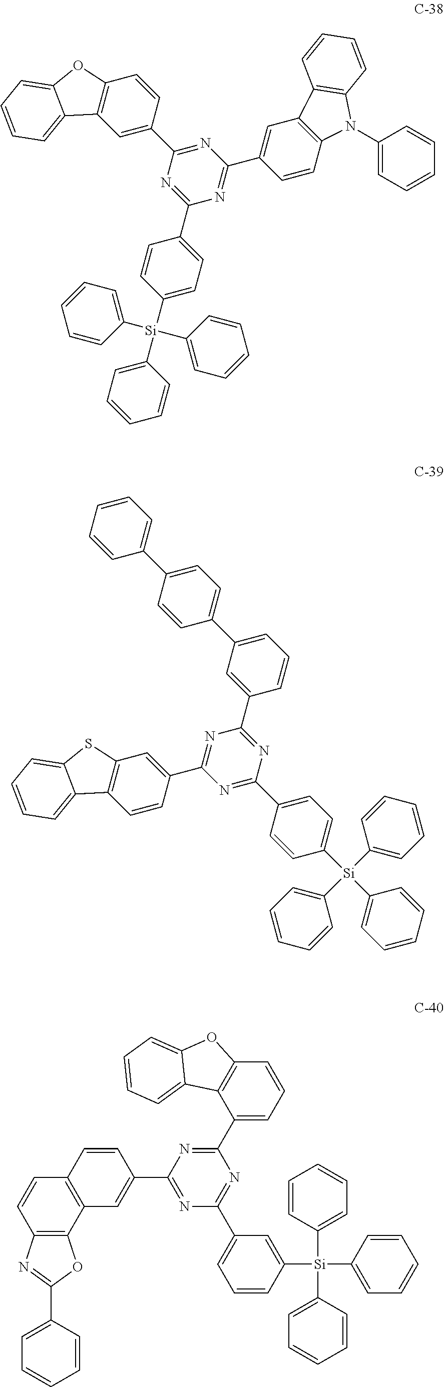 [SMILES: c1ccc(-n2c3ccccc3c3cc(-c4nc(-c5ccc([Si](c6ccccc6)(c6ccccc6)c6ccccc6)cc5)nc(-c5ccc6oc7ccccc7c6c5)n4)ccc32)cc1 ; c1ccc(-c2ccc(-c3cccc(-c4nc(-c5ccc([Si](c6ccccc6)(c6ccccc6)c6ccccc6)cc5)nc(-c5ccc6c(c5)sc5ccccc56)n4)c3)cc2)cc1 ; c1ccc(-c2nc3ccc4ccc(-c5nc(-c6cccc([Si](c7ccccc7)(c7ccccc7)c7ccccc7)c6)nc(-c6cccc7oc8ccccc8c67)n5)cc4c3o2)cc1]

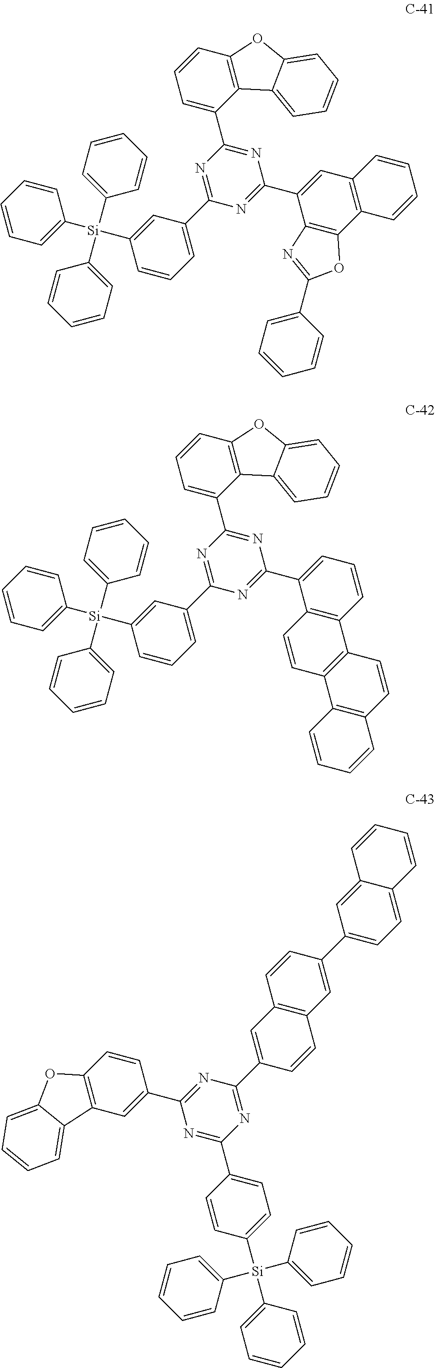 [SMILES: c1ccc(-c2nc3c(-c4nc(-c5cccc([Si](c6ccccc6)(c6ccccc6)c6ccccc6)c5)nc(-c5cccc6oc7ccccc7c56)n4)cc4ccccc4c3o2)cc1 ; c1ccc([Si](c2ccccc2)(c2ccccc2)c2cccc(-c3nc(-c4cccc5c4ccc4c6ccccc6ccc54)nc(-c4cccc5oc6ccccc6c45)n3)c2)cc1 ; c1ccc([Si](c2ccccc2)(c2ccccc2)c2ccc(-c3nc(-c4ccc5cc(-c6ccc7ccccc7c6)ccc5c4)nc(-c4ccc5oc6ccccc6c5c4)n3)cc2)cc1]

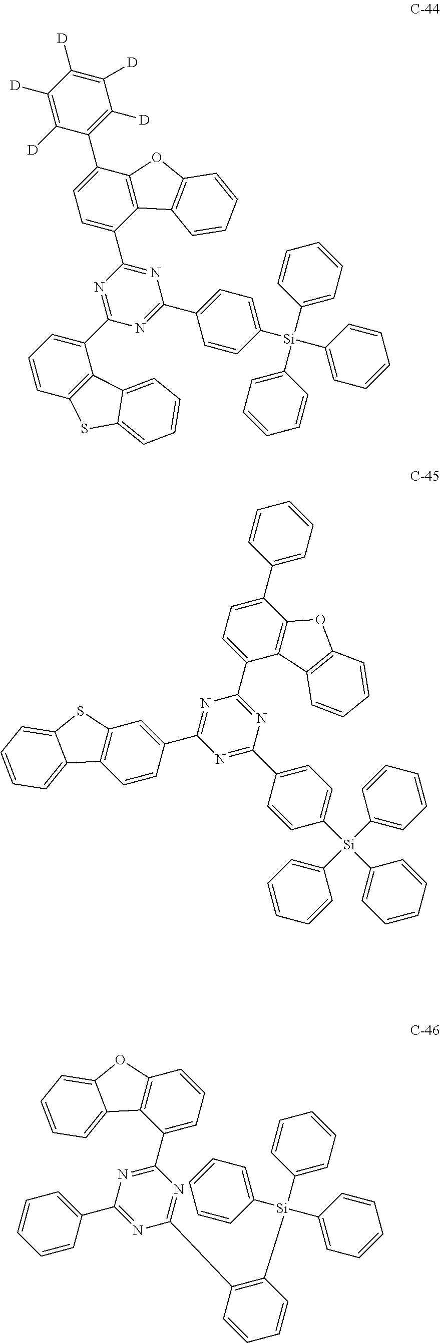 [SMILES: [2H]c1c([2H])c([2H])c(-c2ccc(-c3nc(-c4ccc([Si](c5ccccc5)(c5ccccc5)c5ccccc5)cc4)nc(-c4cccc5sc6ccccc6c45)n3)c3c2oc2ccccc23)c([2H])c1[2H] ; c1ccc(-c2ccc(-c3nc(-c4ccc([Si](c5ccccc5)(c5ccccc5)c5ccccc5)cc4)nc(-c4ccc5c(c4)sc4ccccc45)n3)c3c2oc2ccccc23)cc1 ; c1ccc(-c2nc(-c3ccccc3[Si](c3ccccc3)(c3ccccc3)c3ccccc3)nc(-c3cccc4oc5ccccc5c34)n2)cc1]

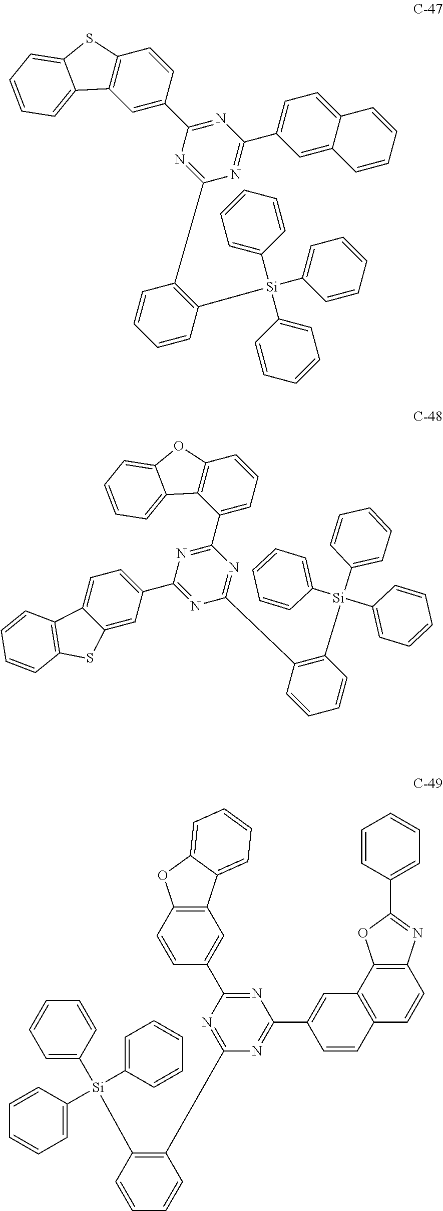 [SMILES: c1ccc([Si](c2ccccc2)(c2ccccc2)c2ccccc2-c2nc(-c3ccc4ccccc4c3)nc(-c3ccc4sc5ccccc5c4c3)n2)cc1 ; c1ccc([Si](c2ccccc2)(c2ccccc2)c2ccccc2-c2nc(-c3ccc4c(c3)sc3ccccc34)nc(-c3cccc4oc5ccccc5c34)n2)cc1 ; c1ccc(-c2nc3ccc4ccc(-c5nc(-c6ccc7oc8ccccc8c7c6)nc(-c6ccccc6[Si](c6ccccc6)(c6ccccc6)c6ccccc6)n5)cc4c3o2)cc1]

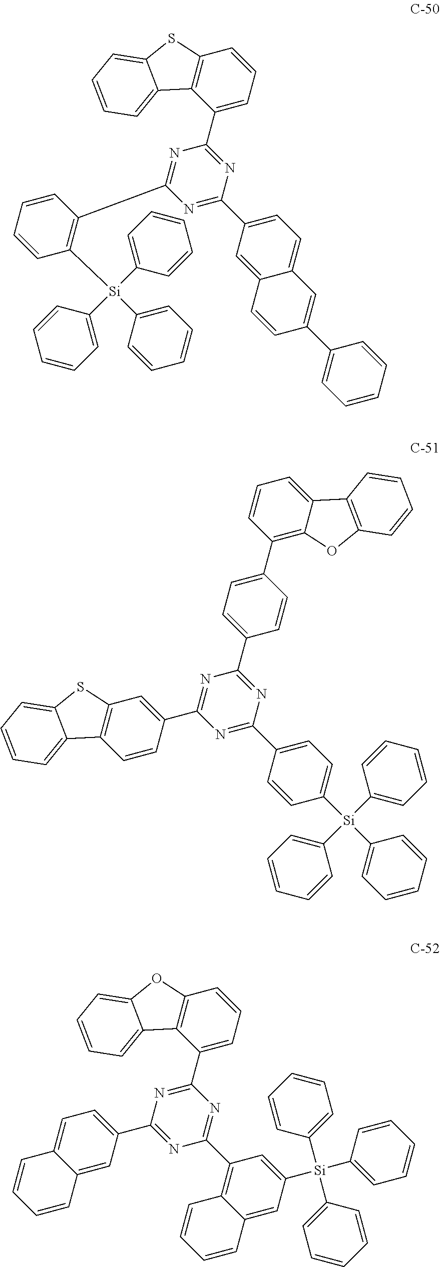 [SMILES: c1ccc(-c2ccc3cc(-c4nc(-c5ccccc5[Si](c5ccccc5)(c5ccccc5)c5ccccc5)nc(-c5cccc6sc7ccccc7c56)n4)ccc3c2)cc1 ; c1ccc([Si](c2ccccc2)(c2ccccc2)c2ccc(-c3nc(-c4ccc(-c5cccc6c5oc5ccccc56)cc4)nc(-c4ccc5c(c4)sc4ccccc45)n3)cc2)cc1 ; c1ccc([Si](c2ccccc2)(c2ccccc2)c2cc(-c3nc(-c4ccc5ccccc5c4)nc(-c4cccc5oc6ccccc6c45)n3)c3ccccc3c2)cc1]

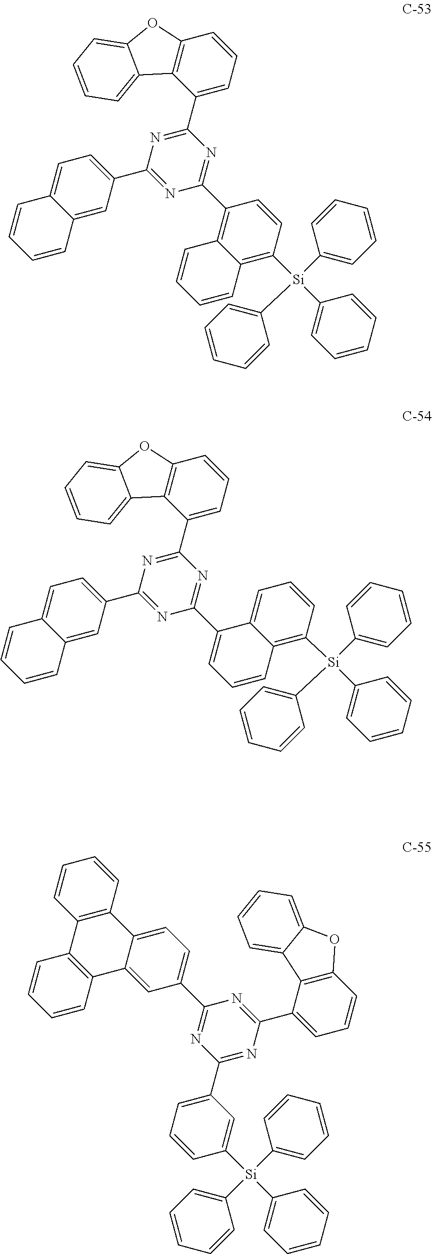 [SMILES: c1ccc([Si](c2ccccc2)(c2ccccc2)c2ccc(-c3nc(-c4ccc5ccccc5c4)nc(-c4cccc5oc6ccccc6c45)n3)c3ccccc23)cc1 ; c1ccc([Si](c2ccccc2)(c2ccccc2)c2cccc3c(-c4nc(-c5ccc6ccccc6c5)nc(-c5cccc6oc7ccccc7c56)n4)cccc23)cc1 ; c1ccc([Si](c2ccccc2)(c2ccccc2)c2cccc(-c3nc(-c4ccc5c6ccccc6c6ccccc6c5c4)nc(-c4cccc5oc6ccccc6c45)n3)c2)cc1]

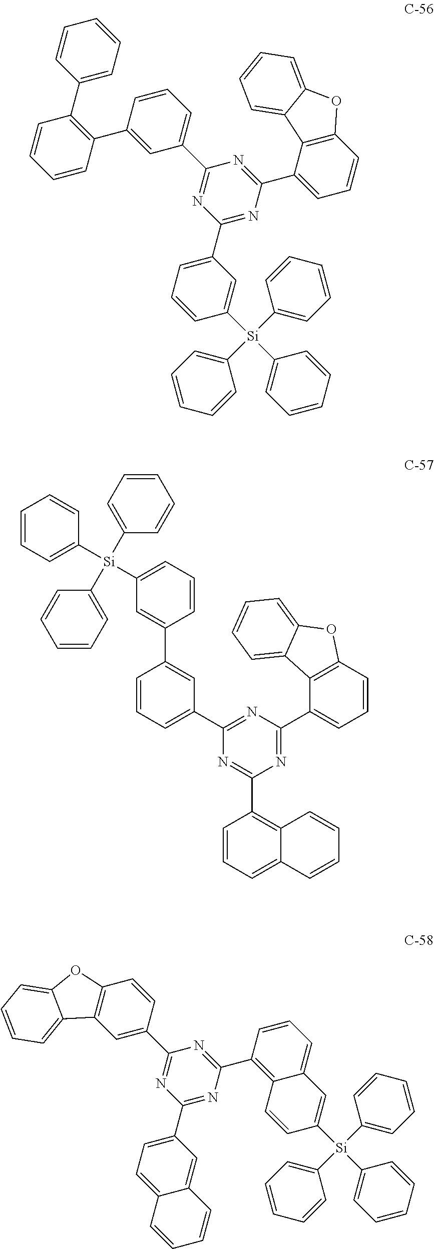 [SMILES: c1ccc(-c2ccccc2-c2cccc(-c3nc(-c4cccc([Si](c5ccccc5)(c5ccccc5)c5ccccc5)c4)nc(-c4cccc5oc6ccccc6c45)n3)c2)cc1 ; c1ccc([Si](c2ccccc2)(c2ccccc2)c2cccc(-c3cccc(-c4nc(-c5cccc6ccccc56)nc(-c5cccc6oc7ccccc7c56)n4)c3)c2)cc1 ; c1ccc([Si](c2ccccc2)(c2ccccc2)c2ccc3c(-c4nc(-c5ccc6ccccc6c5)nc(-c5ccc6oc7ccccc7c6c5)n4)cccc3c2)cc1]

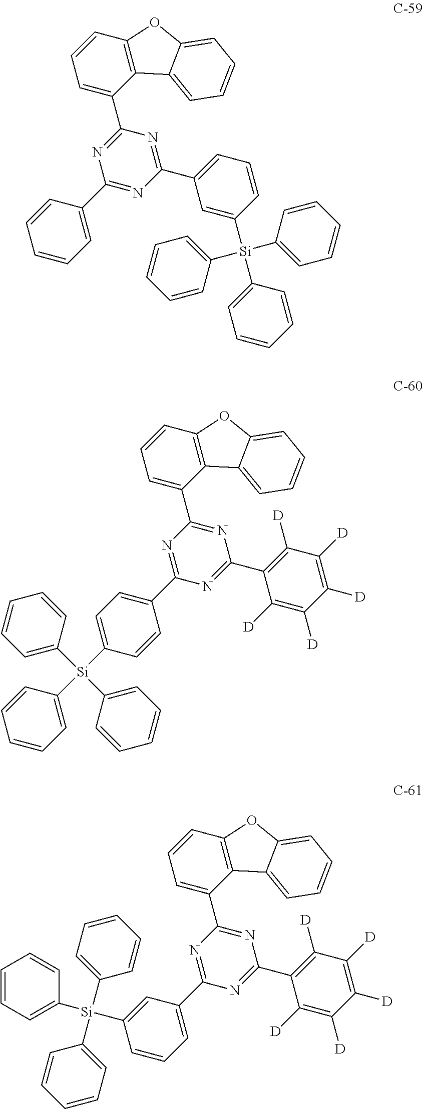 [SMILES: c1ccc(-c2nc(-c3cccc([Si](c4ccccc4)(c4ccccc4)c4ccccc4)c3)nc(-c3cccc4oc5ccccc5c34)n2)cc1 ; [2H]c1c([2H])c([2H])c(-c2nc(-c3ccc([Si](c4ccccc4)(c4ccccc4)c4ccccc4)cc3)nc(-c3cccc4oc5ccccc5c34)n2)c([2H])c1[2H] ; [2H]c1c([2H])c([2H])c(-c2nc(-c3cccc([Si](c4ccccc4)(c4ccccc4)c4ccccc4)c3)nc(-c3cccc4oc5ccccc5c34)n2)c([2H])c1[2H]]

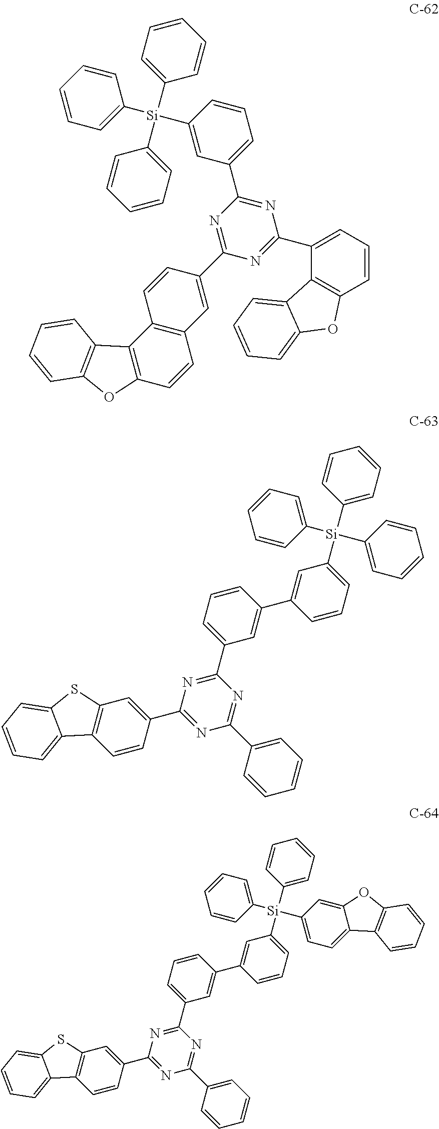 [SMILES: c1ccc([Si](c2ccccc2)(c2ccccc2)c2cccc(-c3nc(-c4ccc5c(ccc6oc7ccccc7c65)c4)nc(-c4cccc5oc6ccccc6c45)n3)c2)cc1 ; c1ccc(-c2nc(-c3cccc(-c4cccc([Si](c5ccccc5)(c5ccccc5)c5ccccc5)c4)c3)nc(-c3ccc4c(c3)sc3ccccc34)n2)cc1 ; c1ccc(-c2nc(-c3cccc(-c4cccc([Si](c5ccccc5)(c5ccccc5)c5ccc6c(c5)oc5ccccc56)c4)c3)nc(-c3ccc4c(c3)sc3ccccc34)n2)cc1]

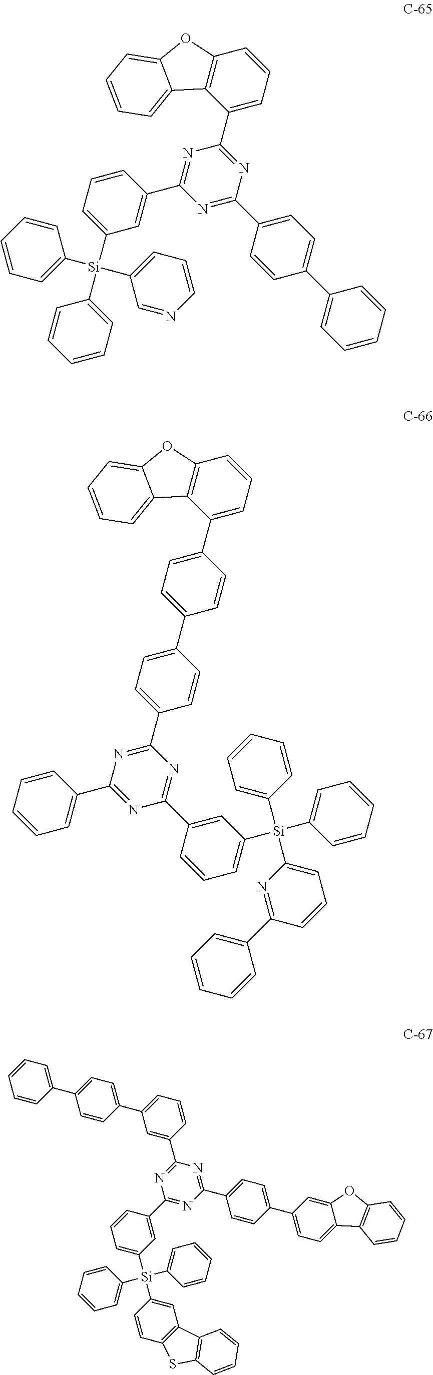 [SMILES: c1ccc(-c2ccc(-c3nc(-c4cccc([Si](c5ccccc5)(c5ccccc5)c5cccnc5)c4)nc(-c4cccc5oc6ccccc6c45)n3)cc2)cc1 ; c1ccc(-c2cccc([Si](c3ccccc3)(c3ccccc3)c3cccc(-c4nc(-c5ccccc5)nc(-c5ccc(-c6ccc(-c7cccc8oc9ccccc9c78)cc6)cc5)n4)c3)n2)cc1 ; c1ccc(-c2ccc(-c3cccc(-c4nc(-c5ccc(-c6ccc7c(c6)oc6ccccc67)cc5)nc(-c5cccc([Si](c6ccccc6)(c6ccccc6)c6ccc7sc8ccccc8c7c6)c5)n4)c3)cc2)cc1]

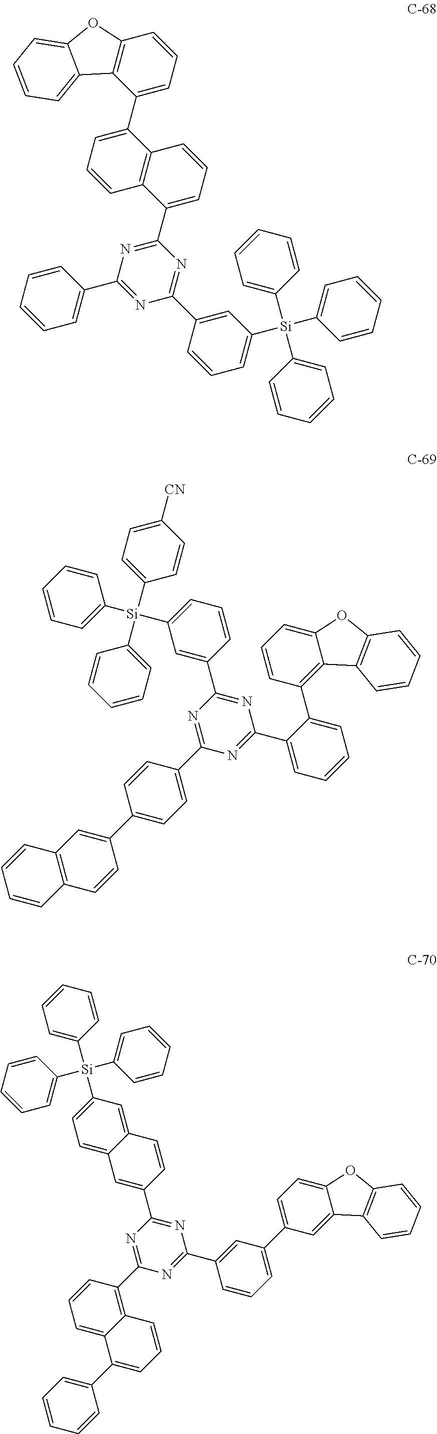 [SMILES: c1ccc(-c2nc(-c3cccc([Si](c4ccccc4)(c4ccccc4)c4ccccc4)c3)nc(-c3cccc4c(-c5cccc6oc7ccccc7c56)cccc34)n2)cc1 ; N#Cc1ccc([Si](c2ccccc2)(c2ccccc2)c2cccc(-c3nc(-c4ccc(-c5ccc6ccccc6c5)cc4)nc(-c4ccccc4-c4cccc5oc6ccccc6c45)n3)c2)cc1 ; c1ccc(-c2cccc3c(-c4nc(-c5cccc(-c6ccc7oc8ccccc8c7c6)c5)nc(-c5ccc6cc([Si](c7ccccc7)(c7ccccc7)c7ccccc7)ccc6c5)n4)cccc23)cc1]

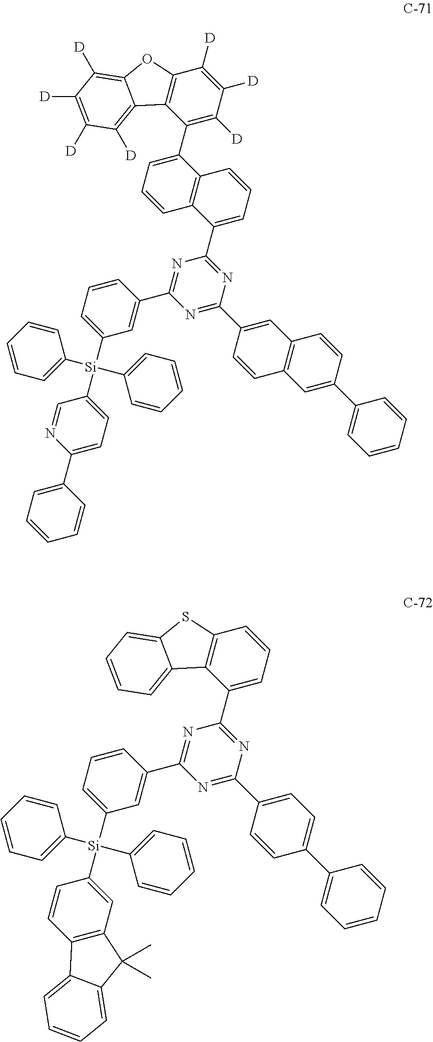 [SMILES: [2H]c1c([2H])c([2H])c2c(oc3c([2H])c([2H])c([2H])c(-c4cccc5c(-c6nc(-c7cccc([Si](c8ccccc8)(c8ccccc8)c8ccc(-c9ccccc9)nc8)c7)nc(-c7ccc8cc(-c9ccccc9)ccc8c7)n6)cccc45)c32)c1[2H] ; CC1(C)c2ccccc2-c2ccc([Si](c3ccccc3)(c3ccccc3)c3cccc(-c4nc(-c5ccc(-c6ccccc6)cc5)nc(-c5cccc6sc7ccccc7c56)n4)c3)cc21]

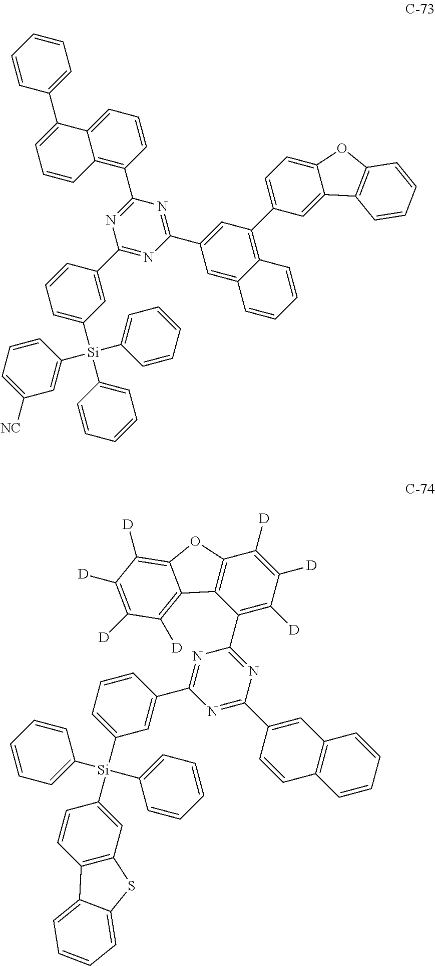 [SMILES: N#Cc1cccc([Si](c2ccccc2)(c2ccccc2)c2cccc(-c3nc(-c4cc(-c5ccc6oc7ccccc7c6c5)c5ccccc5c4)nc(-c4cccc5c(-c6ccccc6)cccc45)n3)c2)c1 ; [2H]c1c([2H])c([2H])c2c(oc3c([2H])c([2H])c([2H])c(-c4nc(-c5cccc([Si](c6ccccc6)(c6ccccc6)c6ccc7c(c6)sc6ccccc67)c5)nc(-c5ccc6ccccc6c5)n4)c32)c1[2H]]

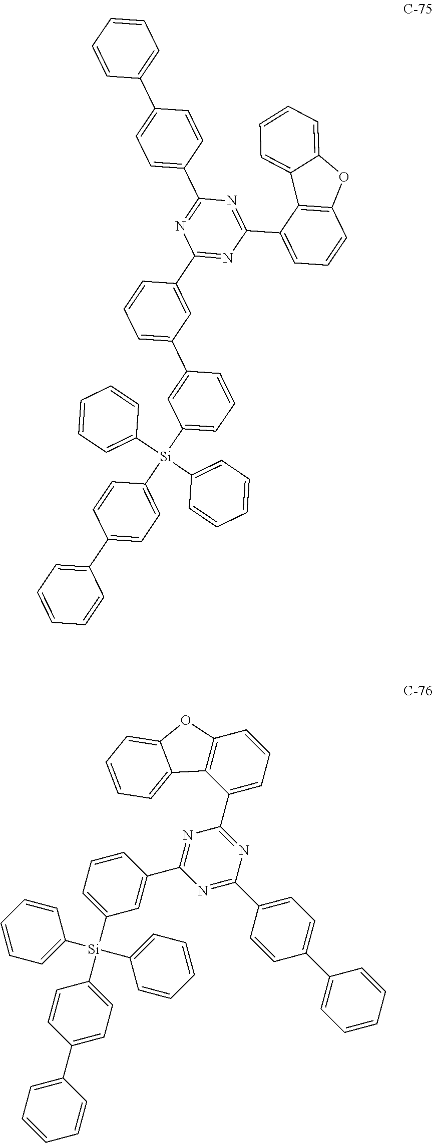 [SMILES: c1ccc(-c2ccc(-c3nc(-c4cccc(-c5cccc([Si](c6ccccc6)(c6ccccc6)c6ccc(-c7ccccc7)cc6)c5)c4)nc(-c4cccc5oc6ccccc6c45)n3)cc2)cc1 ; c1ccc(-c2ccc(-c3nc(-c4cccc([Si](c5ccccc5)(c5ccccc5)c5ccc(-c6ccccc6)cc5)c4)nc(-c4cccc5oc6ccccc6c45)n3)cc2)cc1]

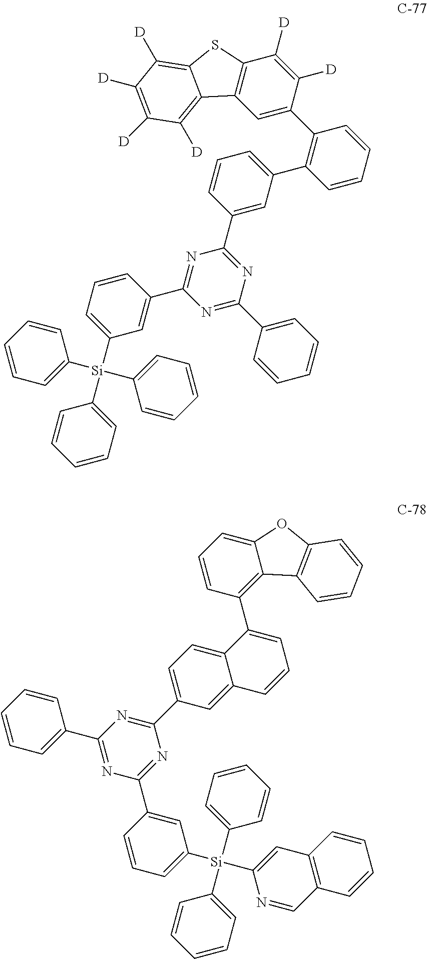 [SMILES: [2H]c1c(-c2ccccc2-c2cccc(-c3nc(-c4ccccc4)nc(-c4cccc([Si](c5ccccc5)(c5ccccc5)c5ccccc5)c4)n3)c2)cc2c(sc3c([2H])c([2H])c([2H])c([2H])c32)c1[2H] ; c1ccc(-c2nc(-c3cccc([Si](c4ccccc4)(c4ccccc4)c4cc5ccccc5cn4)c3)nc(-c3ccc4c(-c5cccc6oc7ccccc7c56)cccc4c3)n2)cc1]

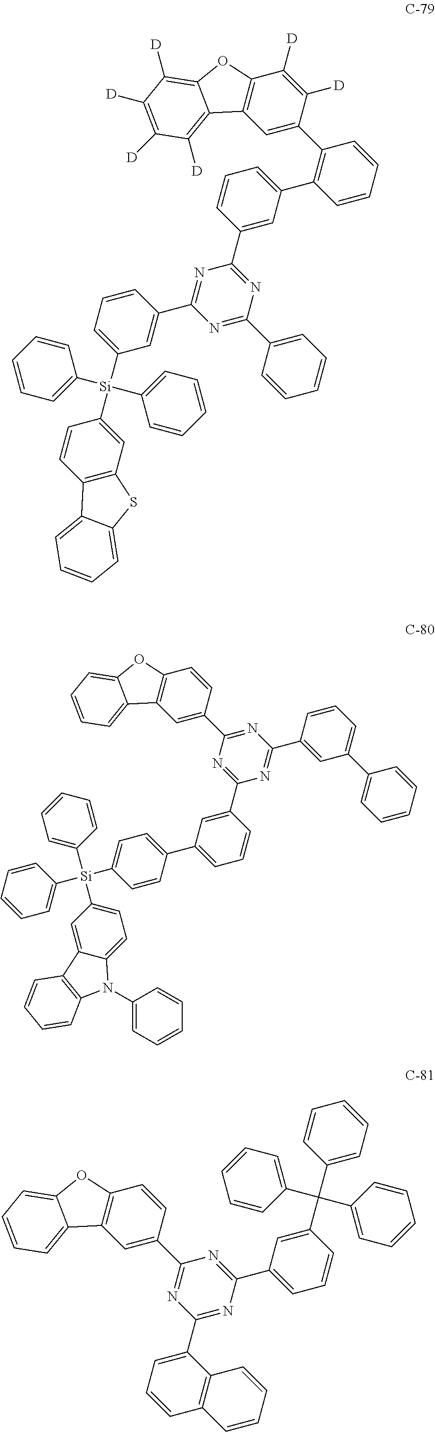 [SMILES: [2H]c1c(-c2ccccc2-c2cccc(-c3nc(-c4ccccc4)nc(-c4cccc([Si](c5ccccc5)(c5ccccc5)c5ccc6c(c5)sc5ccccc56)c4)n3)c2)cc2c(oc3c([2H])c([2H])c([2H])c([2H])c32)c1[2H] ; c1ccc(-c2cccc(-c3nc(-c4cccc(-c5ccc([Si](c6ccccc6)(c6ccccc6)c6ccc7c(c6)c6ccccc6n7-c6ccccc6)cc5)c4)nc(-c4ccc5oc6ccccc6c5c4)n3)c2)cc1 ; c1ccc(C(c2ccccc2)(c2ccccc2)c2cccc(-c3nc(-c4ccc5oc6ccccc6c5c4)nc(-c4cccc5ccccc45)n3)c2)cc1]

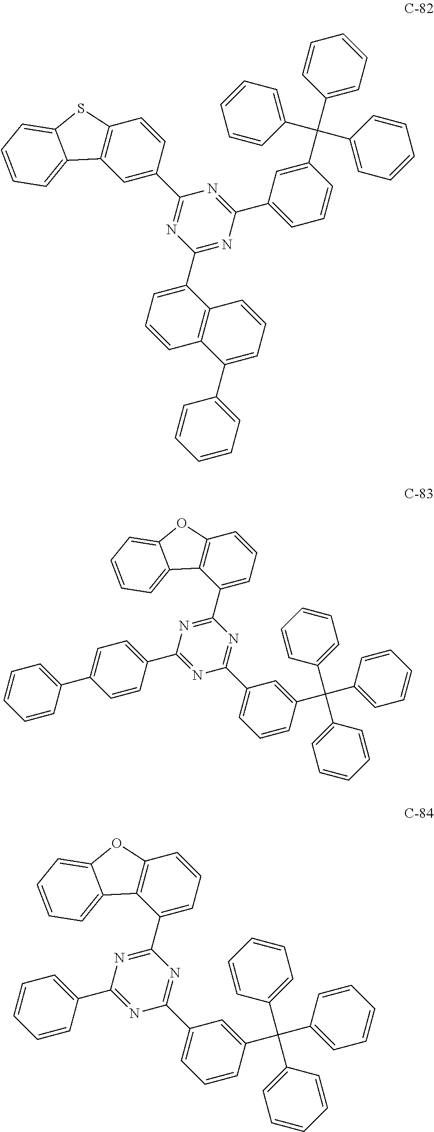 [SMILES: c1ccc(-c2cccc3c(-c4nc(-c5cccc(C(c6ccccc6)(c6ccccc6)c6ccccc6)c5)nc(-c5ccc6sc7ccccc7c6c5)n4)cccc23)cc1 ; c1ccc(-c2ccc(-c3nc(-c4cccc(C(c5ccccc5)(c5ccccc5)c5ccccc5)c4)nc(-c4cccc5oc6ccccc6c45)n3)cc2)cc1 ; c1ccc(-c2nc(-c3cccc(C(c4ccccc4)(c4ccccc4)c4ccccc4)c3)nc(-c3cccc4oc5ccccc5c34)n2)cc1]

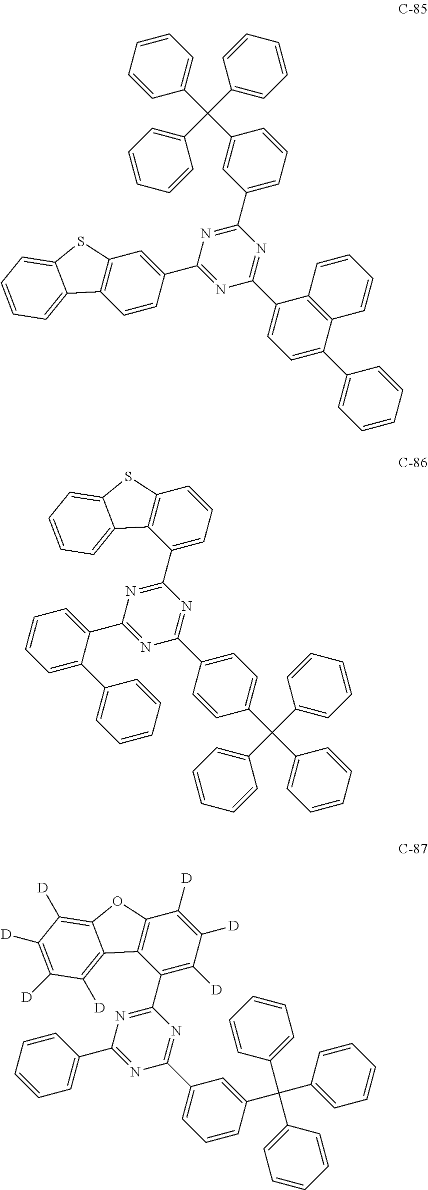 [SMILES: c1ccc(-c2ccc(-c3nc(-c4cccc(C(c5ccccc5)(c5ccccc5)c5ccccc5)c4)nc(-c4ccc5c(c4)sc4ccccc45)n3)c3ccccc23)cc1 ; c1ccc(-c2ccccc2-c2nc(-c3ccc(C(c4ccccc4)(c4ccccc4)c4ccccc4)cc3)nc(-c3cccc4sc5ccccc5c34)n2)cc1 ; [2H]c1c([2H])c([2H])c2c(oc3c([2H])c([2H])c([2H])c(-c4nc(-c5ccccc5)nc(-c5cccc(C(c6ccccc6)(c6ccccc6)c6ccccc6)c5)n4)c32)c1[2H]]

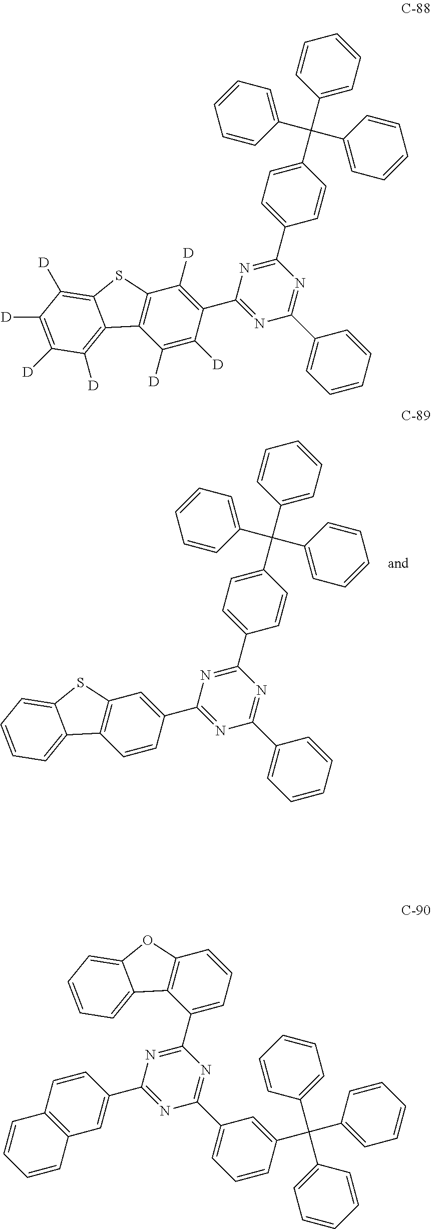 [SMILES: [2H]c1c([2H])c([2H])c2c(sc3c([2H])c(-c4nc(-c5ccccc5)nc(-c5ccc(C(c6ccccc6)(c6ccccc6)c6ccccc6)cc5)n4)c([2H])c([2H])c32)c1[2H] ; c1ccc(-c2nc(-c3ccc(C(c4ccccc4)(c4ccccc4)c4ccccc4)cc3)nc(-c3ccc4c(c3)sc3ccccc34)n2)cc1 ; c1ccc(C(c2ccccc2)(c2ccccc2)c2cccc(-c3nc(-c4ccc5ccccc5c4)nc(-c4cccc5oc6ccccc6c45)n3)c2)cc1]

9. An organic electroluminescent device comprising: a first electrode; a second electrode; and at least one light-emitting layer(s) between the first electrode and the second electrode, wherein the at least one light-emitting layer(s) comprises the plurality of host materials according to claim 1.

10. An organic electroluminescent compound represented by the following formula 3: 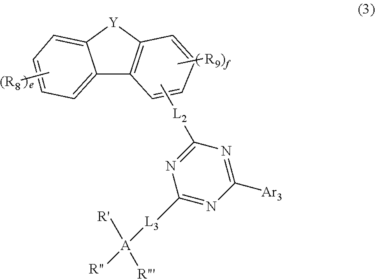
wherein,
Y represents O or S;
A represents Si or C;
L₂ represents a single bond, phenylene unsubstituted or substituted by deuterium, biphenylene unsubstituted or substituted by deuterium, or naphthylene unsubstituted or substituted by deuterium;
R₈ and R₉ each independently represent hydrogen or deuterium;
Ar₃ represents phenyl unsubstituted or substituted by deuterium, biphenyl unsubstituted or substituted by deuterium, terphenyl unsubstituted or substituted by deuterium, naphthyl unsubstituted or substituted by deuterium, triphenylenyl unsubstituted or substituted by deuterium, phenanthrenyl unsubstituted or substituted by deuterium, or a combination thereof;
L₃ represents phenylene unsubstituted or substituted by deuterium, biphenylene unsubstituted or substituted by deuterium, or naphthylene unsubstituted or substituted by deuterium;
R′, R″, and R′″ each independently represent a substituted or unsubstituted (C6-C30)aryl or a substituted or unsubstituted (3- to 30-membered)heteroaryl;
e represents an integer of 1 to 4, and f represents an integer of 1 to 3; and
when e and f are an integer of 2 or more, each of R₈ and each of R₉ may be the same or different.

11. The organic electroluminescent compound according to claim 10, wherein the compound represented by formula 3 is selected from the following compounds:

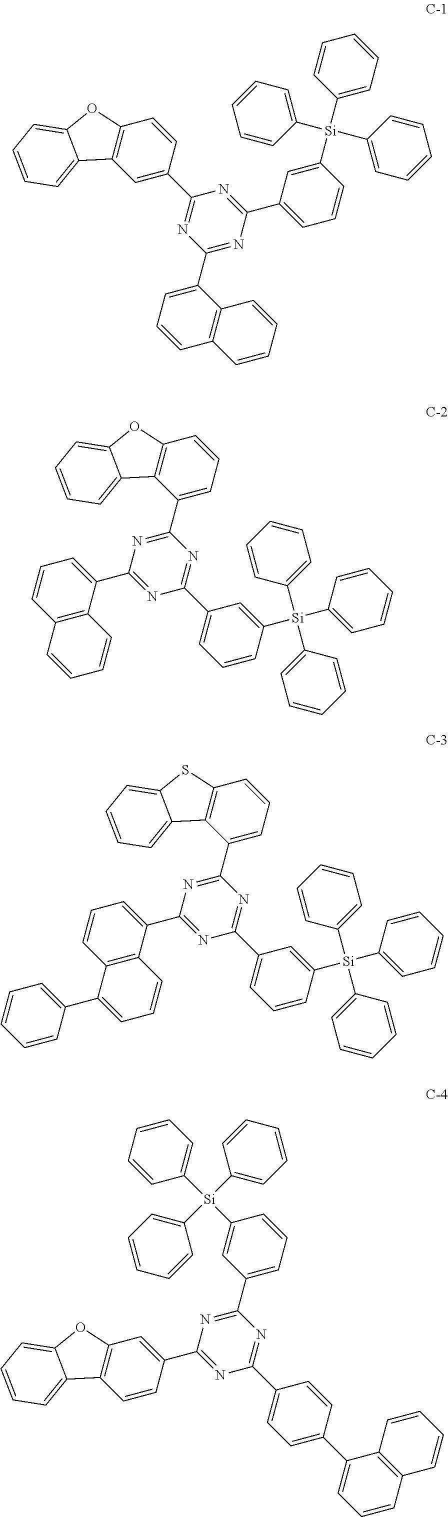 [SMILES: c1ccc([Si](c2ccccc2)(c2ccccc2)c2cccc(-c3nc(-c4ccc5oc6ccccc6c5c4)nc(-c4cccc5ccccc45)n3)c2)cc1 ; c1ccc([Si](c2ccccc2)(c2ccccc2)c2cccc(-c3nc(-c4cccc5ccccc45)nc(-c4cccc5oc6ccccc6c45)n3)c2)cc1 ; c1ccc(-c2cccc3c(-c4nc(-c5cccc([Si](c6ccccc6)(c6ccccc6)c6ccccc6)c5)nc(-c5cccc6sc7ccccc7c56)n4)cccc23)cc1 ; c1ccc([Si](c2ccccc2)(c2ccccc2)c2cccc(-c3nc(-c4ccc(-c5cccc6ccccc56)cc4)nc(-c4ccc5c(c4)oc4ccccc45)n3)c2)cc1]

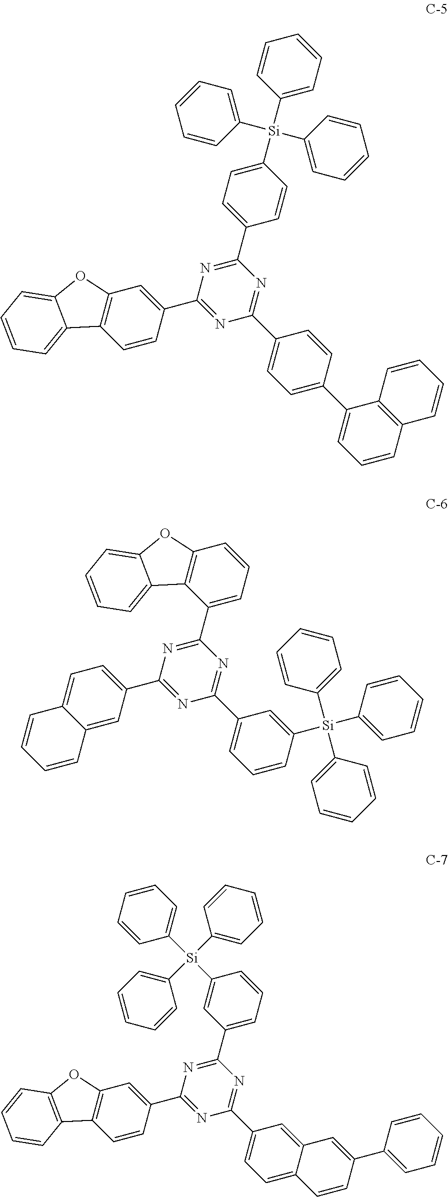 [SMILES: c1ccc([Si](c2ccccc2)(c2ccccc2)c2ccc(-c3nc(-c4ccc(-c5cccc6ccccc56)cc4)nc(-c4ccc5c(c4)oc4ccccc45)n3)cc2)cc1 ; c1ccc([Si](c2ccccc2)(c2ccccc2)c2cccc(-c3nc(-c4ccc5ccccc5c4)nc(-c4cccc5oc6ccccc6c45)n3)c2)cc1 ; c1ccc(-c2ccc3ccc(-c4nc(-c5cccc([Si](c6ccccc6)(c6ccccc6)c6ccccc6)c5)nc(-c5ccc6c(c5)oc5ccccc56)n4)cc3c2)cc1]

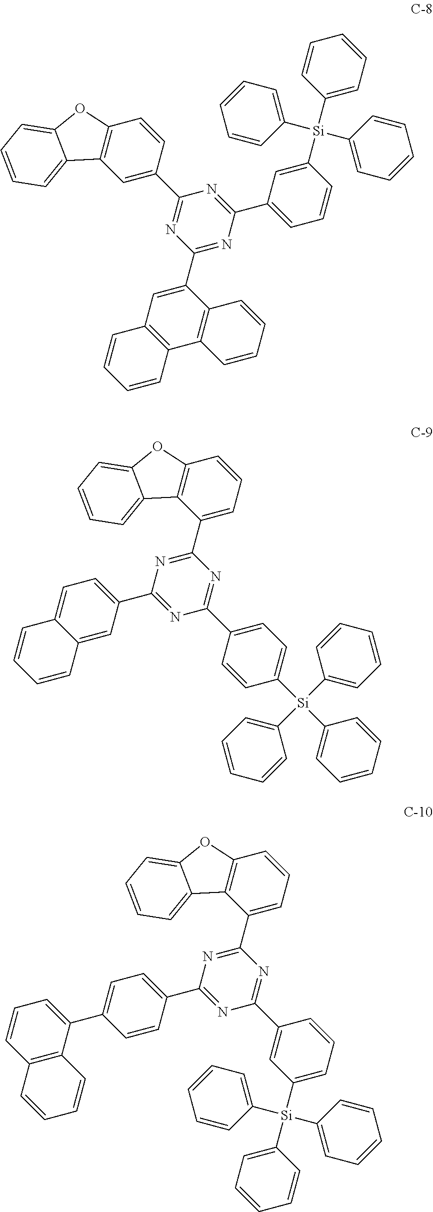 [SMILES: c1ccc([Si](c2ccccc2)(c2ccccc2)c2cccc(-c3nc(-c4ccc5oc6ccccc6c5c4)nc(-c4cc5ccccc5c5ccccc45)n3)c2)cc1 ; c1ccc([Si](c2ccccc2)(c2ccccc2)c2ccc(-c3nc(-c4ccc5ccccc5c4)nc(-c4cccc5oc6ccccc6c45)n3)cc2)cc1 ; c1ccc([Si](c2ccccc2)(c2ccccc2)c2cccc(-c3nc(-c4ccc(-c5cccc6ccccc56)cc4)nc(-c4cccc5oc6ccccc6c45)n3)c2)cc1]

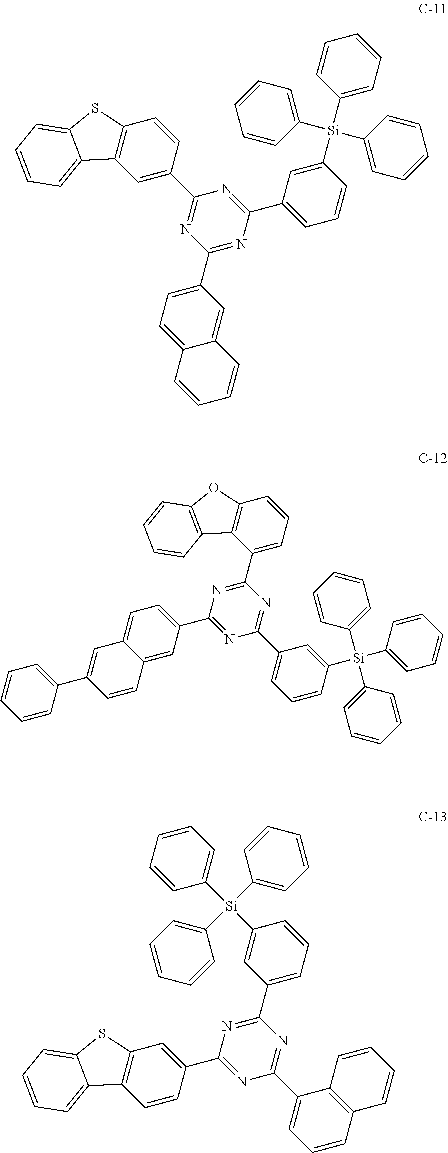 [SMILES: c1ccc([Si](c2ccccc2)(c2ccccc2)c2cccc(-c3nc(-c4ccc5ccccc5c4)nc(-c4ccc5sc6ccccc6c5c4)n3)c2)cc1 ; c1ccc(-c2ccc3cc(-c4nc(-c5cccc([Si](c6ccccc6)(c6ccccc6)c6ccccc6)c5)nc(-c5cccc6oc7ccccc7c56)n4)ccc3c2)cc1 ; c1ccc([Si](c2ccccc2)(c2ccccc2)c2cccc(-c3nc(-c4ccc5c(c4)sc4ccccc45)nc(-c4cccc5ccccc45)n3)c2)cc1]

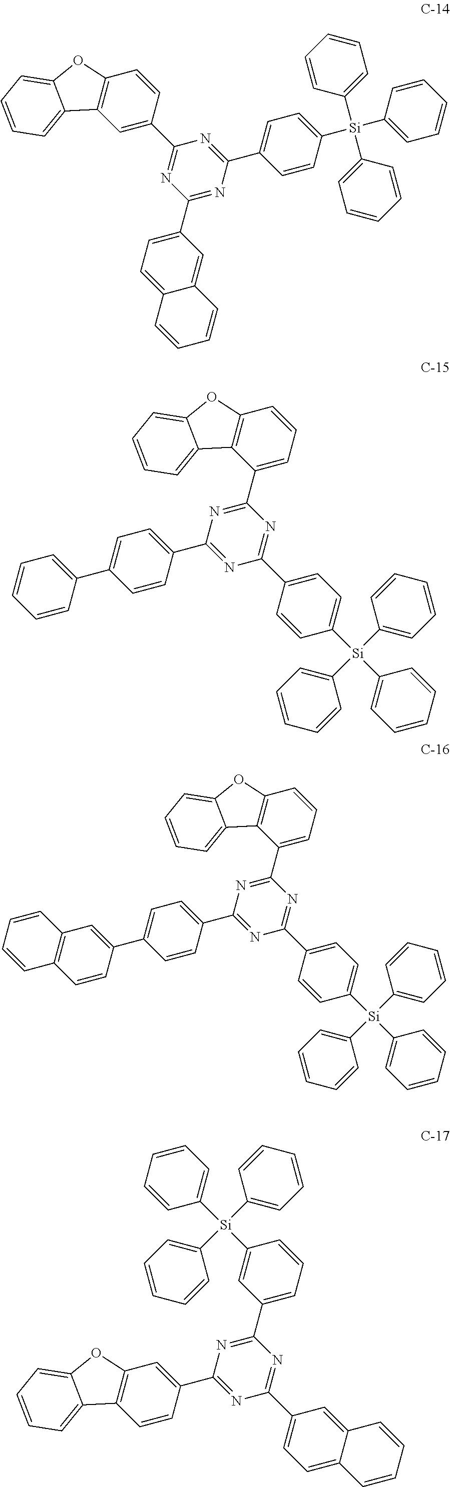 [SMILES: c1ccc([Si](c2ccccc2)(c2ccccc2)c2ccc(-c3nc(-c4ccc5ccccc5c4)nc(-c4ccc5oc6ccccc6c5c4)n3)cc2)cc1 ; c1ccc(-c2ccc(-c3nc(-c4ccc([Si](c5ccccc5)(c5ccccc5)c5ccccc5)cc4)nc(-c4cccc5oc6ccccc6c45)n3)cc2)cc1 ; c1ccc([Si](c2ccccc2)(c2ccccc2)c2ccc(-c3nc(-c4ccc(-c5ccc6ccccc6c5)cc4)nc(-c4cccc5oc6ccccc6c45)n3)cc2)cc1 ; c1ccc([Si](c2ccccc2)(c2ccccc2)c2cccc(-c3nc(-c4ccc5ccccc5c4)nc(-c4ccc5c(c4)oc4ccccc45)n3)c2)cc1]

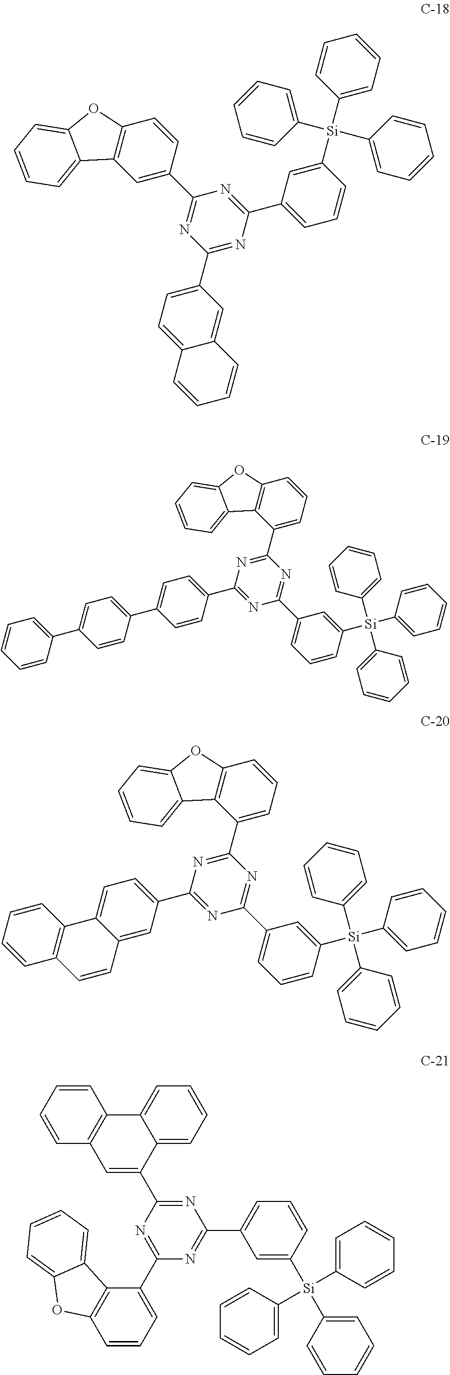 [SMILES: c1ccc([Si](c2ccccc2)(c2ccccc2)c2cccc(-c3nc(-c4ccc5ccccc5c4)nc(-c4ccc5oc6ccccc6c5c4)n3)c2)cc1 ; c1ccc(-c2ccc(-c3ccc(-c4nc(-c5cccc([Si](c6ccccc6)(c6ccccc6)c6ccccc6)c5)nc(-c5cccc6oc7ccccc7c56)n4)cc3)cc2)cc1 ; c1ccc([Si](c2ccccc2)(c2ccccc2)c2cccc(-c3nc(-c4ccc5c(ccc6ccccc65)c4)nc(-c4cccc5oc6ccccc6c45)n3)c2)cc1 ; c1ccc([Si](c2ccccc2)(c2ccccc2)c2cccc(-c3nc(-c4cc5ccccc5c5ccccc45)nc(-c4cccc5oc6ccccc6c45)n3)c2)cc1]

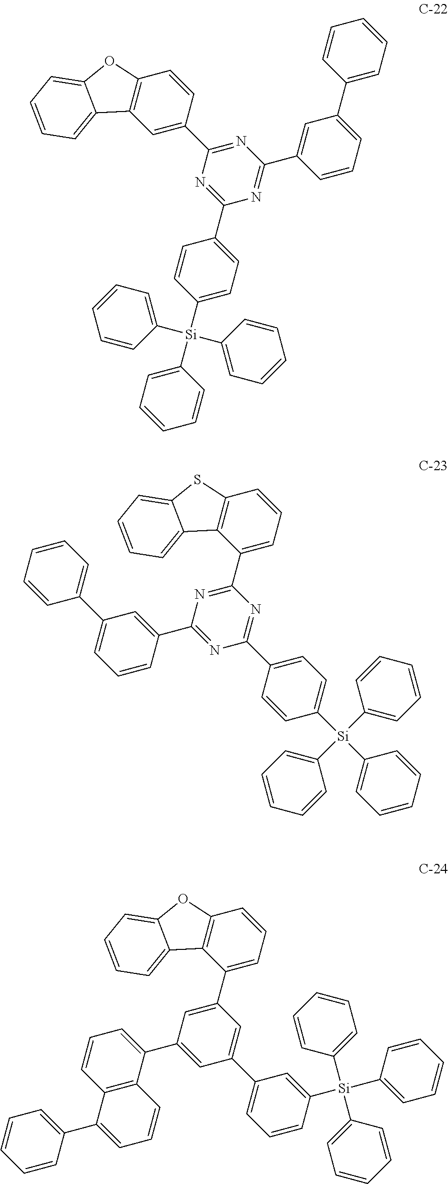 [SMILES: c1ccc(-c2cccc(-c3nc(-c4ccc([Si](c5ccccc5)(c5ccccc5)c5ccccc5)cc4)nc(-c4ccc5oc6ccccc6c5c4)n3)c2)cc1 ; c1ccc(-c2cccc(-c3nc(-c4ccc([Si](c5ccccc5)(c5ccccc5)c5ccccc5)cc4)nc(-c4cccc5sc6ccccc6c45)n3)c2)cc1 ; c1ccc(-c2cccc3c(-c4cc(-c5cccc([Si](c6ccccc6)(c6ccccc6)c6ccccc6)c5)cc(-c5cccc6oc7ccccc7c56)c4)cccc23)cc1]

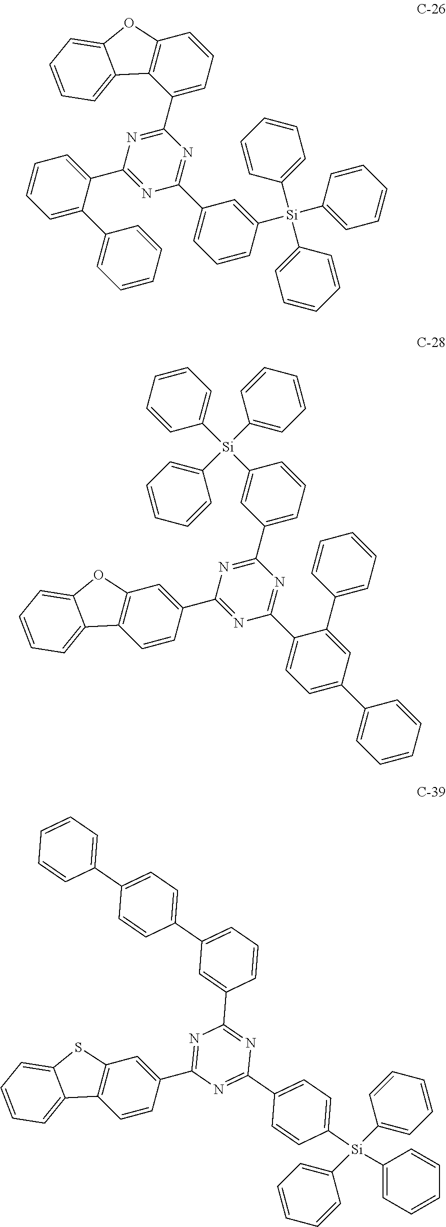 [SMILES: c1ccc(-c2ccccc2-c2nc(-c3cccc([Si](c4ccccc4)(c4ccccc4)c4ccccc4)c3)nc(-c3cccc4oc5ccccc5c34)n2)cc1 ; c1ccc(-c2ccc(-c3nc(-c4cccc([Si](c5ccccc5)(c5ccccc5)c5ccccc5)c4)nc(-c4ccc5c(c4)oc4ccccc45)n3)c(-c3ccccc3)c2)cc1 ; c1ccc(-c2ccc(-c3cccc(-c4nc(-c5ccc([Si](c6ccccc6)(c6ccccc6)c6ccccc6)cc5)nc(-c5ccc6c(c5)sc5ccccc56)n4)c3)cc2)cc1]

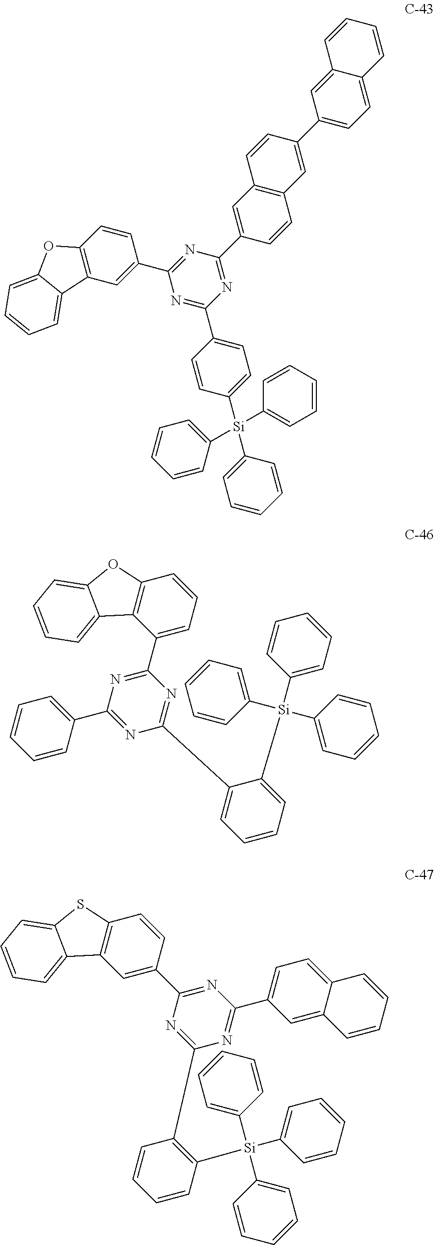 [SMILES: c1ccc([Si](c2ccccc2)(c2ccccc2)c2ccc(-c3nc(-c4ccc5cc(-c6ccc7ccccc7c6)ccc5c4)nc(-c4ccc5oc6ccccc6c5c4)n3)cc2)cc1 ; c1ccc(-c2nc(-c3ccccc3[Si](c3ccccc3)(c3ccccc3)c3ccccc3)nc(-c3cccc4oc5ccccc5c34)n2)cc1 ; c1ccc([Si](c2ccccc2)(c2ccccc2)c2ccccc2-c2nc(-c3ccc4ccccc4c3)nc(-c3ccc4sc5ccccc5c4c3)n2)cc1]

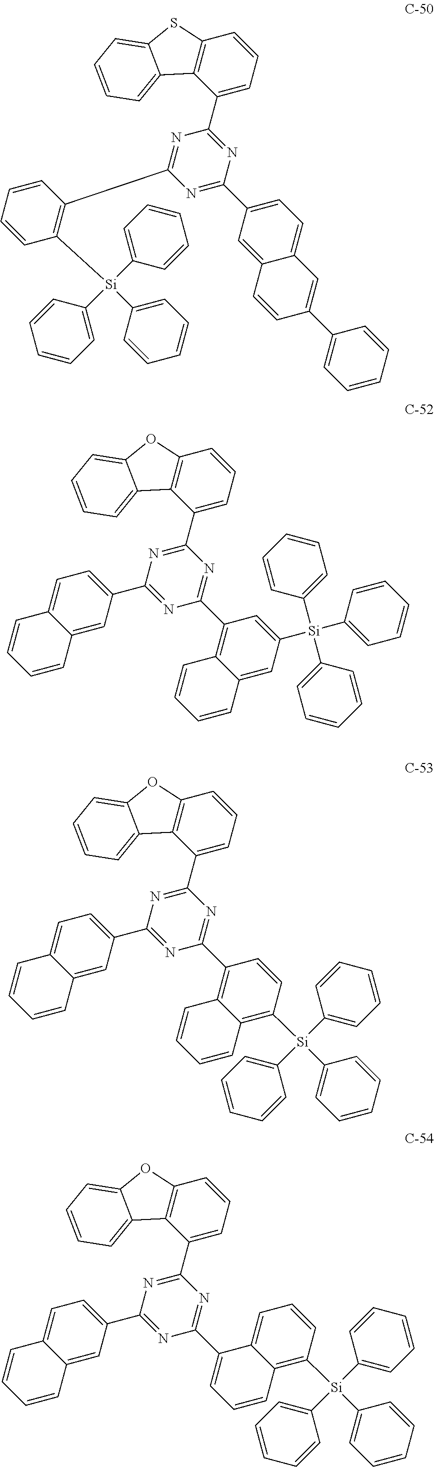 [SMILES: c1ccc(-c2ccc3cc(-c4nc(-c5ccccc5[Si](c5ccccc5)(c5ccccc5)c5ccccc5)nc(-c5cccc6sc7ccccc7c56)n4)ccc3c2)cc1 ; c1ccc([Si](c2ccccc2)(c2ccccc2)c2cc(-c3nc(-c4ccc5ccccc5c4)nc(-c4cccc5oc6ccccc6c45)n3)c3ccccc3c2)cc1 ; c1ccc([Si](c2ccccc2)(c2ccccc2)c2ccc(-c3nc(-c4ccc5ccccc5c4)nc(-c4cccc5oc6ccccc6c45)n3)c3ccccc23)cc1 ; c1ccc([Si](c2ccccc2)(c2ccccc2)c2cccc3c(-c4nc(-c5ccc6ccccc6c5)nc(-c5cccc6oc7ccccc7c56)n4)cccc23)cc1]

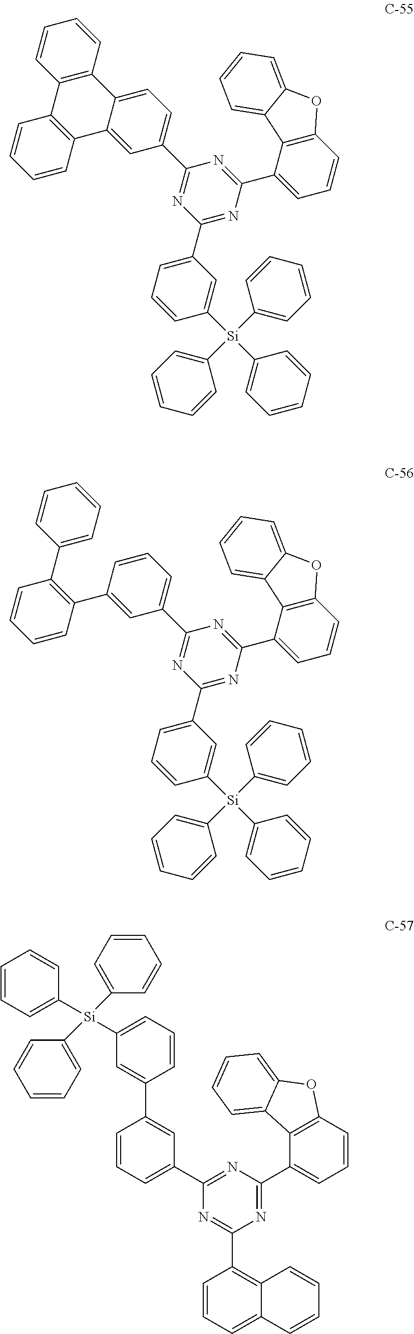 [SMILES: c1ccc([Si](c2ccccc2)(c2ccccc2)c2cccc(-c3nc(-c4ccc5c6ccccc6c6ccccc6c5c4)nc(-c4cccc5oc6ccccc6c45)n3)c2)cc1 ; c1ccc(-c2ccccc2-c2cccc(-c3nc(-c4cccc([Si](c5ccccc5)(c5ccccc5)c5ccccc5)c4)nc(-c4cccc5oc6ccccc6c45)n3)c2)cc1 ; c1ccc([Si](c2ccccc2)(c2ccccc2)c2cccc(-c3cccc(-c4nc(-c5cccc6ccccc56)nc(-c5cccc6oc7ccccc7c56)n4)c3)c2)cc1]

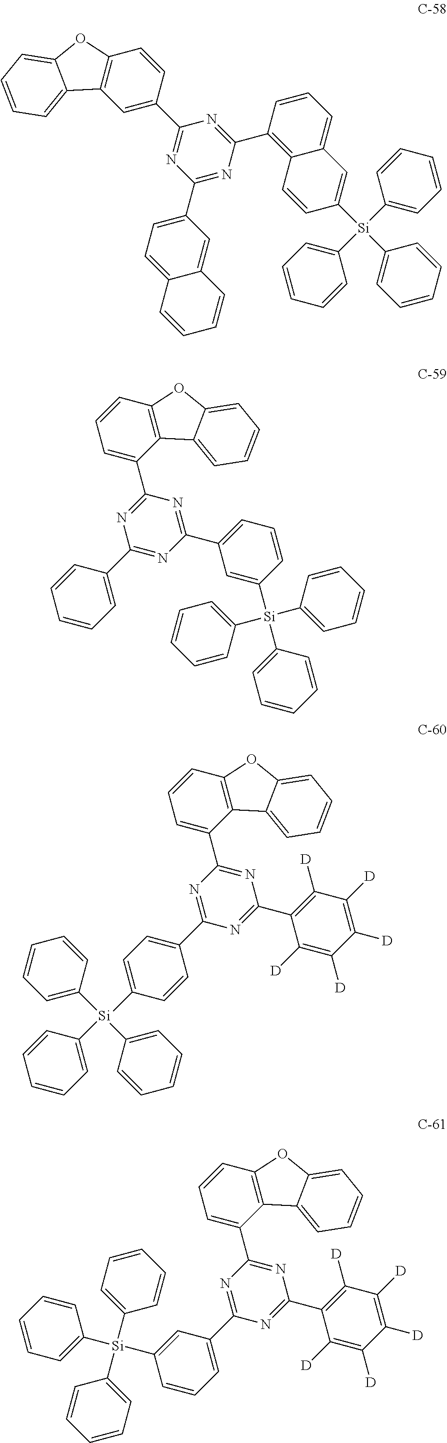 [SMILES: c1ccc([Si](c2ccccc2)(c2ccccc2)c2ccc3c(-c4nc(-c5ccc6ccccc6c5)nc(-c5ccc6oc7ccccc7c6c5)n4)cccc3c2)cc1 ; c1ccc(-c2nc(-c3cccc([Si](c4ccccc4)(c4ccccc4)c4ccccc4)c3)nc(-c3cccc4oc5ccccc5c34)n2)cc1 ; [2H]c1c([2H])c([2H])c(-c2nc(-c3ccc([Si](c4ccccc4)(c4ccccc4)c4ccccc4)cc3)nc(-c3cccc4oc5ccccc5c34)n2)c([2H])c1[2H] ; [2H]c1c([2H])c([2H])c(-c2nc(-c3cccc([Si](c4ccccc4)(c4ccccc4)c4ccccc4)c3)nc(-c3cccc4oc5ccccc5c34)n2)c([2H])c1[2H]]

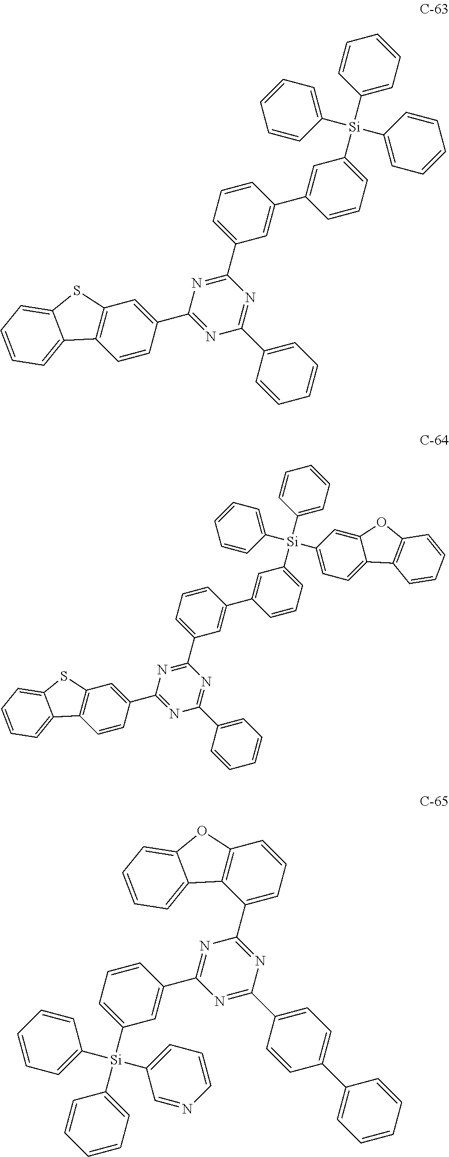 [SMILES: c1ccc(-c2nc(-c3cccc(-c4cccc([Si](c5ccccc5)(c5ccccc5)c5ccccc5)c4)c3)nc(-c3ccc4c(c3)sc3ccccc34)n2)cc1 ; c1ccc(-c2nc(-c3cccc(-c4cccc([Si](c5ccccc5)(c5ccccc5)c5ccc6c(c5)oc5ccccc56)c4)c3)nc(-c3ccc4c(c3)sc3ccccc34)n2)cc1 ; c1ccc(-c2ccc(-c3nc(-c4cccc([Si](c5ccccc5)(c5ccccc5)c5cccnc5)c4)nc(-c4cccc5oc6ccccc6c45)n3)cc2)cc1]

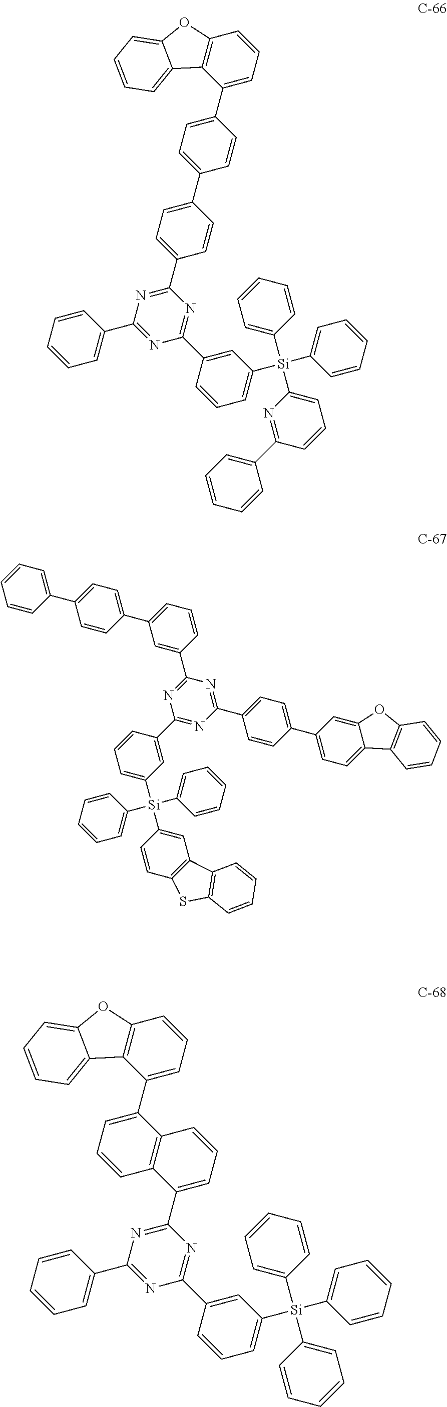 [SMILES: c1ccc(-c2cccc([Si](c3ccccc3)(c3ccccc3)c3cccc(-c4nc(-c5ccccc5)nc(-c5ccc(-c6ccc(-c7cccc8oc9ccccc9c78)cc6)cc5)n4)c3)n2)cc1 ; c1ccc(-c2ccc(-c3cccc(-c4nc(-c5ccc(-c6ccc7c(c6)oc6ccccc67)cc5)nc(-c5cccc([Si](c6ccccc6)(c6ccccc6)c6ccc7sc8ccccc8c7c6)c5)n4)c3)cc2)cc1 ; c1ccc(-c2nc(-c3cccc([Si](c4ccccc4)(c4ccccc4)c4ccccc4)c3)nc(-c3cccc4c(-c5cccc6oc7ccccc7c56)cccc34)n2)cc1]

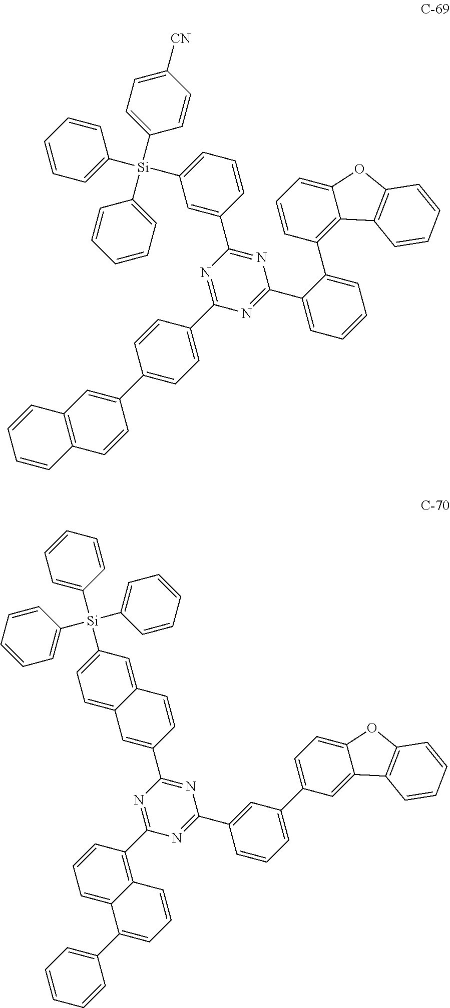 [SMILES: N#Cc1ccc([Si](c2ccccc2)(c2ccccc2)c2cccc(-c3nc(-c4ccc(-c5ccc6ccccc6c5)cc4)nc(-c4ccccc4-c4cccc5oc6ccccc6c45)n3)c2)cc1 ; c1ccc(-c2cccc3c(-c4nc(-c5cccc(-c6ccc7oc8ccccc8c7c6)c5)nc(-c5ccc6cc([Si](c7ccccc7)(c7ccccc7)c7ccccc7)ccc6c5)n4)cccc23)cc1]

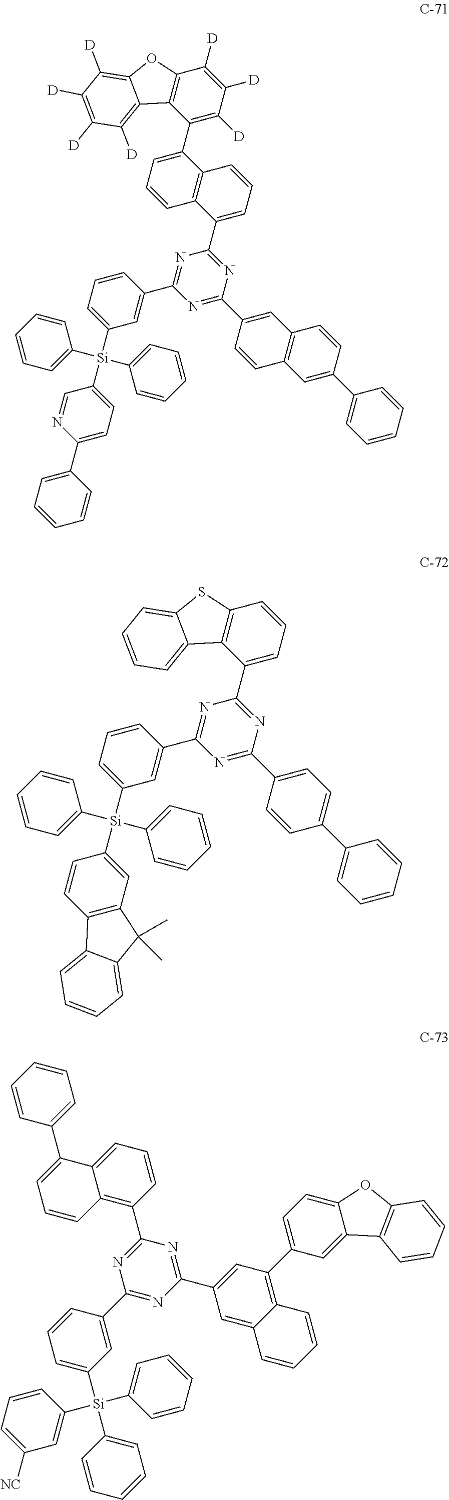 [SMILES: [2H]c1c([2H])c([2H])c2c(oc3c([2H])c([2H])c([2H])c(-c4cccc5c(-c6nc(-c7cccc([Si](c8ccccc8)(c8ccccc8)c8ccc(-c9ccccc9)nc8)c7)nc(-c7ccc8cc(-c9ccccc9)ccc8c7)n6)cccc45)c32)c1[2H] ; CC1(C)c2ccccc2-c2ccc([Si](c3ccccc3)(c3ccccc3)c3cccc(-c4nc(-c5ccc(-c6ccccc6)cc5)nc(-c5cccc6sc7ccccc7c56)n4)c3)cc21 ; N#Cc1cccc([Si](c2ccccc2)(c2ccccc2)c2cccc(-c3nc(-c4cc(-c5ccc6oc7ccccc7c6c5)c5ccccc5c4)nc(-c4cccc5c(-c6ccccc6)cccc45)n3)c2)c1]

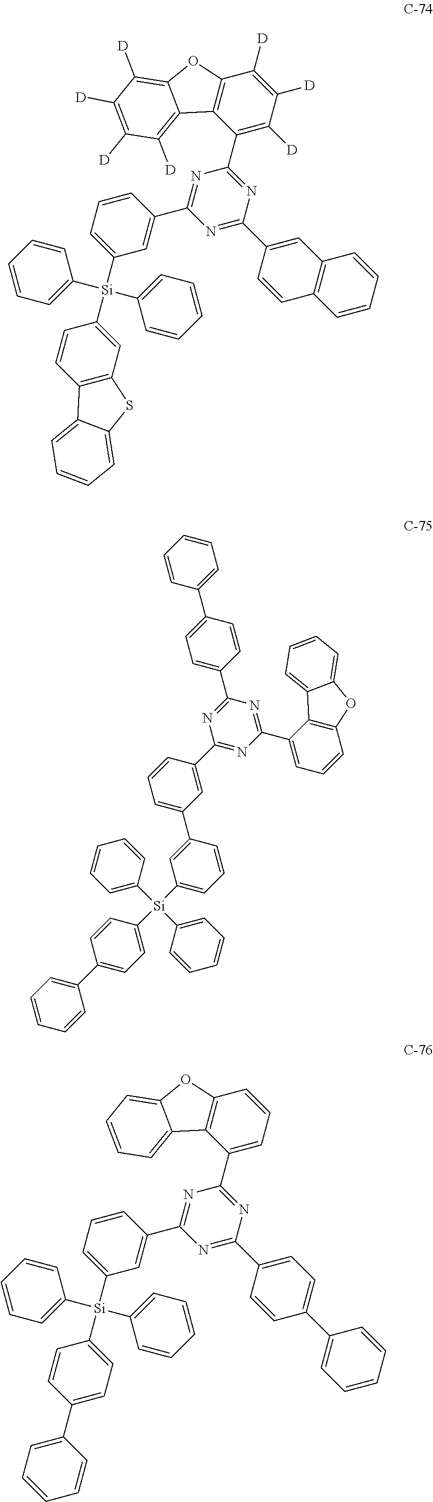 [SMILES: [2H]c1c([2H])c([2H])c2c(oc3c([2H])c([2H])c([2H])c(-c4nc(-c5cccc([Si](c6ccccc6)(c6ccccc6)c6ccc7c(c6)sc6ccccc67)c5)nc(-c5ccc6ccccc6c5)n4)c32)c1[2H] ; c1ccc(-c2ccc(-c3nc(-c4cccc(-c5cccc([Si](c6ccccc6)(c6ccccc6)c6ccc(-c7ccccc7)cc6)c5)c4)nc(-c4cccc5oc6ccccc6c45)n3)cc2)cc1 ; c1ccc(-c2ccc(-c3nc(-c4cccc([Si](c5ccccc5)(c5ccccc5)c5ccc(-c6ccccc6)cc5)c4)nc(-c4cccc5oc6ccccc6c45)n3)cc2)cc1]

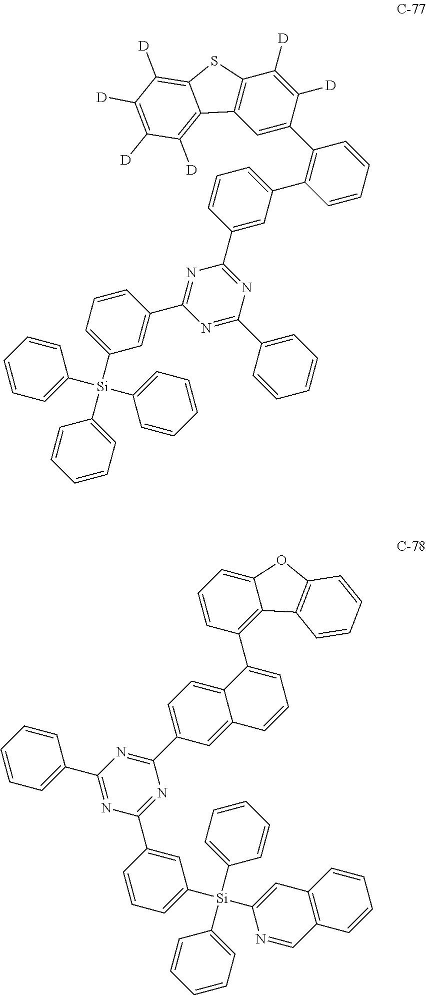 [SMILES: [2H]c1c(-c2ccccc2-c2cccc(-c3nc(-c4ccccc4)nc(-c4cccc([Si](c5ccccc5)(c5ccccc5)c5ccccc5)c4)n3)c2)cc2c(sc3c([2H])c([2H])c([2H])c([2H])c32)c1[2H] ; c1ccc(-c2nc(-c3cccc([Si](c4ccccc4)(c4ccccc4)c4cc5ccccc5cn4)c3)nc(-c3ccc4c(-c5cccc6oc7ccccc7c56)cccc4c3)n2)cc1]

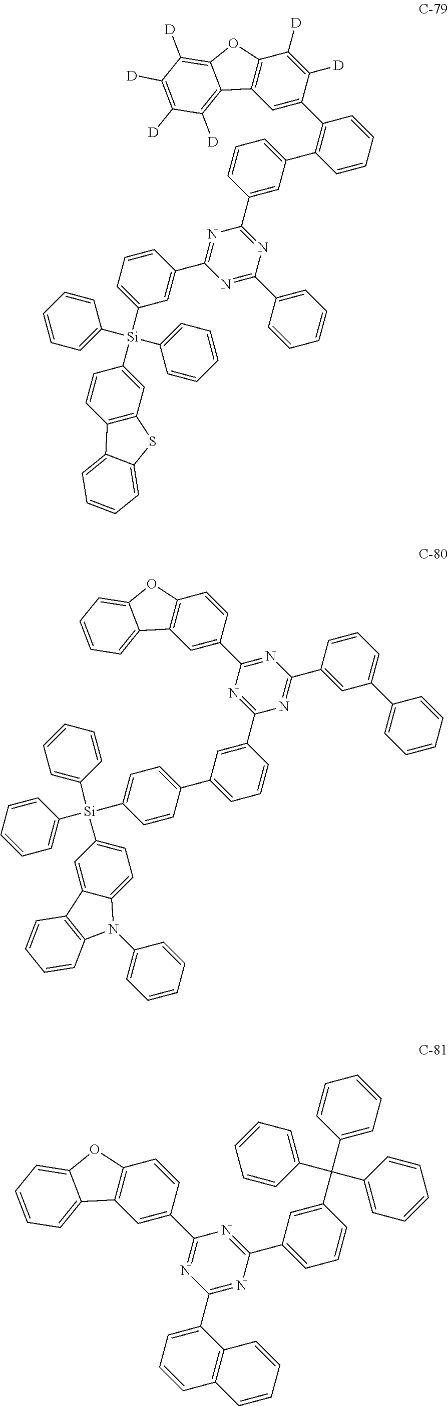 [SMILES: [2H]c1c(-c2ccccc2-c2cccc(-c3nc(-c4ccccc4)nc(-c4cccc([Si](c5ccccc5)(c5ccccc5)c5ccc6c(c5)sc5ccccc56)c4)n3)c2)cc2c(oc3c([2H])c([2H])c([2H])c([2H])c32)c1[2H] ; c1ccc(-c2cccc(-c3nc(-c4cccc(-c5ccc([Si](c6ccccc6)(c6ccccc6)c6ccc7c(c6)c6ccccc6n7-c6ccccc6)cc5)c4)nc(-c4ccc5oc6ccccc6c5c4)n3)c2)cc1 ; c1ccc(C(c2ccccc2)(c2ccccc2)c2cccc(-c3nc(-c4ccc5oc6ccccc6c5c4)nc(-c4cccc5ccccc45)n3)c2)cc1]

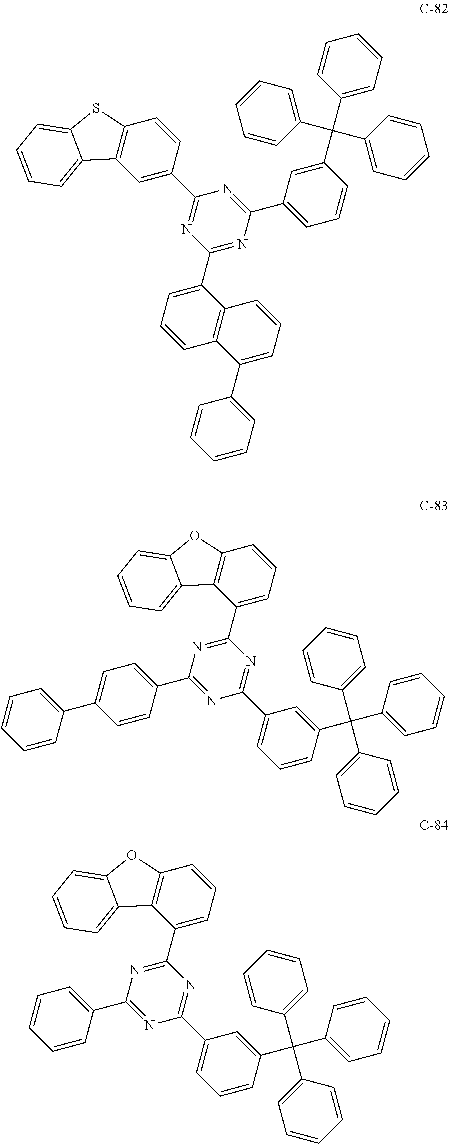 [SMILES: c1ccc(-c2cccc3c(-c4nc(-c5cccc(C(c6ccccc6)(c6ccccc6)c6ccccc6)c5)nc(-c5ccc6sc7ccccc7c6c5)n4)cccc23)cc1 ; c1ccc(-c2ccc(-c3nc(-c4cccc(C(c5ccccc5)(c5ccccc5)c5ccccc5)c4)nc(-c4cccc5oc6ccccc6c45)n3)cc2)cc1 ; c1ccc(-c2nc(-c3cccc(C(c4ccccc4)(c4ccccc4)c4ccccc4)c3)nc(-c3cccc4oc5ccccc5c34)n2)cc1]

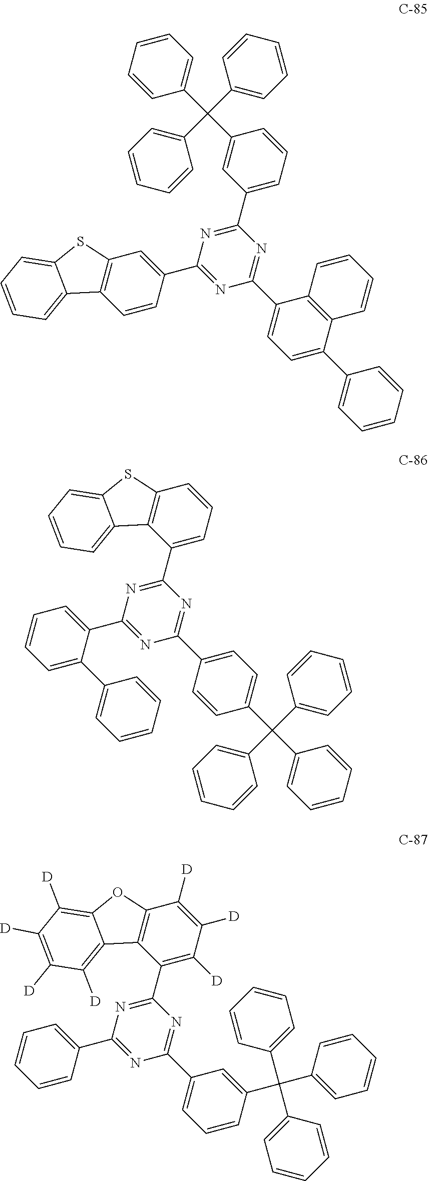 [SMILES: c1ccc(-c2ccc(-c3nc(-c4cccc(C(c5ccccc5)(c5ccccc5)c5ccccc5)c4)nc(-c4ccc5c(c4)sc4ccccc45)n3)c3ccccc23)cc1 ; c1ccc(-c2ccccc2-c2nc(-c3ccc(C(c4ccccc4)(c4ccccc4)c4ccccc4)cc3)nc(-c3cccc4sc5ccccc5c34)n2)cc1 ; [2H]c1c([2H])c([2H])c2c(oc3c([2H])c([2H])c([2H])c(-c4nc(-c5ccccc5)nc(-c5cccc(C(c6ccccc6)(c6ccccc6)c6ccccc6)c5)n4)c32)c1[2H]]

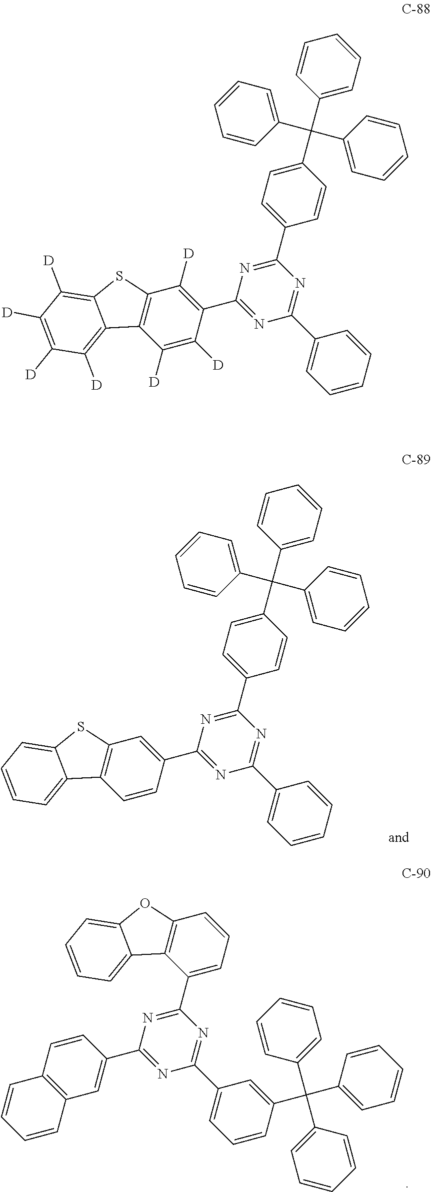 [SMILES: [2H]c1c([2H])c([2H])c2c(sc3c([2H])c(-c4nc(-c5ccccc5)nc(-c5ccc(C(c6ccccc6)(c6ccccc6)c6ccccc6)cc5)n4)c([2H])c([2H])c32)c1[2H] ; c1ccc(-c2nc(-c3ccc(C(c4ccccc4)(c4ccccc4)c4ccccc4)cc3)nc(-c3ccc4c(c3)sc3ccccc34)n2)cc1 ; c1ccc(C(c2ccccc2)(c2ccccc2)c2cccc(-c3nc(-c4ccc5ccccc5c4)nc(-c4cccc5oc6ccccc6c45)n3)c2)cc1]

12. An organic electroluminescent material comprising an organic electroluminescent compound according to claim 10.

13. An organic electroluminescent device comprising an organic electroluminescent material according to claim 12.
